SEQUENCE LISTING (260 sequences)

SEQ ID NO: 1 - protein, 300 aa, synthetic construct
MKRVITLFAVLLMGWSVNAWSFACKTANGTAIPIGGGSANVYVNLAPAVNVGQNLVVDLSTQIFCHNDYPETITDYVTLQRGSAYGGVLSSFSGTVKYNGSSYPFPTTSETPRVVYNSRTDKPWPVALYLTPVSSAGGVAIKAGSLIAVLILRQTNNYNSDDFQFVWNIYANNDVVVPTGGCDASARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQ

SEQ ID NO: 2 - protein, 301 aa, synthetic construct
MKKIIPLFTTLLLLGWSMNAWSFACKTATGATIPIGGGSANVYVNLTPAVNVGQNLVVDLSTQIFCHNDYPETITDYVTLQRGSAYGGVLSSFSGTVKYNGTSYPFPTTTETARVIYDSRTDKPWPAVLYLTPVSTAGGVAITAGSLIAVLILHQTNNYNSDSFQFIWNIYANNDVVVPTGGCDVSARDVTVTLPDYPGSMAVPLTVHCAQSQQLGYYLSGTTADSANAIFTNTASASPAQGIGVQLTRNGSAVPANSTVSLGTVGTSPVNLGLTATYARTTGQVTAGNVQSIIGITFVYQ

SEQ ID NO: 3 - protein, 152 aa, synthetic construct
MKLLKVAAIAAIVFSGSALAGVVPQYGGGGGNHGGGGNNSGPNSELNIYQYGGGNSALAQQADARNSDLTITQHGGGNGADVGQGSDDSSIDLTQRGFGNSATLDQWNGKDSTMTVKQFGGGNGAAVDQTASNSSVNVTQVGFGNNATAHQY

SEQ ID NO: 4 - protein, 353 aa, synthetic construct
MHLRHLFSSRLRGSLLLGSLLVASSFSTQAAEEMLRKAVGKGAYEMAYSQQENALWLATSQSRKLDKGGVVYRLDPVTLEVTQAIHNDLKPFGATINNTTQTLWFGNTVNSAVTAIDAKTGEVKGRLVLDDRKRTEEVRPLQPRELVADDATNTVYISGIGKESVIWVVDGENIKLKTAIQNTGKMSTGLALDSKGKRLYTTNADGELITIDTADNKILSRKKLLDDGKEHFFINISLDTTRQRAFITDSKAAEVLVVDTRNGNILAKVAAPESLAVLFNPARNEAYVTHRQAGKVSVIDAKSYKVVKTFDTPTHPNSLALSADGKTLYVSVKQKSTKQQEATQPDDVIRIAL

SEQ ID NO: 5 - protein, 353 aa, synthetic construct
MSLRHFAAPRLRHSLLVTSLLLAGSFSAHAAEEMLRKAVGKGAYEMAYSQQENALWVATSQSRSLDKGGVVYRLDPTTLDVTQIIHNDLKPFGATINHATGTLWFGNTVDSTVTAIDAKTGAVKGRLVLDERQRSETVRPLQPRELAVNEQTNTVYITGLGKESVIWVVDGATLKLKTTITGTGAMATGLAIDPQAKRLYTTNADGELLTIDSESNTIASRKKLQDDGKAHFYLNLSLDTAGHRAFITDSKQPEVLVVDTRDGKVLEKIAAPESLAVLFNPARNEAYVTHRKAGEVSVIDGKSYKVVKTFKTPTHPNSLALSEDGKTLYVSVKQASSREKEATAPDDVIRIAL

SEQ ID NO: 6 - protein, 336 aa, synthetic construct
MKKWFPALLFSLCVSGESSAWNNIVFYSLGNVNSYQGGNVVITQRPQFITSWRPGIATVTWNQCNGPEFADGSWAYYREYIAWVVFPKKVMTKNGYPLFIEVHNKGSWSEENTGDNDSYFFLKGYKWDERAFDAGNLCQKPGETTRLTEKFNDIIFKVALPADLPLGDYSVTIPYTSGIQRHFASYLGARFKIPYNVAKTLPRENEMLFLFKNIGGCRPSAQSLEIKHGDLSINSANNHYAAQTLSVSCDVPANIRFMLLRNTTPTYSHGKKFSVGLGHGWDSIVSVNGVDTGETTMRWYKAGTQNLTIGSRLYGESSKIQPGVLSGSATLLMILP

SEQ ID NO: 7 - protein, 335 aa, synthetic construct
MKKWLPAFLFLSLSGCNDALAANQSTMFYSFNDNIYRPQLSVKVTDIVQFIVDINSASSTATLSYVACNGFTWIHGLYWSEYFAWLVVPKHVSYNGYNIYLELQSRGSFSLDAEDNDNYYLTKGFAWDEVNTSGQTCFNIGEKRSLAWSFGGVTLNARLPVDLPKGDYTFPVKFLRGIQRNNYDYIGGRYKIPSSLMKTFPFNGTLNFSIKNTGGCRPSAQSLEINHGDLSINSANNHYAAQTLSVSCDVPTNIRFFLLSNTNPAYSHGQQFSVGLGHGWDSIISINGVDTGETTMRWYRAGTQNLTIGSRLYGESSKIQPGVLSGSATLLMILP

SEQ ID NO: 8 - protein, 1416 aa, synthetic construct
MSRYKTDNKQPRFRYSVLARCVAWANISVQVLFPLAVTFTPVMAARAQHAVQPRLSMENTTVTADNNVEKNVASLAANAGTFLSSQPDSDATRNFITGMATAKANQEIQEWLGKYGTARVKLNVDKKFSLKDSSLEMLYPIYDTPTNMLFTQGAIHRTDDRTQSNIGFGWRHFSENDWMAGVNTFIDHDLSRSHTRIGVGAEYWRDYLKLSANGYIRASGWKKSPDVEDYQERPANGWDIRAEGYLPAWPQLGASLMYEQYYGDEVGLFGKDKRQKDPHAITAEVNYTPVPLLTLSAGHKQGKSGENDTRFGLEVNYRIGEPLEKQLDTDSIRERRMLAGSRYDLVERNNNIVLEYRKSEVIRIALPERIEGKGGQTVSLGLVVSKATHGLKNVQWEAPSLLAAGGKITGQGNQWQVTLPAYQAGKDNYYAISAIAYDNKGNASKRVQTEVVISGAGMSADRTALTLDGQSRIQMLANGNEQKPLVLSLRDAEGQPVTGMKDQIKTELTFKPAGNIVTRTLKATKSQAKPTLGEFTETEAGVYQSVFTTGTQSGEATITVSVDDMSKTVTAELRATMMDVANSTLSANEPSGDVVADGQQAYTLTLTAVDSEGNPVTGEASRLRLVPQDTNGVTVGAISEIKPGVYSATVSSTRAGNVVVRAFSEQYQLGTLQQTLKFVAGPLDAAHSSITLNPDKPVVGGTVTAIWTAKDANDNPVTGLNPDAPSLSGAAAAGSTASGWTDNGDGTWTAQISLGTTAGELDVMPKLNGQDAAANAAKVTVVADALSSNQSKVSVAEDHVKAGESTTVTLVAKDAHGNAISGLSLSASLTGTASEGATVSSWTEKGDGSYVATLTTGGKTGELRVMPLFNGQPAATEAAQLTVIAGEMSSANSTLVADNKTPTVKTTTELTFTVKDAYGNPVTGLKPDAPVFSGAASTGSERPSAGNWTEKGNGVYVSTLTLGSAAGQLSVMPRVNGQNAVAQPLVLNVAGDASKAEIRDMTVKVNNQLANGQSANQITLTVVDSYGNPLQGQEVTLTLPQGVTSKTGNTVTTNAAGKVDIELMSTVAGELEIEASVKNSQKTVKVKFKADFSTGQASLEVDAAAQKVANGKDAFTLTATVKDQYGNLLPGAVVVFNLPRGVKPLADGNIMVNADKEGKAELKVVSVTAGTYEITASAGNDQPSNAQSVTFVADKTTATISSIEVIGNRAVADGKTKQTYKVTVTDANNNLLKDSEVTLTASPENLVLTPNGTATTNEQGQAIFTATTTVAATYTLTAKVEQADGQESTKTAESKFVADDKNAVLAASPERVDSLVADGKTTATLTVTLMSGVNPVGGTMWVDIEAPEGVTEADYQFLPSKNDHFASGKITRTFSTNKPGTYTFTFNSLTYGGYEMKPVTVTINAVPADTEGAEEK

SEQ ID NO: 9 - protein, 275 aa, synthetic construct
MFIFKRFPAIFILIAGLFSAPAMASIFSYITESTGTPSNATYTYVIERWDPETSGILNPCYGWPVCYVTVNHKHTVNGTGGNPAFQIARIEKLRTLAEVRDVVLKNRSFPIEGQTTHRGPSLNSNQECVGLFYQPNSSGISPRGKLLPGSLCGIAPPPVGACKISEGAVNLNYGDIDEASLSGAKRSETINVTCNLAMKVLVIASGSDSGRVPLRADKSLYADLYLNNYPGEKGVTVNVPAGGSAPVSVSSTLRTNGRVAPGRFSGSGSIILTMP

SEQ ID NO: 10 - protein, 731 aa, synthetic construct
MRKKYMPRALGPLLLVVLSPAVAQQNDDNEIIVSASRSNRTVAEMAQTTWVIENAELEQQIQGGKELKDALAQLIPGLDVSSQSRTNYGMNMRGRPLVVLIDGVRLNSSRSDSRQLDSVDPFNIDHIEVISGATALYGGGSTGGLINIVTKKGQPETMMEFEAGTKSGFNSSKDHDERIAGAVSGGNDHISGRLSVAYQKFGGWFDGNGDATLLDNTQTGLQHSNRLDIMGTGTLNIDESRQLQLITQYYKSQGDDNYGLNLGKGFSAISGSSTPYVSKGLNSDRIPGTERHLISLQYSDSDFLRQELVGQVYYRDESLRFYPFPTVNANKQATAFSSSQQDTDQYGMKLTLNSQLMDGWQITWGLDAEHERFTSNQMFFDLAQASASGGLNNHKIYTTGRYPSYDITNLAAFLQSSYDINDIFTVSGGVRYQYTENRVDDFIDYTQQQKIAAGKAISADAIPGGSVDYDNFLFNAGLLMHITERQQAWFNFSQGVALPDPGKYYGRGIYGAAVNGHLPLTKSVNVSDSKLEGVKVDSYELGWRFTGDNLRTQIAAYYSLSNKSVERNKDLTISVKDDRRRIYGVEGAVDYLIPDTDWSTGVNFNVLKTESKVNGQWQKYDVKESSPSKATAYINWAPEPWSLRVQSTTSFDVSDAEGNDINGYTTVDFISSWQLPVGTLSFSVENLFDRDYTTVWGQRAPLYYSPGYGPASLYDYKGRGRTFGLNYSVLF

SEQ ID NO: 11 - protein, 729 aa, synthetic construct
MKKRLWVLHPLLLASTLPALAAQSDEDSIIVSANRTHRTVAEMAQTTWVIEGQEIEQQVQGGKEFKDVLAQLIPGIDVSSQGRTNYGMNMRGRAIVVLIDGVRLNSSRTDSRQLDAIDPFNIEHIEVISGATSLYGGGSTGGLINIVTKKGQQDRQVDLEVGSKSGFANSNDHDERVAAAVSGGTDHASGRLSVAYQRFGGWYDGNNDALILDNTQTGLQHSDRLDVMGTGTIEIDDNRQLQLVTQYYKSQGDDDYGLWLGKNMSAVTSGGKAYTTDGLNSDRIPGTERHLISLQYSDADFFGQNLVSQVYYRDESLTFYPFPTLTKGQVSSFSSSQQDTDQYGAKLTLNSQPLAGWDLTWGLDADHETFNANQMFFDLPQSMASGGLHNESIYTTGRYPGYSISNVAPFLQSSYDLNDIFTVSGGVRYQWTENRVDDFVGYAQQQDIANGKARSADAIKGGKTDYDNFLFNAGIVAHLTERQQTWFNFSQGVELPDPGKYYGIGKYGAAVNGHLPLISSVNVDDSPLQGIKVNSYELGWRYTGDNLRTQLAAYYSTSDKTIVVNRTDMTIDVQSDKRRIYGVEGAVDYFIPDSDWSVGGNFNVLKSQVQTDGRWQKWDVTLASPSKATAWVGWAPDPWSLRVQSQQVFDLSDAAGNKLEGYNTVDFIGSYALPVGKLTFSIENLLNEDYVTIWGQRAPLLYSPTYGSSSLYEYKGRGRTFGLNYALTF

SEQ ID NO: 12 - protein, 725 aa, synthetic construct
MRINKILWSLTVLLVGLNSQVSVAKYSDDDNDETLVVEATAEQVLKQQPGVSVITSEDIKKTPPVNDLSDIIRKMPGVNLTGNSASGTRGNNRQIDIRGMGPENTLILIDGVPVTSRNSVRYSWRGERDTRGDTNWVPPEQVERIEVIRGPAAARYGSGAAGGVVNIITKRPTNDWHGSLSLYTNQPESSEEGATRRANFSLSGPLAGDALTTRLYGNLNKTDADSWDINSPVGTKNAAGHEGVRNKDINGVVSWKLNPQQILDFEVGYSRQGNIYAGDTQNSSSSAVTESLAKSGKETNRLYRQNYGITHNGIWDWGQSRFGVYYEKTNNTRMNEGLSGGGEGRILAGEKFTTNRLSSWRTSGELNIPLNVMVDQTLTVGAEWNRDKLDDPSSTSLTVNDRDISGISGSAADRSSKNHSQISALYIEDNIEPVPGTNIIPGLRFDYLSDSGGNFSPSLNLSQELGDYFKVKAGVARTFKAPNLYQSSEGYLLYSKGNGCPKDITSGGCYLIGNKDLDPEISVNKEIGLEFTWEDYHASVTYFRNDYQNKIVAGDNVIGQTASGAYILKWQNGGKALVDGIEASMSFPLVKERLNWNTNATWMITSEQKDTGNPLSVIPKYTINNSLNWTITQAFSASFNWTLYGRQKPRTHAETRSEDTGGLSGKELGAYSLVGTNFNYDINKNLRLNVGVSNILNKQIFRSSEGANTYNEPGRAYYAGVTASF

SEQ ID NO: 13 - protein, 727 aa, synthetic construct
MRSPQYSVPTTLALVISSLLAPNAFAEESEQDTETMIVRSTAEEALKQQPGVSIITAEDIAKQSPVNDLSDIIRKMPGVNLTGNSASGSRGNNRQIDIRGMGPENTLILIDGVPVTSRNAVRYSWRGERDTRGDSNWVPAEMVERIEVLRGPAAARYGSGAAGGVVNIITKRPTNTWHGSLSFFTNQPENNKEGTTNRANFNLSGPLAGEALTMRLYGNINKTEPDAWDINHAQNGSYAAGREGVRNKDINTLLSWKMTPQQILDFSYAYSRQGNIYAGDTQYSNGNLSPSGLVDSLYGHETNRLYRQSWGLTYNGLWDWGQSKAGVYYEKTNNTRLQEGSTGRVEGMINSEDYATSRLESWRTTSELNVPFFWLADQTLTLGMEWNHDQLDDPASMQATNSNGETIPGTSGDPTQRSTKNSATLTGIYLEDNIEAVPGTNLIPGIRFDYHNQFGSNWSPSLNLSQELGDMFTLKAGIARVFKAPNLYQSSKGYLLSTRGNGCPNTIAEGSCYLLGNPDLDPEISINKEIGIEFNLNGYAAGVTWFRNDYKNKIVSGTEVLGYTSSGNNILQWQNGGKAVVEGLEGNLLIPVLRDVLSWRTNATWMLKSESKETGNPLSVIPKYTVNTMLDWQVNDALSANVNWTLYGRQKPRQYAEIRNETGTLATTEVGAYSIVGIGTQYQLNRDIRLNAGISNLFDKQLYRENAGASTYNEPGRAYYAGVTLSF

SEQ ID NO: 14 - protein, 660 aa, synthetic construct
MSRPQFTSLRLSLLALAVSATLPTFAFATETMTVTATGNARSSFEAPMMVSVIDTSAPENQTATSATDLLRHVPGITLDGTGRTNGQDVNMRGYDHRGVLVLVDGVRQGTDTGHLNGTFLDPALIKRVEIVRGPSALLYGSGALGGVISYDTVDAKDLLQEGQSSGFRVFGTGGTGDHSLGLGASAFGRTENLDGIVAWSSRDRGDLRQSNGETAPNDESINNMLAKGTWQIDSAQSLSGLVRYYNNDAREPKNPQTVEASDSSNPMVDRSTIQRDAQLSYKLAPQGNDWLNADAKIYWSEVRINAQNTGSSGEYREQITKGARLENRSTLFADSFASHLLTYGGEYYRQEQHPGGATTGFPQAKIDFSSGWLQDEITLRDLPITLLGGTRYDSYRGSSDGYKDVDADKWSSRAGMTINPTNWLMLFGSYAQAFRAPTMGEMYNDSKHFSIGRFYTNYWVPNPNLRPETNETQEYGFGLRFDDLMLSNDALEFKASYFDTKAKDYISTTVDFAAATTMSYNVPNAKIWGWDVMTKYTTDLFSLDVAYNRTRGKDTDTGEYISSINPDTVTSTLNIPIAHSGFSVGWVGTFADRSTHISSSYSKQPGYGVNDFYVSYQGQQALKGMTTTLVLGNAFDKEYWSPQGIPQDGRNGKIFVSYQW

SEQ ID NO: 15 - protein, 673 aa, synthetic construct
MKMTRLYPLALGGLLLPAIANAQTSQQDESTLVVTASKQSSRSASANNVSSTVVSAPELSDAGVTASDKLPRVLPGLNIENSGNMLFSTISLRGVSSAQDFYNPAVTLYVDGVPQLSTNTIQALTDVQSVELLRGPQGTLYGKSAQGGIINIVTQQPDSTPRGYIEGGVSSRDSYRSKFNLSGPIQDGLLYGSVTLLRQVDDGDMINPATGSDDLGGTRASIGNVKLRLAPDDQPWEMGFAASRECTRATQDAYVGWNDIKGRKLSISDGSPDPYMRRCTDSQTLSGKYTTDDWVFNLISAWQQQHYSRTFPSGSLIVNMPQRWNQDVQELRAATLGDARTVDMVFGLYRQNTREKLNSAYDMPTMPYLSSTGYTTAETLAAYSDLTWHLTDRFDIGGGVRFSHDKSSTQYHGSMLGNPFGDQGKSNDDQVLGQLSAGYMLTDDWRVYTRVAQGYKPSGYNIVPTAGLDAKPFVAEKSINYELGTRYETADVTLQAATFYTHTKDMQLYSGPVGMQTLSNAGKADATGVELEAKWRFAPGWSWDINGNVIRSEFTNDSELYHGNRVPFVPRYGAGSSVNGVIDTRYGALMPRLAVNLVGPHYFDGDNQLRQGTYATLDSSLGWQATERMNISVYVDNLFDRRYRTYGYMNGSSAVAQVNMGRTVGINTRIDFF

SEQ ID NO: 16 - protein, 10 aa, synthetic construct
NGTAIPIGGG

SEQ ID NO: 17 - protein, 10 aa, synthetic construct
HNDYPETITD

SEQ ID NO: 18 - protein, 22 aa, synthetic construct
SYPFPTTSETPRVVYNSRTDKP

SEQ ID NO: 19 - protein, 37 aa, synthetic construct
SALALQADARNSDLTITQHGGGNGADVGQGSDDSSID

SEQ ID NO: 20 - protein, 13 aa, synthetic construct
ATLDQWNGKDSTM

SEQ ID NO: 21 - protein, 12 aa, synthetic construct
NGAAVDQTASNS

SEQ ID NO: 22 - protein, 20 aa, synthetic construct
SQQENALWLATSQSRKLDKG

SEQ ID NO: 23 - protein, 21 aa, synthetic construct
GEVKGRLVLDDRKRTEEVRPL

SEQ ID NO: 24 - protein, 7 aa, synthetic construct
NKILSRK

SEQ ID NO: 25 - protein, 14 aa, synthetic construct
VIDAKSYKVVKTFD

SEQ ID NO: 26 - protein, 14 aa, synthetic construct
KQKSTKQQEATQPD

SEQ ID NO: 27 - protein, 16 aa, synthetic construct
WNQCNGPGFADGSWAY

SEQ ID NO: 28 - protein, 12 aa, synthetic construct
KGSWSEENTGDN

SEQ ID NO: 29 - protein, 18 aa, synthetic construct
AGNLCQKPGETTRLTEKF

SEQ ID NO: 30 - protein, 11 aa, synthetic construct
DLPLGDYSVTI

SEQ ID NO: 31 - protein, 15 aa, synthetic construct
GARFKIPYNVAKTLP

SEQ ID NO: 32 - protein, 23 aa, synthetic construct
IVSVNGVDTGETTMRWYKAGTQN

SEQ ID NO: 33 - protein, 21 aa, synthetic construct
IAYDNKGNASKRVQTEVVISG

SEQ ID NO: 34 - protein, 27 aa, synthetic construct
PTLGEFTETEAGVYQSVFTTGTQSGEA

SEQ ID NO: 35 - protein, 42 aa, synthetic construct
WTAKDANDNPVTGLNPDAPSLSGAAAAGSTASGWTDNGDGTW

SEQ ID NO: 36 - protein, 33 aa, synthetic construct
SWTEKGDGSYVATLTTGGKTGELRVMPLFNGQP

SEQ ID NO: 37 - protein, 34 aa, synthetic construct
AAQLTVIAGEMSSANSTLVADNKTPTVKTTTELT

SEQ ID NO: 38 - protein, 20 aa, synthetic construct
SGAASTGSERPSAGNWTEKG

SEQ ID NO: 39 - protein, 22 aa, synthetic construct
ESTGTPSNATYTYVIERWDPET

SEQ ID NO: 40 - protein, 14 aa, synthetic construct
PIEGQTTHRGPSLN

SEQ ID NO: 41 - protein, 20 aa, synthetic construct
PGSLCGIAPPPVGACKISEG

SEQ ID NO: 42 - protein, 14 aa, synthetic construct
QTTWVIENAELEQQ

SEQ ID NO: 43 - protein, 12 aa, synthetic construct
DPFNIDHIEVIS

SEQ ID NO: 44 - protein, 14 aa, synthetic construct
GGLINIVTKKGQPE

SEQ ID NO: 45 - protein, 12 aa, synthetic construct
TGTLNIDESRQL

SEQ ID NO: 46 - protein, 15 aa, synthetic construct
DESLRFYPFPTVNAN

SEQ ID NO: 47 - protein, 13 aa, synthetic construct
LNFSQGVELPDPG

SEQ ID NO: 48 - protein, 13 aa, synthetic construct
IAAYYSISDKSVV

SEQ ID NO: 49 - protein, 15 aa, synthetic construct
DETLVVEATAEQVLK

SEQ ID NO: 50 - protein, 17 aa, synthetic construct
GPENTLILIDGVPVTSR

SEQ ID NO: 51 - protein, 12 aa, synthetic construct
NDYQNKIVAGDN

SEQ ID NO: 52 - protein, 14 aa, synthetic construct
VDGIEASMSFPLVK

SEQ ID NO: 53 - protein, 14 aa, synthetic construct
INNSLNWTITQAFS

SEQ ID NO: 54 - protein, 15 aa, synthetic construct
NLRLNVGVSNILNKQ

SEQ ID NO: 55 - protein, 14 aa, synthetic construct
APMMVSVIDTSAPE

SEQ ID NO: 56 - protein, 12 aa, synthetic construct
KGTWQIDSAQSL

SEQ ID NO: 57 - protein, 14 aa, synthetic construct
DAKIYWSEVRINAQ

SEQ ID NO: 58 - protein, 14 aa, synthetic construct
NPTNWLMLFGSYAQ

SEQ ID NO: 59 - protein, 16 aa, synthetic construct
VPNAKIWGWDVMTKYT

SEQ ID NO: 60 - protein, 14 aa, synthetic construct
ISSSYSKQPGYGVN

SEQ ID NO: 61 - protein, 13 aa, synthetic construct
DFYVSYQGQQALK

SEQ ID NO: 62 - protein, 13 aa, synthetic construct
GNMLFSTISLRGV

SEQ ID NO: 63 - protein, 14 aa, synthetic construct
DGVPQLSTNTIQAL

SEQ ID NO: 64 - protein, 15 aa, synthetic construct
DVQSVELLRGPQGTL

SEQ ID NO: 65 - protein, 13 aa, synthetic construct
INIVTQQPDSTPR

SEQ ID NO: 66 - protein, 13 aa, synthetic construct
GYTTAETLAAYSD

SEQ ID NO: 67 - protein, 16 aa, synthetic construct
HTKDMQLYSGPVGMQT

SEQ ID NO: 68 - protein, 13 aa, synthetic construct
GNVIRSEFTNDSE

SEQ ID NO: 69 - protein, 16 aa, synthetic construct
NDSELYHGNRVPFVPR

SEQ ID NO: 70 - protein, 14 aa, synthetic construct
QGTYATLDSSLGWQ

SEQ ID NO: 71 - protein, 15 aa, synthetic construct
RVLPGLNIENSGNML

SEQ ID NO: 72 - protein, 15 aa, synthetic construct
IENSGNMLFSTISLR

SEQ ID NO: 73 - protein, 15 aa, synthetic construct
LFSTISLRGVSSAQD

SEQ ID NO: 74 - protein, 15 aa, synthetic construct
RGVSSAQDFYNPAVT

SEQ ID NO: 75 - protein, 15 aa, synthetic construct
DFYNPAVTLYVDGVP

SEQ ID NO: 76 - protein, 15 aa, synthetic construct
TLYVDGVPQLSTNTI

SEQ ID NO: 77 - protein, 15 aa, synthetic construct
PQLSTNTIQALTDVQ

SEQ ID NO: 78 - protein, 15 aa, synthetic construct
IQALTDVQSVELLRG

SEQ ID NO: 79 - protein, 15 aa, synthetic construct
QSVELLRGPQGTLYG

SEQ ID NO: 80 - protein, 15 aa, synthetic construct
GPQGTLYGKSAQGGI

SEQ ID NO: 81 - protein, 15 aa, synthetic construct
GKSAQGGIINIVTQQ

SEQ ID NO: 82 - protein, 15 aa, synthetic construct
IINIVTQQPDSTPRG

SEQ ID NO: 83 - protein, 15 aa, synthetic construct
SSRDSYRSKFNLSGP

SEQ ID NO: 84 - protein, 15 aa, synthetic construct
SKFNLSGPIQDGLLY

SEQ ID NO: 85 - protein, 15 aa, synthetic construct
PIQDGLLYGSVTLLR

SEQ ID NO: 86 - protein, 15 aa, synthetic construct
YGSVTLLRQVDDGDM

SEQ ID NO: 87 - protein, 15 aa, synthetic construct
YTTDDWVFNLISAWQ

SEQ ID NO: 88 - protein, 15 aa, synthetic construct
FNLISAWQQQHYSRT

SEQ ID NO: 89 - protein, 15 aa, synthetic construct
QQQHYSRTFPSGSLI

SEQ ID NO: 90 - protein, 15 aa, synthetic construct
TFPSGSLIVNMPQRW

SEQ ID NO: 91 - protein, 15 aa, synthetic construct
MPYLSSTGYTTAETL

SEQ ID NO: 92 - protein, 15 aa, synthetic construct
GYTTAETLAAYSDLT

SEQ ID NO: 93 - protein, 15 aa, synthetic construct
LAAYSDLTWHLTDRF

SEQ ID NO: 94 - protein, 15 aa, synthetic construct
RFSHDKSSTQYHGSM

SEQ ID NO: 95 - protein, 15 aa, synthetic construct
DQVLGQLSAGYMLTD

SEQ ID NO: 96 - protein, 15 aa, synthetic construct
SAGYMLTDDWRVYTR

SEQ ID NO: 97 - protein, 15 aa, synthetic construct
RVAQGYKPSGYNIVP

SEQ ID NO: 98 - protein, 15 aa, synthetic construct
PSGYNIVPTAGLDAK

SEQ ID NO: 99 - protein, 15 aa, synthetic construct
PTAGLDAKPFVAEKS

SEQ ID NO: 100 - protein, 15 aa, synthetic construct
KPFVAEKSINYELGT

SEQ ID NO: 101 - protein, 15 aa, synthetic construct
SINYELGTRYETADV

SEQ ID NO: 102 - protein, 15 aa, synthetic construct
TRYETADVTLQAATF

SEQ ID NO: 103 - protein, 15 aa, synthetic construct
VTLQAATFYTHTKDM

SEQ ID NO: 104 - protein, 15 aa, synthetic construct
FYTHTKDMQLYSGPV

SEQ ID NO: 105 - protein, 15 aa, synthetic construct
MQLYSGPVGMQTLSN

SEQ ID NO: 106 - protein, 15 aa, synthetic construct
VGMQTLSNAGKADAT

SEQ ID NO: 107 - protein, 15 aa, synthetic construct
NAGKADATGVELEAK

SEQ ID NO: 108 - protein, 15 aa, synthetic construct
TGVELEAKWRFAPGW

SEQ ID NO: 109 - protein, 15 aa, synthetic construct
KWRFAPGWSWDINGN

SEQ ID NO: 110 - protein, 15 aa, synthetic construct
WSWDINGNVIRSEFT

SEQ ID NO: 111 - protein, 15 aa, synthetic construct
NVIRSEFTNDSELYH

SEQ ID NO: 112 - protein, 15 aa, synthetic construct
TNDSELYHGNRVPFV

SEQ ID NO: 113 - protein, 15 aa, synthetic construct
HGNRVPFVPRYGAGS

SEQ ID NO: 114 - protein, 15 aa, synthetic construct
DTRYGALMPRLAVNL

SEQ ID NO: 115 - protein, 15 aa, synthetic construct
MPRLAVNLVGPHYFD

SEQ ID NO: 116 - protein, 15 aa, synthetic construct
LVGPHYFDGDNQLRQ

SEQ ID NO: 117 - protein, 15 aa, synthetic construct
DGDNQLRQGTYATLD

SEQ ID NO: 118 - protein, 15 aa, synthetic construct
QGTYATLDSSLGWQA

SEQ ID NO: 119 - protein, 15 aa, synthetic construct
DSSLGWQATERMNIS

SEQ ID NO: 120 - protein, 15 aa, synthetic construct
ATERMNISVYVDNLF

SEQ ID NO: 121 - protein, 15 aa, synthetic construct
YGYMNGSSAVAQVNM

SEQ ID NO: 122 - protein, 15 aa, synthetic construct
SAVAQVNMGRTVGIN

SEQ ID NO: 123 - protein, 15 aa, synthetic construct
NMGRTVGINTRIDFF

SEQ ID NO: 124 - protein, 4 aa, synthetic construct
GSGS

SEQ ID NO: 125 - protein, 4 aa, synthetic construct
GPGP

SEQ ID NO: 126 - protein, 6 aa, synthetic construct
LLSVGG

SEQ ID NO: 127 - protein, 535 aa, synthetic construct
MWTAKDANDNPVTGLNPDAPSLSGAAAAGSTASGWTDNGDGTWGSGSKGTWQIDSAQSLGSGSAAQLTVIAGEMSSANSTLVADNKTPTVKTTTELTGSGSDESLRFYPFPTVNANGSGSSALALQADARNSDLTITQHGGGNGADVGQGSDDSSIDGSGSGNVIRSEFTNDSEGSGSHNDYPETITDGSGSNLRLNVGVSNILNKQGSGSSYPFPTTSETPRVVYNSRTDKPGSGSDVQSVELLRGPQGTLGSGSWNQCNGPGFADGSWAYGSGSGGLINIVTKKGQPEGSGSIVSVNGVDTGETTMRWYKAGTQNGSGSAPMMVSVIDTSAPEGSGSGEVKGRLVLDDRKRTEEVRPLGSGSVDGIEASMSFPLVKGSGSVIDAKSYKVVKTFDGSGSQGTYATLDSSLGWQGSGSATLDQWNGKDSTMGSGSQTTWVIENAELEQQGSGSESTGTPSNATYTYVIERWDPETGSGSDFYVSYQGQQALKGSGSPIEGQTTHRGPSLNGSGSDETLVVEATAEQVLKHHHHHH

SEQ ID NO: 128 - protein, 219 aa, synthetic construct
MKGTWQIDSAQSLGSGSDESLRFYPFPTVNANGSGSGNVIRSEFTNDSEGSGSNLRLNVGVSNILNKQGSGSDVQSVELLRGPQGTLGSGSGGLINIVTKKGQPEGSGSAPMMVSVIDTSAPEGSGSVDGIEASMSFPLVKGSGSQGTYATLDSSLGWQGSGSQTTWVIENAELEQQGSGSDFYVSYQGQQALKGSGSDETLVVEATAEQVLKHHHHHH

SEQ ID NO: 129 - protein, 420 aa, synthetic construct
MWTAKDANDNPVTGLNPDAPSLSGAAAAGSTASGWTDNGDGTWGSGSKGTWQIDSAQSLGSGSAAQLTVIAGEMSSANSTLVADNKTPTVKTTTELTGSGSDESLRFYPFPTVNANGSGSSALALQADARNSDLTITQHGGGNGADVGQGSDDSSIDGSGSGNVIRSEFTNDSEGSGSHNDYPETITDGSGSNLRLNVGVSNILNKQGSGSSYPFPTTSETPRVVYNSRTDKPGSGSDVQSVELLRGPQGTLGSGSWNQCNGPGFADGSWAYGSGSGGLINIVTKKGQPEGSGSIVSVNGVDTGETTMRWYKAGTQNGSGSAPMMVSVIDTSAPEGSGSGEVKGRLVLDDRKRTEEVRPLGSGSVDGIEASMSFPLVKGSGSVIDAKSYKVVKTFDGSGSQGTYATLDSSLGWQHHHHHH

SEQ ID NO: 130 - protein, 165 aa, synthetic construct
MKGTWQIDSAQSLGSGSDESLRFYPFPTVNANGSGSGNVIRSEFTNDSEGSGSNLRLNVGVSNILNKQGSGSDVQSVELLRGPQGTLGSGSGGLINIVTKKGQPEGSGSAPMMVSVIDTSAPEGSGSVDGIEASMSFPLVKGSGSQGTYATLDSSLGWQHHHHHH

SEQ ID NO: 131 - protein, 581 aa, synthetic construct
MWTAKDANDNPVTGLNPDAPSLSGAAAAGSTASGWTDNGDGTWLLSVGGKGTWQIDSAQSLLLSVGGAAQLTVIAGEMSSANSTLVADNKTPTVKTTTELTLLSVGGDESLRFYPFPTVNANLLSVGGSALALQADARNSDLTITQHGGGNGADVGQGSDDSSIDLLSVGGGNVIRSEFTNDSELLSVGGHNDYPETITDLLSVGGNLRLNVGVSNILNKQLLSVGGSYPFPTTSETPRVVYNSRTDKPLLSVGGDVQSVELLRGPQGTLLLSVGGWNQCNGPGFADGSWAYLLSVGGGGLINIVTKKGQPELLSVGGIVSVNGVDTGETTMRWYKAGTQNLLSVGGAPMMVSVIDTSAPELLSVGGGEVKGRLVLDDRKRTEEVRPLLLSVGGVDGIEASMSFPLVKLLSVGGVIDAKSYKVVKTFDLLSVGGQGTYATLDSSLGWQLLSVGGATLDQWNGKDSTMLLSVGGQTTWVIENAELEQQLLSVGGESTGTPSNATYTYVIERWDPETLLSVGGDFYVSYQGQQALKLLSVGGPIEGQTTHRGPSLNLLSVGGDETLVVEATAEQVLKHHHHHH

SEQ ID NO: 132 - protein, 534 aa, synthetic construct
MWTAKDANDNPVTGLNPDAPSLSGAAAAGSTASGWTDNGDGTWGSGSKGTWQIDSAQSLGSGSAAQLTVIAGEMSSANSTLVADNKTPTVKTTTELTGSGSDESLRFYPFPTVNANGSGSSALALQADARNSDLTITQHGGGNGADVGQGSDDSSIDGSGSHTKDMQLYSGPVGMQTGSGSHNDYPETITDGSGSNLRLNVGVSNILNKQGSGSSYPFPTTSETPRVVYNSRTDKPGSGSFNLISAWQQQHYSRTGSGSWNQCNGPGFADGSWAYGSGSLNFSQGVELPDPGGSGSIVSVNGVDTGETTMRWYKAGTQNGSGSAPMMVSVIDTSAPEGSGSGEVKGRLVLDDRKRTEEVRPLGSGSINNSLNWTITQAFSGSGSVIDAKSYKVVKTFDGSGSGNMLFSTISLRGVGSGSATLDQWNGKDSTMGSGSQTTWVIENAELEQQGSGSESTGTPSNATYTYVIERWDPETGSGSNPTNWLMLFGSYAQGSGSPIEGQTTHRGPSLNGSGSNDYQNKIVAGDNHHHHHH

SEQ ID NO: 133 - protein, 218 aa, synthetic construct
MKGTWQIDSAQSLGSGSDESLRFYPFPTVNANGSGSHTKDMQLYSGPVGMQTGSGSNLRLNVGVSNILNKQGSGSFNLISAWQQQHYSRTGSGSLNFSQGVELPDPGGSGSAPMMVSVIDTSAPEGSGSINNSLNWTITQAFSGSGSGNMLFSTISLRGVGSGSQTTWVIENAELEQQGSGSNPTNWLMLFGSYAQGSGSNDYQNKIVAGDNHHHHHH

SEQ ID NO: 134 - protein, 580 aa, synthetic construct
MWTAKDANDNPVTGLNPDAPSLSGAAAAGSTASGWTDNGDGTWLLSVGGKGTWQIDSAQSLLLSVGGAAQLTVIAGEMSSANSTLVADNKTPTVKTTTELTLLSVGGDESLRFYPFPTVNANLLSVGGSALALQADARNSDLTITQHGGGNGADVGQGSDDSSIDLLSVGGHTKDMQLYSGPVGMQTLLSVGGHNDYPETITDLLSVGGNLRLNVGVSNILNKQLLSVGGSYPFPTTSETPRVVYNSRTDKPLLSVGGFNLISAWQQQHYSRTLLSVGGWNQCNGPGFADGSWAYLLSVGGLNFSQGVELPDPGLLSVGGIVSVNGVDTGETTMRWYKAGTQNLLSVGGAPMMVSVIDTSAPELLSVGGGEVKGRLVLDDRKRTEEVRPLLLSVGGINNSLNWTITQAFSLLSVGGVIDAKSYKVVKTFDLLSVGGGNMLFSTISLRGVLLSVGGATLDQWNGKDSTMLLSVGGQTTWVIENAELEQQLLSVGGESTGTPSNATYTYVIERWDPETLLSVGGNPTNWLMLFGSYAQLLSVGGPIEGQTTHRGPSLNLLSVGGNDYQNKIVAGDNHHHHHH

SEQ ID NO: 135 - protein, 532 aa, synthetic construct
MWTAKDANDNPVTGLNPDAPSLSGAAAAGSTASGWTDNGDGTWGSGSINNSLNWTITQAFSGSGSAAQLTVIAGEMSSANSTLVADNKTPTVKTTTELTGSGSNPTNWLMLFGSYAQGSGSSALALQADARNSDLTITQHGGGNGADVGQGSDDSSIDGSGSNDYQNKIVAGDNGSGSHNDYPETITDGSGSDESLRFYPFPTVNANGSGSSYPFPTTSETPRVVYNSRTDKPGSGSINNSLNWTITQAFSGSGSWNQCNGPGFADGSWAYGSGSNPTNWLMLFGSYAQGSGSIVSVNGVDTGETTMRWYKAGTQNGSGSNDYQNKIVAGDNGSGSGEVKGRLVLDDRKRTEEVRPLGSGSDESLRFYPFPTVNANGSGSVIDAKSYKVVKTFDGSGSINNSLNWTITQAFSGSGSATLDQWNGKDSTMGSGSNPTNWLMLFGSYAQGSGSESTGTPSNATYTYVIERWDPETGSGSNDYQNKIVAGDNGSGSPIEGQTTHRGPSLNGSGSDESLRFYPFPTVNANHHHHHH

SEQ ID NO: 136 - protein, 536 aa, synthetic construct
MWTAKDANDNPVTGLNPDAPSLSGAAAAGSTASGWTDNGDGTWGSGSKGTWQIDSAQSLGSGSAAQLTVIAGEMSSANSTLVADNKTPTVKTTTELTGSGSDESLRFYPFPTVNANGSGSSALALQADARNSDLTITQHGGGNGADVGQGSDDSSIDGSGSHTKDMQLYSGPVGMQTGSGSHNDYPETITDGSGSNLRLNVGVSNILNKQGSGSSYPFPTTSETPRVVYNSRTDKPGSGSFNLISAWQQQHYSRTGSGSWNQCNGPGFADGSWAYGSGSLNFSQGVELPDPGGSGSIVSVNGVDTGETTMRWYKAGTQNGSGSAPMMVSVIDTSAPEGSGSGEVKGRLVLDDRKRTEEVRPLGSGSINNSLNWTITQAFSGSGSVIDAKSYKVVKTFDGSGSPIQDGLLYGSVTLLRGSGSATLDQWNGKDSTMGSGSQTTWVIENAELEQQGSGSESTGTPSNATYTYVIERWDPETGSGSNPTNWLMLFGSYAQGSGSPIEGQTTHRGPSLNGSGSNDYQNKIVAGDNHHHHHH

SEQ ID NO: 137 - protein, 220 aa, synthetic construct
MKGTWQIDSAQSLGSGSDESLRFYPFPTVNANGSGSHTKDMQLYSGPVGMQTGSGSNLRLNVGVSNILNKQGSGSFNLISAWQQQHYSRTGSGSLNFSQGVELPDPGGSGSAPMMVSVIDTSAPEGSGSINNSLNWTITQAFSGSGSPIQDGLLYGSVTLLRGSGSQTTWVIENAELEQQGSGSNPTNWLMLFGSYAQGSGSNDYQNKIVAGDNHHHHHH

SEQ ID NO: 138 - protein, 567 aa, synthetic construct
MWTAKDANDNPVTGLNPDAPSLSGAAAAGSTASGWTDNGDGTWGSGSKGTWQIDSAQSLGSGSAAQLTVIAGEMSSANSTLVADNKTPTVKTTTELTGSGSDESLRFYPFPTVNANGSGSSALALQADARNSDLTITQHGGGNGADVGQGSDDSSIDGSGSHTKDMQLYSGPVGMQTGSGSHNDYPETITDGSGSNLRLNVGVSNILNKQGSGSSYPFPTTSETPRVVYNSRTDKPGSGSFNLISAWQQQHYSRTGSGSWNQCNGPGFADGSWAYGSGSLNFSQGVELPDPGGSGSIVSVNGVDTGETTMRWYKAGTQNGSGSAPMMVSVIDTSAPEGSGSGEVKGRLVLDDRKRTEEVRPLGSGSINNSLNWTITQAFSGSGSVIDAKSYKVVKTFDGSGSGNMLFSTISLRGVGSGSATLDQWNGKDSTMGSGSQTTWVIENAELEQQGSGSESTGTPSNATYTYVIERWDPETGSGSNPTNWLMLFGSYAQGSGSPIEGQTTHRGPSLNGSGSNDYQNKIVAGDNGSGSTGATIPIGGGGSGSPIQDGLLYGSVTLLRHHHHHH

SEQ ID NO: 139 - protein, 237 aa, synthetic construct
MKGTWQIDSAQSLGSGSDESLRFYPFPTVNANGSGSHTKDMQLYSGPVGMQTGSGSNLRLNVGVSNILNKQGSGSFNLISAWQQQHYSRTGSGSLNFSQGVELPDPGGSGSAPMMVSVIDTSAPEGSGSINNSLNWTITQAFSGSGSGNMLFSTISLRGVGSGSQTTWVIENAELEQQGSGSNPTNWLMLFGSYAQGSGSNDYQNKIVAGDNGSGSPIQDGLLYGSVTLLRHHHHHH

SEQ ID NO: 140 - protein, 550 aa, synthetic construct
MWTAKDANDNPVTGLNPDAPSLSGAAAAGSTASGWTDNGDGTWGSGSKGTWQIDSAQSLGSGSAAQLTVIAGEMSSANSTLVADNKTPTVKTTTELTGSGSDESLRFYPFPTVNANGSGSSALALQADARNSDLTITQHGGGNGADVGQGSDDSSIDGSGSHTKDMQLYSGPVGMQTGSGSHNDYPETITDGSGSNLRLNVGVSNILNKQGSGSSYPFPTTSETPRVVYNSRTDKPGSGSFNLISAWQQQHYSRTGSGSWNQCNGPGFADGSWAYGSGSLNFSQGVELPDPGGSGSIVSVNGVDTGETTMRWYKAGTQNGSGSAPMMVSVIDTSAPEGSGSGEVKGRLVLDDRKRTEEVRPLGSGSINNSLNWTITQAFSGSGSVIDAKSYKVVKTFDGSGSPIQDGLLYGSVTLLRGSGSATLDQWNGKDSTMGSGSQTTWVIENAELEQQGSGSESTGTPSNATYTYVIERWDPETGSGSNPTNWLMLFGSYAQGSGSPIEGQTTHRGPSLNGSGSNDYQNKIVAGDNGSGSTGATIPIGGGHHHHHH

SEQ ID NO: 141 - protein, 536 aa, synthetic construct
MWTAKDANDNPVTGLNPDAPSLSGAAAAGSTASGWTDNGDGTWGSGSAAQLTVIAGEMSSANSTLVADNKTPTVKTTTELTGSGSSALALQADARNSDLTITQHGGGNGADVGQGSDDSSIDGSGSHNDYPETITDGSGSKGTWQIDSAQSLGSGSDESLRFYPFPTVNANGSGSHTKDMQLYSGPVGMQTGSGSNLRLNVGVSNILNKQGSGSSYPFPTTSETPRVVYNSRTDKPGSGSWNQCNGPGFADGSWAYGSGSIVSVNGVDTGETTMRWYKAGTQNGSGSGEVKGRLVLDDRKRTEEVRPLGSGSFNLISAWQQQHYSRTGSGSLNFSQGVELPDPGGSGSAPMMVSVIDTSAPEGSGSINNSLNWTITQAFSGSGSVIDAKSYKVVKTFDGSGSATLDQWNGKDSTMGSGSESTGTPSNATYTYVIERWDPETGSGSPIEGQTTHRGPSLNGSGSPIQDGLLYGSVTLLRGSGSQTTWVIENAELEQQGSGSNPTNWLMLFGSYAQGSGSNDYQNKIVAGDNHHHHHH

SEQ ID NO: 142 - protein, 536 aa, synthetic construct
MKGTWQIDSAQSLGSGSDESLRFYPFPTVNANGSGSHTKDMQLYSGPVGMQTGSGSNLRLNVGVSNILNKQGSGSWTAKDANDNPVTGLNPDAPSLSGAAAAGSTASGWTDNGDGTWGSGSAAQLTVIAGEMSSANSTLVADNKTPTVKTTTELTGSGSSALALQADARNSDLTITQHGGGNGADVGQGSDDSSIDGSGSHNDYPETITDGSGSFNLISAWQQQHYSRTGSGSLNFSQGVELPDPGGSGSAPMMVSVIDTSAPEGSGSINNSLNWTITQAFSGSGSSYPFPTTSETPRVVYNSRTDKPGSGSWNQCNGPGFADGSWAYGSGSIVSVNGVDTGETTMRWYKAGTQNGSGSGEVKGRLVLDDRKRTEEVRPLGSGSPIQDGLLYGSVTLLRGSGSQTTWVIENAELEQQGSGSNPTNWLMLFGSYAQGSGSNDYQNKIVAGDNGSGSVIDAKSYKVVKTFDGSGSATLDQWNGKDSTMGSGSESTGTPSNATYTYVIERWDPETGSGSPIEGQTTHRGPSLNHHHHHH

SEQ ID NO: 143 - protein, 536 aa, synthetic construct
MAAQLTVIAGEMSSANSTLVADNKTPTVKTTTELTGSGSKGTWQIDSAQSLGSGSWTAKDANDNPVTGLNPDAPSLSGAAAAGSTASGWTDNGDGTWGSGSDESLRFYPFPTVNANGSGSSALALQADARNSDLTITQHGGGNGADVGQGSDDSSIDGSGSHTKDMQLYSGPVGMQTGSGSHNDYPETITDGSGSNLRLNVGVSNILNKQGSGSSYPFPTTSETPRVVYNSRTDKPGSGSFNLISAWQQQHYSRTGSGSWNQCNGPGFADGSWAYGSGSLNFSQGVELPDPGGSGSIVSVNGVDTGETTMRWYKAGTQNGSGSAPMMVSVIDTSAPEGSGSGEVKGRLVLDDRKRTEEVRPLGSGSINNSLNWTITQAFSGSGSVIDAKSYKVVKTFDGSGSPIQDGLLYGSVTLLRGSGSATLDQWNGKDSTMGSGSQTTWVIENAELEQQGSGSESTGTPSNATYTYVIERWDPETGSGSNPTNWLMLFGSYAQGSGSPIEGQTTHRGPSLNGSGSNDYQNKIVAGDNHHHHHH

SEQ ID NO: 144 - protein, 536 aa, synthetic construct
MWTAKDANDNPVTGLNPDAPSLSGAAAAGSTASGWTDNGDGTWGSGSDESLRFYPFPTVNANGSGSAAQLTVIAGEMSSANSTLVADNKTPTVKTTTELTGSGSKGTWQIDSAQSLGSGSSALALQADARNSDLTITQHGGGNGADVGQGSDDSSIDGSGSHTKDMQLYSGPVGMQTGSGSHNDYPETITDGSGSNLRLNVGVSNILNKQGSGSSYPFPTTSETPRVVYNSRTDKPGSGSFNLISAWQQQHYSRTGSGSWNQCNGPGFADGSWAYGSGSLNFSQGVELPDPGGSGSIVSVNGVDTGETTMRWYKAGTQNGSGSAPMMVSVIDTSAPEGSGSGEVKGRLVLDDRKRTEEVRPLGSGSINNSLNWTITQAFSGSGSVIDAKSYKVVKTFDGSGSPIQDGLLYGSVTLLRGSGSATLDQWNGKDSTMGSGSQTTWVIENAELEQQGSGSESTGTPSNATYTYVIERWDPETGSGSNPTNWLMLFGSYAQGSGSPIEGQTTHRGPSLNGSGSNDYQNKIVAGDNHHHHHH

SEQ ID NO: 145 - protein, 220 aa, synthetic construct
MDESLRFYPFPTVNANGSGSKGTWQIDSAQSLGSGSHTKDMQLYSGPVGMQTGSGSNLRLNVGVSNILNKQGSGSFNLISAWQQQHYSRTGSGSLNFSQGVELPDPGGSGSAPMMVSVIDTSAPEGSGSINNSLNWTITQAFSGSGSPIQDGLLYGSVTLLRGSGSQTTWVIENAELEQQGSGSNPTNWLMLFGSYAQGSGSNDYQNKIVAGDNHHHHHH

SEQ ID NO: 146 - protein, 4 aa, synthetic construct
SGSG

SEQ ID NO: 147 - protein, 8 aa, synthetic construct
SGSGSGSG

SEQ ID NO: 148 - protein, 12 aa, synthetic construct
SGSGSGSGSGSG

SEQ ID NO: 149 - protein, 16 aa, synthetic construct
SGSGSGSGSGSGSGSG

SEQ ID NO: 150 - protein, 20 aa, synthetic construct
SGSGSGSGSGSGSGSGSGSG

SEQ ID NO: 151 - protein, 24 aa, synthetic construct
SGSGSGSGSGSGSGSGSGSGSGSG

SEQ ID NO: 152 - protein, 28 aa, synthetic construct
SGSGSGSGSGSGSGSGSGSGSGSGSGSG

SEQ ID NO: 153 - protein, 32 aa, synthetic construct
SGSGSGSGSGSGSGSGSGSGSGSGSGSGSGSG

SEQ ID NO: 154 - protein, 36 aa, synthetic construct
SGSGSGSGSGSGSGSGSGSGSGSGSGSGSGSGSGSG

SEQ ID NO: 155 - protein, 40 aa, synthetic construct
SGSGSGSGSGSGSGSGSGSGSGSGSGSGSGSGSGSGSGSG

SEQ ID NO: 156 - protein, 4 aa, synthetic construct
SSSS

SEQ ID NO: 157 - protein, 4 aa, synthetic construct
GGGS

SEQ ID NO: 158-159 - skipped (2 entries)

SEQ ID NO: 160 - protein, 24 aa, synthetic construct
GGCGGCGGCGGCGGCGGCGGCGGC

SEQ ID NO: 161 - protein, 15 aa, synthetic construct
GGGGSGGGGSGGGGS

SEQ ID NO: 162 - protein, 5 aa, synthetic construct
GGAAY

SEQ ID NO: 163 - protein, 15 aa, synthetic construct
SALALQADARNSDLT

SEQ ID NO: 164 - protein, 15 aa, synthetic construct
QADARNSDLTITQHG

SEQ ID NO: 165 - protein, 15 aa, synthetic construct
NSDLTITQHGGGNGA

SEQ ID NO: 166 - protein, 15 aa, synthetic construct
ITQHGGGNGADVGQG

SEQ ID NO: 167 - protein, 15 aa, synthetic construct
GGNGADVGQGSDDSS

SEQ ID NO: 168 - protein, 15 aa, synthetic construct
NGADVGQGSDDSSID

SEQ ID NO: 169-170 - skipped (2 entries)

SEQ ID NO: 171 - protein, 15 aa, synthetic construct
IAYDNKGNASKRVQT

SEQ ID NO: 172 - protein, 16 aa, synthetic construct
KGNASKRVQTEVVISG

SEQ ID NO: 173 - protein, 15 aa, synthetic construct
PTLGEFTETEAGVYQ

SEQ ID NO: 174 - protein, 15 aa, synthetic construct
AGVYQSVFTTGTQSG

SEQ ID NO: 175 - protein, 15 aa, synthetic construct
VYQSVFTTGTQSGEA

SEQ ID NO: 176 - protein, 15 aa, synthetic construct
WTAKDANDNPVTGLN

SEQ ID NO: 177 - protein, 15 aa, synthetic construct
ANDNPVTGLNPDAPS

SEQ ID NO: 178 - protein, 15 aa, synthetic construct
VTGLNPDAPSLSGAA

SEQ ID NO: 179 - protein, 15 aa, synthetic construct
PDAPSLSGAAAAGST

SEQ ID NO: 180 - protein, 15 aa, synthetic construct
LSGAAAAGSTASGWT

SEQ ID NO: 181 - protein, 15 aa, synthetic construct
AAGSTASGWTDNGDG

SEQ ID NO: 182 - protein, 15 aa, synthetic construct
GSTASGWTDNGDGTW

SEQ ID NO: 183 - protein, 10 aa, synthetic construct
SWTEKGDGSY

SEQ ID NO: 184 - protein, 15 aa, synthetic construct
GDGSYVATLTTGGKT

SEQ ID NO: 185 - protein, 15 aa, synthetic construct
VATLTTGGKTGELRV

SEQ ID NO: 186 - protein, 15 aa, synthetic construct
TGGKTGELRVMPLFN

SEQ ID NO: 187 - protein, 15 aa, synthetic construct
KTGELRVMPLFNGQP

SEQ ID NO: 188 - protein, 11 aa, synthetic construct
AAQLTVIAGEM

SEQ ID NO: 189 - protein, 15 aa, synthetic construct
VIAGEMSSANSTLVA

SEQ ID NO: 190 - protein, 15 aa, synthetic construct
MSSANSTLVADNKTP

SEQ ID NO: 191 - protein, 15 aa, synthetic construct
STLVADNKTPTVKTT

SEQ ID NO: 192 - protein, 15 aa, synthetic construct
ADNKTPTVKTTTELT

SEQ ID NO: 193 - protein, 15 aa, synthetic construct
SGAASTGSERPSAGN

SEQ ID NO: 194 - protein, 15 aa, synthetic construct
TGSERPSAGNWTEKG

SEQ ID NO: 195 - protein, 10 aa, synthetic construct
TGATIPIGGG

SEQ ID NO: 196 - skipped

SEQ ID NO: 197 - protein, 15 aa, synthetic construct
SYPFPTTSETPRVVY

SEQ ID NO: 198 - protein, 15 aa, synthetic construct
TTSETPRVVYNSRTD

SEQ ID NO: 199 - protein, 15 aa, synthetic construct
SETPRVVYNSRTDKP

SEQ ID NO: 200 - protein, 15 aa, synthetic construct
ESTGTPSNATYTYVI

SEQ ID NO: 201 - protein, 15 aa, synthetic construct
PSNATYTYVIERWDP

SEQ ID NO: 202 - protein, 15 aa, synthetic construct
NATYTYVIERWDPET

SEQ ID NO: 203 - skipped

SEQ ID NO: 204 - protein, 15 aa, synthetic construct
PGSLCGIAPPPVGAC

SEQ ID NO: 205 - protein, 15 aa, synthetic construct
GIAPPPVGACKISEG

SEQ ID NO: 206 - skipped

SEQ ID NO: 207 - protein, 11 aa, synthetic construct
GPGFADGSWAY

SEQ ID NO: 208 - skipped

SEQ ID NO: 209 - protein, 15 aa, synthetic construct
AGNLCQKPGETTRLT

SEQ ID NO: 210 - protein, 15 aa, synthetic construct
LCQKPGETTRLTEKF

SEQ ID NO: 211-212 - skipped (2 entries)

SEQ ID NO: 213 - protein, 15 aa, synthetic construct
IVSVNGVDTGETTMR

SEQ ID NO: 214 - protein, 15 aa, synthetic construct
GVDTGETTMRWYKAG

SEQ ID NO: 215 - protein, 15 aa, synthetic construct
TGETTMRWYKAGTQN

SEQ ID NO: 216 - protein, 15 aa, synthetic construct
SQQENALWLATSQSR

SEQ ID NO: 217 - protein, 15 aa, synthetic construct
ALWLATSQSRKLDKG

SEQ ID NO: 218 - protein, 15 aa, synthetic construct
GEVKGRLVLDDRKRT

SEQ ID NO: 219 - protein, 16 aa, synthetic construct
RLVLDDRKRTEEVRPL

SEQ ID NO: 220-222 - skipped (3 entries)

SEQ ID NO: 223 - protein, 530 aa, synthetic construct
MWTAKDANDNPVTGLNPDAPSLSGAAAAGSTASGWTDNGDGTWGSGSKGTWQIDSAQSLGSGSAAQLTVIAGEMSSANSTLVADNKTPTVKTTTELTGSGSDESLRFYPFPTVNANGSGSSALALQADARNSDLTITQHGGGNGADVGQGSDDSSIDGSGSHTKDMQLYSGPVGMQTGSGSHNDYPETITDGSGSNLRLNVGVSNILNKQGSGSSYPFPTTSETPRVVYNSRTDKPGSGSFNLISAWQQQHYSRTGSGSWNQCNGPGFADGSWAYGSGSLNFSQGVELPDPGGSGSIVSVNGVDTGETTMRWYKAGTQNGSGSAPMMVSVIDTSAPEGSGSGEVKGRLVLDDRKRTEEVRPLGSGSINNSLNWTITQAFSGSGSVIDAKSYKVVKTFDGSGSPIQDGLLYGSVTLLRGSGSATLDQWNGKDSTMGSGSQTTWVIENAELEQQGSGSESTGTPSNATYTYVIERWDPETGSGSNPTNWLMLFGSYAQGSGSPIEGQTTHRGPSLNGSGSNDYQNKIVAGDN

SEQ ID NO: 224 - protein, 214 aa, synthetic construct
MKGTWQIDSAQSLGSGSDESLRFYPFPTVNANGSGSHTKDMQLYSGPVGMQTGSGSNLRLNVGVSNILNKQGSGSFNLISAWQQQHYSRTGSGSLNFSQGVELPDPGGSGSAPMMVSVIDTSAPEGSGSINNSLNWTITQAFSGSGSPIQDGLLYGSVTLLRGSGSQTTWVIENAELEQQGSGSNPTNWLMLFGSYAQGSGSNDYQNKIVAGDN

SEQ ID NO: 225 - protein, 561 aa, synthetic construct
MWTAKDANDNPVTGLNPDAPSLSGAAAAGSTASGWTDNGDGTWGSGSKGTWQIDSAQSLGSGSAAQLTVIAGEMSSANSTLVADNKTPTVKTTTELTGSGSDESLRFYPFPTVNANGSGSSALALQADARNSDLTITQHGGGNGADVGQGSDDSSIDGSGSHTKDMQLYSGPVGMQTGSGSHNDYPETITDGSGSNLRLNVGVSNILNKQGSGSSYPFPTTSETPRVVYNSRTDKPGSGSFNLISAWQQQHYSRTGSGSWNQCNGPGFADGSWAYGSGSLNFSQGVELPDPGGSGSIVSVNGVDTGETTMRWYKAGTQNGSGSAPMMVSVIDTSAPEGSGSGEVKGRLVLDDRKRTEEVRPLGSGSINNSLNWTITQAFSGSGSVIDAKSYKVVKTFDGSGSGNMLFSTISLRGVGSGSATLDQWNGKDSTMGSGSQTTWVIENAELEQQGSGSESTGTPSNATYTYVIERWDPETGSGSNPTNWLMLFGSYAQGSGSPIEGQTTHRGPSLNGSGSNDYQNKIVAGDNGSGSTGATIPIGGGGSGSPIQDGLLYGSVTLLR

SEQ ID NO: 226 - protein, 231 aa, synthetic construct
MKGTWQIDSAQSLGSGSDESLRFYPFPTVNANGSGSHTKDMQLYSGPVGMQTGSGSNLRLNVGVSNILNKQGSGSFNLISAWQQQHYSRTGSGSLNFSQGVELPDPGGSGSAPMMVSVIDTSAPEGSGSINNSLNWTITQAFSGSGSGNMLFSTISLRGVGSGSQTTWVIENAELEQQGSGSNPTNWLMLFGSYAQGSGSNDYQNKIVAGDNGSGSPIQDGLLYGSVTLLR

SEQ ID NO: 227 - protein, 214 aa, synthetic construct
MDESLRFYPFPTVNANGSGSKGTWQIDSAQSLGSGSHTKDMQLYSGPVGMQTGSGSNLRLNVGVSNILNKQGSGSFNLISAWQQQHYSRTGSGSLNFSQGVELPDPGGSGSAPMMVSVIDTSAPEGSGSINNSLNWTITQAFSGSGSPIQDGLLYGSVTLLRGSGSQTTWVIENAELEQQGSGSNPTNWLMLFGSYAQGSGSNDYQNKIVAGDN

SEQ ID NO: 228 - protein, 529 aa, synthetic construct
MWTAKDANDNPVTGLNPDAPSLSGAAAAGSTASGWTDNGDGTWGSGSKGTWQIDSAQSLGSGSAAQLTVIAGEMSSANSTLVADNKTPTVKTTTELTGSGSDESLRFYPFPTVNANGSGSSALALQADARNSDLTITQHGGGNGADVGQGSDDSSIDGSGSGNVIRSEFTNDSEGSGSHNDYPETITDGSGSNLRLNVGVSNILNKQGSGSSYPFPTTSETPRVVYNSRTDKPGSGSDVQSVELLRGPQGTLGSGSWNQCNGPGFADGSWAYGSGSGGLINIVTKKGQPEGSGSIVSVNGVDTGETTMRWYKAGTQNGSGSAPMMVSVIDTSAPEGSGSGEVKGRLVLDDRKRTEEVRPLGSGSVDGIEASMSFPLVKGSGSVIDAKSYKVVKTFDGSGSQGTYATLDSSLGWQGSGSATLDQWNGKDSTMGSGSQTTWVIENAELEQQGSGSESTGTPSNATYTYVIERWDPETGSGSDFYVSYQGQQALKGSGSPIEGQTTHRGPSLNGSGSDETLVVEATAEQVLK

SEQ ID NO: 229 - protein, 213 aa, synthetic construct
MKGTWQIDSAQSLGSGSDESLRFYPFPTVNANGSGSGNVIRSEFTNDSEGSGSNLRLNVGVSNILNKQGSGSDVQSVELLRGPQGTLGSGSGGLINIVTKKGQPEGSGSAPMMVSVIDTSAPEGSGSVDGIEASMSFPLVKGSGSQGTYATLDSSLGWQGSGSQTTWVIENAELEQQGSGSDFYVSYQGQQALKGSGSDETLVVEATAEQVLK

SEQ ID NO: 230 - protein, 414 aa, synthetic construct
MWTAKDANDNPVTGLNPDAPSLSGAAAAGSTASGWTDNGDGTWGSGSKGTWQIDSAQSLGSGSAAQLTVIAGEMSSANSTLVADNKTPTVKTTTELTGSGSDESLRFYPFPTVNANGSGSSALALQADARNSDLTITQHGGGNGADVGQGSDDSSIDGSGSGNVIRSEFTNDSEGSGSHNDYPETITDGSGSNLRLNVGVSNILNKQGSGSSYPFPTTSETPRVVYNSRTDKPGSGSDVQSVELLRGPQGTLGSGSWNQCNGPGFADGSWAYGSGSGGLINIVTKKGQPEGSGSIVSVNGVDTGETTMRWYKAGTQNGSGSAPMMVSVIDTSAPEGSGSGEVKGRLVLDDRKRTEEVRPLGSGSVDGIEASMSFPLVKGSGSVIDAKSYKVVKTFDGSGSQGTYATLDSSLGWQ

SEQ ID NO: 231 - protein, 159 aa, synthetic construct
MKGTWQIDSAQSLGSGSDESLRFYPFPTVNANGSGSGNVIRSEFTNDSEGSGSNLRLNVGVSNILNKQGSGSDVQSVELLRGPQGTLGSGSGGLINIVTKKGQPEGSGSAPMMVSVIDTSAPEGSGSVDGIEASMSFPLVKGSGSQGTYATLDSSLGWQ

SEQ ID NO: 232 - protein, 575 aa, synthetic construct
MWTAKDANDNPVTGLNPDAPSLSGAAAAGSTASGWTDNGDGTWLLSVGGKGTWQIDSAQSLLLSVGGAAQLTVIAGEMSSANSTLVADNKTPTVKTTTELTLLSVGGDESLRFYPFPTVNANLLSVGGSALALQADARNSDLTITQHGGGNGADVGQGSDDSSIDLLSVGGGNVIRSEFTNDSELLSVGGHNDYPETITDLLSVGGNLRLNVGVSNILNKQLLSVGGSYPFPTTSETPRVVYNSRTDKPLLSVGGDVQSVELLRGPQGTLLLSVGGWNQCNGPGFADGSWAYLLSVGGGGLINIVTKKGQPELLSVGGIVSVNGVDTGETTMRWYKAGTQNLLSVGGAPMMVSVIDTSAPELLSVGGGEVKGRLVLDDRKRTEEVRPLLLSVGGVDGIEASMSFPLVKLLSVGGVIDAKSYKVVKTFDLLSVGGQGTYATLDSSLGWQLLSVGGATLDQWNGKDSTMLLSVGGQTTWVIENAELEQQLLSVGGESTGTPSNATYTYVIERWDPETLLSVGGDFYVSYQGQQALKLLSVGGPIEGQTTHRGPSLNLLSVGGDETLVVEATAEQVLK

SEQ ID NO: 233 - protein, 528 aa, synthetic construct
MWTAKDANDNPVTGLNPDAPSLSGAAAAGSTASGWTDNGDGTWGSGSKGTWQIDSAQSLGSGSAAQLTVIAGEMSSANSTLVADNKTPTVKTTTELTGSGSDESLRFYPFPTVNANGSGSSALALQADARNSDLTITQHGGGNGADVGQGSDDSSIDGSGSHTKDMQLYSGPVGMQTGSGSHNDYPETITDGSGSNLRLNVGVSNILNKQGSGSSYPFPTTSETPRVVYNSRTDKPGSGSFNLISAWQQQHYSRTGSGSWNQCNGPGFADGSWAYGSGSLNFSQGVELPDPGGSGSIVSVNGVDTGETTMRWYKAGTQNGSGSAPMMVSVIDTSAPEGSGSGEVKGRLVLDDRKRTEEVRPLGSGSINNSLNWTITQAFSGSGSVIDAKSYKVVKTFDGSGSGNMLFSTISLRGVGSGSATLDQWNGKDSTMGSGSQTTWVIENAELEQQGSGSESTGTPSNATYTYVIERWDPETGSGSNPTNWLMLFGSYAQGSGSPIEGQTTHRGPSLNGSGSNDYQNKIVAGDN

SEQ ID NO: 234 - protein, 212 aa, synthetic construct
MKGTWQIDSAQSLGSGSDESLRFYPFPTVNANGSGSHTKDMQLYSGPVGMQTGSGSNLRLNVGVSNILNKQGSGSFNLISAWQQQHYSRTGSGSLNFSQGVELPDPGGSGSAPMMVSVIDTSAPEGSGSINNSLNWTITQAFSGSGSGNMLFSTISLRGVGSGSQTTWVIENAELEQQGSGSNPTNWLMLFGSYAQGSGSNDYQNKIVAGDN

SEQ ID NO: 235 - protein, 574 aa, synthetic construct
MWTAKDANDNPVTGLNPDAPSLSGAAAAGSTASGWTDNGDGTWLLSVGGKGTWQIDSAQSLLLSVGGAAQLTVIAGEMSSANSTLVADNKTPTVKTTTELTLLSVGGDESLRFYPFPTVNANLLSVGGSALALQADARNSDLTITQHGGGNGADVGQGSDDSSIDLLSVGGHTKDMQLYSGPVGMQTLLSVGGHNDYPETITDLLSVGGNLRLNVGVSNILNKQLLSVGGSYPFPTTSETPRVVYNSRTDKPLLSVGGFNLISAWQQQHYSRTLLSVGGWNQCNGPGFADGSWAYLLSVGGLNFSQGVELPDPGLLSVGGIVSVNGVDTGETTMRWYKAGTQNLLSVGGAPMMVSVIDTSAPELLSVGGGEVKGRLVLDDRKRTEEVRPLLLSVGGINNSLNWTITQAFSLLSVGGVIDAKSYKVVKTFDLLSVGGGNMLFSTISLRGVLLSVGGATLDQWNGKDSTMLLSVGGQTTWVIENAELEQQLLSVGGESTGTPSNATYTYVIERWDPETLLSVGGNPTNWLMLFGSYAQLLSVGGPIEGQTTHRGPSLNLLSVGGNDYQNKIVAGDN

SEQ ID NO: 236 - protein, 526 aa, synthetic construct
MWTAKDANDNPVTGLNPDAPSLSGAAAAGSTASGWTDNGDGTWGSGSINNSLNWTITQAFSGSGSAAQLTVIAGEMSSANSTLVADNKTPTVKTTTELTGSGSNPTNWLMLFGSYAQGSGSSALALQADARNSDLTITQHGGGNGADVGQGSDDSSIDGSGSNDYQNKIVAGDNGSGSHNDYPETITDGSGSDESLRFYPFPTVNANGSGSSYPFPTTSETPRVVYNSRTDKPGSGSINNSLNWTITQAFSGSGSWNQCNGPGFADGSWAYGSGSNPTNWLMLFGSYAQGSGSIVSVNGVDTGETTMRWYKAGTQNGSGSNDYQNKIVAGDNGSGSGEVKGRLVLDDRKRTEEVRPLGSGSDESLRFYPFPTVNANGSGSVIDAKSYKVVKTFDGSGSINNSLNWTITQAFSGSGSATLDQWNGKDSTMGSGSNPTNWLMLFGSYAQGSGSESTGTPSNATYTYVIERWDPETGSGSNDYQNKIVAGDNGSGSPIEGQTTHRGPSLNGSGSDESLRFYPFPTVNAN

SEQ ID NO: 237 - protein, 544 aa, synthetic construct
MWTAKDANDNPVTGLNPDAPSLSGAAAAGSTASGWTDNGDGTWGSGSKGTWQIDSAQSLGSGSAAQLTVIAGEMSSANSTLVADNKTPTVKTTTELTGSGSDESLRFYPFPTVNANGSGSSALALQADARNSDLTITQHGGGNGADVGQGSDDSSIDGSGSHTKDMQLYSGPVGMQTGSGSHNDYPETITDGSGSNLRLNVGVSNILNKQGSGSSYPFPTTSETPRVVYNSRTDKPGSGSFNLISAWQQQHYSRTGSGSWNQCNGPGFADGSWAYGSGSLNFSQGVELPDPGGSGSIVSVNGVDTGETTMRWYKAGTQNGSGSAPMMVSVIDTSAPEGSGSGEVKGRLVLDDRKRTEEVRPLGSGSINNSLNWTITQAFSGSGSVIDAKSYKVVKTFDGSGSPIQDGLLYGSVTLLRGSGSATLDQWNGKDSTMGSGSQTTWVIENAELEQQGSGSESTGTPSNATYTYVIERWDPETGSGSNPTNWLMLFGSYAQGSGSPIEGQTTHRGPSLNGSGSNDYQNKIVAGDNGSGSTGATIPIGGG

SEQ ID NO: 238 - protein, 530 aa, synthetic construct
MWTAKDANDNPVTGLNPDAPSLSGAAAAGSTASGWTDNGDGTWGSGSAAQLTVIAGEMSSANSTLVADNKTPTVKTTTELTGSGSSALALQADARNSDLTITQHGGGNGADVGQGSDDSSIDGSGSHNDYPETITDGSGSKGTWQIDSAQSLGSGSDESLRFYPFPTVNANGSGSHTKDMQLYSGPVGMQTGSGSNLRLNVGVSNILNKQGSGSSYPFPTTSETPRVVYNSRTDKPGSGSWNQCNGPGFADGSWAYGSGSIVSVNGVDTGETTMRWYKAGTQNGSGSGEVKGRLVLDDRKRTEEVRPLGSGSFNLISAWQQQHYSRTGSGSLNFSQGVELPDPGGSGSAPMMVSVIDTSAPEGSGSINNSLNWTITQAFSGSGSVIDAKSYKVVKTFDGSGSATLDQWNGKDSTMGSGSESTGTPSNATYTYVIERWDPETGSGSPIEGQTTHRGPSLNGSGSPIQDGLLYGSVTLLRGSGSQTTWVIENAELEQQGSGSNPTNWLMLFGSYAQGSGSNDYQNKIVAGDN

SEQ ID NO: 239 - protein, 530 aa, synthetic construct
MKGTWQIDSAQSLGSGSDESLRFYPFPTVNANGSGSHTKDMQLYSGPVGMQTGSGSNLRLNVGVSNILNKQGSGSWTAKDANDNPVTGLNPDAPSLSGAAAAGSTASGWTDNGDGTWGSGSAAQLTVIAGEMSSANSTLVADNKTPTVKTTTELTGSGSSALALQADARNSDLTITQHGGGNGADVGQGSDDSSIDGSGSHNDYPETITDGSGSFNLISAWQQQHYSRTGSGSLNFSQGVELPDPGGSGSAPMMVSVIDTSAPEGSGSINNSLNWTITQAFSGSGSSYPFPTTSETPRVVYNSRTDKPGSGSWNQCNGPGFADGSWAYGSGSIVSVNGVDTGETTMRWYKAGTQNGSGSGEVKGRLVLDDRKRTEEVRPLGSGSPIQDGLLYGSVTLLRGSGSQTTWVIENAELEQQGSGSNPTNWLMLFGSYAQGSGSNDYQNKIVAGDNGSGSVIDAKSYKVVKTFDGSGSATLDQWNGKDSTMGSGSESTGTPSNATYTYVIERWDPETGSGSPIEGQTTHRGPSLN

SEQ ID NO: 240 - protein, 530 aa, synthetic construct
MAAQLTVIAGEMSSANSTLVADNKTPTVKTTTELTGSGSKGTWQIDSAQSLGSGSWTAKDANDNPVTGLNPDAPSLSGAAAAGSTASGWTDNGDGTWGSGSDESLRFYPFPTVNANGSGSSALALQADARNSDLTITQHGGGNGADVGQGSDDSSIDGSGSHTKDMQLYSGPVGMQTGSGSHNDYPETITDGSGSNLRLNVGVSNILNKQGSGSSYPFPTTSETPRVVYNSRTDKPGSGSFNLISAWQQQHYSRTGSGSWNQCNGPGFADGSWAYGSGSLNFSQGVELPDPGGSGSIVSVNGVDTGETTMRWYKAGTQNGSGSAPMMVSVIDTSAPEGSGSGEVKGRLVLDDRKRTEEVRPLGSGSINNSLNWTITQAFSGSGSVIDAKSYKVVKTFDGSGSPIQDGLLYGSVTLLRGSGSATLDQWNGKDSTMGSGSQTTWVIENAELEQQGSGSESTGTPSNATYTYVIERWDPETGSGSNPTNWLMLFGSYAQGSGSPIEGQTTHRGPSLNGSGSNDYQNKIVAGDN

SEQ ID NO: 241 - protein, 530 aa, synthetic construct
MWTAKDANDNPVTGLNPDAPSLSGAAAAGSTASGWTDNGDGTWGSGSDESLRFYPFPTVNANGSGSAAQLTVIAGEMSSANSTLVADNKTPTVKTTTELTGSGSKGTWQIDSAQSLGSGSSALALQADARNSDLTITQHGGGNGADVGQGSDDSSIDGSGSHTKDMQLYSGPVGMQTGSGSHNDYPETITDGSGSNLRLNVGVSNILNKQGSGSSYPFPTTSETPRVVYNSRTDKPGSGSFNLISAWQQQHYSRTGSGSWNQCNGPGFADGSWAYGSGSLNFSQGVELPDPGGSGSIVSVNGVDTGETTMRWYKAGTQNGSGSAPMMVSVIDTSAPEGSGSGEVKGRLVLDDRKRTEEVRPLGSGSINNSLNWTITQAFSGSGSVIDAKSYKVVKTFDGSGSPIQDGLLYGSVTLLRGSGSATLDQWNGKDSTMGSGSQTTWVIENAELEQQGSGSESTGTPSNATYTYVIERWDPETGSGSNPTNWLMLFGSYAQGSGSPIEGQTTHRGPSLNGSGSNDYQNKIVAGDN

SEQ ID NO: 242 - protein, 529 aa, synthetic construct
WTAKDANDNPVTGLNPDAPSLSGAAAAGSTASGWTDNGDGTWGSGSKGTWQIDSAQSLGSGSAAQLTVIAGEMSSANSTLVADNKTPTVKTTTELTGSGSDESLRFYPFPTVNANGSGSSALALQADARNSDLTITQHGGGNGADVGQGSDDSSIDGSGSHTKDMQLYSGPVGMQTGSGSHNDYPETITDGSGSNLRLNVGVSNILNKQGSGSSYPFPTTSETPRVVYNSRTDKPGSGSFNLISAWQQQHYSRTGSGSWNQCNGPGFADGSWAYGSGSLNFSQGVELPDPGGSGSIVSVNGVDTGETTMRWYKAGTQNGSGSAPMMVSVIDTSAPEGSGSGEVKGRLVLDDRKRTEEVRPLGSGSINNSLNWTITQAFSGSGSVIDAKSYKVVKTFDGSGSPIQDGLLYGSVTLLRGSGSATLDQWNGKDSTMGSGSQTTWVIENAELEQQGSGSESTGTPSNATYTYVIERWDPETGSGSNPTNWLMLFGSYAQGSGSPIEGQTTHRGPSLNGSGSNDYQNKIVAGDN

SEQ ID NO: 243 - protein, 213 aa, synthetic construct
KGTWQIDSAQSLGSGSDESLRFYPFPTVNANGSGSHTKDMQLYSGPVGMQTGSGSNLRLNVGVSNILNKQGSGSFNLISAWQQQHYSRTGSGSLNFSQGVELPDPGGSGSAPMMVSVIDTSAPEGSGSINNSLNWTITQAFSGSGSPIQDGLLYGSVTLLRGSGSQTTWVIENAELEQQGSGSNPTNWLMLFGSYAQGSGSNDYQNKIVAGDN

SEQ ID NO: 244 - protein, 560 aa, synthetic construct
WTAKDANDNPVTGLNPDAPSLSGAAAAGSTASGWTDNGDGTWGSGSKGTWQIDSAQSLGSGSAAQLTVIAGEMSSANSTLVADNKTPTVKTTTELTGSGSDESLRFYPFPTVNANGSGSSALALQADARNSDLTITQHGGGNGADVGQGSDDSSIDGSGSHTKDMQLYSGPVGMQTGSGSHNDYPETITDGSGSNLRLNVGVSNILNKQGSGSSYPFPTTSETPRVVYNSRTDKPGSGSFNLISAWQQQHYSRTGSGSWNQCNGPGFADGSWAYGSGSLNFSQGVELPDPGGSGSIVSVNGVDTGETTMRWYKAGTQNGSGSAPMMVSVIDTSAPEGSGSGEVKGRLVLDDRKRTEEVRPLGSGSINNSLNWTITQAFSGSGSVIDAKSYKVVKTFDGSGSGNMLFSTISLRGVGSGSATLDQWNGKDSTMGSGSQTTWVIENAELEQQGSGSESTGTPSNATYTYVIERWDPETGSGSNPTNWLMLFGSYAQGSGSPIEGQTTHRGPSLNGSGSNDYQNKIVAGDNGSGSTGATIPIGGGGSGSPIQDGLLYGSVTLLR

SEQ ID NO: 245 - protein, 230 aa, synthetic construct
KGTWQIDSAQSLGSGSDESLRFYPFPTVNANGSGSHTKDMQLYSGPVGMQTGSGSNLRLNVGVSNILNKQGSGSFNLISAWQQQHYSRTGSGSLNFSQGVELPDPGGSGSAPMMVSVIDTSAPEGSGSINNSLNWTITQAFSGSGSGNMLFSTISLRGVGSGSQTTWVIENAELEQQGSGSNPTNWLMLFGSYAQGSGSNDYQNKIVAGDNGSGSPIQDGLLYGSVTLLR

SEQ ID NO: 246 - protein, 213 aa, synthetic construct
DESLRFYPFPTVNANGSGSKGTWQIDSAQSLGSGSHTKDMQLYSGPVGMQTGSGSNLRLNVGVSNILNKQGSGSFNLISAWQQQHYSRTGSGSLNFSQGVELPDPGGSGSAPMMVSVIDTSAPEGSGSINNSLNWTITQAFSGSGSPIQDGLLYGSVTLLRGSGSQTTWVIENAELEQQGSGSNPTNWLMLFGSYAQGSGSNDYQNKIVAGDN

SEQ ID NO: 247 - protein, 528 aa, synthetic construct
WTAKDANDNPVTGLNPDAPSLSGAAAAGSTASGWTDNGDGTWGSGSKGTWQIDSAQSLGSGSAAQLTVIAGEMSSANSTLVADNKTPTVKTTTELTGSGSDESLRFYPFPTVNANGSGSSALALQADARNSDLTITQHGGGNGADVGQGSDDSSIDGSGSGNVIRSEFTNDSEGSGSHNDYPETITDGSGSNLRLNVGVSNILNKQGSGSSYPFPTTSETPRVVYNSRTDKPGSGSDVQSVELLRGPQGTLGSGSWNQCNGPGFADGSWAYGSGSGGLINIVTKKGQPEGSGSIVSVNGVDTGETTMRWYKAGTQNGSGSAPMMVSVIDTSAPEGSGSGEVKGRLVLDDRKRTEEVRPLGSGSVDGIEASMSFPLVKGSGSVIDAKSYKVVKTFDGSGSQGTYATLDSSLGWQGSGSATLDQWNGKDSTMGSGSQTTWVIENAELEQQGSGSESTGTPSNATYTYVIERWDPETGSGSDFYVSYQGQQALKGSGSPIEGQTTHRGPSLNGSGSDETLVVEATAEQVLK

SEQ ID NO: 248 - protein, 212 aa, synthetic construct
KGTWQIDSAQSLGSGSDESLRFYPFPTVNANGSGSGNVIRSEFTNDSEGSGSNLRLNVGVSNILNKQGSGSDVQSVELLRGPQGTLGSGSGGLINIVTKKGQPEGSGSAPMMVSVIDTSAPEGSGSVDGIEASMSFPLVKGSGSQGTYATLDSSLGWQGSGSQTTWVIENAELEQQGSGSDFYVSYQGQQALKGSGSDETLVVEATAEQVLK

SEQ ID NO: 249 - protein, 413 aa, synthetic construct
WTAKDANDNPVTGLNPDAPSLSGAAAAGSTASGWTDNGDGTWGSGSKGTWQIDSAQSLGSGSAAQLTVIAGEMSSANSTLVADNKTPTVKTTTELTGSGSDESLRFYPFPTVNANGSGSSALALQADARNSDLTITQHGGGNGADVGQGSDDSSIDGSGSGNVIRSEFTNDSEGSGSHNDYPETITDGSGSNLRLNVGVSNILNKQGSGSSYPFPTTSETPRVVYNSRTDKPGSGSDVQSVELLRGPQGTLGSGSWNQCNGPGFADGSWAYGSGSGGLINIVTKKGQPEGSGSIVSVNGVDTGETTMRWYKAGTQNGSGSAPMMVSVIDTSAPEGSGSGEVKGRLVLDDRKRTEEVRPLGSGSVDGIEASMSFPLVKGSGSVIDAKSYKVVKTFDGSGSQGTYATLDSSLGWQ

SEQ ID NO: 250 - protein, 158 aa, synthetic construct
KGTWQIDSAQSLGSGSDESLRFYPFPTVNANGSGSGNVIRSEFTNDSEGSGSNLRLNVGVSNILNKQGSGSDVQSVELLRGPQGTLGSGSGGLINIVTKKGQPEGSGSAPMMVSVIDTSAPEGSGSVDGIEASMSFPLVKGSGSQGTYATLDSSLGWQ

SEQ ID NO: 251 - protein, 574 aa, synthetic construct
WTAKDANDNPVTGLNPDAPSLSGAAAAGSTASGWTDNGDGTWLLSVGGKGTWQIDSAQSLLLSVGGAAQLTVIAGEMSSANSTLVADNKTPTVKTTTELTLLSVGGDESLRFYPFPTVNANLLSVGGSALALQADARNSDLTITQHGGGNGADVGQGSDDSSIDLLSVGGGNVIRSEFTNDSELLSVGGHNDYPETITDLLSVGGNLRLNVGVSNILNKQLLSVGGSYPFPTTSETPRVVYNSRTDKPLLSVGGDVQSVELLRGPQGTLLLSVGGWNQCNGPGFADGSWAYLLSVGGGGLINIVTKKGQPELLSVGGIVSVNGVDTGETTMRWYKAGTQNLLSVGGAPMMVSVIDTSAPELLSVGGGEVKGRLVLDDRKRTEEVRPLLLSVGGVDGIEASMSFPLVKLLSVGGVIDAKSYKVVKTFDLLSVGGQGTYATLDSSLGWQLLSVGGATLDQWNGKDSTMLLSVGGQTTWVIENAELEQQLLSVGGESTGTPSNATYTYVIERWDPETLLSVGGDFYVSYQGQQALKLLSVGGPIEGQTTHRGPSLNLLSVGGDETLVVEATAEQVLK

SEQ ID NO: 252 - protein, 527 aa, synthetic construct
WTAKDANDNPVTGLNPDAPSLSGAAAAGSTASGWTDNGDGTWGSGSKGTWQIDSAQSLGSGSAAQLTVIAGEMSSANSTLVADNKTPTVKTTTELTGSGSDESLRFYPFPTVNANGSGSSALALQADARNSDLTITQHGGGNGADVGQGSDDSSIDGSGSHTKDMQLYSGPVGMQTGSGSHNDYPETITDGSGSNLRLNVGVSNILNKQGSGSSYPFPTTSETPRVVYNSRTDKPGSGSFNLISAWQQQHYSRTGSGSWNQCNGPGFADGSWAYGSGSLNFSQGVELPDPGGSGSIVSVNGVDTGETTMRWYKAGTQNGSGSAPMMVSVIDTSAPEGSGSGEVKGRLVLDDRKRTEEVRPLGSGSINNSLNWTITQAFSGSGSVIDAKSYKVVKTFDGSGSGNMLFSTISLRGVGSGSATLDQWNGKDSTMGSGSQTTWVIENAELEQQGSGSESTGTPSNATYTYVIERWDPETGSGSNPTNWLMLFGSYAQGSGSPIEGQTTHRGPSLNGSGSNDYQNKIVAGDN

SEQ ID NO: 253 - protein, 211 aa, synthetic construct
KGTWQIDSAQSLGSGSDESLRFYPFPTVNANGSGSHTKDMQLYSGPVGMQTGSGSNLRLNVGVSNILNKQGSGSFNLISAWQQQHYSRTGSGSLNFSQGVELPDPGGSGSAPMMVSVIDTSAPEGSGSINNSLNWTITQAFSGSGSGNMLFSTISLRGVGSGSQTTWVIENAELEQQGSGSNPTNWLMLFGSYAQGSGSNDYQNKIVAGDN

SEQ ID NO: 254 - protein, 573 aa, synthetic construct
WTAKDANDNPVTGLNPDAPSLSGAAAAGSTASGWTDNGDGTWLLSVGGKGTWQIDSAQSLLLSVGGAAQLTVIAGEMSSANSTLVADNKTPTVKTTTELTLLSVGGDESLRFYPFPTVNANLLSVGGSALALQADARNSDLTITQHGGGNGADVGQGSDDSSIDLLSVGGHTKDMQLYSGPVGMQTLLSVGGHNDYPETITDLLSVGGNLRLNVGVSNILNKQLLSVGGSYPFPTTSETPRVVYNSRTDKPLLSVGGFNLISAWQQQHYSRTLLSVGGWNQCNGPGFADGSWAYLLSVGGLNFSQGVELPDPGLLSVGGIVSVNGVDTGETTMRWYKAGTQNLLSVGGAPMMVSVIDTSAPELLSVGGGEVKGRLVLDDRKRTEEVRPLLLSVGGINNSLNWTITQAFSLLSVGGVIDAKSYKVVKTFDLLSVGGGNMLFSTISLRGVLLSVGGATLDQWNGKDSTMLLSVGGQTTWVIENAELEQQLLSVGGESTGTPSNATYTYVIERWDPETLLSVGGNPTNWLMLFGSYAQLLSVGGPIEGQTTHRGPSLNLLSVGGNDYQNKIVAGDN

SEQ ID NO: 255 - protein, 525 aa, synthetic construct
WTAKDANDNPVTGLNPDAPSLSGAAAAGSTASGWTDNGDGTWGSGSINNSLNWTITQAFSGSGSAAQLTVIAGEMSSANSTLVADNKTPTVKTTTELTGSGSNPTNWLMLFGSYAQGSGSSALALQADARNSDLTITQHGGGNGADVGQGSDDSSIDGSGSNDYQNKIVAGDNGSGSHNDYPETITDGSGSDESLRFYPFPTVNANGSGSSYPFPTTSETPRVVYNSRTDKPGSGSINNSLNWTITQAFSGSGSWNQCNGPGFADGSWAYGSGSNPTNWLMLFGSYAQGSGSIVSVNGVDTGETTMRWYKAGTQNGSGSNDYQNKIVAGDNGSGSGEVKGRLVLDDRKRTEEVRPLGSGSDESLRFYPFPTVNANGSGSVIDAKSYKVVKTFDGSGSINNSLNWTITQAFSGSGSATLDQWNGKDSTMGSGSNPTNWLMLFGSYAQGSGSESTGTPSNATYTYVIERWDPETGSGSNDYQNKIVAGDNGSGSPIEGQTTHRGPSLNGSGSDESLRFYPFPTVNAN

SEQ ID NO: 256 - protein, 543 aa, synthetic construct
WTAKDANDNPVTGLNPDAPSLSGAAAAGSTASGWTDNGDGTWGSGSKGTWQIDSAQSLGSGSAAQLTVIAGEMSSANSTLVADNKTPTVKTTTELTGSGSDESLRFYPFPTVNANGSGSSALALQADARNSDLTITQHGGGNGADVGQGSDDSSIDGSGSHTKDMQLYSGPVGMQTGSGSHNDYPETITDGSGSNLRLNVGVSNILNKQGSGSSYPFPTTSETPRVVYNSRTDKPGSGSFNLISAWQQQHYSRTGSGSWNQCNGPGFADGSWAYGSGSLNFSQGVELPDPGGSGSIVSVNGVDTGETTMRWYKAGTQNGSGSAPMMVSVIDTSAPEGSGSGEVKGRLVLDDRKRTEEVRPLGSGSINNSLNWTITQAFSGSGSVIDAKSYKVVKTFDGSGSPIQDGLLYGSVTLLRGSGSATLDQWNGKDSTMGSGSQTTWVIENAELEQQGSGSESTGTPSNATYTYVIERWDPETGSGSNPTNWLMLFGSYAQGSGSPIEGQTTHRGPSLNGSGSNDYQNKIVAGDNGSGSTGATIPIGGG

SEQ ID NO: 257 - protein, 529 aa, synthetic construct
WTAKDANDNPVTGLNPDAPSLSGAAAAGSTASGWTDNGDGTWGSGSAAQLTVIAGEMSSANSTLVADNKTPTVKTTTELTGSGSSALALQADARNSDLTITQHGGGNGADVGQGSDDSSIDGSGSHNDYPETITDGSGSKGTWQIDSAQSLGSGSDESLRFYPFPTVNANGSGSHTKDMQLYSGPVGMQTGSGSNLRLNVGVSNILNKQGSGSSYPFPTTSETPRVVYNSRTDKPGSGSWNQCNGPGFADGSWAYGSGSIVSVNGVDTGETTMRWYKAGTQNGSGSGEVKGRLVLDDRKRTEEVRPLGSGSFNLISAWQQQHYSRTGSGSLNFSQGVELPDPGGSGSAPMMVSVIDTSAPEGSGSINNSLNWTITQAFSGSGSVIDAKSYKVVKTFDGSGSATLDQWNGKDSTMGSGSESTGTPSNATYTYVIERWDPETGSGSPIEGQTTHRGPSLNGSGSPIQDGLLYGSVTLLRGSGSQTTWVIENAELEQQGSGSNPTNWLMLFGSYAQGSGSNDYQNKIVAGDN

SEQ ID NO: 258 - protein, 529 aa, synthetic construct
KGTWQIDSAQSLGSGSDESLRFYPFPTVNANGSGSHTKDMQLYSGPVGMQTGSGSNLRLNVGVSNILNKQGSGSWTAKDANDNPVTGLNPDAPSLSGAAAAGSTASGWTDNGDGTWGSGSAAQLTVIAGEMSSANSTLVADNKTPTVKTTTELTGSGSSALALQADARNSDLTITQHGGGNGADVGQGSDDSSIDGSGSHNDYPETITDGSGSFNLISAWQQQHYSRTGSGSLNFSQGVELPDPGGSGSAPMMVSVIDTSAPEGSGSINNSLNWTITQAFSGSGSSYPFPTTSETPRVVYNSRTDKPGSGSWNQCNGPGFADGSWAYGSGSIVSVNGVDTGETTMRWYKAGTQNGSGSGEVKGRLVLDDRKRTEEVRPLGSGSPIQDGLLYGSVTLLRGSGSQTTWVIENAELEQQGSGSNPTNWLMLFGSYAQGSGSNDYQNKIVAGDNGSGSVIDAKSYKVVKTFDGSGSATLDQWNGKDSTMGSGSESTGTPSNATYTYVIERWDPETGSGSPIEGQTTHRGPSLN

SEQ ID NO: 259 - protein, 529 aa, synthetic construct
AAQLTVIAGEMSSANSTLVADNKTPTVKTTTELTGSGSKGTWQIDSAQSLGSGSWTAKDANDNPVTGLNPDAPSLSGAAAAGSTASGWTDNGDGTWGSGSDESLRFYPFPTVNANGSGSSALALQADARNSDLTITQHGGGNGADVGQGSDDSSIDGSGSHTKDMQLYSGPVGMQTGSGSHNDYPETITDGSGSNLRLNVGVSNILNKQGSGSSYPFPTTSETPRVVYNSRTDKPGSGSFNLISAWQQQHYSRTGSGSWNQCNGPGFADGSWAYGSGSLNFSQGVELPDPGGSGSIVSVNGVDTGETTMRWYKAGTQNGSGSAPMMVSVIDTSAPEGSGSGEVKGRLVLDDRKRTEEVRPLGSGSINNSLNWTITQAFSGSGSVIDAKSYKVVKTFDGSGSPIQDGLLYGSVTLLRGSGSATLDQWNGKDSTMGSGSQTTWVIENAELEQQGSGSESTGTPSNATYTYVIERWDPETGSGSNPTNWLMLFGSYAQGSGSPIEGQTTHRGPSLNGSGSNDYQNKIVAGDN

SEQ ID NO: 260 - protein, 529 aa, synthetic construct
WTAKDANDNPVTGLNPDAPSLSGAAAAGSTASGWTDNGDGTWGSGSDESLRFYPFPTVNANGSGSAAQLTVIAGEMSSANSTLVADNKTPTVKTTTELTGSGSKGTWQIDSAQSLGSGSSALALQADARNSDLTITQHGGGNGADVGQGSDDSSIDGSGSHTKDMQLYSGPVGMQTGSGSHNDYPETITDGSGSNLRLNVGVSNILNKQGSGSSYPFPTTSETPRVVYNSRTDKPGSGSFNLISAWQQQHYSRTGSGSWNQCNGPGFADGSWAYGSGSLNFSQGVELPDPGGSGSIVSVNGVDTGETTMRWYKAGTQNGSGSAPMMVSVIDTSAPEGSGSGEVKGRLVLDDRKRTEEVRPLGSGSINNSLNWTITQAFSGSGSVIDAKSYKVVKTFDGSGSPIQDGLLYGSVTLLRGSGSATLDQWNGKDSTMGSGSQTTWVIENAELEQQGSGSESTGTPSNATYTYVIERWDPETGSGSNPTNWLMLFGSYAQGSGSPIEGQTTHRGPSLNGSGSNDYQNKIVAGDN

(19) United States
(12) Patent Application Publication
(10) Pub. No.: US 2026/0306601 A1
(43) Pub. Date: Oct. 8, 2026

(54) COMPOSITIONS AND METHODS FOR GENERATING IMMUNITY TO BACTERIAL INFECTION

(71) Applicant: Syntiron LLC, St. Paul, MN (US)

(72) Inventors: Leah Randles, St. Paul, MN (US); Lisa Herron-Olson, St. Paul, MN (US); Patricia Ellen Tam, Golden Valley, MN (US)

(21) Appl. No.: 19/490,833

(22) PCT Filed: Jun. 5, 2024
(86) PCT No.: PCT/US2024/032547

§ 371 Date: Dec. 5, 2025

Related U.S. Application Data
(60) Provisional application No. 63/471,365, filed on Jun. 6, 2023.
(60) Provisional application No. 63/535,969, filed on Aug. 31, 2023.

(51) Int. Cl. A61K 39/02; A61K 39/00; A61K 39/39; A61P 31/04; C07K 14/245; C07K 14/705

(52) U.S. Cl. CPC A61K 39/02; A61K 39/39; A61P 31/04; C07K 14/245; C07K 14/705; A61K 2039/545; A61K 2039/55; A61K 2039/55505; A61K 2039/55544; A61K 2039/55561; A61K 2039/55566; A61K 2039/575; C07K 2319/00

(57) ABSTRACT

The present disclosure relates, in part, to a polypeptide of formula (I), or a salt or solvate thereof, and a vaccine composition thereof further comprising at least one pharmaceutically acceptable excipient. The present disclosure further relates to isolated mRNA and/or isolated polynucleotides encoding the polypeptide of formula (I), vectors and/or LNPs comprising the same, and pharmaceutical compositions thereof. The present disclosure further relates to methods of treating, preventing, and/or ameliorating a bacterial infection, and/or generating immunity to infection by one or more pathogenic bacteria, in a subject in need thereof, the method comprising administering to the subject at least one composition of the present disclosure. In certain embodiments, the bacterial infection is a urinary tract infection, sepsis (e.g., neonatal sepsis), or pneumonia.

### CROSS-REFERENCE TO RELATED APPLICATIONS

This application claims priority under 35 U.S.C. § 119(e) to U.S. Provisional Patent Application No. 63/471,365, filed Jun. 6, 2023, and U.S. Provisional Patent Application No. 63/535,969, filed Aug. 31, 2023, all of which applications are incorporated herein by reference in their entireties.

### REFERENCE TO AN ELECTRONIC SEQUENCE LISTING

The XML filed named “375836_7000WO1_SequenceListing.xml” created on Jun. 5, 2024, comprising 245 KB, is hereby incorporated herein by reference in its entirety.

### BACKGROUND

Bacterial infections (e.g., urinary tract infection (UTI), sepsis, and pneumonia, inter alia) pose a significant threat to human health, leading to substantial morbidity and mortality worldwide, including infant mortality (e.g., neonatal sepsis).

While infections such as UTIs, sepsis (e.g., neonatal sepsis), and pneumonia may be caused by any of a number of bacteria, viruses, and/or fungi, these infections most commonly have a bacterial origin (e.g., Escherichia coli, Klebsiella pneumoniae, and Proteus mirabilis are generally considered the most common cause of UTI). Accordingly, antibiotics are generally prescribed as a first line treatment (e.g., ciprofloxacin). However, in addition to the risks associated with overdependence on antibiotics (e.g., development of antibiotic resistance), such antibiotics may cause side effects including, but not limited to, nausea, vomiting, stomach pain, heartburn, diarrhea, and fatigue.

Thus, there is a need in the art for compositions and/or methods for preventing a bacterial infection (e.g., UTI, sepsis, and/or pneumonia), and/or generating immunity to infection by one or more bacterial pathogens, non-limiting examples including E. coli. The present disclosure addresses this need.

### BRIEF SUMMARY

In one aspect, the disclosure provides a polypeptide comprising formula (I), or a salt or solvate thereof, wherein n and each occurrence of B¹, B², B³, L¹, L², L³, L⁴, L⁵, L⁶, T¹, T², and T³ are defined elsewhere herein:

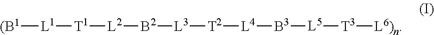

In another aspect, the disclosure provides a polypeptide comprising B¹-L¹-T¹-L²-B²-L³-T²-L⁴-B³-L⁵-T³-L⁶-B⁴-L⁷-T⁴-L⁸-B⁵-L⁹-T⁵-L¹⁰-B⁶-L¹¹-T⁶-L¹²-B⁷-L¹³-T⁷-L¹⁴-B⁸-L¹⁵-T⁸-L¹⁶-B⁹-L¹⁷-T⁹-L¹⁸-B¹⁰-L¹⁹-T¹⁰-L²⁰-B¹¹-L²¹-T¹¹-L²²-B¹²-L²³-T¹², wherein B¹, B², B³, B⁴, B⁵, B⁶, B⁷, B⁸, B⁹, B¹⁰, B¹¹, B¹², L¹, L², L³, L⁴, L⁵, L⁶, L⁷, L⁸, L⁹, L¹⁰, L¹¹, L¹², L¹³, L¹⁴, L¹⁵, L¹⁶, L¹⁷, L¹⁸, L¹⁹, L²⁰, L²¹, L²², L²³, T¹, T², T³, T⁴, T⁵, T⁶, T⁷, T⁸, T⁹, T¹⁰, T¹¹, and T¹² are defined elsewhere herein.

In another aspect, the disclosure provides a polypeptide comprising T¹-L¹-T²-L²-T³-L³-T⁴-L⁴-T⁵-L⁵-T⁶-L⁶-T⁷-L⁷-T⁸-L⁸-T⁹-L⁹-T¹⁰-L¹⁰-T¹¹-L¹¹-T¹², wherein L¹, L², L³, L⁴, L⁵, L⁶, L⁷, L⁸, L⁹, L¹⁰, L¹¹, T¹, T², T³, T⁴, T⁵, T⁶, T⁷, T⁸, T⁹, T¹⁰, T¹¹, and T¹² are defined elsewhere herein.

In another aspect, the disclosure provides a polypeptide comprising B¹-L¹-T¹-L²-B²-L³-T²-L⁴-B³-L⁵-T³-L⁶-B⁴-L⁷-T⁴-L⁸-B⁵-L⁹-T⁵-L¹⁰-B⁶-L¹¹-T⁶-L¹²-B⁷-L¹³-T⁷-L¹⁴-B⁸-L¹⁵-T⁸-L¹⁶-B⁹-L¹⁷-T⁹, wherein B¹, B², B³, B⁴, B⁵, B⁶, B⁷, B⁸, B⁹, L¹, L², L³, L⁴, L⁵, L⁶, L⁷, L⁸, L⁹, L¹⁰, L¹¹, L¹², L¹³, L¹⁴, L¹⁵, L¹⁶, L¹⁷, T¹, T², T³, T⁴, T⁵, T⁶, T⁷, T⁸, and T⁹ are defined elsewhere herein.

In another aspect, the disclosure provides a polypeptide comprising T¹-L¹-T²-L²-T³-L³-T⁴-L⁴-T⁵-L⁵-T⁶-L⁶-T⁷-L⁷-T⁸-L⁸-T⁹, wherein L¹, L², L³, L⁴, L⁵, L⁶, L⁷, L⁸, T¹, T², T³, T⁴, T⁵, T⁶, T⁷, T⁸, and T⁹ are defined elsewhere herein.

In another aspect, the disclosure provides a polypeptide comprising B¹-L¹-T¹-L²-B²-L³-T²-L⁴-B³-L⁵-T³-L⁶-B⁴-L⁷-T⁴-L⁸-B⁵-L⁹-T⁵-L¹⁰-B⁶-L¹¹-T⁶-L¹²-B⁷-L¹³-T⁷-L¹⁴-B⁸-L¹⁵-T⁸-L¹⁶-B⁹-L¹⁷-T¹-L¹⁸-B¹⁰-L¹⁹-T²-L²⁰-B¹¹-L²¹-T³-L²²-B⁴-L²³-T⁴, wherein B¹, B², B³, B⁴, B⁵, B⁶, B⁷, B⁸, B⁹, B¹⁰, B¹¹, B¹², L¹, L², L³, L⁴, L⁵, L⁶, L⁷, L⁸, L⁹, L¹⁰, L¹¹, L¹², L¹³, L¹⁴, L¹⁵, L¹⁶, L¹⁷, L¹⁸, L¹⁹, L²⁰, L²¹, L²², L²³, T¹, T², T³, and T⁴ are defined elsewhere herein.

In another aspect, the disclosure provides a polypeptide comprising B¹-L¹-T¹-L²-B²-L³-T²-L⁴-B³-L⁵-T³-L⁶-B⁴-L⁷-T⁴-L⁸-B⁵-L⁹-T⁵-L¹⁰-B⁶-L¹¹-T⁶-L¹²-B⁷-L¹³-T⁷-L¹⁴-B⁸-L¹⁵-T⁸-L¹⁶-B⁹-L¹⁷-T⁹-L¹⁸-B¹⁰-L¹⁹-T¹⁰-L²⁰-B¹¹-L²¹-T¹¹-L²²-B¹²-L²³-T¹²-L²⁴-B¹³-L²⁵-T¹³, wherein B¹, B², B³, B⁴, B⁵, B⁶, B⁷, B⁸, B⁹, B¹⁰, B¹¹, B¹², B¹³, L¹, L², L³, L⁴, L⁵, L⁶, L⁷, L⁸, L⁹, L¹⁰, L¹¹, L¹², L¹³, L¹⁴, L¹⁵, L¹⁶, L¹⁷, L¹⁸, L¹⁹, L²⁰, L²¹, L²², L²³, L²⁴, L²⁵, T¹, T², T³, T⁴, T⁵, T⁶, T⁷, T⁸, T⁹, T¹⁰, T¹¹, T¹², and T¹³ are defined elsewhere herein.

In another aspect, the disclosure provides a polypeptide comprising T¹-L¹-T²-L²-T³-L³-T⁴-L⁴-T⁵-L⁵-T⁶-L⁶-T⁷-L⁷-T⁸-L⁸-T⁹-L⁹-T¹⁰-L¹⁰-T¹¹-L¹¹-T¹²-L¹²-T¹³, wherein L¹, L², L³, L⁴, L⁵, L⁶, L⁷, L⁸, L⁹, L¹⁰, L¹¹, L¹², T¹, T², T³, T⁴, T⁵, T⁶, T⁷, T⁸, T⁹, T¹⁰, T¹¹, T¹², and T¹³ are defined elsewhere herein.

In another aspect, the disclosure provides a polypeptide comprising B¹-L¹-T¹-L²-B²-L³-T²-L⁴-B³-L⁵-T³-L⁶-B⁴-L⁷-T⁴-L⁸-B⁵-L⁹-T⁵-L¹⁰-B⁶-L¹¹-T⁶-L¹²-B⁷-L¹³-T⁷-L¹⁴-B⁸-L¹⁵-T⁸-L¹⁶-B⁹-L¹⁷-T⁹-L¹⁸-B¹⁰-L¹⁹-T¹⁰-L²⁰-B¹¹-L²¹-T¹¹-L²²-B¹²-L²³-T¹²-L²⁴-B¹³, wherein B¹, B², B³, B⁴, B⁵, B⁶, B⁷, B⁸, B⁹, B¹⁰, B¹¹, B¹², B¹³, L¹, L², L³, L⁴, L⁵, L⁶, L⁷, L⁸, L⁹, L¹⁰, L¹¹, L¹², L¹³, L¹⁴, L¹⁵, L¹⁶, L¹⁷, L¹⁸, L¹⁹, L²⁰, L²¹, L²², L²³, L²⁴, T¹, T², T³, T⁴, T⁵, T⁶, T⁷, T⁹, T¹⁰, T¹¹, and T¹² are defined elsewhere herein.

In another aspect, the disclosure provides a polypeptide comprising B¹-L¹-B²-L²-B³-L³-B⁴-L⁴-T¹-L⁵-T²-L⁶-T³-L⁷-T⁴-L⁸-B⁵-L⁹-B⁶-L¹⁰-B⁷-L¹¹-B⁸-L¹²-T⁵-L¹³-T⁶-L¹⁴-T⁷-L¹⁵-T⁸-L¹⁶-B⁹-L¹⁷-B¹⁰-L¹⁸-B¹¹-L¹⁹-B¹²-L²⁰-T⁹-L²¹-T¹⁰-L²²-T¹¹-L²³-T¹², wherein B¹, B², B³, B⁴, B⁵, B⁶, B⁷, B⁸, B⁹, B¹⁰, B¹¹, B¹², L¹, L², L³, L⁴, L⁵, L⁶, L⁷, L⁸, L⁹, L¹⁰, L¹¹, L¹², L¹³, L¹⁴, L¹⁵, L¹⁶, L¹⁷, L¹⁸, L¹⁹, L²⁰, L²¹, L²², L²³, T¹, T², T³, T⁴, T⁵, T⁶, T⁷, T⁸, T⁹, T¹⁰, T¹¹, and T¹² are defined elsewhere herein.

In another aspect, the disclosure provides a polypeptide comprising T¹-L¹-T²-L²-T³-L³-T⁴-L⁴-B¹-L⁵-B²-L⁶-B³-L⁷-B⁴-L⁸-T⁵-L⁹-T⁶-L¹⁰-T⁷-L¹¹-T⁸-L¹²-B⁵-L¹³-B⁶-L¹⁴-B⁷-L¹⁵-B⁸-L¹⁶-T⁹-L¹⁷-T¹⁰-L¹⁸-T¹¹-L¹⁹-T¹²-L²⁰-B⁹-L²¹-B¹⁰-L²²-B¹¹-L²³-B¹², wherein B¹, B², B³, B⁴, B⁵, B⁶, B⁷, B⁸, B⁹, B¹⁰, B¹¹, B¹², L¹, L², L³, L⁴, L⁵, L⁶, L⁷, L⁸, L⁹, L¹⁰, L¹¹, L¹², L¹³, L¹⁴, L¹⁵, L¹⁶, L¹⁷, L¹⁸, L¹⁹, L²⁰, L²¹, L²², L²³, T¹, T², T³, T⁴, T⁵, T⁶, T⁷, T⁸, T⁹, T¹⁰, T¹¹, and T¹² are defined elsewhere herein.

In another aspect, the disclosure provides an isolated messenger ribonucleic acid (mRNA) encoding a polypeptide of the disclosure.

In another aspect, the disclosure provides an isolated deoxyribonucleic acid (DNA) encoding a polypeptide of the disclosure.

In another aspect, the disclosure provides an isolated polynucleotide encoding an mRNA of the disclosure, wherein the polynucleotide comprises one or more promoters and/or a polyadenylation signal operably linked to a sequence encoding the mRNA.

In another aspect, the disclosure provides a vector comprising an isolated mRNA of the disclosure, an isolated DNA of the disclosure, and/or an isolated polynucleotide of the disclosure.

In another aspect, the disclosure provides a lipid nanoparticle (LNP) composition comprising an isolated mRNA of the disclosure, an isolated DNA of the disclosure, and/or an isolated polynucleotide of the disclosure.

In another aspect, the disclosure provides a pharmaceutical composition comprising an LNP of the disclosure and a pharmaceutically acceptable carrier.

In another aspect, the disclosure provides a vaccine composition comprising an LNP of the disclosure and/or a pharmaceutical composition of the disclosure.

In another aspect, the disclosure provides a vaccine composition comprising a polypeptide of the disclosure and at least one pharmaceutically acceptable excipient.

In another aspect, the disclosure provides a method of treating, preventing, and/or ameliorating a bacterial infection in a subject in need thereof, the method comprising administering to the subject a polypeptide of the disclosure.

In another aspect, the disclosure provides a method of treating, preventing, and/or ameliorating a bacterial infection in a subject in need thereof, the method comprising administering to the subject an isolated mRNA of the disclosure.

In another aspect, the disclosure provides a method of treating, preventing, and/or ameliorating a bacterial infection in a subject in need thereof, the method comprising administering to the subject an isolated DNA of the disclosure.

In another aspect, the disclosure provides a method of treating, preventing, and/or ameliorating a bacterial infection in a subject in need thereof, the method comprising administering to the subject an isolated polynucleotide of the disclosure.

In another aspect, the disclosure provides a method of treating, preventing, and/or ameliorating a bacterial infection in a subject in need thereof, the method comprising administering to the subject a vector of the disclosure.

In another aspect, the disclosure provides a method of treating, preventing, and/or ameliorating a bacterial infection in a subject in need thereof, the method comprising administering to the subject a LNP of the disclosure.

In another aspect, the disclosure provides a method of treating, preventing, and/or ameliorating a bacterial infection in a subject in need thereof, the method comprising administering to the subject a pharmaceutical composition of the disclosure.

In another aspect, the disclosure provides a method of treating, preventing, and/or ameliorating a bacterial infection in a subject in need thereof, the method comprising administering to the subject a vaccine composition of the disclosure.

In another aspect, the disclosure provides a method of generating immunity to infection by one or more pathogenic bacteria in a subject, the method comprising administering to the subject a polypeptide of the disclosure.

In another aspect, the disclosure provides a method of generating immunity to infection by one or more pathogenic bacteria in a subject, the method comprising administering to the subject an isolated mRNA of the disclosure.

In another aspect, the disclosure provides a method of generating immunity to infection by one or more pathogenic bacteria in a subject, the method comprising administering to the subject an isolated DNA of the disclosure.

In another aspect, the disclosure provides a method of generating immunity to infection by one or more pathogenic bacteria in a subject, the method comprising administering to the subject an isolated polynucleotide of the disclosure.

In another aspect, the disclosure provides a method of generating immunity to infection by one or more pathogenic bacteria in a subject, the method comprising administering to the subject a vector of the disclosure.

In another aspect, the disclosure provides a method of generating immunity to infection by one or more pathogenic bacteria in a subject, the method comprising administering to the subject a LNP of the disclosure.

In another aspect, the disclosure provides a method of generating immunity to infection by one or more pathogenic bacteria in a subject, the method comprising administering to the subject a pharmaceutical composition of the disclosure.

In another aspect, the disclosure provides a method of generating immunity to infection by one or more pathogenic bacteria in a subject, the method comprising administering to the subject a vaccine composition of the disclosure.

### BRIEF DESCRIPTION OF THE FIGURES

The drawings illustrate generally, by way of example, but not by way of limitation, various embodiments of the present application.

FIG. 1 provides a graph showing serum IL-6 concentration in HLA-DR4 mice 24 h after 30 second vaccination, wherein mice were vaccinated as follows: no vaccination (i.e., naïve; Group 1); SEQ ID NO:127 (AlOH+dmLT adjuvant; Group 2); SEQ ID NO:128 (AlOH+dmLT adjuvant; Group 3); SEQ ID NO:127 (AlOH+CpG adjuvant; Group 4); and SEQ ID NO:128 (AlOH+CpG adjuvant; Group 5); following the vaccination protocol described elsewhere herein (Exp. No. 1).

FIGS. 2A-2D provide graphs showing serum titers of IgG1 (FIG. 2A), IgG2b (FIG. 2B), and levels for serum IgA (FIG. 2C) and urine IgG(H) (FIG. 2D) in HLA-DR4 mice approximately 14 days after second vaccination (i.e., day 35) with: no vaccination (i.e., naïve; Group 1); SEQ ID NO:127 (AlOH+dmLT adjuvant; Group 2); and SEQ ID NO:127 (AlOH+CpG adjuvant; Group 4); as determined by standard ELISA, following the vaccination protocol described elsewhere herein (Exp. No. 1).

FIGS. 3A-3B provide graphs showing serum titers of IgG1 (FIG. 3A) and IgG2b (FIG. 3B) in HLA-DR4 mice approximately 14 days after second vaccination (i.e., day 35) with: no vaccination (i.e., naïve; Group 1); SEQ ID NO:128 (AlOH+dmLT adjuvant; Group 3); and SEQ ID NO:128 (AlOH+CpG adjuvant; Group 5); as determined by standard ELISA, following the vaccination protocol described elsewhere herein (Exp. No. 1).

FIGS. 4A-4C provide graphs showing serum (FIG. 4A) and urine (FIGS. 4B-4C) levels of IgA (FIGS. 4A-4B) and IgG(H) (FIG. 4C) in HLA-DR4 mice approximately 14 days after second vaccination (i.e., day 35) with: no vaccination (i.e., naïve; Group 1); SEQ ID NO:128 (AlOH+dmLT adjuvant; Group 3); and SEQ ID NO:128 (AlOH+CpG adjuvant; Group 5); as determined by standard ELISA, following the vaccination protocol described elsewhere herein (Exp. No. 1).

FIG. 5 provides a bar graph showing the results of a peptide ELISA, wherein wells were coated with certain polypeptides (i.e., SEQ ID NOs:17, 20, 24, 40, 197-199, 163-168, 176-182, 188-192, 200-202, 206-207, 213-215, 218-219, and 127-128) and controls (i.e., tetanus toxin (TTX), serum albumin (SA) and no coating) and utilized sera from HLA-DR4 mice approximately 14 days after second vaccination with: no vaccination (Group 1); SEQ ID NO:127 (AlOH+dmLT adjuvant; Group 2); and SEQ ID NO:128 (AlOH+CpG adjuvant; Group 4); following the vaccination protocol described elsewhere herein (Exp. No. 1). Three bars are depicted for each coating indicated along the x-axis (e.g., SEQ ID NO:17), wherein the left, center, and right bars represent data corresponding to Groups 1, 2, and 4 of Exp. No. 1, respectively.

FIGS. 6A-6C provides bar graphs showing production of Th1/Th17/pro-inflammatory cytokines IL-17A (FIG. 6A), IL-2 (FIG. 6B), and IL-6 (FIG. 6C) by splenocytes from HLA-DR4 mice, approximately 22 days after the second vaccination (i.e., day 43), immunized with: no vaccination (i.e., naïve; Group 1); SEQ ID NO:127 (AlOH+dmLT adjuvant; Group 2); SEQ ID NO:128 (AlOH+dmLT adjuvant; Group 3); SEQ ID NO:127 (AlOH+CpG adjuvant; Group 4); and SEQ ID NO:128 (AlOH+CpG adjuvant; Group 5); upon restimulation with SEQ ID NO:127 and SEQ ID NO:128, as determined by Cytometric Bead Array (CBA) (BD Biosciences).

FIGS. 7A-7B provide bar graphs showing production of Th1/pro-inflammatory cytokines TNF-α (FIG. 7A) and IFN-γ (FIG. 7B) by splenocytes from HLA-DR4 mice, approximately 22 days after the second vaccination (i.e., day 43), immunized with: no vaccination (i.e., naïve; Group 1); SEQ ID NO:127 (AlOH+dmLT adjuvant; Group 2); SEQ ID NO:128 (AlOH+dmLT adjuvant; Group 3); SEQ ID NO:127 (AlOH+CpG adjuvant; Group 4); and SEQ ID NO:128 (AlOH+CpG adjuvant; Group 5); upon restimulation with SEQ ID NO:127 and SEQ ID NO:128, as determined by Cytometric Bead Array (CBA) (BD Biosciences).

FIGS. 8A-8B provide bar graphs showing production of Th2/anti-inflammatory cytokines IL-4 (FIG. 8A) and IL-10 (FIG. 8B) by splenocytes from HLA-DR4 mice, approximately 22 days after the second vaccination (i.e., day 43), immunized with: no vaccination (i.e., naïve; Group 1); SEQ ID NO:127 (AlOH+dmLT adjuvant; Group 2); SEQ ID NO:128 (AlOH+dmLT adjuvant; Group 3); SEQ ID NO:127 (AlOH+CpG adjuvant; Group 4); and SEQ ID NO:128 (AlOH+CpG adjuvant; Group 5); upon restimulation with SEQ ID NO:127 and SEQ ID NO:128, as determined by Cytometric Bead Array (CBA) (BD Biosciences).

FIGS. 9A-9B provide graphs showing serum titers of IgG1 (FIG. 9A) and IgG2b (FIG. 9B) in HLA-DR4 mice approximately 14 days after second vaccination (i.e., day 28) with: placebo (i.e., AlOH+CpG adjuvant; Group A); or SEQ ID NO:132 (AlOH+CpG adjuvant; Group D); as determined by standard ELISA, following the vaccination protocol described elsewhere herein (Exp. No. 2).

FIGS. 10A-10B provide graphs showing serum titers of IgG1 (FIG. 10A) and IgG2b (FIG. 10B) in HLA-DR4 mice approximately 14 days after second vaccination (i.e., day 28) with: placebo (i.e., AlOH+CpG adjuvant; Group A); or SEQ ID NO:133 (AlOH+CpG adjuvant; Group E); as determined by standard ELISA, following the vaccination protocol described elsewhere herein (Exp. No. 2).

FIGS. 11A-11B provide graphs showing serum titers of IgG1 (FIG. 11A) and IgG2b (FIG. 11B) in HLA-DR4 mice approximately 14 days after second vaccination (i.e., day 28) with: placebo (i.e., AlOH+CpG adjuvant; Group A); or SEQ ID NO:135 (AlOH+CpG adjuvant; Group F); as determined by standard ELISA, following the vaccination protocol described elsewhere herein (Exp. No. 2).

FIG. 12 provides a bar graph showing the results of a peptide ELISA, wherein wells were coated with certain polypeptides (i.e., SEQ ID NOs: 17, 20, 24, 40, 197-199, 163-168, 176-182, 188-192, 200-202, 206-207, 213-215, 218-219, 132-133, and 135) and controls (i.e., tetanus toxin (TTX), serum albumin (SA) and no coating) and utilized sera from HLA-DR4 mice approximately 28 days after initial vaccination with: placebo (i.e., AlOH+CpG adjuvant; Group A); or SEQ ID NO:132 (AlOH+CpG adjuvant; Group D); following the vaccination protocol described elsewhere herein (Exp. No. 2). Two bars are depicted for each coating indicated along the x-axis (e.g., SEQ ID NO:17), wherein the left and right bars represent data corresponding to Groups A and D of Exp. No. 2, respectively.

FIGS. 13A-13C provide bar graphs showing production of Th1/Th17/pro-inflammatory cytokines IL-17A (FIG. 13A), IL-2 (FIG. 13B), and IL-6 (FIG. 13C) by splenocytes from HLA-DR4 mice immunized with: placebo (i.e., AlOH+CpG adjuvant; Group A); SEQ ID NO:127 (AlOH+CpG adjuvant; Group B); SEQ ID NO:128 (AlOH+CpG adjuvant; Group C); SEQ ID NO:132 (AlOH+CpG adjuvant; Group D); SEQ ID NO:133 (AlOH+CpG adjuvant; Group E); or SEQ ID NO:135 (AlOH+CpG adjuvant; Group F); upon restimulation with SEQ ID NO:132, SEQ ID NO:133, or SEQ ID NO:135, as determined by Cytometric Bead Array (CBA) (BD Biosciences).

FIGS. 14A-14B provide bar graphs showing production of Th1/pro-inflammatory cytokines TNF-α (FIG. 14A) and IFN-γ (FIG. 14B) by splenocytes from HLA-DR4 mice immunized with: placebo (i.e., AlOH+CpG adjuvant; Group A); SEQ ID NO:132 (AlOH+CpG adjuvant; Group D); SEQ ID NO:133 (AlOH+CpG adjuvant; Group E); or SEQ ID NO:135 (AlOH+CpG adjuvant; Group F); upon restimulation with SEQ ID NO:132, SEQ ID NO:133, or SEQ ID NO:135, as determined by Cytometric Bead Array (CBA) (BD Biosciences).

FIGS. 15A-15B provide bar graphs showing production of Th2/anti-inflammatory cytokines IL-4 (FIG. 15A) and IL-10 (FIG. 15B) by splenocytes from HLA-DR4 mice immunized with: placebo (i.e., AlOH+CpG adjuvant: Group A); SEQ ID NO:132 (AlOH+CpG adjuvant; Group D); SEQ ID NO:133 (AlOH+CpG adjuvant; Group E); or SEQ ID NO:135 (AlOH+CpG adjuvant; Group F); upon restimulation with SEQ ID NO:132, SEQ ID NO:133, or SEQ ID NO:135, as determined by Cytometric Bead Array (CBA) (BD Biosciences).

FIGS. 16A-16B provide graphs showing serum titers of IgG1 (FIG. 16A) and IgG2b (FIG. 16B) in HLA-DR4 mice as a function of time (days) after vaccination with SEQ ID NO:132 (AddaS03™+CpG adjuvant); as determined by standard ELISA, following the vaccination protocol described herein (Exp. No. 3).

FIGS. 17A-17C provide graphs showing serum (FIG. 17A) and urine (FIGS. 17B-17C) levels of IgA (FIGS. 17A-17B) and IgG(H) (FIG. 17C) in HLA-DR4 mice as a function of time (days) after vaccination with SEQ ID NO:132 (AddaS03™+CpG adjuvant); as determined by standard ELISA, following the vaccination protocol described herein (Exp. No. 3).

FIG. 18 provides a bar graph showing the results of a peptide ELISA, wherein wells were coated with certain polypeptides (i.e., SEQ ID NOs: 17, 20, 24, 40, 197-199, 163-168, 176-182, 188-192, 200-202, 206-207, 213-215, 218-219, 132-133, and 135) and controls (i.e., tetanus toxin (TTX), serum albumin (SA) and no coating) and utilized sera from HLA-DR4 mice approximately 21 and 41 days after vaccination with SEQ ID NO:132 (AddaS03™+CpG adjuvant); following the vaccination protocol described elsewhere herein (Exp. No. 3). Two bars are depicted for each coating indicated along the x-axis (e.g., SEQ ID NO:17), wherein the left and right bars represent data corresponding to samples collected from mice 21 days after vaccination and 41 days after vaccination, respectively.

FIGS. 19A-19B provide graphs showing serum titers of IgG1 (FIG. 19A) and IgG2b (FIG. 19B) in HLA-DR4 mice approximately 14 days after second vaccination with: placebo (i.e., AlOH adjuvant; Group A); or SEQ ID NO:132 (AlOH adjuvant; Group B); as determined by standard ELISA, following the vaccination protocol described herein (Exp. No. 8).

FIGS. 20A-20C provide graphs showing serum (FIG. 20A) and urine (FIGS. 20B-20C) levels of IgA (FIGS. 20A-20B) and IgG(H) (FIG. 20C) in HLA-DR4 mice approximately 14 days after second vaccination with: placebo (i.e., AlOH adjuvant; Group A); or SEQ ID NO:132 (AlOH adjuvant; Group B); as determined by standard ELISA, following the vaccination protocol described herein (Exp. No. 8).

FIGS. 21A-21B provide graphs showing serum titers of IgG1 (FIG. 21A) and IgG2b (FIG. 21B) in HLA-DR4 mice approximately 14 days after second vaccination with: placebo (i.e., AlOH adjuvant; Group A); or SEQ ID NO:135 (AlOH adjuvant; Group C); as determined by standard ELISA, following the vaccination protocol described herein (Exp. No. 8).

FIGS. 22A-22C provide graphs showing serum (FIG. 22A) and urine (FIGS. 22B-22C) levels of IgA (FIGS. 22A-22B) and IgG(H) (FIG. 22C) in HLA-DR4 mice approximately 14 days after second vaccination with: placebo (i.e., AlOH adjuvant; Group A); or SEQ ID NO:135 (AlOH adjuvant; Group C); as determined by standard ELISA, following the vaccination protocol described herein (Exp. No. 8).

FIG. 23 provides a bar graph showing the results of a peptide ELISA, wherein wells were coated with certain polypeptides (i.e., SEQ ID NOs: 17, 20, 24, 40, 197-199, 163-168, 176-182, 188-192, 200-202, 206-207, 213-215, 218-219, 132-133, and 135) and controls (i.e., tetanus toxin (TTX), serum albumin (SA) and no coating) and utilized sera from HLA-DR4 mice approximately 14 days after second vaccination with: placebo (i.e., AlOH adjuvant; Group A); or SEQ ID NO:132 (AlOH adjuvant; Group B); following the vaccination protocol described elsewhere herein (Exp. No. 8). Two bars are depicted for each coating indicated along the x-axis (e.g., SEQ ID NO:17), wherein the left and right bars represent data corresponding to Groups A and B of Exp. No. 8, respectively.

FIG. 24 provides a bar graph showing the results of a peptide ELISA, wherein wells were coated with certain polypeptides (i.e., SEQ ID NOs: 17, 20, 24, 40, 197-199, 163-168, 176-182, 188-192, 200-202, 206-207, 213-215, 218-219, 132-133, and 135) and controls (i.e., tetanus toxin (TTX), serum albumin (SA) and no coating) and utilized sera from CD1 mice approximately 14 days after second vaccination with: placebo (i.e., AlOH adjuvant; Group A); or SEQ ID NO:135 (AlOH adjuvant; Group C); following the vaccination protocol described elsewhere herein (Exp. No. 8). Two bars are depicted for each coating indicated along the x-axis (e.g., SEQ ID NO:17), wherein the left and right bars represent data corresponding to Groups A and C of Exp. No. 8, respectively.

FIGS. 25A-25C provides bar graphs showing production of Th1/Th17/pro-inflammatory cytokines IL-17A (FIG. 25A), IL-2 (FIG. 25B), and IL-6 (FIG. 25C) by CD1 splenocytes immunized with: placebo (i.e., AlOH adjuvant; Group A); SEQ ID NO:132 (AlOH adjuvant; Group B); or SEQ ID NO:135 (AlOH adjuvant; Group C); upon restimulation with SEQ ID NO:132 or SEQ ID NO:135, as determined by Cytometric Bead Array (CBA) (BD Biosciences).

FIGS. 26A-26B provide bar graphs showing production of Th1/pro-inflammatory cytokines TNF-α (FIG. 26A) and IFN-γ (FIG. 26B) by CD1 splenocytes immunized with: placebo (i.e., AlOH adjuvant; Group A); SEQ ID NO:132 (AlOH adjuvant; Group B); or SEQ ID NO:135 (AlOH adjuvant; Group C); upon restimulation with SEQ ID NO:132 or SEQ ID NO:135, as determined by Cytometric Bead Array (CBA) (BD Biosciences).

FIGS. 27A-27B provide bar graphs showing production of Th2/anti-inflammatory cytokines IL-4 (FIG. 27A) and IL-10 (FIG. 27B) by CD1 splenocytes immunized with: placebo (i.e., AlOH adjuvant; Group A); SEQ ID NO:132 (AlOH adjuvant; Group B); or SEQ ID NO:135 (AlOH adjuvant; Group C); upon restimulation with SEQ ID NO:132 or SEQ ID NO:135, as determined by Cytometric Bead Array (CBA) (BD Biosciences).

FIGS. 28A-28E provide graphs showing serum titers or levels of IgG1 (FIG. 28A), IgG2b (FIG. 28B), and IgA (FIG. 28C), and urine levels of IgA (FIG. 28D and IgG(H) (FIG. 28E) in HLA-DR4 mice as a function of time (days) after vaccination with SEQ ID NO:136 (AddaS03™+CpG adjuvant) or a placebo; as determined by standard ELISA, following the vaccination protocol described herein (Exp. No. 4).

FIGS. 29A-29E provide graphs showing serum titers or levels of IgG1 (FIG. 29A), IgG2b (FIG. 29B), and IgA (FIG. 29C), and urine levels of IgA (FIG. 29D and IgG(H) (FIG. 29E) in HLA-DR4 mice as a function of time (days) after vaccination with SEQ ID NO:137 (AddaS03™+CpG adjuvant) or a placebo; as determined by standard ELISA, following the vaccination protocol described herein (Exp. No. 4).

FIGS. 30A-30E provide graphs showing serum titers or levels of IgG1 (FIG. 30A), IgG2b (FIG. 30B), and IgA (FIG. 30C), and urine levels of IgA (FIG. 30D and IgG(H) (FIG. 30E) in HLA-DR4 mice as a function of time (days) after vaccination with SEQ ID NO:138 (AddaS03™+CpG adjuvant) or a placebo; as determined by standard ELISA, following the vaccination protocol described herein (Exp. No. 4).

FIGS. 31A-31E provide graphs showing serum titers or levels of IgG1 (FIG. 31A), IgG2b (FIG. 31B), and IgA (FIG. 31C), and urine levels of IgA (FIG. 31D and IgG(H) (FIG. 31E) in HLA-DR4 mice as a function of time (days) after vaccination with SEQ ID NO:139 (AddaS03™+CpG adjuvant) or a placebo; as determined by standard ELISA, following the vaccination protocol described herein (Exp. No. 4).

FIGS. 32A-32E provide graphs showing serum titers or levels of IgG1 (FIG. 32A), IgG2b (FIG. 32B), and IgA (FIG. 32C), and urine levels of IgA (FIG. 32D and IgG(H) (FIG. 32E) in HLA-DR4 mice as a function of time (days) after vaccination with SEQ ID NO:140 (AddaS03™+CpG adjuvant) or a placebo; as determined by standard ELISA, following the vaccination protocol described herein (Exp. No. 4).

FIGS. 33A-33C provides a bar graph showing the results of a peptide ELISA, wherein wells were coated with certain polypeptides (i.e., SEQ ID NOs: 17, 20, 25, 27, 195, 197-199, 163-168, 176-182, 188-192, 207, 213-215, 218-219, 200-203, and 136-140) and controls (i.e., tetanus toxin (TTX), serum albumin (SA) and no coating) and utilized sera from HLA-DR4 mice approximately 14 days after second vaccination with: placebo (Group A); SEQ ID NO:136 (Group B) (FIG. 33A); SEQ ID NO:138 (Group D) (FIG. 33B); or SEQ ID NO:140 (Group F) (FIG. 33C); following the vaccination protocol described elsewhere herein (Exp. No. 4). Two bars are depicted for each coating indicated along the x-axis (e.g., SEQ ID NO:17), wherein the left and right bars represent data corresponding to Groups A and B (FIG. 33A), respectively; Groups A and D (FIG. 33B), respectively; and Groups A and F (FIG. 33C), respectively; of Exp. No. 4.

FIGS. 34A-34F provide bar graphs showing production of Th1/Th17/pro-inflammatory cytokines or Th2/anti-inflammatory cytokines: IFN-γ (FIG. 34A), IL-4 (FIG. 34B), IL-6 (FIG. 34C), IL-22 (FIG. 34D), IL-10 (FIG. 34E), and IL-17A (FIG. 34F) by splenocytes from HLA-DR4 mice immunized with placebo (AddaS03™+CpG; Group A); SEQ ID NO:136 (AddaS03™+CpG; Group B); SEQ ID NO:137 (AddaS03™+CpG; Group C); SEQ ID NO:138 (AddaS03™+CpG; Group D); SEQ ID NO:139 (AddaS03™+CpG; Group E); and SEQ ID NO:140 (AddaS03™+CpG; Group F) (Fxp. No. 4), upon restimulation with SEQ ID NO:136, SEQ ID NO:137, SEQ ID NO:138, SEQ ID NO:139, SEQ ID NO:140, or no stimulation, approximately 49 days post first vaccination and approximately 3 days after UPEC25 challenge, as determined by Cytometric Bead Array (CBA) (MAGPIX® system).

FIGS. 35A-35C provides bar graphs depicting the percentage of activation induced markers (AIMs) OX40 and PD-L1 (FIG. 35A), CD69 and PD-L1 (FIG. 35B), and CD25 and PD-L1 (FIG. 35C) in splenocytes approximately 3 days post challenge with UPEC25, and approximately 49 days after first vaccination with placebo (Group A); SEQ ID NO:136 (Group B); SEQ ID NO:137 (Group C); SEQ ID NO:138 (Group D); SEQ ID NO:139 (Group E); and SEQ ID NO:140 (Group F); after restimulation with SEQ ID NO:136; SEQ ID NO:137; SEQ ID NO:138; SEQ ID NO:139; and SEQ ID NO:140; or no stimulation; following the vaccination protocol described elsewhere herein (Exp. No. 4).

FIGS. 36A-36E provide graphs showing serum titers or levels of IgG1 (FIG. 36A), IgG2b (FIG. 36B), and IgA (FIG. 36C), and urine levels of IgA (FIG. 36D and IgG(H) (FIG. 36E) in HLA-DR4 mice as a function of time (days) after vaccination with SEQ ID NO:136 (Group B); SEQ ID NO:136 (Group E); or a placebo (Group A); as determined by standard ELISA, following the vaccination protocol described herein (Exp. No. 5).

FIGS. 37A-37E provide graphs showing serum titers or levels of IgG1 (FIG. 37A), IgG2b (FIG. 37B), and IgA (FIG. 37C), and urine levels of IgA (FIG. 37D and IgG(H) (FIG. 37E) in HLA-DR4 mice as a function of time (days) after vaccination with SEQ ID NO:138 (Group C); SEQ ID NO:138 (Group F); or a placebo (Group D); as determined by standard ELISA, following the vaccination protocol described herein (Exp. No. 5).

FIGS. 38A-38B provides a bar graph showing the results of a peptide ELISA, wherein wells were coated with certain polypeptides (i.e., SEQ ID NOs: 16-17, 20, 25, 27, 195, 197-199, 163-168, 176-182, 188-192, 207, 213-215, 218-219, 200-203 and 136-140) and controls (i.e., tetanus toxin (TTX), serum albumin (SA) and no coating) and utilized sera from HLA-DR4 mice approximately 14 days after second vaccination with: SEQ ID NO:136 (Group B) or SEQ ID NO:138 (Group C) (FIG. 38A); and SEQ ID NO:136 (Group E) or SEQ ID NO:138 (Group F) (FIG. 38B); following the vaccination protocol described elsewhere herein (Exp. No. 5). Two bars are depicted for each coating indicated along the x-axis (e.g., SEQ ID NO:17), wherein the left and right bars represent data corresponding to Groups B and C (FIG. 38A), respectively; and Groups E and F (FIG. 38B), respectively; of Exp. No. 5.

FIGS. 39A-39F provide bar graphs showing production Th1/Th17/pro-inflammatory cytokines or Th2/anti-inflammatory cytokines: IFN-γ (FIG. 39A), IL-4 (FIG. 39B), IL-6 (FIG. 39C), IL-22 (FIG. 39D), IL-10 (FIG. 39E), and IL-17A (FIG. 39F) by splenocytes from HLA-DR4 mice immunized with each of: placebo (Group A); SEQ ID NO:136 (Group B); SEQ ID NO:138 (Group C); placebo (Group D); SEQ ID NO:136 (Group E); and SEQ ID NO:138 (Group F); (Erp. No. S), upon restimulation with SEQ ID NO:136, SEQ ID NO:137, SEQ ID NO:138, SEQ ID NO:139, SEQ ID NO:140, or no stimulation, approximately 33 days post first vaccination, as determined by Cytometric Bead Array (CBA).

FIGS. 40A-40C provides bar graphs depicting the percentage of activation induced markers (AIMs) OX40 and PD-L1 (FIG. 40A), CD69 and PD-L1 (FIG. 40B), and CD69 and CD86 (FIG. 40C) in splenocytes approximately 7 days post challenge with UPEC25, and approximately 53 days after first vaccination with placebo (Group A); SEQ ID NO:136 (Group B); SEQ ID NO:138 (Group C); placebo (Group D); SEQ ID NO:136 (Group E); and SEQ ID NO:138 (Group F); after restimulation with SEQ ID NO:136; SEQ ID NO:137; SEQ ID NO:138; and SEQ ID NO:139; or no stimulation; following the vaccination protocol described elsewhere herein (Exp. No. 5).

FIGS. 41A-41E provide graphs showing serum titers or levels of IgG1 (FIG. 41A), IgG2b (FIG. 41B), and IgA (FIG. 41C), and urine levels of IgA (FIG. 41D and IgG(H) (FIG. 41E) in HLA-DR4 mice approximately 34 days after vaccination with SEQ ID NO:141 (Group B); SEQ ID NO:142 (Group C); SEQ ID NO:143 (Group D); SEQ ID NO:144 (Group E); and SEQ ID NO:145 (Group F); as compared to placebo (Group A); as determined by standard ELISA, following the vaccination protocol described herein (Exp. No. 6).

FIGS. 42A-42D provides a bar graph showing the results of a peptide ELISA, wherein wells were coated with certain polypeptides (i.e., SEQ ID NOs: 16-17, 20, 25, 27, 195, 197-199, 163-168, 176-182, 188-192, 207, 213-215, 218-219, 200-203, and 141-145) and controls (i.e., tetanus toxin (TTX), serum albumin (SA) and no coating) and utilized sera from HLA-DR4 mice approximately 34 days after first vaccination with: placebo (Group A) and SEQ ID NO:141 (Group B) (FIG. 42A); SEQ ID NO:142 (Group C) (FIG. 42B); SEQ ID NO:143 (Group D) (FIG. 42C); or SEQ ID NO:144 (Group E) (FIG. 42D); following the vaccination protocol described elsewhere herein (Exp. No. 6). Two bars are depicted for each coating indicated along the x-axis (e.g., SEQ ID NO:17), wherein the left and right bars represent data corresponding to Groups A and B (FIG. 42A), respectively; Groups A and C (FIG. 42B), respectively; Groups A and D (FIG. 42C), respectively; and Groups A and E (FIG. 42D), respectively; of Exp. No. 6.

FIG. 43A-43F provide bar graphs showing production Th1/Th17/pro-inflammatory cytokines or Th2/anti-inflammatory cytokines: IFN-γ (FIG. 43A), IL-4 (FIG. 43B), IL-6 (FIG. 43C), IL-22 (FIG. 43D), IL-10 (FIG. 43E), and IL-17A (FIG. 43F) by splenocytes from HLA-DR4 mice immunized with each of: placebo (Group A); SEQ ID NO:141 (Group B); SEQ ID NO:142 (Group C); SEQ ID NO:143 (Group D); SEQ ID NO:144 (Group E); and SEQ ID NO:145 (Group F) as described elsewhere herein (Erp. No. 6), upon restimulation with SEQ ID NO:141; SEQ ID NO:142; SEQ ID NO:143; SEQ ID NO:144; SEQ ID NO:145; and SEQ ID NO:137, approximately 47 days post first vaccination, and approximately 7 days after UPEC25 challenge as determined by Cytometric Bead Array (CBA) Magpix®.

FIGS. 44A-44C provides bar graphs depicting the percentage of activation induced markers (AIMs) OX40 and PD-L1 (FIG. 44A), CD69 and PD-L1 (FIG. 44B), and CD69 and CD86 (FIG. 44C) in splenocytes approximately 7 days post challenge with UPEC25, and approximately 47 days after first vaccination with placebo (Group A); SEQ ID NO:141 (Group B); SEQ ID NO:142 (Group C); SEQ ID NO:143 (Group D); SEQ ID NO:144 (Group E); and SEQ ID NO:145 (Group F); after restimulation with SEQ ID NO:141; SEQ ID NO:142; SEQ ID NO:143; SEQ ID NO:144; SEQ ID NO:145; or no stimulation; following the vaccination protocol described elsewhere herein (Exp. No. 6).

FIGS. 45A-45E provide graphs showing serum titers or levels of IgG1 (FIG. 45A), IgG2b (FIG. 45B), and IgA (FIG. 45C), and urine levels of IgA (FIG. 45D and IgG(H) (FIG. 45E) in HLA-DR4 mice approximately 35 days after vaccination with placebo (Group A); SEQ ID NO:138(1) (Group B); SEQ ID NO:138(2) (Group C); placebo (Group D); SEQ ID NO:138(2) (Group E); placebo (Group F); or SEQ ID NO:138(2) (Group G); as determined by standard ELISA, following the vaccination protocol described herein (Exp. No. 7).

FIG. 46 provides a bar graph showing the results of a peptide ELISA, wherein wells were coated with certain polypeptides (i.e., SEQ ID NOs: 16-17, 20, 25, 27, 195, 197-199, 163-168, 176-182, 188-192, 207, 213-215, 218-219, 200-203, and 141-145) and controls (i.e., tetanus toxin (TTX), serum albumin (SA) and no coating) and utilized sera from HLA-DR4 mice approximately 35 days after first vaccination with: placebo (Group A); SEQ ID NO:138 (Group B); or SEQ ID NO:138 (Group C); following the vaccination protocol described elsewhere herein (Exp. No. 7). Three bars are depicted for each coating indicated along the x-axis (e.g., SEQ ID NO:17), wherein the left, center, and right bars represent data corresponding to Groups A, B, and C of Exp. No. 7, respectively.

FIGS. 47A-47F provide bar graphs showing production Th1/Th17/pro-inflammatory cytokines or Th2/anti-inflammatory cytokines: IFN-γ (FIG. 47A), IL-4 (FIG. 47B), IL-6 (FIG. 47C), IL-22 (FIG. 47D), IL-10 (FIG. 47E), and IL-17A (FIG. 47F) by splenocytes of HLA-DR4 mice immunized with each of: placebo (Group A); SEQ ID NO:138(1) (Group B); SEQ ID NO:138(2) (Group C); placebo (Group D); SEQ ID NO:138(2) Group E); placebo (Group F); or SEQ ID NO:138(2) (Group G); as described elsewhere herein (Erp. No. 7), upon restimulation with SEQ ID NO:138(1); SEQ ID NO:138(2); or SEQ ID NO:139; approximately 55 days post first vaccination and approximately 7 days after UPEC25 challenge as determined by Cytometric Bead Array (CBA) Magpix®.

FIGS. 48A-48F provide bar graphs showing production Th1/Th17/pro-inflammatory cytokines or Th2/anti-inflammatory cytokines: IFN-γ (FIG. 48A), IL-4 (FIG. 48B), IL-6 (FIG. 48C), IL-22 (FIG. 48D), IL-10 (FIG. 48E), and IL-17A (FIG. 48F) by bladder cells of HLA-DR4 mice immunized with each of: placebo (Group A); SEQ ID NO:138(1) (Group B); SEQ ID NO:138(2) (Group C); placebo (Group D); SEQ ID NO:138(2) (Group E); placebo (Group F); or SEQ ID NO:138(2) (Group G); as described elsewhere herein (Erp. No. 7), upon restimulation with SEQ ID NO:138(1); or SEQ ID NO:138(2); approximately 55 days post first vaccination and approximately 7 days after UPEC25 challenge as determined by Cytometric Bead Array (CBA) Magpix®.

FIGS. 49A-49C provides bar graphs depicting the percentage of activation induced markers (AIMs) OX40 and PD-L1 (FIG. 49A), CD69 and PD-L1 (FIG. 49B), and CD69 and CD86 (FIG. 49C) in splenocytes of HLA-DR4 mice approximately 7 days post challenge with UPEC25, and approximately 55 days after first vaccination with placebo (Group A); SEQ ID NO:138(1) (Group B); SEQ ID NO:138(2) (Group C); placebo (Group D); SEQ ID NO:138(2) (Group E); placebo (Group F); or SEQ ID NO:138(2) (Group G); after restimulation with SEQ ID NO:138(1); SEQ ID NO:138(2); SEQ ID NO:139; or no stimulation; following the vaccination protocol described elsewhere herein (Exp. No. 7).

FIGS. 50A-50B provide bar graphs depicting the percentage of activation induced markers (AIMs) OX40 and PD-L1 (FIG. 50A) and CD69 and PD-L1 (FIG. 50B) bladder cells of HLA-DR4 mice approximately 7 days post challenge with UPEC25, and approximately 55 days after first vaccination with placebo (Group A); SEQ ID NO:138(1) (Group B); SEQ ID NO:138(2) (Group C); placebo (Group D); SEQ ID NO:138(2) (Group E); placebo (Group F); or SEQ ID NO:138(2) (Group G); after restimulation with SEQ ID NO:138(1); SEQ ID NO:138(2); or no stimulation; following the vaccination protocol described elsewhere herein (Exp. No. 7).

FIGS. 51A-51E provide graphs showing serum titers or levels of IgG1 (FIG. 51A), IgG2b (FIG. 51B), and IgA (FIG. 51C), and urine levels of IgA (FIG. 51D and IgG(H) (FIG. 51E) in C57BL/6 mice approximately 34 days after vaccination with placebo (Group A); SEQ ID NO:136 (Group B); SEQ ID NO:137 (Group C); SEQ ID NO:138 (Group D); and SEQ ID NO:139 (Group E); as determined by standard ELISA, following the vaccination protocol described herein (Exp. No. 9).

FIGS. 52A-52B provide a bar graph showing the results of a peptide ELISA, wherein wells were coated with certain polypeptides (i.e., SEQ ID NOs: 17, 20, 25, 27, 195, 197-199, 163-168, 176-182, 188-192, 207, 213-215, 218-219, 200-203, and 136-139) and controls (i.e., tetanus toxin (TTX), serum albumin (SA) and no coating) and utilized sera from C57BL/6 mice 34 approximately days after first vaccination with: placebo (Group A); SEQ ID NO:136 (Group B); or SEQ ID NO:138 (Group D) (FIG. 52A); and placebo (Group A); SEQ ID NO:137 (Group C) (FIG. 52B); or SEQ ID NO:139 (Group E); following the vaccination protocol described elsewhere herein (Exp. No. 9). Three bars are depicted for each coating indicated along the x-axis (e.g., SEQ ID NO:17), wherein the left, center, and right bars represent data corresponding to Groups A, B, and D (FIG. 52A), respectively; and Groups A, C, and E (FIG. 52B), respectively; of Exp. No. 9.

FIGS. 53A-53F provide bar graphs showing production Th1/Th17/pro-inflammatory cytokines or Th2/anti-inflammatory cytokines: IL-17A (FIG. 53A), IL-6 (FIG. 53B), TNF-α (FIG. 53C), IFN-γ (FIG. 53D), IL-4 (FIG. 53E), and IL-10 (FIG. 53F) by splenocytes from C57BL/6 mice immunized with each of: placebo (Group A); SEQ ID NO:136 (Group B); or SEQ ID NO:138 (Group D) (FIG. 52A); and placebo (Group A); SEQ ID NO:137 (Group C) (FIG. 52B); or SEQ ID NO:139 (Group E); as described elsewhere herein (Erp. No. 9), upon restimulation with SEQ ID NO:136; SEQ ID NO:137; SEQ ID NO:138; (FIG. 52B); or SEQ ID NO:139; or no stimulus; approximately 52 days post first vaccination, approximately 7 days after UPEC25 challenge as determined by Cytometric Bead Array (CBA) Magpix®.

FIGS. 54A-54B provide bar graphs depicting the percentage of activation induced markers (AIMs) OX40 and PD-L1 (FIG. 54A) and CD86 and CD69 (FIG. 54B) in splenocytes of C57BL/6 mice approximately 7 days post challenge with UPEC25, and approximately 52 days after first vaccination with placebo (Group A); SEQ ID NO:136 (Group B); SEQ ID NO:137 (Group C); SEQ ID NO:138 (Group D); and SEQ ID NO:139 (Group E); after restimulation with SEQ ID NO:136; SEQ ID NO:137; SEQ ID NO:138; SEQ ID NO:139; or no stimulation; following the vaccination protocol described elsewhere herein (Exp. No. 9).

FIG. 55 provides a bar graph depicting the percentage of activation induced markers (AIMs) OX40 and PD-L1 in bladder cells of C57BL/6 mice approximately 7 days post challenge with UPEC25, and approximately 52 days after first vaccination with placebo (Group A); SEQ ID NO:136 (Group B); SEQ ID NO:137 (Group C); SEQ ID NO:138 (Group D); and SEQ ID NO:139 (Group E); after restimulation with SEQ ID NO:136; SEQ ID NO:138; or no stimulation; following the vaccination protocol described elsewhere herein (Exp. No. 9).

FIGS. 56A-56E provide graphs showing serum titers or levels of IgG1 (FIG. 56A), IgG2a (FIG. 56B), and IgA (FIG. 56C), and urine levels of IgA (FIG. 56D and IgG(H) (FIG. 56E) in C3H/HeN mice approximately 33 days after first vaccination with placebo (Group A); SEQ ID NO:225 (Group B); placebo (Group C); and SEQ ID NO:225 (Group D); as determined by standard ELISA, following the vaccination protocol described herein (Exp. No. 12).

FIGS. 57A-57B provide bar graphs showing the results of a peptide ELISA, wherein wells were coated with certain polypeptides (i.e., SEQ ID NOs: 17, 20, 25, 27, 40, 163-168, 176-182, 188-192, 195, 197-202, 207, 213-215, 218-219, and 225) and controls (i.e., tetanus toxin (TTX), serum albumin (SA) and no coating) and utilized sera from C3H/HeN mice approximately 33 days after first vaccination with: placebo (Group A; FIG. 57A); SEQ ID NO:225 (Group B; FIG. 57A); placebo (Group C; FIG. 57B); and SEQ ID NO:225 (Group D; FIG. 57B); following the vaccination protocol described elsewhere herein (Exp. No. 12). Two bars are depicted for each coating indicated along the x-axis (e.g., SEQ ID NO:17), wherein the left and right bars represent data corresponding to Groups A and B (FIG. 57A), respectively; and Groups C and D (FIG. 57B), respectively; of Exp. No. 12.

FIG. 58 provides a bar graph showing production of IFN-γ in the spleen, lymph node, and bladder of C3H/HeN mice immunized with each of placebo (Group A); SEQ ID NO:225 (Group B); placebo (Group C); and SEQ ID NO:225 (Group D); as described elsewhere herein (Exp. No. 12), upon restimulation with SEQ ID NO:225 or no stimulus (i.e., Unstim); approximately 33 days post first vaccination, as determined by Cytometric Bead Array (CBA) Magpix®.

FIGS. 59A-59E provide graphs showing serum titers or levels of IgG1 (FIG. 59A), IgG2b (FIG. 59B), and IgA (FIG. 59C), and urine levels of IgA (FIG. 59D and IgG(H) (FIG. 59E) in C57BL/6 mice approximately 33 days after first vaccination with placebo (Group A); SEQ ID NO:225 (Group B); placebo (Group C); and SEQ ID NO:225 (Group D); as determined by standard ELISA, following the vaccination protocol described herein (Exp. No. 10).

FIGS. 60A-60B provide bar graphs showing the results of a peptide ELISA, wherein wells were coated with certain polypeptides (i.e., SEQ ID NOs: 17, 20, 25, 27, 40, 163-168, 176-182, 188-192, 195, 197-202, 207, 213-215, 218-219, and 225) and controls (i.e., tetanus toxin (TTX), serum albumin (SA) and no coating) and utilized sera from C57BL/6 mice 33 approximately days after first vaccination with: placebo (Group A; FIG. 60A); SEQ ID NO:225 (Group B; FIG. 60A); placebo (Group C; FIG. 60B); and SEQ ID NO:225 (Group D; FIG. 60B); following the vaccination protocol described elsewhere herein (Exp. No. 10). Two bars are depicted for each coating indicated along the x-axis (e.g., SEQ ID NO:17), wherein the left and right bars represent data corresponding to Groups A and B (FIG. 60A), respectively; and Groups C and D (FIG. 60B), respectively; of Exp. No. 10.

FIG. 61 provides a bar graph showing production of IFN-γ in the spleen, lymph node, and bladder of C57BL/6 mice immunized with each of placebo (Group A); SEQ ID NO:225 (Group B); placebo (Group C); and SEQ ID NO:225 (Group D); as described elsewhere herein (Exp. No. 10), upon restimulation with SEQ ID NO:225 or no stimulus (i.e., Unstim); approximately 33 days post first vaccination, as determined by Cytometric Bead Array (CBA) Magpix®.

FIGS. 62A-62E provide graphs showing serum titers or levels of IgG1 (FIG. 62A), IgG2b (FIG. 62B), and IgA (FIG. 62C), urine levels of IgA (FIG. 62D) and IgG(H) (FIG. 62E) in C57BL/6 mice approximately 33 days after first vaccination (approximately 12 days after second vaccination) with SEQ ID NO:225 (Groups A, C, E, G, and I) or placebo (Groups B, D, F, and H); as determined by standard ELISA, following the vaccination protocol described herein (Exp. No. 11).

FIGS. 63A-63E provide bar graphs showing the results of a peptide ELISA, wherein wells were coated with certain polypeptides (i.e., SEQ ID NOs: 17, 20, 25, 27, 40, 163-168, 176-182, 188-192, 195, 197-202, 207, 213-215, 218-219, and 225) and controls (i.e., tetanus toxin (TTX), serum albumin (SA) and no coating) and utilized sera from C57BL/6 mice approximately 33 days after first vaccination (approximately 12 days after second vaccination) with: SEQ ID NO:225 (Group A; FIG. 63A); placebo (Group B; FIG. 63B); SEQ ID NO:225 (Group C; FIG. 63B); placebo (Group D; FIG. 63C); SEQ ID NO:225 (Group E; FIG. 63C); placebo (Group F; FIG. 63D); SEQ ID NO:225 (Group G; FIG. 63D); placebo (Group H; FIG. 63E); and SEQ ID NO:225 (Group I; Group 63E); following the vaccination protocol described herein (Exp. No. 11). For FIGS. 63B-63E, two bars are depicted for each coating indicated along the x-axis (e.g., SEQ ID NO:17), wherein the left and right bars represent data corresponding to Groups B and C (FIG. 63B), respectively; Groups D and E (FIG. 63C), respectively; Groups F and G (FIG. 63D), respectively; and Groups H and I (FIG. 63E), respectively; of Exp. No. 11.

FIG. 64 provides a bar graph depicting the percentage of activation induced markers (AIMs) OX40 and PDL1 in splenocytes obtained from C57BL/6 mice approximately 45 days after first vaccination with: SEQ ID NO:225 (Group A); placebo (Group B); SEQ ID NO:225 (Group C); placebo (Group D); SEQ ID NO:225 (Group E); placebo (Group F); SEQ ID NO:225 (Group G); placebo (Group H); and SEQ ID NO:225 (Group I); following the vaccination protocol described herein (Exp. No. 11). Splenocytes derived from the designated treatment or placebo groups (i.e., Groups A-I) were either restimulated (+) or not restimulated (−) with SEQ ID NO:225.

FIG. 65 provides a bar graph showing production of IFN-γ in the spleen of C57BL/6 mice immunized with: SEQ ID NO:225 (Group A); placebo (Group B); SEQ ID NO:225 (Group C); placebo (Group D); SEQ ID NO:225 (Group E); placebo (Group F); SEQ ID NO:225 (Group G); placebo (Group H); and SEQ ID NO:225 (Group I); upon restimulation with SEQ ID NO:225 or no stimulus (i.e., Unstim); approximately 12 days post second vaccination, following the vaccination protocol described herein (Exp. No. 11), as determined by Cytometric Bead Array (CBA) Magpix®.

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In this document, the term “each” is used to refer to every one of two or more members of a group, and is further used to refer to a single element where a group has only one member. For example, where Group 1 consists of A, B, and C, the term “each,” as applied to Group 1, refers to every one of A, B, and C. Further, where Group 2 consists of A, the term “each,” as applied to Group 2, refers to A.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “adjuvant” as used herein refers to a substance that increases and/or modulates the immune response to a vaccine. In certain embodiments, an adjuvant may act to accelerate, prolong, and/or enhance antigen-specific immune responses in a subject when administered in combination with one or more antigens.

An infection, disease, or disorder is “ameliorated” if the severity of a symptom of the disease or disorder, the frequency with which such a symptom is experienced by a patient, or both, is reduced.

The term “anionic lipid” refers to any lipid that is negatively charged at physiological pH (e.g., pH of about 7.0). These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

The term “antigen” as used herein refers to a substance capable of eliciting an immune response, for example, by a major histocompatibility complex (MHC) cell surface protein, or capable of binding to an antigen binding region of an immunoglobulin molecule. A T cell-mediated immune response achieved by presenting the antigen. An “antigen” as used herein includes, but is not limited to, an antigenic determinant, a hapten, and an immunogen, which may be a peptide, a small molecule, a carbohydrate, a lipid, a nucleic acid, or a combination thereof. A skilled immunologist should recognize that when discussing an antigen that is processed for presentation to a T cell, the term “antigen” refers to a portion of an antigen that is a T cell epitope that is presented by MHC to a T cell receptor (e.g., a peptide fragment). When used in the context of a B cell-mediated immune response in the form of an antibody specific for an “antigen”, the portion (i.e., binding portion) of the complementarity determining region of the variable domain of the binding antibody (i.e., light and heavy chains) in the antigen may be a linear or three-dimensional epitope.

The term “antigenic peptide” as used herein refers to a portion of a polypeptide antigen that is specifically recognized by a B cell or a T cell. B cells respond to foreign antigenic determinants via antibodies, while T-lymphocytes mediate cellular immunity. Thus, an antigenic peptide is a portion of an antigen that is recognized by an antibody or by a T cell receptor in the case of MHC.

The term “bacterial infection” as used herein refers to any infection(s) caused, directly or indirectly, by one or more species of gram-negative, gram-positive, or atypical bacteria. The term is not limited to infections which are caused exclusively by bacteria. Non-limiting examples of bacterial infections contemplated within the scope of the present disclosure include urinary tract infection (UTI), sepsis (e.g., neonatal sepsis), and pneumonia.

The term “cationic lipid” refers to any of a number of lipid species that carry a net positive charge at a selected pH, such as physiological pH (e.g., pH of about 7.0). It has been found that cationic lipids comprising alkyl chains with multiple sites of unsaturation, e.g., at least two or three sites of unsaturation, are particularly useful for forming lipid particles with increased membrane fluidity. Certain cationic lipids and related analogs, which are also useful in the present disclosure, have been described in U.S. Patent Publication Nos. 20060083780 and 20060240554; U.S. Pat. Nos. 5,208,036; 5,264,618; 5,279,833; 5,283,185; 5,753,613; and 5,785,992; and PCT Publication No. WO 96/10390, the disclosures of which are herein incorporated by reference in their entirety for all purposes. Non-limiting examples of cationic lipids are described in detail herein. In some cases, the cat-ionic lipids comprise a protonatable tertiary amine (e.g., pH titratable) head group, C₁₈ alkyl chains, ether linkages between the head group and alkyl chains, and 0 to 3 double bonds. Such lipids include, e.g., DSDMA, DLinDMA, DLenDMA, and DODMA.

As used herein, the term “composition” or “pharmaceutical composition” refers to a mixture of at least one polypeptide or compound useful within the invention with a pharmaceutically acceptable carrier. The pharmaceutical composition facilitates administration of the polypeptide or compound to a patient or subject. Multiple techniques of administering a compound exist in the art including, but not limited to, intravenous, subcutaneous, oral, aerosol, parenteral, ophthalmic, pulmonary and topical administration.

The term “conjugated lipid” as used herein refers to a lipid which is conjugated to one or more polymeric groups, which inhibits aggregation of lipid particles. Such lipid conjugates include, but are not limited to, polyamide oligomers (e.g., ATTA-lipid conjugates), PEG-lipid conjugates, such as PEG coupled to dialkyloxypropyls, PEG coupled to diacylglycerols, PEG coupled to cholesterol, PEG coupled to phosphatidylethanolamines, PEG conjugated to ceramides (e.g., U.S. Pat. No. 5,885,613, the disclosure of which is herein incorporated by reference in its entirety for all purposes), cationic PEG lipids, and mixtures thereof. PEG can be conjugated directly to the lipid or may be linked to the lipid via a linker moiety. Any linker moiety suitable for coupling the PEG to a lipid can be used including, e.g., non-ester containing linker moieties and ester-containing linker moieties. In preferred embodiments, non-ester containing linker moieties are used.

An “effective amount” or “therapeutically effective amount” of a compound is that amount of compound that is sufficient to provide a beneficial effect to the subject to which the polypeptide or compound is administered. An “effective amount” of a delivery vehicle is that amount sufficient to effectively bind or deliver a compound.

In particular, in the case of a mRNA, and “effective amount” or “therapeutically effective amount” of a therapeutic nucleic acid as relating to a mRNA is an amount sufficient to produce the desired effect, e.g., mRNA-directed expression of an amount of a polypeptide or protein that causes a desirable biological effect in the organism within which the polypeptide or protein is expressed. Suitable assays for measuring the expression of an mRNA or protein include, but are not limited to dot blots, Northern blots, in situ hybridization, ELISA, immunoprecipitation, enzyme function, as well as phenotypic assays known to those of skill in the art.

The term “encode” as used herein refers to the product specified (e.g., protein and RNA) by a given sequence of nucleotides in a nucleic acid (i.e., DNA and/or RNA), upon transcription or translation of the DNA or RNA, respectively. In certain embodiments, the term “encode” refers to the RNA sequence specified by transcription of a DNA sequence. In certain embodiments, the term “encode” refers to the amino acid sequence (e.g., polypeptide or protein) specified by translation of mRNA. In certain embodiments, the term “encode” refers to the amino acid sequence specified by transcription of DNA to mRNA and subsequent translation of the mRNA encoded by the DNA sequence. In certain embodiments, the encoded product may comprise a direct transcription or translation product. In certain embodiments, the encoded product may comprise post-translational modifications understood or reasonably expected by one skilled in the art.

The term “fetus” as used herein refers to any prenatal organism between conception and birth which is normally developed in utero (e.g., zygote and embryo). This definition also includes a prenatal organism which is first conceived in vitro and later implanted in a uterus. Upon birth, and for a period of about 28 days thereafter, the fetus is referred to herein as a “neonate” (e.g., “neonate thereof”).

The term “fully encapsulated” indicates that the active agent or therapeutic agent in the lipid particle is not significantly degraded after exposure to serum or a nuclease or protease assay that would significantly degrade free DNA, RNA, or protein. In a fully encapsulated system, preferably less than about 25% of the active agent or therapeutic agent in the particle is degraded in a treatment that would normally degrade 100% of free active agent or therapeutic agent, more preferably less than about 10%, and most preferably less than about 5% of the active agent or therapeutic agent in the particle is degraded. In the context of nucleic acid therapeutic agents, full encapsulation may be determined by an OLIGREEN® assay. OLIGREEN® is an ultra-sensitive fluorescent nucleic acid stain for quantitating oligonucleotides and single-stranded DNA or RNA in solution (available from Invitrogen Corporation; Carlsbad, Calif.). “Fully encapsulated” also indicates that the lipid particles are serum stable, that is, that they do not rapidly decompose into their component parts upon in vivo administration.

The term “helper lipid” as used herein refers to a lipid capable of increasing the effectiveness of delivery of lipid-based particles such as cationic lipid-based particles to a target, preferably into a cell. The helper lipid can be neutral, positively charged, or negatively charged. In certain embodiments, the helper lipid is neutral or negatively charged. Non-limiting examples of helper lipids include 1,2-distearoyl-sn-glycero-3-phosphocholine (DSPC), 1,2-di-(9Z-octadecenoyl)-sn-glycero-3-phosphoethanolamine (DOPE), 1-palmitoyl-2-oleoyl-sn-glycero-3-phosphocholin (POPC) and 1,2-dioleoyl-sn-glycero-3-phosphocholine (DOPC).

The term “immune cell,” as used herein refers to any cell involved in the mounting of an immune response. Such cells include, but are not limited to, T cells, B cells, NK cells, antigen-presenting cells (e.g., dendritic cells and macrophages), monocytes, neutrophils, eosinophils, basophils, and the like.

The term “immunogenic fragment” as used herein refers to a portion of a polypeptide sequence which specifically binds to, or is specifically bound by, an antibody produced in an immune response.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X2 are the same but X³ is different, and other analogous permutations.

The term “ionizable lipid” as used herein refers to a lipid (e.g., a cationic lipid) having at least one protonatable or deprotonatable group, such that the lipid is positively charged at a pH at or below physiological pH (e.g., pH 7.4), and neutral at a second pH, preferably at or above physiological pH. It will be understood by one of ordinary skill in the art that the addition or removal of protons as a function of pH is an equilibrium process, and that the reference to a charged or neutral lipid refers to the nature of the predominant species and does not require that all of the lipid be present in the charged or neutral form. Generally, ionizable lipids have a pKₐ of the protonatable group in the range of about 4 to about 7.

“Isolated” means altered or removed from the natural state. An isolated nucleic acid can exist in substantially purified form, or can exist in a non-native environment such as, for example, a host cell. An “isolated” nucleic acid encompasses a nucleic acid segment or fragment which has been separated from sequences which flank it in a naturally occurring state, e.g., a DNA fragment which has been removed from the sequences which are normally adjacent to the fragment in a genome in which it naturally occurs. The term also applies to nucleic acids which have been substantially purified from other components which naturally accompany the nucleic acid (e.g., RNA or DNA or proteins, which naturally accompany it in the cell). The term therefore includes, for example, a mRNA, or recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. Isolated does not require absolute purity, and can include protein, peptide, nucleic acid, or virus molecules that are at least 50% isolated, such as at least 75%, 80%, 90%, 95%, 98%, 990, or even 99.9% isolated.

The term “lipid” refers to a group of organic compounds that include, but are not limited to, esters of fatty acids and are characterized by being insoluble in water, but soluble in many organic solvents. They are usually divided into at least three classes: (1)“simple lipids,” which include fats and oils as well as waxes; (2) “compound lipids,” which include phospholipids and glycolipids; and (3)“derived lipids” such as steroids.

As used herein, “lipid encapsulated” can refer to a lipid particle that provides an active agent or therapeutic agent, such as a nucleic acid (e.g., a mRNA cargo), with full encapsulation or partial encapsulation. In a preferred embodiment, the nucleic acid is fully encapsulated in the lipid particle.

The term “lipid nanoparticle” refers to a particle having at least one dimension on the order of nanometers (e.g., 1-1,000 nm) which includes one or more lipids and/or additional agents.

The term “lipid particle” is used herein to refer to a lipid formulation that can be used to deliver an active agent or therapeutic agent, such as a nucleic acid (e.g., mRNA), to a target site of interest. In the lipid particle of the disclosure, which is typically formed from one or more cationic lipids or ionizable lipids, one or more non-cationic lipids (e.g., helper lipids and/or cholesterol), and one or more conjugated lipids that prevent aggregation of the particle, the active agent or therapeutic agent may be encapsulated in the lipid, thereby protecting the agent from enzymatic degradation.

The term “mRNA” or “messenger RNA” as used herein refers to a ribonucleic acid sequences which encodes a peptide or protein. In certain embodiments, the mRNA may comprise a “transcript” that is produced by using a DNA template and encodes a peptide or protein. Typically, mRNA comprises 5′-UTR, protein coding region and 3′-UTR. mRNA can be produced by in vitro transcription from a DNA template. Methods of in vitro transcription are known to those of skill in the art. For example, various in vitro transfer kits are commercially available. According to the present invention, mRNA can be modified by further stabilizing modifications and cap formation in addition to the modifications according to the invention.

The term “neutral amino acid” refers to any of a number of amino acids having a side chain which comprises a substituent that is uncharged at physiologically relevant pH (e.g., H, methyl, iso-propyl, iso-butyl, hydroxyl, and thiol, inter alia). Non-limiting examples of neutral amino acids and/or amino acids having a neutral side chain include glycine, alanine, valine, leucine, isoleucine, methionine, serine, threonine, cysteine, proline, glutamine, phenylalanine, tyrosine, tryptophane, asparagine, and glutamine.

The term “neonate” as used herein refers to an infant (e.g., human infant) which has an age of 0 to about 28 days.

The term “neutral lipid” refers to any of a number of lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides, and diacylglycerols.

The term “non-cationic lipid” refers to any amphipathic lipid as well as any other neutral lipid or anionic lipid.

The term “nucleic acid” as used herein refers to a polymer containing at least two deoxyribonucleotides or ribonucleotides in either single- or double-stranded form and includes DNA and RNA. DNA may be in the form of, e.g., antisense molecules, plasmid DNA, pre-condensed DNA, a PCR product, vectors (Pl, PAC, BAC, YAC, artificial chromosomes), expression cassettes, chimeric sequences, chromosomal DNA, or derivatives and combinations of these groups. RNA may be in the form of siRNA, asymmetrical interfering RNA (aiRNA), microRNA (miRNA), mRNA, tRNA, rRNA, tRNA, viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mal. Cell. Probes, 8:91-98 (1994)).

As used herein, the term “nucleic acid” includes any oligonucleotide or polynucleotide, with fragments containing up to 60 nucleotides generally termed oligonucleotides, and longer fragments termed polynucleotides. In particular embodiments, oligonucleotides of the disclosure are from about 15 to about 60 nucleotides in length. Nucleic acid may be administered alone in the lipid particles of the disclosure, or in combination (e.g., co-administered) with lipid particles of the disclosure comprising peptides, polypeptides, or small molecules such as conventional drugs. In other embodiments, the nucleic acid may be administered in a viral vector.

“Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkyl halides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)).

The term “oil-in-water emulsion adjuvant” as used herein refers to a biocompatible formulation comprising fine droplets (e.g., micro- and/or nanodroplets) of oil (e.g., squalene) dispersed within an aqueous phase, stabilized by surfactants or emulsifying agents (e.g., polysorbate 80). In certain embodiments, the oil phase comprises metabolizable oils (e.g., squalene and/or α-tocopherol). In certain embodiments, the aqueous phase comprises water or buffered saline (e.g., phosphate buffered saline). In certain embodiments, the surfactant comprises a nonionic, cationic, or anionic agent suitable to stabilize the emulsion and ensure uniform distribution of oil droplets. Non-limiting examples of oil-in-water emulsion adjuvants contemplated for use in the present invention include AddaS03™ and AS03®. In certain embodiments, AddaS03™ and/or AS03® comprise a nano-emulsion of DL-α-tocopherol (5% v/v) (i.e., racemic α-tocopherol) in squalene oil (5% v/v) and Tween® 80 (1.8% v/v) in phosphate buffered saline (PBS) (pH 6.8). In certain embodiments, the nano-emulsion is produced using a microfluidizer and filtered through a 0.22 μm filter to substantially reduce or remove large droplets from the final product, sterilize the final product, and/or substantially reduce or remove endotoxins from the final product.

The term “operably linked” or “operationally linked” refers to functional linkage between a regulatory sequence and a heterologous nucleic acid sequence permitting them to function in their intended manner (e.g., resulting in expression of the latter). The term encompasses positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

The terms “peptide,” “polypeptide,” and “protein” are used interchangeably herein, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the polypeptide or compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the polypeptide or compound useful within the invention, and are physiologically acceptable to the patient. Supplementary active compounds may also be incorporated into the compositions. The “pharmaceutically acceptable carrier” may further include a pharmaceutically acceptable salt of the polypeptide or compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

The term “pneumonia” as used herein refers to an inflammatory condition of the lung primarily affecting the small air sacs known as alveoli. Symptoms typically include some combination of productive or dry cough, chest pain, fever, and difficulty breathing. Pneumonia is usually caused by a bacterial or viral infection. Non-limiting, exemplary bacteria which are commonly isolated from subjects with pneumonia include Klebsiella pneumoniae, Streptococcus pneumoniae, Haemophilus influenzae, Chlamydophila pneumoniae, Mycoplasma pneumoniae, Staphylococcus aureus, Moraxella catarrhalis, and Legionella pneumophila.

The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG), DSPE-PEG-DBCO, DOPE-PEG-Azide, DSPE-PEG-Azide, DPPE-PEG-Azide, DSPE-PEG-Carboxy-NHS, DOPE-PEG-Carboxylic Acid, DSPE-PEG-Carboxylic acid and the like.

The term “prevent,” “preventing,” or “prevention” as used herein means avoiding or delaying the onset or recurrence of symptoms associated with an infection, disease, or condition in a subject not presenting such symptoms at the time the administering of an agent or compound commences. Infection, disease, condition and/or disorder may be used interchangeably herein.

The term “sepsis” as used herein refer to a life-threatening condition that arises when the body's response to infection causes injury to its own tissues and organs. Common symptoms include fever, increased heart rate, increasing breathing rate, confusion, cough, and painful urination, inter alia. Infections leading to sepsis generally comprise bacterial infections, although fungal, parasitic, and/or viral infections may also lead to sepsis. In certain embodiments, the bacterial infection which causes and/or contributes to the onset of sepsis may comprise gram-positive or gram-negative bacteria. Non-limiting, exemplary bacteria which may cause and/or contribute to the onset of sepsis include Staphylococci species, Klebsiella species, Streptococcus pyogenes, Escherichia coli, and Pseudomonas aeruginosa, inter alia. The term “neonatal sepsis” as used herein specifically refers to the occurrence of sepsis in a new born infant (i.e., neonate).

The terms “sequence homology,” “percent identity (%),” “sequence identity,” “sequence identity percent,” or “percent identity” in the context of amino acid sequences refers to a quantitative measurement of the similarity between two amino acid sequences over an aligned region of the two amino acid sequences.

By the term “specifically bind” or “specifically binds” as used herein is meant that a first molecule (e.g., antibody) preferentially binds to a second molecule (e.g., antigen and/or immunogenic fragment), but does not necessarily bind only to that second molecule.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The terms “treat,” “treating” and “treatment,” as used herein, means reducing the frequency or severity with which symptoms of a disease or condition are experienced by a subject by virtue of administering an agent (e.g., polypeptide or compound) to the subject.

The terms “UPEC” or “UPEC25” as used herein refers to “uropathogenic Escherichia coli” having a diameter of about 25 μm to about 250 μm, which is a pathotype of E. coli to which urinary tract infections (UTIs) are commonly attributed.

As used herein, the term “urinary tract infection” or “UTI” refers to a bacterial infection that affects parts of the body that produce and/or carry urine (i.e., the urinary tract), including the kidney, ureter, bladder and/or urethra. When it affects the lower urinary tract it is also known as a bladder infection (cystitis), and when it infects the upper urinary tract it is also known as kidney infection (pyelonephritis). Symptoms from a lower UTI can include pain with urination, frequent urination, and feeling the need to urinate despite having an empty bladder, while symptoms of kidney infection can include fever and flank pain usually in combination with the symptoms of a lower UTI. UTI can also lead to life-threatening invasive E. coli disease (e.g., bacteremia, sepsis, or urosepsis). The most common cause of UTI is E. coli. However, UTI can also be caused by other gram-negative bacteria (e.g., Klebsiella pneumonia and Proteus mirabilis, inter alia). Risk factors include female anatomy, sexual intercourse, diabetes, obesity and family history. UTIs are more common in women than in men, and occur frequently between the ages of 16 and 35 years. UTIs also occur frequently in elderly men and women.

A “vector” is a composition of matter which comprises an isolated nucleic acid and/or polypeptide and which can be used to deliver the isolated nucleic acid and/or polypeptide to the interior of a cell. Examples of vectors include but are not limited to, linear polynucleotides, polynucleotides associated with ionic or amphiphilic compounds, plasmids, and viruses. Thus, the term “vector” includes an autonomously replicating plasmid or a virus. The term is also construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like. “Expression vector” refers to a vector containing a polynucleotide having expression control sequences operatively linked to a nucleotide sequence to be expressed. An expression vector contains sufficient cis-acting elements for expression; other elements for expression can be supplied by the host cell or in an in vitro expression system. Expression vectors include all those known in the art, such as cosmids, plasmids (e.g., naked or contained in liposomes), phagemids, BACs, YACs, and viral vectors (e.g., vectors derived from lentiviruses, retroviruses, adenoviruses, and adeno-associated viruses) that incorporate the recombinant polynucleotide.

### DESCRIPTION

As described elsewhere herein, bacterial infections, including urinary tract infections (UTIs), sepsis (e.g., neonatal sepsis), and pneumonia pose a substantial public health risk, infecting millions of individuals each year. Prophylactic agents against such infections, often caused by E. coli, K. pneumonia, and/or P. mirabilis, are lacking, thereby creating an unmet need for compositions and/or methods of treating, preventing, and/or ameliorating a bacterial infection (e.g., urinary tract infection (UTI), sepsis and/or neonatal sepsis, and pneumonia), and/or generating immunity to infection by one or more pathogenic bacteria broadly.

Thus, the present disclosure relates, in one aspect, to compositions and/or methods for treating, preventing, and/or ameliorating a bacterial infection and/or generating immunity to infection by one or more bacterial pathogens in a subject. In one aspect, the present disclosure relates to vaccine compositions, and methods of use thereof, to address this unmet need.

In certain embodiments, the compositions and/or methods described herein are suitable for maternal immunization (i.e., use in a pregnant subject). In certain embodiments, the compositions and/or methods described herein are useful for treating, preventing, and/or ameliorating a bacterial infection and/or generating immunity to infection by one or more bacterial pathogens in a subject and/or fetus, or the corresponding neonate thereof. In one aspect, maternal immunization utilizing the compositions and/or methods described herein is useful for decreasing infant morbidity and/or mortality associated with bacterial infections (e.g., neonatal sepsis).

Vaccination has been widely used as a method for preventing viral infections (e.g., SARS-CoV-2 virus and COVID-19 infection), but has been employed to only a modest extent in the prevention of bacterial infection (e.g., Mycobacterium tuberculosis bacterium and tuberculosis infection). However, vaccinations operate in an analogous manner in both cases.

Briefly, vaccination comprises exposure of a subject to one or more antigens typically present on the surface of a foreign particle (e.g., bacterium), thereby stimulating an immune response. The first exposure of a subject to one or more of such antigens results in a primary immune response, wherein B cells create antibodies specific to the antigen, ultimately leading to destruction of the foreign particle by the host immune system (e.g., T cells). Additionally, B cells create memory cells which facilitate a more rapid response upon iterative exposures to the one or more antigens.

The surface of E. coli comprises certain proteins which may be detected by a host immune system, including iron receptor proteins, flagellar proteins, and non-flagellar proteins (e.g., pili, curli, and/or fimbriae), inter alia, which may result in an immune response and elimination of the infectious bacteria. Non-limiting examples of proteins which may be detected by a host immune system include AfaD, Afa/Dr, Ag43, BmaE, CfaE, CFA/I, ChuA, Cnfl, CsgA, dmLT, EatA, ECOK1_3385, EibD, EstA, F17G, FdeC, FimH, FliC, FmlH, FyuA, GspK, Hia, HlyA, HRA-1, IatA, IatB, IatC, IatD, Iha, intimin, IroN, IutA, MrpH, NaIP, OmpA, OmpT, OmpX, PapG, PapC, pertactin, PNAG, SfaS, SsIE, S pili, TolC, TosA, UpaB, UpaC, YadC, Yad Pili, YeeJ, YghA, YghJ, YgiL, Ygi pili, and YncE.

Thus, in one aspect, the present disclosure relates to compositions comprising bacterial surface proteins, and/or immunogenic fragments thereof, suitable to elicit an immune response in a subject, thereby generating immunity to infection by one or more pathogenic bacteria, and/or treating, preventing, and/or ameliorating a bacterial infection (e.g., urinary tract infection, sepsis, or pneumonia, inter alia) in a subject. In certain embodiments, the present disclosure relates to maternal immunization. In other embodiments, the present disclosure relates to compositions encoding bacterial surface proteins, and/or immunogenic fragments thereof, which upon delivery and translation, are suitable to elicit an immune response in a subject, thereby generating immunity to infection by one or more pathogenic bacteria, and/or to treat, prevent, and/or ameliorate a bacterial infection (e.g., urinary tract infection) in a subject.

### Lipids

Ionizable and/or Cationic Lipids

The terms “cationic lipid” and “ionizable lipid” are used interchangeably herein. Non-limiting examples of ionizable and/or cationic lipids contemplated for use in the lipid nanoparticles of the present disclosure include 1,2-dilinoleyloxy-N,N-dimethylaminopropane (DLinDMA), 1,2-dilinolenyloxy-N,N-dimethylaminopropane (DLenDMA), 2,2-dilinoleyl-4-(2-dimethylaminoethyl)-[1,3]-dioxolane (DLin-K-C2-DMA; “XTC2”), 2,2-dilinoleyl-4-(3-45 dimethylaminopropyl)-[1,3]-dioxolane (D Lin-K-C3-D MA), 2,2-dilinoleyl-4-(4-dimethylaminobutyl)-[1,3]-dioxolane (DLin-K-C4-DMA), 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 2,2-dilinoleyl-4-dimethylaminomethyl-[1,3]-dioxolane (DLin-KDMA), 1,2-dilinoleylcarbamoyloxy-3-dimethylaminopropane (D Lin-C-DAP), 1,2-dilinoleyoxy-3-(dimethylaminoacetoxypropane (DLin-DAC), 1-2dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (D Lin-MPZ), 3-(N,Ndilinoley lamino)-1,2-propanediol (D LinAP), 3-(N,Ndioley lamino)-1,2-propanedio (DOAP), 1,2-dilinoleyloxo-3-(2-N,N-dimethylamino)ethoxypropane (D Lin-EG-DMA), N,N-dioleyl-N,N-dimethylammonium chloride (DODAC), 1,2-dioleyloxy-N,N-dimethylaminopropane (DODMA), 1,2-distearyloxy-N,N-dimethylaminopropane (DSDMA), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N, N-trimethylammonium chloride (DOTAP), 3-(N-(N′,N′dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2 (spermine-carboxamidoethyl]-N,N-dimethyl-1-propanaminium trifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1(cis,cis-9′,1-2′-octadecadienoxy) propane (CpLinDMA), N,N-dimethyl-3,4-dioleyloxybenzylamine (DMOBA), 1,2-N,N′dioleylcarbamyl-3-dimethylaminopropane (DOcarbDAP), 1,2-N,N′-dilinoleylcarbamyl-3-dimethylaminopropane (DLincarbDAP), or mixtures thereof. In certain embodiments, the cationic lipid is DLinDMA, DLin-K-C2-DMA (“XTC2”), or mixtures thereof.

The synthesis of cationic lipids such as DLin-K-C2-DMA (“XTC2”), DLin-K-C3-DMA, DLin-K-C4-DMA, DLin-K6-DMA, and DLin-K-MPZ, as well as additional cationic lipids, is described in U.S. Provisional Application No. 61/104,212, filed Oct. 9, 2008, the disclosure of which is herein incorporated by reference in its entirety for all purposes. The synthesis of cationic lipids such as DLin-K-DMA, DLin-CDAP, DLin-DAC, DLin-MA, DLinDAP, DLin-S-DMA, DLin-2-DMAP, DLin-TMA.Cl, DLin-TAP.Cl, DLin-MPZ, DLinAP, DOAP, and DLin-EG-DMA, as well as additional cationic lipids, is described in PCT Application No. PCT/US08/88676, filed Dec. 31, 2008, the disclosure of which is herein incorporated by reference in its entirety for all purposes. The synthesis of cationic lipids such as CLinDMA, as well as additional cationic lipids, is described in U.S. Patent Publication No. 20060240554, the disclosure of which is herein incorporated by reference in its entirety for all purposes.

The scope of ionizable and/or cationic lipids contemplated for use in the lipid nanoparticles of the present disclosure is not limited to the species recited herein and may include any ionizable and/or cationic lipid known to one skilled in the art.

### Non-Cationic Lipid

In the lipid nanoparticles of the present disclosure, the non-cationic lipid may comprise, e.g., one or more anionic lipids, helper lipids and/or neutral lipids. In some embodiments, the non-cationic lipid comprises one of the following neutral lipid components: (1) cholesterol or a derivative thereof (2) a phospholipid; or (3) a mixture of a phospholipid and cholesterol or a derivative thereof.

Examples of cholesterol derivatives include, but are not limited to, cholestanol, cholestanone, cholestenone, coprostanol, cholesteryl-2′-hydroxyethyl ether, cholesteryl-4′-hydroxybutyl ether, and mixtures thereof. The synthesis of cholesteryl-2′-hydroxyethyl ether is known to one skilled in the art and described in U.S. Pat. Nos. 8,058,069, 8,492,359, 8,822,668, 9,364,435, 9,504,651, and 11,141,378, all of which are hereby incorporated herein in their entireties for all purposes.

Non-limiting examples of non-cationic lipids or helper lipids include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), ioleoylphosphatidylethanolamine (DOPE), palmitoyloleoylphosphatidylcholine (POPC), palmitoyloleoylphosphatidylethanolamine (POPE), palmitoyloleyolphosphatidylglycerol (POPG), dioleoylphosphatidylethanolamine 4-(N-maleimidomethyl)-cyclohexane-1-carboxylate DOPE-mal), dipalmitoylphosphatidylethanolamine (DPPE), dimyristoylphosphatidylethanolamine (DMPE), distearoylphosphatidylethanolamine (DSPE), monomethylphosphatidylethanolamine, dimethylphosphatidylethanolamine, dielaidoylphosphatidylethanolamine (DEPE), stearoyloleoylphosphatidylethanolamine (SOPE), lysophosphatidylcholine, dilinoleoylphosphatidylcholine, and mixtures thereof.

Other diacylphosphatidylcholine and diacylphosphatidylethanolamine phospholipids can also be used. The acyl groups in these lipids can be, for example, acyl groups derived from fatty acids having C₁₀-C₂₄ carbon chains, e.g., lauroyl, myristoyl, palmitoyl, stearoyl, or oleoyl. Additional examples of non-cationic lipids include sterols such as cholesterol and derivatives thereof such as cholestanol, cholestanone, cholestenone, coprostanol, cholesteryl-2′-hydroxyethyl ether, cholesteryl-4′-hydroxybutyl ether, and mixtures thereof. In certain embodiments, the phospholipid is DPPC, DSPC, or mixtures thereof.

### Conjugated Lipid

In the lipid nanoparticles of the present disclosure, the conjugated lipid that inhibits aggregation of particles may comprise, e.g., one or more of the following: a polyethyleneglycol (PEG)lipid conjugate, a polyamide (ATTA)-lipid conjugate, a cationic-polymer-lipid conjugates (CPLs), or mixtures thereof. In some embodiments, the nucleic acid-lipid particles comprise either a PEG-lipid conjugate or an ATTA-lipid conjugate.

PEG is a linear, water-soluble polymer of ethylene PEG repeating units with two terminal hydroxyl groups. PEGs are classified by their molecular weights; for example, PEG 2000 has an average molecular weight of about 2,000 daltons, and PEG 5000 has an average molecular weight of about 5,000 daltons. PEGs are commercially available from Sigma Chemical Co. and other companies and include, for example, the following: monomethoxypolyethylene glycol (MePEGOH), monomethoxypolyethylene glycolsuccinate (MePEGS), monomethoxypolyethylene glycolsuccinimidyl succinate (MePEG-S-NHS), monomethoxypolyethylene glycolamine (MePEG-NH₂), monomethoxypolyethylene glycoltresylate (MePEG-TRES), and monomethoxypolyethylene glycolimidazolylcarbonyl (MePEG-IM). Other PEGs such as those described in U.S. Pat. Nos. 6,774,180 and 7,053,150 (e.g., mPEG (20 KDa) amine) are also useful for preparing the PEG-lipid conjugates of the present disclosure. The disclosures of these patents are herein incorporated by reference in their entirety for all purposes. In addition, monomethoxypolyethyleneglycolacetic acid (MePEG-CH₂COOH) is particularly useful for preparing PEG-lipid conjugates including, e.g., PEG-DAA conjugates.

In certain embodiments, the PEG-lipid conjugate or ATTA-lipid conjugate is used together with a CPL. The conjugated lipid that inhibits aggregation of particles may comprise a PEG-lipid including, e.g., a PEG-diacylglycerol (DAG), a PEG dialkyloxypropyl (DAA), a PEG-phospholipid, a PEG-ceramide (Cer), or mixtures thereof. The PEGDAA conjugate may be PEG-dilauryloxypropyl (C₁₂), a PEG-dimyristyloxypropyl (C₁₄), a PEG-dipalmityloxypropyl (C₁₆), a PEG-distearyloxypropyl (C₁₈), or mixtures thereof.

Additional PEG-lipid conjugates suitable for use in the disclosure include, but are not limited to, mPEG2000-1,2-diO-alkyl-sn3-carbomoylglyceride (PEG-C-DOMG). The synthesis of PEG-C-DOMG is described in PCT Application No. PCT/US08/88676, filed Dec. 31, 2008, the disclosure of which is herein incorporated by reference in its entirety for all purposes. Yet additional PEG-lipid conjugates suitable for use in the disclosure include, without limitation, 1-[8′-(0,2-dimyristoyl-3-propanoxy)-carboxamido-3′,6′-dioxaoctanyl]carbamoyl-methyl-poly(ethylene glycol) (2 KPEG-DMG). The synthesis of 2 KPEG-DMG is described in U.S. Pat. No. 7,404,969, the disclosure of which is herein incorporated by reference in its entirety for all purposes.

The PEG moiety of the PEG-lipid conjugates described herein may comprise an average molecular weight ranging from about 550 daltons to about 10,000 daltons. In certain instances, the PEG moiety has an average molecular weight of from about 750 daltons to about 5,000 daltons (e.g., from about 1,000 daltons to about 5,000 daltons, from about 1,500 daltons to about 3,000 daltons, from about 750 daltons to about 3,000 daltons, from about 750 daltons to about 2,000 daltons, etc.). In some embodiments, the PEG moiety has an average molecular weight of about 2,000 daltons or about 750 daltons.

In addition to the foregoing, it will be readily apparent to those of skill in the art that other hydrophilic polymers can be used in place of PEG. Examples of suitable polymers that can be used in place of PEG include, but are not limited to, polyvinylpyrrolidone, polymethyloxazoline, polyethyloxazoline, polyhydroxypropyl methacrylamide, polymethacrylamide and polydimethylacrylamide, polylactic acid, polyglycolic acid, and derivatized celluloses such as hydroxymethylcellulose or hydroxyethylcellulose.

In addition to the foregoing components, the particles (e.g., LNP) of the present disclosure can further comprise cationic poly(ethylene glycol) (PEG) lipids or CPLs (e.g., Chen et al., Bioconj. Chem., 11:433-437 (2000)). Suitable SPLPs and SPLP-CPLs for use in the present disclosure, and methods of making and using SPLPs and SPLP-CPLs, are disclosed, e.g., in U.S. Pat. No. 6,852,334 and PCT Publication No. WO 00/62813, the disclosures of which are herein incorporated by reference in their entirety for all purposes.

In certain instances, the conjugated lipid that inhibits aggregation of particles (e.g., PEG-lipid conjugate) may comprise from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 1 mol % to about 1.8 mol %, from about 1.2 mol % to about 1.8 mol %, from about 1.2 mol % to about 1.7 mol %, from about 1.3 mol % to about 1.6 mol %, from about 1.4 mol % to about 1.5 mol %, or about 1, 1.1, 1.2, 1.3, 1.4, 1.5, 1.6, 1.7, 1.8, 1.9, or 2 mol % (or any fraction thereof or range therein) of the total lipid present in the particle.

In the lipid nanoparticles of the present disclosure, the active agent or therapeutic agent may be fully encapsulated within the lipid portion of the particle, thereby protecting the active agent or therapeutic agent from enzymatic degradation. In some embodiments, a nucleic acid-lipid particle comprising a nucleic acid such as a messenger RNA (i.e., mRNA) is fully encapsulated within the lipid portion of the particle, thereby protecting the nucleic acid from nuclease degradation. In certain instances, the nucleic acid in the nucleic acid-lipid particle is not substantially degraded after exposure of the particle to a nuclease at 37° C. for at least about 20, 30, 45, or 60 minutes. In certain other instances, the nucleic acid in the nucleic acid-lipid particle is not substantially degraded after incubation of the particle in serum at 37° C. for at least about 30, 45, or 60 minutes or at least about 2, 3, 4, 5, 6, 7, 8, 9, 10, 12, 14, 16, 18, 20, 22, 24, 26, 28, 30, 32, 34, or 36 hours. In other embodiments, the active agent or therapeutic agent (e.g., nucleic acid such as siRNA) is complexed with the lipid portion of the particle. One of the benefits of the formulations of the present disclosure is that the lipid particle compositions are substantially non-toxic to mammals such as humans.

### Lipid Nanoparticles (LNPs)

In another aspect, the present disclosure provides a lipid nanoparticle (LNP) composition comprising an isolated nucleic acid of the present disclosure. In certain embodiments, the isolated nucleic acid of the present disclosure is an isolated mRNA. In certain embodiments, the isolated nucleic acid of the present disclosure is an isolated DNA. In certain embodiments, the LNP has a ratio of lipid to isolated mRNA ranging from about 5:1 to about 25:1. In certain embodiments, the isolated mRNA is at least partially encapsulated in the LNP. In certain embodiments, the LNP has a ratio of lipid to isolated DNA ranging from about 5:1 to about 25:1. In certain embodiments, the isolated DNA is at least partially encapsulated in the LNP.

In certain embodiments, the isolated polynucleotide is at least partially encapsulated in the LNP. In certain embodiments, the isolated polynucleotide is fully encapsulated in the LNP.

In another aspect, the present disclosure provides a lipid nanoparticle (LNP) composition comprising the isolated polynucleotide of the present disclosure. In certain embodiments, the LNP has a ratio of lipid to isolated polynucleotide ranging from about 5:1 to about 25:1. In certain embodiments, the isolated mRNA is fully encapsulated in the LNP. In certain embodiments, the isolated DNA is fully encapsulated in the LNP.

In certain embodiments, the LNP comprises:
(a) at least one ionizable lipid;
(b) at least one helper lipid;
(c) cholesterol, or a modified derivative thereof, and any combinations thereof, and
(d) at least one conjugated lipid.

In certain embodiments, the ionizable lipid is at least one selected from the group consisting of DLinDMA, DLenDMA, DLin-K-C2-DMA, D Lin-K-C3-D MA, DLin-K-C4-DMA, DLin-K6-DMA, DLin-K-MPZ, DLin-KDMA, D Lin-C-DAP, DLin-DAC, DLin-MA, DLinDAP, DLin-2-DMAP, DLin-TMA.Cl, DLin-TAP.Cl, D Lin-MPZ, D LinAP, DOAP, D Lin-EG-D MA, DODAC, DODMA, DSD MA, DOTMA, DDAB, DOTAP, DC-Chol, DMRIE, DOSPA, DOGS, CLinDMA, CpLinDMA, DMOBA, DOcarbDAP, and DLincarbDAP.

In certain embodiments, the at least one ionizable lipid comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 99 mol % of the LNP.

In certain embodiments, the at least one ionizable lipid comprises less than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 99 mol % of the LNP.

In certain embodiments, the at least one ionizable lipid comprises more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 99 mol % of the LNP.

In certain embodiments, the helper lipid is at least one selected from the group consisting of 1,2-distearoyl-sn-glycero-3-phosphocholine (DSPC), 1,2-di-(9Z-octadecenoyl)-sn-glycero-3-phosphoethanolamine (DOPE), 1-palmitoyl-2-oleoyl-sn-glycero-3-phosphocholin (POPC), and 1,2-dioleoyl-sn-glycero-3-phosphocholine (DOPC).

In certain embodiments, the at least one helper lipid comprises about 1, 2, 3, 4, 5, 6, 7, 8, 9, 10, 11, 12, 13, 14, 15, 16, 17, 18, 19, 20, 21, 22, 23, 24, or about 25 mol % of the LNP.

In certain embodiments, the at least one helper lipid comprises less than about 1, 2, 3, 4, 5, 6, 7, 8, 9, 10, 11, 12, 13, 14, 15, 16, 17, 18, 19, 20, 21, 22, 23, 24, or about 25 mol % of the LNP.

In certain embodiments, the at least one helper lipid comprises more than about 1, 2, 3, 4, 5, 6, 7, 8, 9, 10, 11, 12, 13, 14, 15, 16, 17, 18, 19, 20, 21, 22, 23, 24, or about 25 mol % of the LNP.

In certain embodiments, cholesterol comprises about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or about 60 mol % of the LNP.

In certain embodiments, cholesterol comprises less than about 20, 21, 22, 23, 24, 25, 26, 27, 28, 29, 30, 31, 32, 33, 34, 35, 36, 37, 38, 39, 40,41,42,43,44,45,46,47, 48, 49, 50, 51, 52, 53, 54, 55, 56, 57, 58, 59, or about 60 mol % of the LNP.

In certain embodiments, cholesterol comprises more than about 20, 21, 22, 23, 24, 25, 26, 27, 28, 29, 30, 31, 32, 33, 34, 35, 36, 37, 38, 39, 40,41,42,43,44,45,46,47, 48, 49, 50, 51, 52, 53, 54, 55, 56, 57, 58, 59, or about 60 mol % of the LNP.

In certain embodiments, the conjugated lipid is at least one selected from the group consisting of 1,2-dimyristoyl-rac-glycero-3-methoxypolyethylene glycol-2000 (PEG-DMG), 1,2-Distearoyl-sn-glycerol, methoxypolyethylene glycol (PEG-DSG), 1,2-Dipalmitoyl-sn-glycerol, methoxypolyethylene glycol (PEG-DPG), mPEG-OH, mPEG-AA (mPEG-CM), mPEG-CH₂CH₂CH₂—NH₂, mPEG-DMG, mPEG-N,N-Ditetradecylacetamide (ALC-0159), mPEG-DSPE, and mPEG-DPPE.

In certain embodiments, the at least one conjugated lipid comprises about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or about 5.0 mol % of the LNP.

In certain embodiments, the at least one conjugated lipid comprises less than about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or about 5.0 mol % of the LNP.

In certain embodiments, the at least one conjugated lipid comprises more than about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or about 5.0 mol % of the LNP.

### Nucleic Acids

Messenger RNA (mRNA)

In one aspect, the present disclosure provides an isolated messenger ribonucleic acid (mRNA) encoding the polypeptide of the present disclosure. In certain embodiments, the mRNA is codon optimized for expression in a mammal. In certain embodiments, the mammal is a human. In certain embodiments, the mRNA is codon optimized for expression in a prokaryote. In certain embodiments, the prokaryote is E. coli.

In another aspect, the present disclosure provides an isolated polynucleotide encoding the mRNA of the present disclosure, wherein the polynucleotide comprises one or more promoters and/or a polyadenylation signal operably linked to a sequence encoding the mRNA.

In certain embodiments, the disclosed nucleic acids are, or include, ribonucleic acids. A non-limiting ribonucleic acid is messenger RNA (mRNA). The term messenger RNA (mRNA) can refer to any ribonucleic acid which directly encodes a polypeptide of interest. Thus, the disclosed mRNAs are capable of being translated to produce one or more encoded polypeptides of interest. In certain non-limiting embodiments, the mRNAs are produced by in vitro transcription.

The mRNAs can be of any suitable length. For example, the length can vary depending upon the size of the encoded polypeptide. mRNA molecules are typically between 200 and 10,000 nucleotides in length. In certain non-limiting embodiments, a mRNA includes about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000, 4,000, 5,000, 6,000, 7,000, 8,000, 9,000, 10,000, 20,000, 30,000, 40,000, 50,000, 60,000, 70,000, 80,000, 90,000 or 100,000 nucleotides, with or without the poly(A) tail, 5′ UTR, and/or 3′ UTR.

The mRNAs can be codon optimized. For example, the mRNAs can be codon optimized for expression in a eukaryotic cell or prokaryotic cell (e.g., E. coli). The eukaryotic cell can be those of or derived from a particular organism, such as a plant or a mammal, including but not limited to human, or non-human eukaryote or animal or mammal, e.g., mouse, rat, rabbit, dog, livestock, or non-human mammal or primate. Codon-optimization describes gene engineering approaches that use changes of rare codons to synonymous codons that are more frequently used in the cell type of interest with the aim of increasing protein production. In general, codon optimization involves modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA, which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See for example, Nakamura, Y., et al., Nucl. Acids Res., 28:292 (2000). Computer algorithms for codon optimizing a particular sequence for expression in a particular host cell are also available, such as Gene Forge (Aptagen; Jacobus, PA). In certain non-limiting embodiments, one or more codons (e.g., 1, 2, 3, 4, 5, 10, 15, 20, 25, 50, or more, or all codons) in a mRNA corresponds to the most frequently used codon for a particular amino acid.

Typically, the disclosed isolated messenger ribonucleic acids (mRNAs) contain a 5′ untranslated region (UTR), a 3′ UTR, and an open reading frame (also referred to as coding region). In certain non-limiting embodiments, the mRNAs further include a 5′ cap or an analog thereof, a poly(A) tail, one or more modified nucleotides, or a combination thereof. In certain embodiments, the mRNAs include at least a 5′ cap or analog thereof, a 5′ UTR, a 3′ UTR, one or more open reading frames, and a poly(A) tail. In certain embodiments, the mRNAs include at least a 5′ cap or analog thereof, a 5′ UTR, a 3′ UTR, one or more open reading frames, a poly(A) tail, and one or more modified nucleotides.

The mRNA can include different caps or cap analogs (e.g., ARCA). The body of the mRNA can use modified nucleosides. The one or more coding sequences or open reading frames can include various elements such as signal peptides, localization signals (e.g., NLSs), inteins, etc. The structures of the mRNA can be engineered to optimize GC motifs, folding, circularization signals, and/or structured UTR elements.

### 5′ Cap

Typically, the 5′ cap of an mRNA is involved in nuclear export, increasing mRNA stability and binding the mRNA Cap Binding Protein (CBP), which is responsible for mRNA stability in the cell and translation competency through the association of CBP with poly(A) binding protein to form the mature cyclic mRNA species. Endogenous mRNA molecules may be 5′-end capped generating a 5′-ppp-5′-triphosphate linkage between a terminal guanosine cap residue and the 5′-terminal transcribed sense nucleotide of the mRNA molecule. This 5′-guanylate cap may then be methylated to generate an N7-methyl-guanylate residue. In certain non-limiting embodiments, the mRNA contains a non-hydrolyzable cap, which can prevent or hinder decapping and thus increase the mRNA half-life. Because cap structure hydrolysis requires cleavage of 5′-ppp-5′ phosphodiester linkages, the 5′ cap can include modified nucleotides to prevent such hydrolysis.

The 5′ cap may be a single nucleotide or a series of nucleotides. For example, the cap may include from 1 to 10, e.g., 2-9, 3-8, 4-7, 1-5, 5-10, or at least 1 or 2, or 10 or fewer nucleotides in length. In certain non-limiting embodiments, the cap is absent.

Cap analogs differ from natural (e.g., endogenous, wild-type or physiological) 5′-caps in their chemical structure, while retaining cap function. Cap analogs may be chemically (e.g., non-enzymatically) or enzymatically synthesized and/or linked to a nucleic acid molecule. For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m⁷G-3′mppp-G; which may equivalently be designated 3′ O-Me-m7G(5′)ppp(5′)G). The 3′-0 atom of the other, unmodified, guanine becomes linked to the 5′-terminal nucleotide of the capped nucleic acid molecule (e.g., mRNA). The N7- and 3′-O-methylated guanine provides the terminal moiety of the capped nucleic acid molecule. Another exemplary cap is mCAP, which is similar to ARCA but has a 2′-O-methyl group on guanosine (i.e., N7,2′-O-dimethyl-guanosine-5′-triphosphate-5′-guanosine, m⁷Gm-ppp-G).

In certain non-limiting embodiments, a 5′ cap may include endogenous caps or cap analogs. For example, a 5′ cap may include a guanine analog. Useful guanine analogs include, but are not limited to, inosine, N1-methyl-guanosine, 2′-fluoro-guanosine, 7-deaza-guanosine, 8-oxo-guanosine, 2-amino-guanosine, LNA-guanosine, and 2-azido-guanosine.

Suitable 5′ caps or analogs that can be included in the mRNAs are known in the art and include, without limitation, 7mG(5′)ppp(5′)N,pN2p (cap 0), 7mG(5′)ppp(5′)NlmpNp (cap 1), 7mG(5′)-ppp(5′)NlmpN2mp (cap 2), ARCA, beta-S-ARCA, m7G, mCAP, inosine, N1-methyl-guanosine, 2′-fluoro-guanosine, 7-deaza-guanosine, 8-oxo-guanosine, 2-amino-guanosine, LNA-guanosine, tri-methylgranosine (TMG), nicotinamide adenine dinucleotide (NAD), cap AG, cap AU, cap GG, and 2-azido-guanosine.

### Deoxyribonucleic Acid (DNA)

In one aspect, the present disclosure provides an isolated deoxyribonucleic acid (DNA) encoding the polypeptide of the present disclosure. In certain embodiments, the DNA is codon optimized for expression in a mammal. In certain embodiments, the mammal is a human.

The DNAs can be of any suitable length. For example, the length can vary depending upon the size of the encoded polypeptide. DNA molecules are typically between 200 and 10,000 nucleotides in length. In certain non-limiting embodiments, a DNA includes about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000, 4,000, 5,000, 6,000, 7,000, 8,000, 9,000, 10,000, 20,000, 30,000, 40,000, 50,000, 60,000, 70,000, 80,000, 90,000 or 100,000 nucleotides, with or without the poly(A) tail, 5′ UTR, and/or 3′ UTR.

The DNAs can be codon optimized. For example, the DNAs can be codon optimized for expression in a prokaryotic cell. In certain embodiments, the prokaryotic cell is E. coli. In other embodiments, the DNAs can be codon optimized for expression in a eukaryotic cell. The eukaryotic cell can be those of or derived from a particular organism, such as a plant or a mammal, including but not limited to human, or non-human eukaryote or animal or mammal, e.g., mouse, rat, rabbit, dog, livestock, or non-human mammal or primate.

In certain embodiments, the DNA (e.g., DNA which is codon optimized for mammalian expression) can be administered to a mammal in a vector, wherein expression may occur in the mammal (e.g., human) to impart therapeutic effects. In other embodiments, the DNA (e.g., DNA which is codon optimized for prokaryotic expression) can be administered and/or delivered to a prokaryotic cell (e.g., E. coli) for manufacture and isolation of the protein product thereof.

### Untranslated Regions

Untranslated regions (UTRs) are regions of a gene that are transcribed, but not translated. Generally, the 5′UTR starts at the transcription start site and continues to the start codon but does not include the start codon; whereas the 3′UTR starts immediately following the stop codon and continues until the transcriptional termination signal. 5′ UTRs can harbor specific regions, like Kozak sequences which are be involved in the initiation of translation by the ribosome. 5′ UTRs also have been known to form secondary structures which are involved in elongation factor binding. The UTRs can have important regulatory effects on an associated mRNA, for example impacting stability and/or translation of the mRNA. Generally, translational efficiency (including activation or inhibition of translation) of mRNAs can be controlled by the UTRs. In certain non-limiting embodiments, the regulatory features of a UTR can be incorporated into the disclosed mRNAs, to enhance the stability of the molecule. In certain non-limiting embodiments, the mRNAs are engineered to contain the UTRs found in abundantly expressed genes to enhance the enhance the stability and protein production from the mRNA. For example, introduction of 5′ UTR of liver-expressed mRNA, such as albumin, serum amyloid A, Apolipoprotein A/B/E, transferrin, alpha fetoprotein, erythropoietin, or Factor VIII, could be used to enhance expression of an mRNA. Likewise, use of 5′ UTR from other tissue-specific mRNA to improve expression in that tissue is possible for muscle (MyoD, Myosin, Myoglobin, Myogenin, Herculin), for endothelial cells (Tie-1, CD36), for myeloid cells (C/EBP, AML1, G-CSF, GM-CSF, CD11b, MSR, Fr-1, i-NOS), for leukocytes (CD45, CD18), for adipose tissue (CD36, GLUT4, ACRP30, adiponectin) and for lung epithelial cells (SP-A/B/C/D).

### Poly a Tails

During RNA processing, a long chain of adenine nucleotides, referred to as the poly(A) tail, may be added to a polynucleotide such as an mRNA in order to increase stability. Immediately after transcription, the 3′ end of the transcript may be cleaved to free a 3′ hydroxyl. Then, poly-A polymerase adds a chain of adenine nucleotides to the RNA. The process, called polyadenylation, adds a poly(A) tail that can be between, for example, approximately 100 and 250 residues long.

In certain non-limiting embodiments, the poly(A) tail includes about 10-100, about 100-300, about 100-250, or about 100-200 adenines. In certain non-limiting embodiments, the poly(A) tail contains about 10, 15, 20, 25, 30, 35, 40, 45, 50, 55, 60, 65, 70, 75, 80, 85, 90, 95, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, or 3,000 nucleotides.

### Open Reading Frame (ORF) Encoded Polypeptides

The mRNAs contain sequences that encode polypeptides of interest. For example, an mRNA can contain one or more open reading frames, each of which encodes one or more polypeptides. Typically, the open reading frame encodes an antigen (e.g., protein or peptide) from a pathogenic microorganism, such as bacteria, fungi, protozoa, or virus. In certain non-limiting embodiments, the open reading frame encodes one or more proteins from a virus, or an immune-response inducing fragment or variant thereof.

Suitable variants can include at least one point mutation or substitution (e.g., 1, 2, 3, 4, 5 or more mutations) at any amino acid residue relative to a reference. Amino acid substitutions in certain non-limiting embodiments include conservative amino acid substitutions, although non-conservative substitutions can also be used. Examples of conservative amino acid substitutions include those in which the substitution is within one of the five following groups: 1) small aliphatic, nonpolar or slightly polar residues (Ala, Ser, Thr, Pro, Gly); 2) polar, negatively charged residues and their amides (Asp, Asn, Glu, Gln); polar, positively charged residues (His, Arg, Lys); large aliphatic, nonpolar residues (Met, Leu, Ile, Val, Cys); and large aromatic resides (Phe, Tyr, Trp). Examples of non-conservative amino acid substitutions are those where 1) a hydrophilic residue, e.g., seryl or threonyl, is substituted for (or by) a hydrophobic residue, e.g., leucyl, isoleucyl, phenylalanyl, valyl, or alanyl; 2) a cysteine or proline is substituted for (or by) any other residue; 3) a residue having an electropositive side chain, e.g., lysyl, arginyl, or histidyl, is substituted for (or by) an electronegative residue, e.g., glutamyl or aspartyl; or 4) a residue having a bulky side chain, e.g., phenylalanine, is substituted for (or by) a residue that does not have a side chain, e.g., glycine.

### Other Polynucleotides

The polynucleotides of the present disclosure may further comprise functional polynucleotide regions (e.g., non-coding polynucleotides). The polynucleotide can include one or more promoters and/or a polyadenylation signal operably linked to a sequence encoding the mRNA. In certain non-limiting embodiments, the polynucleotide is, or is contained within, a plasmid. In certain non-limiting embodiments, the polynucleotide is, or is contained within, a vector, such as an expression vector.

Expression vectors include all those known in the art, such as cosmids, plasmids (e.g., naked or contained in liposomes), phagemids, artificial chromosomes (e.g., BACs, YACs), and viral vectors (e.g., vectors derived from lentiviruses, retroviruses, adenoviruses, and adeno-associated viruses) that incorporate the polynucleotide.

In certain non-limiting embodiments, a polynucleotide (e.g., the portion thereof encoding a mRNA) is operably linked to a control element, e.g., a transcriptional control element, such as a promoter. The transcriptional control element may be functional in either a eukaryotic cell, e.g., a mammalian cell, or a prokaryotic cell (e.g., bacterial or archaeal cell). In certain non-limiting embodiments, a polynucleotide (e.g., the portion thereof encoding a mRNA thereof) is operably linked to multiple control elements that allow expression of the polynucleotide sequence encoding a mRNA in either prokaryotic or eukaryotic cells. Depending on the host/vector system utilized, any of certain suitable transcription and translation control elements, including constitutive and inducible promoters, transcription enhancer elements, transcription terminators, etc. may be used in the expression vector (e.g., U6 promoter, HI promoter, CMV promoter, T7 promoter, SV40 promoter, bGH poly(A) signal, SV40 poly(A) signal, etc.).

Numerous vectors and expression systems are commercially available from commercial vendors including Addgene, Novagen (Madison, WI), Clontech (Palo Alto, CA), Stratagene (La Jolla, CA), and Invitrogen/Life Technologies (Carlsbad, CA). Suitable expression vectors include, but are not limited to, viral vectors such as viral vectors based on vaccinia virus, poliovirus, adenovirus, adeno-associated virus, SV40, herpes simplex virus, human immunodeficiency virus, retroviral vectors (e.g., Murine Leukemia Virus, spleen necrosis virus, and vectors derived from retroviruses such as Rous Sarcoma Virus, Harvey Sarcoma Virus, avian leukosis virus, lentivirus, human immunodeficiency virus, myeloproliferative sarcoma virus, and mammary tumor virus), and the like. The viral vector can be derived from a DNA virus (e.g., dsDNA or ssDNA virus) or an RNA virus (e.g., a ssRNA virus).

Numerous suitable expression vectors are known to those of skill in the art, and many are commercially available, including, pET29 (Novagen), pXTl, pSG5 (Stratagene), pSVK3, pBPV, pMSG, pCDNA 3.1, and pSVLSV40 (Pharmacia). However, any other vector may be used so long as it is compatible with the host cell.

Any cell may be used in accordance with the foregoing. In certain non-limiting embodiments, the cell is a prokaryotic cell (e.g., an archaeal or bacterial cell). In certain non-limiting embodiments, the cell is E. coli. In other forms, the cell is a eukaryotic cell. For example, the cell can be a cell of a single-cell eukaryotic organism, a plant cell, an algal cell, a fungal cell (e.g., a yeast cell). The cell can be a mammalian cell. The mammalian cell can be human or non-human mammal, e.g., primate, bovine, ovine, porcine, canine, rodent, monkey, rat, or mouse cell.

Generation of the polynucleotides can be accomplished using any suitable genetic engineering techniques well known in the art, including, without limitation, the standard techniques of restriction endonuclease digestion, ligation, transformation, plasmid purification, and DNA sequencing, for example as described in Sambrook et al. (Molecular Cloning: A Laboratory Manual. Cold Spring Harbor Laboratory Press, N.Y. (1989)).

### Vectors

In another aspect, the present disclosure provides a vector comprising the isolated nucleic acid of the present disclosure. In certain embodiments, the isolated nucleic acid comprises RNA. In certain embodiments, the nucleic acid comprises DNA. In certain embodiments, the vector is a viral vector. In certain embodiments, the viral vector is a Adeno-Associated Virus (AAV). In certain embodiments, the AAV is AAV9.

In another aspect, the present disclosure provides a vector comprising the isolated polynucleotide of the present disclosure. In certain embodiments, the vector is a viral vector. In certain embodiments, the viral vector is a Adeno-Associated Virus (AAV). In certain embodiments, the AAV is AAV9.

In certain non-limiting embodiments, the vector encoding a vaccine antigen (e.g., mRNA and/or polypeptide) is a viral vector. In certain non-limiting embodiments, the viral vector is an adeno-associated virus (AAV) vector. In certain embodiments, an AAV2-derived ITR sequence, or a deleted form thereof (ΔITR), is used for convenience and for accelerated regulatory approval. However, ITRs from other AAV sources may be selected. If the source of the ITR is from AAV2 and the AAV capsid is from another source of AAV, the resulting vector can be referred to as a pseudoform.

AAV is a non-pathogenic, single-stranded DNA virus that has been actively employed over the years for delivering therapeutic genes in both in vitro and in vivo systems (Choi, et al., Curr. Gene Ther., 5:299-310, (2005)). AAV belongs to the parvovirus family and is dependent on co-infection with other viruses, mainly adenoviruses, in order to replicate. Each end of the single-stranded DNA genome contains an inverted terminal repeat (ITR), which is the only cis-acting element required for genome replication and packaging. The single-stranded AAV genome contains three genes, Rep (Replication), Cap (Capsid), and aap (Assembly). These three genes give rise to at least nine gene products through the use of three promoters, alternative translation start sites, and differential splicing. These coding sequences are flanked by the ITRs. The Rep gene encodes four proteins (Rep78, Rep68, Rep52, and Rep40), while Cap expression gives rise to the viral capsid proteins (VP; VP1/VP2/VP3), which form the outer capsid shell that protects the viral genome, as well as being actively involved in cell binding and internalization. It is estimated that the viral coat is comprised of 60 proteins arranged into an icosahedral structure with the capsid proteins in a molar ratio of 1:1:10 (VP1:VP2:VP3).

Recombinant AAV vectors having no Rep and/or Cap genes can be non-integrating. In the absence of Rep proteins, ITR-flanked transgenes encoded within rAAV can form circular concatemers that persist as episomes in the nucleus of transduced cells. Because recombinant episomal DNA does not integrate into host genomes, it will eventually be diluted over time as the cell undergoes repeated rounds of replication. This will eventually result in the loss of the transgene and transgene expression.

The sequences placed between the ITRs will typically include a promoter, gene of interest (e.g., encoding a disclosed mRNA), and a terminator. The promoter can be naturally-occurring or non-naturally occurring. In many cases, strong, constitutively active promoters are desired for high-level expression of the gene of interest. Examples of promoters, include, but are not limited to, viral promoters, plant promoters and mammalian promoters. Commonly used promoters include the CMV (cytomegalovirus) promoter/enhancer, EFla (elongation factor 1a), SV40 (simian virus 40), chicken β-actin and CAG (CMV, chicken β-actin, rabbit β-globin) and variants thereof. All of these promoters provide constitutively active, high-level gene expression in most cell types. Some of these promoters are subject to silencing in certain cell types, therefore this consideration can be evaluated for each application.

Examples of terminators include, but are not limited to, polyadenylation signal sequences. Examples of polyadenylation signal sequences include, but are not limited to, Bovine growth hormone (BGH) poly(A), SV40 late poly(A), rabbit beta-globin (RBG) poly(A), thymidine kinase (TK) poly(A) sequences, and any variants thereof.

The viral vectors (e.g., AAV vector) can also have one or more restriction site(s) located near the promoter sequence to provide for the insertion of nucleic acid sequences encoding a mRNA/protein of interest.

The AAV vector used in the disclosed compositions and methods can be a naturally occurring serotype of AAV including, but not limited to, AAV1, AAV2, AAV3, AAV4, AAV5, AAV6, AAV7, AAV8, AAV9, AAV10, AAV11, AAV12, artificial variants such as AAV.rhlO, AAV.rh32/33, AAV.rh43, AAV.rh64Rl, rAAV2-retro, AAV-DJ, AAV-PHP.B, AAV-PHP.S, AAV-PHP.eB, or other engineered versions of AAV. In a particular form, the AAV vector is AAV9. These serotypes differ in their tropism, or the types of cells they infect, making AAV a very useful system for in certain embodiments transducing specific cell types. Typically, AAV vectors have a packaging limit of ~4.7 kb. The AAV itself may be immunogenic, which in some settings, can be used for its adjuvant effects.

Expression cassettes for AAV vectors typically include AAV 5′-ITRs, coding sequences and arbitrary control sequences, as well as AAV 3′-ITRs. However, other arrangements of these elements may be appropriate. A shortened version of the 5′-ITR called ΔITR, which lacks the D sequence and the terminal resolution site (trs), has been described. In other embodiments, the full length AAV 5′-ITR and 3′-ITR are used.

The expression cassette usually contains, for example, a promoter sequence as part of an expression control sequence located between the selected 5′-ITR sequence and the coding sequence.

In addition to promoters, expression cassettes and/or vectors include one or more other suitable transcription initiation, termination, enhancer sequences, efficient RNA processing signals such as splicing and polyadenylation (polyA) signals. It may contain a sequence that stabilizes mRNA; a sequence that enhances translation efficiency (i.e., a Kozak consensus sequence); a sequence that enhances protein stability; and, if desired, a sequence that enhances the secretion of the encoding product.

Examples of suitable poly A sequences include, for example, SV40, SV50, bovine growth hormone (bGH), human growth hormone, and synthetic poly A.

Examples of suitable enhancers include, for example, α-fetoprotein enhancer, TTR minimum promoter/enhancer, LSP (TH-binding globulin promoter/α-microglobulin/bikunin enhancer). In certain embodiments, the expression cassette comprises one or more expression enhancers. In certain embodiments, the expression cassette contains two or more expression enhancers. These enhancers may be the same or different from each other. The enhancer can be present in two copies located adjacent to each other. Alternatively, the dual copy of the enhancer can be separated by one or more sequences. In yet another embodiment, the expression cassette further contains an intron, such as a Promega intron. Other suitable introns include those known in the art, such as those described in International Patent Application No. WO 2011/126808, which is incorporated herein by reference in its entirety for all purposes.

Recombinant AAV viral vectors are well suited for delivery of the coding sequences described herein. Such AAV vectors are ITRs derived from the same AAV source as the capsid. Alternatively, the AAV ITR may be derived from an AAV source different from that supplying the capsid.

Yet other promoters, including tissue-specific promoters, may be selected. Methods for making and isolating AAV viral vectors suitable for delivery to subjects are known in the art. For example, U.S. Patent Application Publication No. US2007/0036760 (Feb. 15, 2007), U.S. Pat. Nos. 7,790,449; 7,282,199; 7,588,772; and International Publication Nos. WO2003/042397; WO2005/033321; WO2006/11689; all of which are incorporated herein by reference in their entireties for all purposes. The sequence of AAV8 and the method for producing a vector based on AAV8 capsid are described in U.S. Pat. Nos. 7,282,199; 7,790,449; and 8,318,480; all of which are incorporated herein by reference in their entireties. In certain embodiments, the vector is based on a AAV9 capsid.

In certain embodiments, the vector is a bacterial expression vector. In certain embodiments, the bacterial expression vector comprises E. coli. In certain embodiments, the bacterial expression vector comprises a pET E. coli T7 expression vector. In certain embodiments, the pET E. coli T7 expression vector comprises pET29b (Novagen).

### Compositions

In one aspect, the present disclosure provides a polypeptide comprising o instances of B, p instances of T, q instances of L, wherein:
each occurrence of B, if present, independently comprises an immunogenic fragment of a bacterial surface protein,
   wherein each instance of B has a C-terminus and a N-terminus;
each occurrence of T, if present, independently comprises an immunogenic fragment of an iron receptor protein,
   wherein each instance of T has a C-terminus and a N-terminus;
each occurrence of L independently comprises a polypeptide of 1 to 40 amino acids,
   wherein each instance of L has a C-terminus and a N-terminus;
each instance of B is covalently linked to one or two independent instances of L by a covalent peptide bond between the C-terminus of the B and the N-terminus of the L and/or the N-terminus of the B and the C-terminus of the L;
each instance of T is covalently linked to one or two independent instances of L by a covalent peptide bond between the C-terminus of the T and the N-terminus of the L and/or the N-terminus of the T and the C-terminus of the L;
o is an integer ranging from 0 to 15;
p is an integer ranging from 0 to 15;
q is an integer ranging from 5 to 30; and
wherein o+p≥6.

In certain embodiments, the C-terminus of the polypeptide may comprise one or more amino acids associated with polypeptide synthesis and/or purification (e.g., poly-His tag or HHHHHH). It is understood by those of ordinary skill in the art that the design of poly-His tags (i.e., terminal position and relatively short length of 6-10 residues) does not confer any biological activity (e.g., immunogenicity) to the polypeptides to which they are covalently linked. It is further understood by those of ordinary skill in the art that the design of poly-His tags minimizes and/or eliminates interference with the biological activity of the polypeptides to which they are covalently linked. As one of ordinary skill in the art would recognize, particularly in view of the application disclosure, the disclosure is not limited to the exemplary polypeptides described herein which comprise a poly-His tag (e.g., SEQ ID NOs:127-145), but further encompasses analogues thereof which lack poly-His functionalization (e.g., SEQ ID NOs:223-241).

In certain embodiments, the N-terminus of a polypeptide of the disclosure may comprise one or more amino acids associated with polypeptide synthesis and/or purification (e.g., methionine). In certain embodiments, certain exemplary polypeptides of the disclosure comprise an N-terminal methionine residue. As one of ordinary skill in the art appreciates, the N-terminal methionine residue present in certain exemplary polypeptides is an artifact of the method by which the polypeptides are synthetized, wherein the AUG start codon of the mRNA transcript (i.e., adenine-uracil-guanine) encodes methionine. Accordingly, as one of ordinary skill in the art would recognize, particularly in view of the application disclosure, the disclosure is not limited to the exemplary polypeptides described herein which comprise an N-terminal methionine residue (e.g., SEQ ID NOs:127-145 and SEQ ID NOs:223-241), but further encompasses analogues thereof which lack an N-terminal methionine residue (e.g., SEQ ID NOs:242-260). Further, one of ordinary skill in the art is apprised of the methods by which N-terminal methionine residues are post-translationally removed (i.e., N-terminal methionine excision or cleavage). In certain embodiments, N-terminal methionine excision and/or cleavage is catalyzed by methionine aminopeptidase (MetAP).

In certain embodiments, each occurrence of L independently comprises a polypeptide of 1 to 10 amino acids. In certain embodiments, each occurrence of L independently comprises a polypeptide of 3 to 10 amino acids. In certain embodiments, each occurrence of L independently comprises a polypeptide of 3 to 25 amino acids. In certain embodiments, multiple occurrences of L may be positioned adjacent to one another.

In certain embodiments, each occurrence of L independently comprises a polypeptide having an amino acid sequence selected from the group consisting of SEQ ID NO:124 (GSGS), SEQ ID NO:125 (GPGP), SEQ ID NO:126 (LLSVGG), SEQ ID NOs:146-155 ((SGSG)₁₋₁₀), SEQ ID NO:156 (SSSS), SEQ ID NO:157 (GGGS), SEQ ID NO:158 (GGC), SEQ ID NO:159 (GGS), SEQ ID NO:160 ((GGC)₈), SEQ ID NO:161 ((GGGGS)₃), and SEQ ID NO:162 (GGAAY). In certain embodiments, each occurrence of L may be cleavable by a protease.

In certain embodiments, B is absent. In certain embodiments, 1 to 15 instances of B are denoted as B¹, B², B³, B⁴, B⁵, B⁶, B⁷, B⁸, B⁹, B¹⁰, B¹¹, B¹², B¹³, B¹⁴, and B¹⁵, respectively. In certain embodiments, each instance of B is independently denoted B¹, B², B³, B⁴, B⁵, B⁶, B⁷, B⁸, B⁹, B¹⁰, B¹¹, B¹², B¹³, B¹⁴, or B¹⁵.

In certain embodiments, T is absent. In certain embodiments, 1 to 15 instances of T are denoted as T¹, T², T³, T⁴, T⁵, T⁶, T⁷, T⁸, T⁹, T¹⁰, T¹¹, T¹², T¹³, T¹⁴, and T¹⁵, respectively. In certain embodiments, each instance of T is independently denoted T¹, T², T³, T⁴, T⁵, T⁶, T⁷, T⁸, T⁹, T¹⁰, T¹¹, T¹², T¹³, T¹⁴, or T¹⁵.

In certain embodiments, 5 to 30 instances of L are denoted as L¹, L², L³, L⁴, L⁵, L⁶, L⁷, L⁸, L⁹, L¹⁰, L¹¹, L¹², L¹³, L¹⁴, L¹⁵, L¹⁶, L¹⁷, L¹⁸, L¹⁹, L²⁰, L²¹, L²², L²³, L²⁴, L²⁵, L²⁶, L²⁷, L²⁸, L²⁹, and L³⁰, respectively. In certain embodiments, each instance of L is independently denoted L¹, L², L³, L⁴, L⁵, L⁶, L⁷, L⁸, L⁹, L¹⁰, L¹¹, L¹², L¹³, L¹⁴, L¹⁵, L¹⁶, L¹⁷, L¹⁸, L¹⁹, L²⁰, L²¹, L²², L²³, L²⁴, L²⁵, L²⁶, L²⁷, L²⁸, L²⁹, or L³⁰.

In certain embodiments, the polypeptide has a structure of B¹-L¹-T¹-L²-B²-L³-T²-L⁴-B³-L⁵-T³-L⁶-B⁴-L⁷-T⁴-L⁸-B⁵-L⁹-T⁵-L¹⁰-B⁶-L¹¹-T⁶-L¹²-B⁷-L¹³-T⁷-L¹⁴-B⁸-L¹⁵-T⁸-L¹⁶-B⁹-L¹⁷-T⁹-L¹⁸-B¹⁰-L¹⁹-T¹⁰-L²⁰-B¹¹-L²¹-T¹¹-L²²-B¹²-L²³-T¹²-L²⁴-B¹³-L²⁵-T¹³-L²⁶-B¹⁴-L²⁷-T¹⁴-L²⁸-B¹⁵-L²⁹-T¹⁵S.

In certain embodiments, the polypeptide may comprise an instance of L which is not adjacent to two instances of B, two instances of T, or one instance of each of B and T (i.e., a terminal L), as represented in the following non-limiting embodiment: B¹-L¹-T¹-L²-B²-L³-T²-L⁴-B³-L⁵-T³-L⁶-B⁴-L⁷-T⁴-L⁸-B⁵-L⁹-T⁵-L¹⁰-B⁶-L¹¹-T⁶-L¹²-B⁷-L¹³-T⁷-L¹⁴-B⁸-L¹⁵-T⁸-L¹⁶-B⁹-L¹⁷-T⁹-L¹⁸-B¹⁰-L¹⁹-T¹⁰-L²⁰-B¹¹-L²¹-T¹¹-L²²-B¹²-L²³-T¹²-L²⁴-B¹³-L²⁵-T¹³-L²⁶-B¹⁴-L²⁷-T¹⁴-L²⁸-B¹⁵-L²⁹-T¹⁵-L³⁰.

The present disclosure encompasses any arrangement of any instance of B and any instance of T.

In certain embodiments, the polypeptide may be initiated (i.e., N-terminus of the polypeptide) by an instance of T, as represented in the following non-limiting embodiment: T¹-L¹-B¹-L²-T²-L³-B²-L⁴-T³-L⁵-B³-L⁶-T⁴-L⁷-B⁴-L⁸-T⁵-L⁹-B⁵-L¹⁰-T⁶-L¹¹-B⁶-L¹²-T⁷-L¹³-B⁷-L¹⁴-T⁸-L¹⁵-B⁸-L¹⁶-T⁹-L¹⁷-B⁹-L¹⁸-T¹⁰-L¹⁹-B¹⁰-L²⁰-T¹¹-L²¹-B¹¹-L²²-T¹²-L²³-B¹²-L²⁴-T¹³-L²⁵-B¹³-L²⁶-T¹⁴-L²⁷-B¹⁴-L²⁸-T¹⁵-L²⁹-B¹³.

In certain embodiments, two instances of B and/or two instances of T may be separated by an instance of L (e.g., B¹-L¹-B² and/or T¹-L²-T²), as represented in the following non-limiting embodiments: B¹-L¹-B²-L²-T¹-L³-T²-L⁴-B³-L⁵-B⁴-L⁶-T³-L⁷-T⁴-L⁸-B⁵-L⁹-B⁶-L¹⁰-T⁵-L¹¹-T⁶-L¹²-B⁷-L¹³-B⁸-L¹⁴-T⁷-L¹⁵-T⁸-L¹⁶-B⁹-L¹⁷-B¹⁰-L¹⁸-T⁹-L¹⁹-T¹⁰-L²⁰-B¹¹-L²¹-B¹²-L²²-T¹¹-L²³-T¹²-L²⁴-B¹³-L²⁵-B¹⁴-L²⁶-T¹³-L²⁷-T¹⁴-L²⁸-B¹⁵-L²⁹-T¹⁵; B¹-L¹-B²-L²-B³-L³-B⁴-L⁴-B⁵-L⁵-B⁶-L⁶-B⁷-L⁷-B⁸-L⁸-B⁹-L⁹-B¹⁰-L¹⁰-B¹¹-L¹¹-B¹²-L¹²-B¹³-L¹³-B¹⁴-L¹⁴-B¹⁵-L¹⁵-T¹-L¹⁶-T²-L¹⁷-T³-L¹⁸-T⁴-L¹⁹-T⁵-L²⁰-T⁶-L²¹-T⁷-L²²-T⁸-L²³-T⁹-L²⁴-T¹⁰-L²⁵-T¹¹-L²⁶-T¹²-L²⁷-T¹³-L²⁸-T¹⁴-L²⁹-T¹⁵; or many other embodiments in which the arrangement of instances of B and T are interchanged, all of which are contemplated herein.

In certain embodiments, any of B¹, B², B³, B⁴, B⁵, B⁶, B⁷, B⁸, B⁹, B¹⁰, B¹¹, B¹², B¹³, B¹⁴, B¹⁵, T¹, T², T³, T⁴, T⁵, T⁶, T⁷, T⁸, T⁹, T¹⁰, T¹¹, T¹², T¹³, T¹⁴, T¹⁵, L¹, L², L³, L⁴, L⁵, L⁶, L⁷, L⁸, L⁹, L¹⁰, L¹¹, L¹², L¹³, L¹⁴, L¹⁵, L¹⁶, L¹⁷, L¹⁸, L¹⁹, L²⁰, L²¹, L²², L²³, L²⁴, L²⁵, L²⁶, L²⁷, L²⁸, L²⁹, or L³⁰, may be present or absent, such that at least 5 instances of L are present, and at a total of 6 instances of B and/or T are present, as represented in the following non-limiting embodiments: B¹-L-B²-L²-B³-L³-B⁴-L⁴-B⁵-L⁵-B⁶; T¹-L¹-T²-L²-T³-L³-T⁴-L⁴-T⁵-L⁵-T⁶; B¹-L¹-B²-L²-B³-L³-B⁴-L⁴-B⁵-L⁵-T¹; T¹-L¹-T²-L²-T³-L³-T⁴-L⁴-T⁵-L⁵-B¹; B¹-L¹-B²-L²-B³-L³-B⁴-L⁴-T¹-L⁵-T²; T¹-L¹-T²-L²-T³-L³-T⁴-L⁴-B¹-L⁵-B²; B¹⁰-L¹¹-B¹¹-L¹²-B¹²-L¹³-B¹³-L¹⁴-B¹⁴-L¹⁵-T²⁵; or many other embodiments of truncated polypeptides in which at least 5 instances of L are present, and a total of 6 instances among B and T are present, all of which are contemplated herein.

In certain embodiments, wherein the identity of each of B¹, B², B³, B⁴, B⁵, B⁶, B⁷, B⁸, B⁹, B¹⁰, B¹¹, B¹², B¹³, B¹⁴, B¹⁵, T¹, T², T³, T⁴, T⁵, T⁶, T⁷, T⁸, T⁹, T¹⁰, T¹¹, T¹², T¹³, T¹⁴, and T¹⁵ are defined, the position of any two identical or unidentical instances of B (e.g., B¹ and B²) and/or any two identical or unidentical instances of T (e.g., T¹ and T²) can be substituted, as represented in the following pairs of non-limiting embodiments:
B¹-L¹-T¹-L²-B²-L³-T²-L⁴-B³-L⁵-T³-L⁶-B⁴-L⁷-T⁴-L⁸-B⁵-L⁹-T¹⁰-L¹¹-B⁶-L¹¹-T⁶-L¹²-B⁷-L¹³-T⁷-L¹⁴-B⁸-L¹⁵-T⁸-L¹⁶-B⁹-L¹⁷-T⁹-L¹⁸-B¹⁰-L¹⁹-T¹⁰-L²⁰-B¹¹-L²¹-T¹¹-L²²-B¹²-L²³-T¹² and B²-L¹-T¹-L²-B¹-L³-T²-L⁴-B³-L⁵-T³-L⁶-B⁴-L⁷-T⁴-L⁸-B⁵-L⁹-T⁵-L¹⁰-B⁶-L¹¹-T⁶-L¹²-B⁷-L¹³-T⁷-L¹⁴-B⁸-L¹⁵-T⁸-L¹⁶-B⁹-L¹⁷-T⁹-L¹-B¹⁰-L¹⁹-T¹⁰-L²⁰-B¹¹-L²¹-T¹¹-L²²-B¹²-L²³-T¹² (e.g., the position of B¹ and B² are substituted); and
B¹-L¹-T¹-L²-B²-L³-T²-L⁴-B³-L⁵-T³-L⁶-B⁴-L⁷-T⁴-L⁸-B⁵-L⁹-T⁵-L¹⁰-B⁶-L¹¹-T⁶-L¹²-B⁷-L¹³-T⁷-L¹⁴-B⁸-L¹⁵-T⁸-L¹⁶-B⁹-L¹⁷-T⁹-L¹⁸-B¹⁰-L¹⁹-T¹⁰-L²⁰-B¹¹-L²¹-T¹¹-L²²-B¹²-L²³-T¹² and B¹-L¹-T²-L²-B²-L³-T¹-L⁴-B³-L⁵-T³-L⁶-B⁴-L⁷-T⁴-L⁸-B⁵-L⁹-T⁵-L¹⁰-B⁶-L¹¹-T⁶-L¹²-B⁷-L¹³-T⁷-L¹⁴-B⁸-L¹⁵-T⁸-L¹⁶-B⁹-L¹⁷-T⁹-L¹⁸-B¹⁰-L¹⁹-T¹⁰-L²⁰-B¹¹-L²¹-T¹¹-L²²-B¹²-L²³-T¹² (e.g., the position of T¹ and T² are substituted).

In certain embodiments, the polypeptide is a polypeptide of structure B¹-L¹-T¹-L²-B²-L³-T²-L⁴-B³-L⁵-T³-L⁶-B⁴-L⁷-T⁴-L⁸-B⁵-L⁹-T⁵-L¹⁰-B⁶-L¹¹-T⁶-L¹²-B⁷-L¹³-T⁷-L¹⁴-B⁸-L¹⁵-T⁸-L¹⁶-B⁹-L¹⁷-T⁹-L¹⁸-B¹⁰-L¹⁹-T¹⁰-L²⁰-B¹¹-L²¹-T¹¹-L²²-B²¹-L²³-T¹².

In certain embodiments, the polypeptide is a polypeptide of structure B¹-L¹-T¹-L²-B²-L³-T²-L⁴-B³-L-T³-L⁶-B⁴-L⁷-T⁴-L⁸-B⁵-L⁹-T⁵-L¹⁰-B⁶-L¹¹-T⁶-L¹²-B⁷-L¹³-T⁷-L¹⁴-B⁸-L¹⁵-T⁸-L¹⁶-B⁹-L¹⁷-T⁹.

In certain embodiments, the polypeptide is a polypeptide of structure T¹-L¹-T²-L²-T³-L³-T⁴-L⁴-T⁵-L⁵-T⁶-L⁶-T⁷-L⁷-T⁸-L⁸-T⁹-L⁹-T¹⁰-L¹⁰-T¹¹-L¹²-T¹².

In certain embodiments, the polypeptide is a polypeptide of structure T¹-L¹-T²-L²-T³-L³-T⁴-L⁴-T⁵-L⁵-T⁶-L⁶-T⁷-L⁷-T⁸-L⁸-T⁹.

In certain embodiments, the polypeptide is a polypeptide of structure B¹-L¹-T¹-L²-B²-L³-T²-L⁴-B³-L⁵-T³-L⁶-B⁴-L⁷-T⁴-L⁸-B⁵-L⁹-T⁵-L¹⁰-B⁶-L¹¹-T⁶-L¹²-B⁷-L¹³-T⁷-L¹⁴-B⁸-L¹⁵-T⁸-L¹⁶-B⁹-L¹⁷-T⁹-L¹⁸-B¹⁰-L¹⁹-T¹⁰-L²⁰-B¹¹-L²¹-T¹¹-L²²-B¹²-L²³-T¹²-L²⁴-B¹³-L²⁵-T¹³.

In certain embodiments, the polypeptide is a polypeptide of structure T¹-L¹-T²-L²-T³-L³-T⁴-L⁴-T⁵-L⁵-T⁶-L⁶-T⁷-L⁷-T⁸-L⁸-T⁹-L⁹-T¹⁰-L¹⁰-T¹¹-L¹¹-T¹²-L¹²-T¹³.

In certain embodiments, the polypeptide is a polypeptide of structure B¹-L¹-T¹-L²-B²-L³-T²-L⁴-B³-L⁵-T³-L⁶-B⁴-L⁷-T⁴-L⁸-B⁵-L⁹-T⁵-L¹⁰-B⁶-L¹¹-T⁶-L¹²-B⁷-L¹³-T⁷-L¹⁴-B⁸-L¹⁵-T⁸-L¹⁶-B⁹-L¹⁷-T⁹-L¹⁸-B¹⁰-L¹⁹-T¹⁰-L²⁰-B¹¹-L²¹-T¹¹-L²²-B¹²-L²³-T¹²-L²⁴-B¹³.

In certain embodiments, the polypeptide is a polypeptide of structure B¹-L¹-B²-L²-B³-L³-B⁴-L⁴-T¹-L⁵-T²-L⁶-T³-L⁷-T⁴-L⁸-B⁵-L⁹-B⁶-L¹⁰-B⁷-L¹¹-B⁸-L¹²-T⁵-L¹³-T⁶-L¹⁴-T⁷-L¹⁵-T⁸-L¹⁶-B⁹-L¹⁷B¹⁰-L¹⁸-B¹¹-L¹⁹-B¹²-L²⁰-T⁹-L²¹-T¹⁰-L²²-T¹¹-L²³-T¹².

In certain embodiments, the polypeptide is a polypeptide of structure T¹-L¹--L²-T³-L³-T⁴-L⁴-B¹-L⁵-B²-L⁶-B³-L⁷-B⁴-L⁸-T⁵-L⁹-T⁶-L¹⁰-T⁷-L¹¹-T⁸-L¹²-B⁵-L¹³-B⁶-L¹⁴-B⁷-L¹⁵-B⁸-L¹⁶-T⁹-L¹⁷-T¹⁰-L¹⁸-T¹¹-L¹⁹-T¹²-L²⁰-B⁹-L²¹-B¹⁰-L²²-B¹¹-L²³-B¹².

One skilled in the art would appreciate that the present disclosure is not limited to the embodiments explicitly disclosed herein.

In certain embodiments, the polypeptide comprises a polypeptide of formula (I), or a salt or solvate thereof:

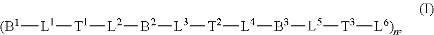

wherein:
each occurrence of B¹, B², B³, L¹, L², L³, L⁴, L⁵, L⁶, T¹, T², and T³ can be present or absent;
each occurrence of B¹, B², and B³, if present, independently comprise an immunogenic fragment of a bacterial surface protein;
each occurrence of T¹, T², and T³, if present, independently comprise an immunogenic fragment of an iron receptor protein,
   wherein at least two of T¹, T², and T³ are present or at least one of T¹, T² and T³ is present and n is at least 2;
each occurrence of L¹, L², L³, L⁴, L⁵, and L⁶, if present, independently comprise a polypeptide of 1-10 amino acids,
   wherein at least one of L¹, L², L³, L⁴, L⁵, and L⁶ is present, and
   wherein each occurrence of B¹, B², B³, T¹, T², and T³ are separated from one another by at least one of L¹, L², L³, L⁴, L⁵, and L⁶; and
n is an integer selected from the group consisting of 1, 2, 3, 4, and 5.

In certain embodiments, the polypeptide of formula (I) comprises n (i.e., 1, 2, 3, 4, or 5) independent occurrences of B¹-L¹-T¹-L²-B²-L³-T²-L⁴-B³-L⁵-T³-L⁶. In certain non-limiting embodiments, wherein n is 2, a first independent occurrence of B¹-L¹-T¹-L²-B²-L³-T²-L⁴-B³-L⁵-T³-L⁶ is covalently linked to a second independent occurrence of B¹-L¹-T¹-L²-B²-L³-T²-L⁴-B³-L⁵-T³-L⁶ via an amide (peptide) bond. In certain embodiments, the covalent linkage comprises a linear arrangement of each occurrence of B¹-L¹-T¹-L²-B²-L³-T²-L⁴-B³-L⁵-T³-L⁶. For example, in certain embodiments, a terminal B¹, B², B³, T¹, T², or T³ of a first occurrence of B¹-L¹-T¹-L²-B²-L³-T²-L⁴-B³-L⁵-T³-L⁶ is covalently linked to a terminal L¹, L², L³, L⁴, L⁵, or L⁶ of a second occurrence of B¹-L¹-T¹-L²-B²-L³-T²-L⁴-B³-L⁵-T³-L⁶*. For example, in one non-limiting embodiment, wherein n is 2 and each of B¹, B², B³, L¹, L², L³, L⁴, L⁵, L⁶, T¹, T², and T³ are present in both occurrences of B¹-L¹-T¹-L²-B²-L³-T²-L⁴-B³-L⁵-T³-L⁶, the polypeptide of formula (I) has the following structure (e.g., wherein a terminal L⁶ of a first occurrence is covalently bound to a terminal B¹ of a second occurrence):

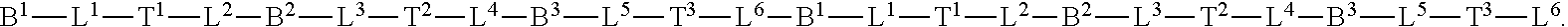

That said, whether any one of B¹, B², B³, L¹, L², L³, L⁴, L⁵, L⁶, T¹, T², or T³ is present or absent in a given occurrence of B¹-L¹-T¹-L²-B²-L³-T²-L⁴-B³-L⁵-T³-L⁶, the number of occurrences of which is defined by n, is independent of each group's presence or absence in any other occurrence of B¹-L¹-T¹-L²-B²-L³-T²-L⁴-B³-L⁵-T³-L⁶. For example, in one non-limiting embodiment, wherein n is 2, a first occurrence of B¹-L¹-T¹-L²-B²-L³-T²-L⁴-B³-L⁵-T³-L⁶ may comprise only B¹, L¹, T¹, and L² (B¹-L¹-T¹-L²) and a second occurrence of B¹-L¹-T¹-L²-B²-L³-T²-L4-B³-L⁵-T³-L⁶ may comprise only T¹, L⁵, and T³ (T¹-L⁵-T³). In the aforementioned non-limiting embodiment, the polypeptide of formula (I) has the following structure: B¹-L¹-T¹-L²-T¹-L⁵-T³. Thus, in the aforementioned non-limiting embodiment, B¹, L¹, and L² are present only in the first occurrence of B¹-L¹-T¹-L²-B²-L³-T²-L0-B³-L⁵-T³-L⁶, L⁵ and T³ are present only in the second occurrence of B¹-L¹-T¹-L²-B²-L³-T²-L⁴-B³-L⁵-T³-L⁶, and T¹ is present in both the first and second occurrences of B¹-L¹-T¹-L²-B²-L³-T²-L0-B³-L⁵-T³-L.

Further, the identity of each of B¹, B², B³, L¹, L², L³, L⁴, L⁵, L⁶, T¹, T², and T³ in each occurrence of B¹-L¹-T¹-L²-B²-L³-T²-L⁴-B³-L⁵-T³-L⁶, the number of occurrences of which is defined by n, are independent from one another. For example, in the aforementioned non-limiting embodiment, wherein n is 2 and T¹ is present in both occurrences of B¹-L¹-T¹-L²-B²-L³-T²-L⁴-B³-L⁵-T³-L⁶, T¹ in the first occurrence of B¹-L¹-T¹-L²-B²-L³-T²-L0-B³-L⁵-T³-L⁰ may comprise an immunogenic fragment of the iron receptor protein defined by SEQ ID NO:10, and T¹ in the second occurrence of B¹-L¹-T¹-L²-B²-L³-T²-L⁴-B³-L⁵-T³-L⁶ may comprise an immunogenic fragment of the iron receptor protein defined by SEQ ID NO:12.

In the polypeptide of formula (I), each occurrence of B¹, B², and B³, if present, independently comprises an immunogenic fragment of a bacterial surface protein. In certain embodiments, each occurrence of B¹, B², and/or B³ is independently present or absent. In certain embodiments, each occurrence of B¹, B², and B³, may comprise an identical immunogenic fragment of a bacterial surface protein. In certain embodiments, each occurrence of B¹, B², and B³, may comprise a different immunogenic fragment of a bacterial surface protein. Thus, in certain non-limiting embodiments, wherein a single occurrence of each of B¹ and B² are present, and the bacterial surface protein of both B¹ and B² may correspond to SEQ ID NO:1, the immunogenic fragment of the bacterial surface protein in B¹ may comprise SEQ ID NO:16, and the immunogenic fragment of the bacterial surface protein in B² may comprise SEQ ID NO:17. Additionally, the identity of the bacterial surface protein need not be the same in each occurrence of B¹, B², and B³. In certain embodiments, each occurrence of B¹, B², and B³, may comprise immunogenic fragments of different bacterial surface proteins. For example, in certain non-limiting embodiments, wherein a single occurrence of each of B¹ and B² are present, the bacterial surface protein of B¹ may comprise SEQ ID NO:1 and the bacterial surface protein of B² may comprise SEQ ID NO:2. Similarly, in certain non-limiting embodiments, wherein the polypeptide of formula (I) comprises two occurrences of B¹, the first occurrence of B¹ may comprise bacterial surface protein SEQ ID NO:1 and the second occurrence of B¹ may comprise bacterial surface protein SEQ ID NO:2. Alternatively, in certain non-limiting embodiments, wherein the polypeptide of formula (I) comprises two occurrences of B¹, the first and second occurrences of B¹ may both comprise bacterial surface protein SEQ ID NO:1.

In the polypeptide of formula (I), each occurrence of T¹, T², and T³, if present, independently comprises an immunogenic fragment of an iron receptor protein. In certain embodiments, each occurrence of T¹, T², and/or T³ is independently present or absent. In certain embodiments, each occurrence of T¹, T², and T³, may comprise an identical immunogenic fragment of an iron receptor protein. In certain embodiments, each occurrence of T¹, T², and T³, may comprise a different immunogenic fragment of an iron receptor protein. Thus, in certain non-limiting embodiments, wherein a single occurrence of each of T¹ and T² are present, and the iron receptor protein of both T¹ and T² may correspond to SEQ ID NO:10, the immunogenic fragment of the iron receptor protein in T¹ may comprise SEQ ID NO:42, and the immunogenic fragment of the iron receptor protein in T² may comprise SEQ ID NO:43. Additionally, the identity of the iron receptor protein need not be the same in each occurrence of T¹, T², and T³. In certain embodiments, each occurrence of T¹, T², and T³, may comprise immunogenic fragments of different iron receptor proteins. For example, in certain non-limiting embodiments, wherein a single occurrence of each of T¹ and T² are present, the iron receptor protein of T¹ may comprise SEQ ID NO:10 and the iron receptor protein of T² may comprise SEQ ID NO:12. Similarly, in certain non-limiting embodiments, wherein the polypeptide of formula (I) comprises two occurrences of T¹, the first occurrence of T may comprise iron receptor protein SEQ ID NO:10 and the second occurrence of T may comprise iron receptor protein SEQ ID NO:12. Alternatively, in certain non-limiting embodiments, wherein the polypeptide of formula (I) comprises two occurrences of T¹, the first and second occurrences of T¹ may both comprise iron receptor protein SEQ ID NO:10.

In the polypeptide of formula (I), each occurrence of L¹, L², L³, L⁴, L⁵, and L⁶, if present, independently comprise a polypeptide of 1-10 amino acids. In certain embodiments, each occurrence of L¹, L², L³, L⁴, L⁵, and/or L⁶ is independently present or absent. In certain embodiments, each occurrence of L¹, L², L³, L⁴, L⁵, and/or L⁶, may comprise a different 1-10 amino acid polypeptide. Thus, in certain non-limiting embodiments, wherein a single occurrence of each of L¹ and L² are present, L¹ may comprise SEQ ID NO:124 and L² may comprise SEQ ID NO:125. Alternatively, in certain non-limiting embodiments, wherein a single occurrence of each of L¹ and L² are present, L¹ and L² may both comprise SEQ ID NO:124. Similarly, in certain non-limiting embodiments, wherein n is 2 and the polypeptide of formula (I) comprises two occurrences of L¹, the first occurrence of L¹ may comprise SEQ ID NO:124 and the second occurrence of L¹ may comprise SEQ ID NO:125. Alternatively, in certain non-limiting embodiments, wherein n is 2 and the polypeptide of formula (I) comprises two occurrences of L¹, both the first and second occurrences of L¹ may comprise SEQ ID NO:124.

In certain embodiments, at least one of B¹, B², and B³ is present.

In certain embodiments, each bacterial surface protein shares at least 85%, 90%, 95%, 96%, 97%, 98%, 99%, or 100% sequence homology with a polypeptide independently selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, and SEQ ID NO:9.

In certain embodiments, each immunogenic fragment of the bacterial surface protein shares at least 85%, 90%, 95%, 96%, 97%, 98%, 99%, or 100% sequence homology with a polypeptide independently selected from the group consisting of SEQ ID NO:16, SEQ ID NO:195, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, and SEQ ID NO:41.

In certain embodiments, each immunogenic fragment of the bacterial surface protein shares at least 85%, 90%, 95%, 96%, 97%, 98%, 99%, or 100% sequence homology with a polypeptide independently selected from the group consisting of SEQ ID NO:16, SEQ ID NO:195, SEQ ID NO:17, SEQ ID NO:18, SEQ ID NO:19, SEQ ID NO:20, SEQ ID NO:23, SEQ ID NO:25, SEQ ID NO:27, SEQ ID NO:32, SEQ ID NO:35, SEQ ID NO:37, SEQ ID NO:39, and SEQ ID NO:40.

In certain embodiments, each of B¹, B², B³, B⁴, B⁵, B⁶, B⁷, B⁸, B⁹, B¹⁰, B¹¹, B¹², B¹³, B¹⁴, and B¹⁵, if present, shares at least 85%, 90%, 95%, 96%, 97%, 98%, 99%, or 100% sequence homology with a polypeptide independently selected from the group consisting of SEQ ID NO:16, SEQ ID NO:195, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, and SEQ ID NO:41.

In certain embodiments, each iron receptor protein shares at least 85%, 90%, 95%, 96%, 97%, 98%, 99%, or 100% sequence homology with a polypeptide independently selected from the group consisting of SEQ ID NO:10, SEQ ID NO:11, SEQ ID NO:12, SEQ ID NO:13, SEQ ID NO:14, and SEQ ID NO:15.

In certain embodiments, each immunogenic fragment of the iron receptor protein shares at least 85%, 90%, 95%, 96%, 97%, 98%, 99%, or 100% sequence homology with a polypeptide independently selected from the group consisting of SEQ ID NO:42, SEQ ID NO:43, SEQ ID NO:4, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, and SEQ ID NO:123.

In certain embodiments, each immunogenic fragment of the iron receptor protein shares at least 85%, 90%, 95%, 96%, 97%, 98%, 99%, or 100% sequence homology with a polypeptide independently selected from the group consisting of SEQ ID NO:42, SEQ ID NO:46, SEQ ID NO:47, SEQ ID NO:51, SEQ ID NO:53, SEQ ID NO:54, SEQ ID NO:55, SEQ ID NO:56, SEQ ID NO:58, SEQ ID NO:62, SEQ ID NO:67, SEQ ID NO:85, and SEQ ID NO:88.

In certain embodiments, each of T¹, T², T³, T⁴, T⁵, T⁶, T⁷, T⁸, T⁹, T¹⁰, T¹¹, T¹², T¹³, T¹⁴, and T¹⁵, if present, shares at least 85%, 90%, 95%, 96%, 97%, 98%, 99%, or 100% sequence homology with a polypeptide independently selected from the group consisting of SEQ ID NO:42, SEQ ID NO:43, SEQ ID NO:4, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, and SEQ ID NO:123.

In certain embodiments, each of L¹, L², L³, L⁴, L⁵, and L⁶, if present, independently comprise a polypeptide wherein each amino acid residue side chain comprises a neutral (i.e., non-charged) substituent.

In certain embodiments, each of L¹, L², L³, L⁴, L⁵, and L⁶, if present, independently comprise a polypeptide of 4 amino acids. In certain embodiments, each of L¹, L², L³, L⁴, L⁵, and L⁶, if present, independently comprise a polypeptide of 5 amino acids. In certain embodiments, each of L¹, L², L³, L⁴, L⁵, and L⁶, if present, independently comprise a polypeptide of 6 amino acids.

In certain embodiments, each of L¹, L², L³, L⁴, L⁵, and L⁶, if present, is independently selected from the group consisting of GSGS (SEQ ID NO:124), GPGP (SEQ ID NO:125), LLSVGG (SEQ ID NO:126), (SGSG)₁₋₂ (SEQ ID NOs:146-147), SSSS (SEQ ID NO:156), GGGS (SEQ ID NO:157), GGC (SEQ ID NO:158), GGS (SEQ ID NO:159), (GGC)₈ (SEQ ID NO:160), (GGGGS)₃ (SEQ ID NO:161), and GGAAY (SEQ ID NO:162).

In certain embodiments, n is 1. In certain embodiments, n is 2. In certain embodiments, n is 3. In certain embodiments, n is 4. In certain embodiments, n is 5.

In certain embodiments, the polypeptide shares at least 85%, 90%, 95%, 96%, 97%, 98%, 99%, or 100% sequence homology with a polypeptide selected from the group consisting of SEQ ID NO:130 and SEQ ID NO:129.

In certain embodiments, the polypeptide shares at least 85%, 90%, 95%, 96%, 97%, 98%, 99%, or 100% sequence homology with a polypeptide selected from the group consisting of SEQ ID NO:127, SEQ ID NO:128, SEQ ID NO:131, SEQ ID NO:132, SEQ ID NO:133, SEQ ID NO:134, SEQ ID NO:135, SEQ ID NO:136, SEQ ID NO:137, SEQ ID NO:143, SEQ ID NO:144, SEQ ID NO:145, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, SEQ ID NO:242, SEQ ID NO:243, SEQ ID NO:244, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:253, SEQ ID NO:254, SEQ ID NO:255, SEQ ID NO:256, SEQ ID NO:257, SEQ ID NO:258, SEQ ID NO:259, and SEQ ID NO:260.

In certain embodiments, the polypeptide shares at least 85%, 90%, 95%, 96%, 97%, 98%, 99%, or 100% sequence homology with SEQ ID NO:135.

In certain embodiments, the polypeptide shares at least 85%, 90%, 95%, 96%, 97%, 98%, 99%, or 100% sequence homology with SEQ ID NO:225.

In certain embodiments, the polypeptide shares at least 85%, 90%, 95%, 96%, 97%, 98%, 99%, or 100% sequence homology with SEQ ID NO:226.

In certain embodiments, the polypeptide comprises a polypeptide selected from the group consisting of:

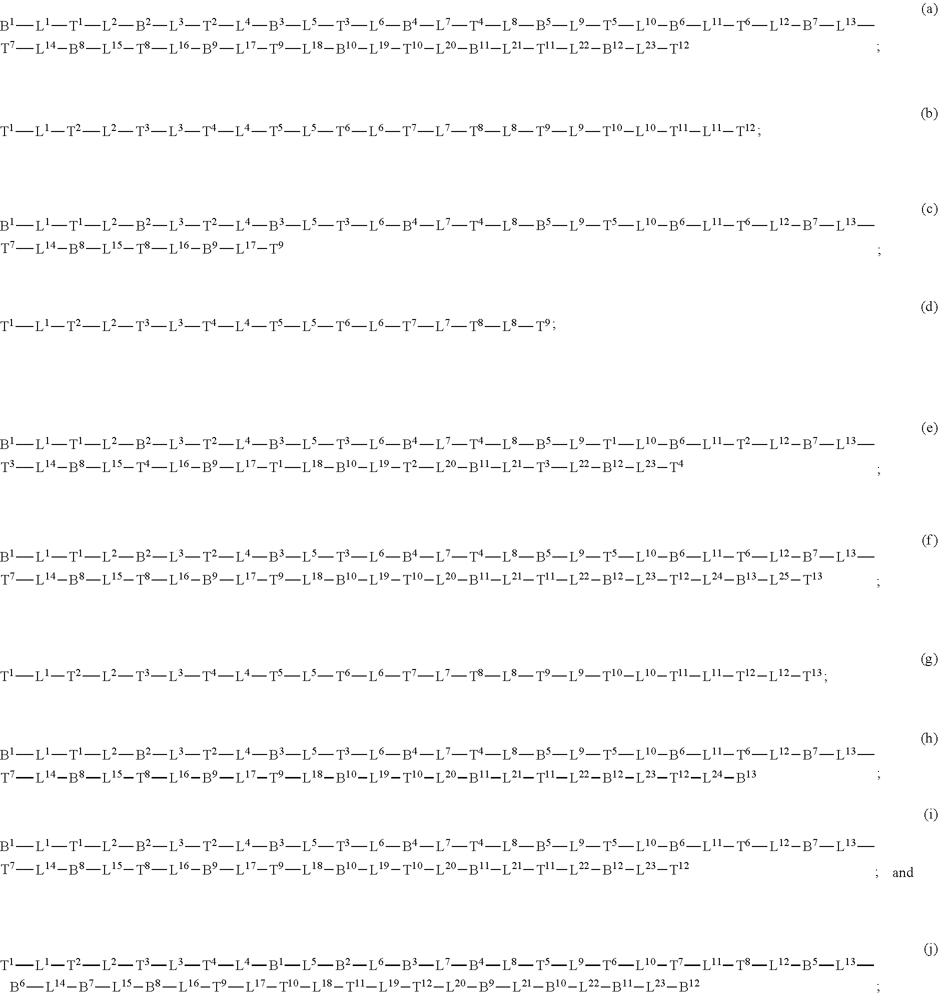

wherein:
   each occurrence of B, if present, independently comprises an immunogenic fragment of a bacterial surface protein,
      wherein each instance of B has a C-terminus and a N-terminus;
   each occurrence of T independently comprises an immunogenic fragment of an iron receptor protein,
      wherein each instance of T has a C-terminus and a N-terminus;
   each occurrence of L independently comprises a polypeptide of 1-10 amino acids,
      wherein each instance of L has a C-terminus and a N-terminus;
   each instance of B is covalently linked to one or two independent instances of L by a covalent peptide bond between the C-terminus of the B and the N-terminus of the L and/or the N-terminus of the B and the C-terminus of the L; and
   each instance of T is covalently linked to one or two independent instances of L by a covalent peptide bond between the C-terminus of the T and the N-terminus of the L and/or the N-terminus of the T and the C-terminus of the L;
or a salt or solvate thereof.

In certain embodiments, each immunogenic fragment of the bacterial surface protein shares at least 85%, 90%, 95%, 96%, 97%, 98%, 99%, or 100% sequence homology with a polypeptide independently selected from the group consisting of SEQ ID NO:16, SEQ ID NO:195, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, and SEQ ID NO:41.

In certain embodiments, each immunogenic fragment of the bacterial surface protein shares at least 85%, 90%, 95%, 96%, 97%, 98%, 99%, or 100% sequence homology with a polypeptide independently selected from the group consisting of SEQ ID NO:16, SEQ ID NO:195, SEQ ID NO:17, SEQ ID NO:18, SEQ ID NO:19, SEQ ID NO:20, SEQ ID NO:23, SEQ ID NO:25, SEQ ID NO:27, SEQ ID NO:32, SEQ ID NO:35, SEQ ID NO:37, SEQ ID NO:39, and SEQ ID NO:40.

In certain embodiments, each iron receptor protein shares at least 85%, 90%, 95%, 96%, 97%, 98%, 99%, or 100% sequence homology with a polypeptide independently selected from the group consisting of SEQ ID NO:10, SEQ ID NO:11, SEQ ID NO:12, SEQ ID NO:13, SEQ ID NO:14, and SEQ ID NO:15.

In certain embodiments, each immunogenic fragment of the iron receptor protein shares at least 85%, 90%, 95%, 96%, 97%, 98%, 99%, or 100% sequence homology with a polypeptide independently selected from the group consisting of SEQ ID NO:42, SEQ ID NO:43, SEQ ID NO:4, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, and SEQ ID NO:123.

In certain embodiments, each immunogenic fragment of the iron receptor protein shares at least 85%, 90%, 95%, 96%, 97%, 98%, 99%, or 100% sequence homology with a polypeptide independently selected from the group consisting of SEQ ID NO:42, SEQ ID NO:46, SEQ ID NO:47, SEQ ID NO:51, SEQ ID NO:53, SEQ ID NO:54, SEQ ID NO:55, SEQ ID NO:56, SEQ ID NO:58, SEQ ID NO:62, SEQ ID NO:67, SEQ ID NO:85, and SEQ ID NO:88.

In certain embodiments, each of L¹, L², L³, L⁴, L⁵, L⁶, L⁷, L⁸, L⁹, L¹⁰, L¹¹, L¹², L¹³, L¹⁴, L¹⁵, L¹⁶, L¹⁷, L¹⁸, L¹⁹, L²⁰, L²¹, L²², L²³, L²⁴, and L²⁵, if present, independently comprise a polypeptide wherein each amino acid residue side chain comprises a neutral (i.e., non-charged) substituent.

In certain embodiments, each of L¹, L², L³, L⁴, L⁵, L⁶, L⁷, L⁸, L⁹, L¹⁰, L¹¹, L¹², L¹³, L¹⁴, L¹⁵, L¹⁶, L¹⁷, L¹⁸, L¹⁹, L²⁰, L²¹, L²², L²³, L²⁴, and L²⁵, if present, independently comprise a polypeptide of 4 to 6 amino acids.

In certain embodiments, each of L¹, L², L³, L⁴, L⁵, L⁶, L⁷, L⁸, L⁹, L¹⁰, L¹¹, L¹², L¹³, L¹⁴, L¹⁵, L¹⁶, L¹⁷, L¹⁸, L¹⁹, L²⁰, L²¹, L²², L²³, L²⁴, and L²⁵, if present, is independently selected from the group consisting of GSGS (SEQ ID NO:124), GPGP (SEQ ID NO:125), LLSVGG (SEQ ID NO:126), (SGSG)₁₋₂(SEQ ID NOs:146-147), SSSS (SEQ ID NO:156), GGGS (SEQ ID NO:157), GGC (SEQ ID NO:158), GGS (SEQ ID NO:159), (GGC)s (SEQ ID NO:160), (GGGGS)₃ (SEQ ID NO:161), and GGAAY (SEQ ID NO:162).

In certain embodiments, the polypeptide shares at least 85%, 90%, 95%, 96%, 97%, 98%, 99%, or 100% sequence homology with a polypeptide selected from the group consisting of SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, SEQ ID NO:242, SEQ ID NO:243, SEQ ID NO:244, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:253, SEQ ID NO:254, SEQ ID NO:255, SEQ ID NO:256, SEQ ID NO:257, SEQ ID NO:258, SEQ ID NO:259, and SEQ ID NO:260.

In certain embodiments, the polypeptide shares at least 85%, 90%, 95%, 96%, 97%, 98%, 99%, or 100% sequence homology with SEQ ID NO:225.

In certain embodiments, the polypeptide shares at least 85%, 90%, 95%, 96%, 97%, 98%, 99%, or 100% sequence homology with SEQ ID NO:226.

### Methods

In another aspect, the present disclosure provides a method of treating, preventing, and/or ameliorating a bacterial infection in a subject in need thereof. In certain embodiments, the method comprises administering to the subject at least one polypeptide of the present disclosure.

In another aspect, the present disclosure provides a method of treating, preventing, and/or ameliorating a bacterial infection in a subject in need thereof. In certain embodiments, the method comprises administering to the subject at least one isolated mRNA of the present disclosure.

In another aspect, the present disclosure provides a method of treating, preventing, and/or ameliorating a bacterial infection in a subject in need thereof. In certain embodiments, the method comprises administering to the subject at least one isolated DNA of the present disclosure.

In another aspect, the present disclosure provides a method of treating, preventing, and/or ameliorating a bacterial infection in a subject in need thereof. In certain embodiments, the method comprises administering to the subject at least one isolated polynucleotide of the present disclosure.

In another aspect, the present disclosure provides a method of treating, preventing, and/or ameliorating a bacterial infection in a subject in need thereof. In certain embodiments, the method comprises administering to the subject at least one vector of the present disclosure.

In another aspect, the present disclosure provides a method of treating, preventing, and/or ameliorating a bacterial infection in a subject in need thereof. In certain embodiments, the method comprises administering to the subject at least one LNP of the present disclosure.

In another aspect, the present disclosure provides a method of treating, preventing, and/or ameliorating a bacterial infection in a subject in need thereof. In certain embodiments, the method comprises administering to the subject at least one the pharmaceutical composition of the present disclosure.

In another aspect, the present disclosure provides a method of treating, preventing, and/or ameliorating a bacterial infection in a subject in need thereof. In certain embodiments, the method comprises administering to the subject at least one vaccine composition of the present disclosure.

In certain embodiments, the bacterial infection is a urinary tract infection. In certain embodiments, the bacterial infection comprises a urinary tract infection and sepsis. In certain embodiments, the bacterial infection is sepsis. In certain embodiments, the sepsis is neonatal sepsis. In certain embodiments, the bacterial infection is pneumonia.

In another aspect, the present disclosure provides a method of generating immunity to infection by one or more pathogenic bacteria in a subject. In certain embodiments, the method comprises administering to the subject at least one polypeptide of the present disclosure.

In another aspect, the present disclosure provides a method of generating immunity to infection by one or more pathogenic bacteria in a subject. In certain embodiments, the method comprises administering to the subject at least one isolated mRNA of the present disclosure. In certain embodiments, the method comprises administering to the subject at least one isolated DNA of the present disclosure.

In another aspect, the present disclosure provides a method of generating immunity to infection by one or more pathogenic bacteria in a subject. In certain embodiments, the method comprises administering to the subject at least one isolated polynucleotide of the present disclosure.

In another aspect, the present disclosure provides a method of generating immunity to infection by one or more pathogenic bacteria in a subject. In certain embodiments, the method comprises administering to the subject at least one vector of the present disclosure.

In another aspect, the present disclosure provides method of generating immunity to infection by one or more pathogenic bacteria in a subject. In certain embodiments, the method comprises administering to the subject at least one LNP of the present disclosure.

In another aspect, the present disclosure provides a method of generating immunity to infection by one or more pathogenic bacteria in a subject. In certain embodiments, the method comprises administering to the subject at least one the pharmaceutical composition of the present disclosure.

In another aspect, the present disclosure provides a method of generating immunity to infection by one or more pathogenic bacteria in a subject. In certain embodiments, the method comprises administering to the subject at least one vaccine composition of the present disclosure. In certain embodiments, the one or more pathogenic bacteria comprises at least one selected from the group consisting of Escherichia coli, Klebsiella pneumoniae, Proteus mirabilis, Shigella dysenteriae, Salmonella enterica, Streptococcus pneumoniae, Haemophilus influenzae, Chlamydophila pneumoniae, Mycoplasma pneumoniae, Staphylococcus aureus, Moraxella catarrhalis, and Legionella pneumophila, Streptococcus pyogenes, and Pseudomonas aeruginosa, and Salmonella bongori.

In certain embodiments, the immunity prevents a bacterial infection.

In certain embodiments, the bacterial infection is a urinary tract infection (UTI). In certain embodiments, the bacterial infection comprises a urinary tract infection and sepsis. In certain embodiments, the bacterial infection is sepsis. In certain embodiments, the sepsis is neonatal sepsis. In certain embodiments, the bacterial infection is pneumonia.

In certain embodiments, the subject is administered one or more additional doses of the polypeptide of the present disclosure after initial administration. In certain embodiments, the subject is administered a second, third, or fourth dose of the polypeptide of the present disclosure after initial administration.

In certain embodiments, the subject is administered one or more additional doses of the isolated mRNA of the present disclosure after initial administration. In certain embodiments, the subject is administered a second, third, or fourth dose of the isolated mRNA of the present disclosure after initial administration.

In certain embodiments, the subject is administered one or more additional doses of the isolated polynucleotide of the present disclosure after initial administration. In certain embodiments, the subject is administered a second, third, or fourth dose of the isolated polynucleotide of the present disclosure after initial administration.

In certain embodiments, the subject is administered one or more additional doses of the vector of the present disclosure after initial administration. In certain embodiments, the subject is administered a second, third, or fourth dose of the vector of the present disclosure after initial administration.

In certain embodiments, the subject is administered one or more additional doses of the LNP of the present disclosure after initial administration. In certain embodiments, the subject is administered a second, third, or fourth dose of the LNP of the present disclosure after initial administration.

In certain embodiments, the subject is administered one or more additional doses of the pharmaceutical composition of the present disclosure after initial administration. In certain embodiments, the subject is administered a second, third, or fourth dose of the pharmaceutical composition of the present disclosure after initial administration.

In certain embodiments, the subject is administered one or more additional doses of the vaccine composition of the present disclosure after initial administration. In certain embodiments, the subject is administered a second, third, or fourth dose of the vaccine composition of the present disclosure after initial administration.

In certain embodiments, each additional dose is administered with an interval ranging from about 1 to about 365 days. In certain embodiments, the interval between dose administrations is selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, and 365 days apart.

In certain embodiments, the subject is a mammal. In certain embodiments, the mammal is a human.

In certain embodiments, the administration comprises maternal immunization.

In certain embodiments, subject is pregnant with a fetus.

In certain embodiments, the bacterial infection is treated, prevented, and/or ameliorated in at least one of the subject and the fetus, or a neonate thereof. In certain embodiments, the bacterial infection is treated, prevented, and/or ameliorated in both the subject and the fetus, or a neonate thereof. In certain embodiments, the bacterial infection is treated, prevented, and/or ameliorated in the neonate or infant after birth of the fetus.

In certain embodiments, the immunity to infection by one or more pathogenic bacteria is generated in at least one of the subject and the fetus, or a neonate thereof. In certain embodiments, the immunity to infection by one or more pathogenic bacteria is generated in both the subject and the fetus, or a neonate thereof. In certain embodiments, the immunity to infection by one or more pathogenic bacteria is generated in the neonate or infant after birth of the fetus.

### Pharmaceutical Compositions and Formulations

In another aspect, the present disclosure provides a vaccine composition comprising the polypeptide of the present disclosure and at least one pharmaceutically acceptable excipient.

In another aspect, the present disclosure provides a vaccine composition comprising at least one LNP of the present disclosure and a pharmaceutically acceptable excipient.

In certain embodiments, the polypeptide shares at least 85%, 90%, 95%, 96%, 97%, 98%, 99%, or 100% sequence homology with a polypeptide selected from the group consisting of SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, SEQ ID NO:242, SEQ ID NO:243, SEQ ID NO:244, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:253, SEQ ID NO:254, SEQ ID NO:255, SEQ ID NO:256, SEQ ID NO:257, SEQ ID NO:258, SEQ ID NO:259, and SEQ ID NO:260.

In certain embodiments, the polypeptide shares at least 85%, 90%, 95%, 96%, 97%, 98%, 99%, or 100% sequence homology with SEQ ID NO:225.

In certain embodiments, the polypeptide shares at least 85%, 90%, 95%, 96%, 97%, 98%, 99%, or 100% sequence homology with SEQ ID NO:226.

In certain embodiments, the vaccine composition further comprises an adjuvant. In certain embodiments, the adjuvant is at least one selected from the group consisting of aluminum hydroxide (AlOH), double mutant heat labile toxin (dmLt), CpG, AddaS03™, AddaVax™, MF59®, W₂₀5EC and/or W₈₀5EC (NanoBio®), Matrix-M™, Quil-A®, monophosphoryl lipid A (MPLA; e.g., PHAD® (synthetic) or E. coli derived), monophosphoryl lipid A Salmonella Minnesota (MPLA-SM; e.g., MPLA-SM VacciGrade™), Alhydrogel®, aluminum phosphate (ALPO₄; e.g., Imject® Alum Adjuvant), chitosan, Class A CpG adjuvant (e.g., OND 1585, ODN 2216, and ODN 2336), Class B CpG adjuvant (e.g., ODN 1018, ODN 1668, ODN 1826, ODN 2006, ODN 2007, ODN BW006, ODN D-SL01, and ODN 7909), Class C CpG adjuvant (e.g., ODN 2395, ODN M362, and ODN D-SL03), cCpG, compositions comprising one or more cationic liposomes and/or one or more immunomodulators (e.g., CAF01), AS₀₁b, gardisil adjuvant, AS02, AS03®, AS04, GLA-SE (i.e., glucopyranosyl lipid adjuvant; synthetic TLR4 agonist), IC31®, Montanide™ ISA-51 VG, Montamide™ ISA-70 VG, immune stimulating complexes (ISCOMs) (e.g., H5N1), cholera toxin (CT), cholera toxin subunit B (CTB), subunit B of heat-labile enterotoxin (LTB), imiquimod, resiquimod (R848), double stranded RNA (dsRNA) hairpin(s) (e.g., polyinosinic:polycytidylic acid (i.e., poly I:C)), Riboxxim®, rintatolimod (Ampligen®), poly ICLC (e.g., Hiltonol®), AF03, polyphosphazene (PCEP and/or PCPP), cyclic diguanylate monophosphate (c-di-GMP or CDG), CAF01, CAF05, CAF09, trehalose dibehenate (TDB), furfurman, curdlan, retinoic acid, protollin, proteosomes (ID Biomedical), Endocine™ (L3B), N30A, N30ASq, NanoVax (NanoBio®), inulin, delta inulin (e.g., Advax™), Advax-CpG55.2, polyactin and/or alpha-polyactin, mannatide, β-glucosylceramide, α-galactosylceramide, cholesterol, IFN-α, flagellin, MALT chemokines (e.g., CCL25, CCL27, and CCL28), glucosylpyranosyl lipid adjuvant, ADP ribosylating toxins, trehalose, and hydroxypropyl methacrylamide (HPMA) co-polymers.

In certain embodiments, the adjuvant comprises aluminum hydroxide. In certain embodiments, the adjuvant comprises dmLT. In certain embodiments, the adjuvant comprises CpG. In certain embodiments, the adjuvant comprises AddaS03™. In certain embodiments, the adjuvant comprises AS03®.

In certain embodiments, the adjuvant comprises AlOH and CpG. In certain embodiments, the adjuvant comprises AddaS03™ and/or AS03® and CpG. In certain embodiments, the adjuvant comprises AddaS03™ and CpG. In certain embodiments, the adjuvant comprises AS03® and CpG.

In certain embodiments, the adjuvant in compositions of the disclosure suitable for treatment and/or prevention of a urinary tract infection in a subject comprise AddaS03™ and/or AS03® and CpG. In certain embodiments, the adjuvant in compositions of the disclosure suitable for treatment and/or prevention of a urinary tract infection in a subject comprise AddaS03™ and CpG. In certain embodiments, the adjuvant in compositions of the disclosure suitable for treatment and/or prevention of a urinary tract infection in a subject comprise AS03® and CpG.

In certain embodiments, the adjuvant in compositions of the disclosure suitable for treatment and/or prevention of neonatal sepsis in a subject comprises AlOH. In certain embodiments, the adjuvant in compositions of the disclosure suitable for treatment and/or prevention of neonatal sepsis in a subject comprises AlOH and CpG.

In certain embodiments, the compositions of the disclosure comprise about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 μg of the polypeptide.

In certain embodiments, the compositions of the disclosure comprise about 10, 20, 30, 40, 50, 60, 70, 80, 90, 100, 110, 120, 130, 140, 150, 160, 170, 180,190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or about 500 μg of the polypeptide.

In certain embodiments, the vaccine composition has a concentration of the polypeptide of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or about 5.0 μg/μL.

In certain embodiments, the vaccine composition is administered with a volume selected from the group consisting of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or about 500 μL.

In certain embodiments, the compositions of the disclosure comprise about 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, or about 6000 μg of CpG.

In certain embodiments, the compositions of the disclosure comprise about 250, 300, 350, 400, 450, 500, 550, 600, 650, 700, or about 750 μg of AlOH.

In certain embodiments, the compositions of the disclosure comprise about 1, 2, 3, 4, 5, 6, 7, 8, 9, 10, 11, 12, 13, 14, 15, 16, 17, 18, 19, 20, 21, 22, 23, 24, or about 25 μg of dmLT.

In certain embodiments, the polypeptide and CpG have a mass ratio in the pharmaceutical or vaccine composition of about 10:6000, 15:6000, 20:6000, 25:6000, 30:6000, 35:6000, 40:6000, 45:6000, 50:6000, 55:6000, 60:6000, 65:6000, 70:6000, 75:6000, 80:6000, 85:6000, 90:6000, 95:6000, 100:6000, 10:5750, 15:5750, 20:5750, 25:5750, 30:5750, 35:5750, 40:5750, 45:5750, 50:5750, 55:5750, 60:5750, 65:5750, 70:5750, 75:5750, 80:5750, 85:5750, 90:5750, 95:5750, 100:5750, 10:5500, 15:5500, 20:5500, 25:5500, 30:5500, 35:5500, 40:5500, 45:5500, 50:5500, 55:5500, 60:5500, 65:5500, 70:5500, 75:5500, 80:5500, 85:5500, 90:5500, 95:5500, 100:5500, 10:5250, 15:5250, 20:5250, 25:5250, 30:5250, 35:5250, 40:5250, 45:5250, 50:5250, 55:5250, 60:5250, 65:5250, 70:5250, 75:5250, 80:5250, 85:5250, 90:5250, 95:5250, 100:5250, 10:5000, 15:5000, 20:5000, 25:5000, 30:5000, 35:5000, 40:5000, 45:5000, 50:5000, 55:5000, 60:5000, 65:5000, 70:5000, 75:5000, 80:5000, 85:5000, 90:5000, 95:5000, 100:5000, 10:4750, 15:4750, 20:4750, 25:4750, 30:4750, 35:4750, 40:4750, 45:4750, 50:4750, 55:4750, 60:4750, 65:4750, 70:4750, 75:4750, 80:4750, 85:4750, 90:4750, 95:4750, 100:4750, 10:4500, 15:4500, 20:4500, 25:4500, 30:4500, 35:4500, 40:4500, 45:4500, 50:4500, 55:4500, 60:4500, 65:4500, 70:4500, 75:4500, 80:4500, 85:4500, 90:4500, 95:4500, 100:4500, 10:4250, 15:4250, 20:4250, 25:4250, 30:4250, 35:4250, 40:4250, 45:4250, 50:4250, 55:4250, 60:4250, 65:4250, 70:4250, 75:4250, 80:4250, 85:4250, 90:4250, 95:4250, 100:4250, 10:4000, 15:4000, 20:4000, 25:4000, 30:4000, 35:4000, 40:4000, 45:4000, 50:4000, 55:4000, 60:4000, 65:4000, 70:4000, 75:4000, 80:4000, 85:4000, 90:4000, 95:4000, 100:4000, 10:3750, 15:3750, 20:3750, 25:3750, 30:3750, 35:3750, 40:3750, 45:3750, 50:3750, 55:3750, 60:3750, 65:3750, 70:3750, 75:3750, 80:3750, 85:3750, 90:3750, 95:3750, 100:3750, 10:3500, 15:3500, 20:3500, 25:3500, 30:3500, 35:3500, 40:3500, 45:3500, 50:3500, 55:3500, 60:3500, 65:3500, 70:3500, 75:3500, 80:3500, 85:3500, 90:3500, 95:3500, 100:3500, 10:3250, 15:3250, 20:3250, 25:3250, 30:3250, 35:3250, 40:3250, 45:3250, 50:3250, 55:3250, 60:3250, 65:3250, 70:3250, 75:3250, 80:3250, 85:3250, 90:3250, 95:3250, 100:3250, 10:3000, 15:3000, 20:3000, 25:3000, 30:3000, 35:3000, 40:3000, 45:3000, 50:3000, 55:3000, 60:3000, 65:3000, 70:3000, 75:3000, 80:3000, 85:3000, 90:3000, 95:3000, 100:3000, 10:2750, 15:2750, 20:2750, 25:2750, 30:2750, 35:2750, 40:2750, 45:2750, 50:2750, 55:2750, 60:2750, 65:2750, 70:2750, 75:2750, 80:2750, 85:2750, 90:2750, 95:2750, 100:2750, 10:2500, 15:2500, 20:2500, 25:2500, 30:2500, 35:2500, 40:2500, 45:2500, 50:2500, 55:2500, 60:2500, 65:2500, 70:2500, 75:2500, 80:2500, 85:2500, 90:2500, 95:2500, 100:2500, 10:2250, 15:2250, 20:2250, 25:2250, 30:2250, 35:2250, 40:2250, 45:2250, 50:2250, 55:2250, 60:2250, 65:2250, 70:2250, 75:2250, 80:2250, 85:2250, 90:2250, 95:2250, 100:2250, 10:2000, 15:2000, 20:2000, 25:2000, 30:2000, 35:2000, 40:2000, 45:2000, 50:2000, 55:2000, 60:2000, 65:2000, 70:2000, 75:2000, 80:2000, 85:2000, 90:2000, 95:2000, 100:2000, 10:1750, 15:1750, 20:1750, 25:1750, 30:1750, 35:1750, 40:1750, 45:1750, 50:1750, 55:1750, 60:1750, 65:1750, 70:1750, 75:1750, 80:1750, 85:1750, 90:1750, 95:1750, 100:1750, 10:1500, 15:1500, 20:1500, 25:1500, 30:1500, 35:1500, 40:1500, 45:1500, 50:1500, 55:1500, 60:1500, 65:1500, 70:1500, 75:1500, 80:1500, 85:1500, 90:1500, 95:1500, 100:1500, 10:1250, 15:1250, 20:1250, 25:1250, 30:1250, 35:1250, 40:1250, 45:1250, 50:1250, 55:1250, 60:1250, 65:1250, 70:1250, 75:1250, 80:1250, 85:1250, 90:1250, 95:1250, 100:1250, 10:1000, 15:1000, 20:1000, 25:1000, 30:1000, 35:1000, 40:1000, 45:1000, 50:1000, 55:1000, 60:1000, 65:1000, 70:1000, 75:1000, 80:1000, 85:1000, 90:1000, 95:1000, 100:1000, 10:750, 15:750, 20:750, 25:750, 30:750, 35:750, 40:750, 45:750, 50:750, 55:750, 60:750, 65:750, 70:750, 75:750, 80:750, 85:750, 90:750, 95:750, 100:750, 10:500, 15:500, 20:500, 25:500, 30:500, 35:500, 40:500, 45:500, 50:500, 55:500, 60:500, 65:500, 70:500, 75:500, 80:500, 85:500, 90:500, 95:500, 100:500, 10:250, 15:250, 20:250, 25:250, 30:250, 35:250, 40:250, 45:250, 50:250, 55:250, 60:250, 65:250, 70:250, 75:250, 80:250, 85:250, 90:250, 95:250, 100:250, 10:100, 15:100, 20:100, 25:100, 30:100, 35:100, 40:100, 45:100, 50:100, 55:100, 60:100, 65:100, 70:100, 75:100, 80:100, 85:100, 90:100, 95:100, or about 100:100 (polypeptide:CpG).

In certain embodiments, the pharmaceutical composition comprises about 0.4% AlOH (w/w %). In certain embodiments, the polypeptide and AlOH have a mass ratio in the pharmaceutical or vaccine composition of about 10:750, 15:750, 20:750, 25:750, 30:750, 35:750, 40:750, 45:750, 50:750, 55:750, 60:750, 65:750, 70:750, 75:750, 80:750, 85:750, 90:750, 95:750, 100:750, 10:700, 15:700, 20:700, 25:700, 30:700, 35:700, 40:700, 45:700, 50:700, 55:700, 60:700, 65:700, 70:700, 75:700, 80:700, 85:700, 90:700, 95:700, 100:700, 10:650, 15:650, 20:650, 25:650, 30:650, 35:650, 40:650, 45:650, 50:650, 55:650, 60:650, 65:650, 70:650, 75:650, 80:650, 85:650, 90:650, 95:650, 100:650, 10:600, 15:600, 20:600, 25:600, 30:600, 35:600, 40:600, 45:600, 50:600, 55:600, 60:600, 65:600, 70:600, 75:600, 80:600, 85:600, 90:600, 95:600, 100:600, 10:550, 15:550, 20:550, 25:550, 30:550, 35:550, 40:550, 45:550, 50:550, 55:550, 60:550, 65:550, 70:550, 75:550, 80:550, 85:550, 90:550, 95:550, 100:550, 10:500, 15:500, 20:500, 25:500, 30:500, 35:500, 40:500, 45:500, 50:500, 55:500, 60:500, 65:500, 70:500, 75:500, 80:500, 85:500, 90:500, 95:500, 100:500, 10:450, 15:450, 20:450, 25:450, 30:450, 35:450, 40:450, 45:450, 50:450, 55:450, 60:450, 65:450, 70:450, 75:450, 80:450, 85:450, 90:450, 95:450, 100:450, 10:400, 15:400, 20:400, 25:400, 30:400, 35:400, 40:400, 45:400, 50:400, 55:400, 60:400, 65:400, 70:400, 75:400, 80:400, 85:400, 90:400, 95:400, 100:400, 10:350, 15:350, 20:350, 25:350, 30:350, 35:350, 40:350, 45:350, 50:350, 55:350, 60:350, 65:350, 70:350, 75:350, 80:350, 85:350, 90:350, 95:350, 100:350, 10:300, 15:300, 20:300, 25:300, 30:300, 35:300, 40:300, 45:300, 50:300, 55:300, 60:300, 65:300, 70:300, 75:300, 80:300, 85:300, 90:300, 95:300, 100:300, 10:250, 15:250, 20:250, 25:250, 30:250, 35:250, 40:250, 45:250, 50:250, 55:250, 60:250, 65:250, 70:250, 75:250, 80:250, 85:250, 90:250, 95:250, or about 100:250 (polypeptide:AlOH).

In certain embodiments, the pharmaceutical composition comprises about 50% AddaS03™.

In certain embodiments, polypeptide and dmLT have a mass ratio in the pharmaceutical or vaccine composition of about 10:1, 15:1, 20:1, 25:1, 30:1, 35:1, 40:1, 45:1, 50:1, 55:1, 60:1, 65:1, 70:1, 75:1, 80:1, 85:1, 90:1, 95:1, 100:1, 10:2, 15:2, 20:2, 25:2, 30:2, 35:2, 40:2, 45:2, 50:2, 55:2, 60:2, 65:2, 70:2, 75:2, 80:2, 85:2, 90:2, 95:2, 100:2, 10:3, 15:3, 20:3, 25:3, 30:3, 35:3, 40:3, 45:3, 50:3, 55:3, 60:3, 65:3, 70:3, 75:3, 80:3, 85:3, 90:3, 95:3, 100:3, 10:4, 15:4, 20:4, 25:4, 30:4, 35:4, 40:4, 45:4, 50:4, 55:4, 60:4, 65:4, 70:4, 75:4, 80:4, 85:4, 90:4, 95:4, 100:4, 10:5, 15:5, 20:5, 25:5, 30:5, 35:5, 40:5, 45:5, 50:5, 55:5, 60:5, 65:5, 70:5, 75:5, 80:5, 85:5, 90:5, 95:5, 100:5, 10:10, 15:10, 20:10, 25:10, 30:10, 35:10, 40:10, 45:10, 50:10, 55:10, 60:10, 65:10, 70:10, 75:10, 80:10, 85:10, 90:10, 95:10, 100:10, 10:15, 15:15, 20:15, 25:15, 30:15, 35:15, 40:15, 45:15, 50:15, 55:15, 60:15, 65:15, 70:15, 75:15, 80:15, 85:15, 90:15, 95:15, 100:15, 10:20, 15:20, 20:20, 25:20, 30:20, 35:20, 40:20, 45:20, 50:20, 55:20, 60:20, 65:20, 70:20, 75:20, 80:20, 85:20, 90:20, 95:20, 100:20, 10:25, 15:25, 20:25, 25:25, 30:25, 35:25, 40:25, 45:25, 50:25, 55:25, 60:25, 65:25, 70:25, 75:25, 80:25, 85:25, 90:25, 95:25, or about 100:25 (polypeptide:dmLT).

In certain embodiments, the oil-in-water emulsion adjuvant comprises about 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or about 10.0 mg polysorbate 80. In certain embodiments, the oil-in-water emulsion adjuvant comprises about 2.43 mg polysorbate 80. In certain embodiments, the oil-in-water emulsion adjuvant comprises about 4.86 mg polysorbate 80. In certain embodiments, the oil-in-water emulsion adjuvant comprises about 9.72 mg polysorbate 80.

In certain embodiments, the oil-in-water emulsion adjuvant comprises about 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, 24.0, 24.5, or about 25.0 mg squalene. In certain embodiments, the oil-in-water emulsion adjuvant comprises about 5.35 mg squalene. In certain embodiments, the oil-in-water emulsion adjuvant comprises about 10.69 mg squalene. In certain embodiments, the oil-in-water emulsion adjuvant comprises about 21.38 mg squalene.

In certain embodiments, the oil-in-water emulsion adjuvant comprises about 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, 24.0, 24.5, or about 25.0 mg α-tocopherol (e.g., racemic α-tocopherol). In certain embodiments, the oil-in-water emulsion adjuvant comprises about 5.93 α-tocopherol. In certain embodiments, the oil-in-water emulsion adjuvant comprises about 11.86 mg α-tocopherol. In certain embodiments, the oil-in-water emulsion adjuvant comprises about 23.72 mg α-tocopherol.

In certain embodiments, the oil-in-water emulsion adjuvant comprises about 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, or about 75% (v/v) of the vaccine composition of the disclosure.

In certain embodiments, the disclosure provides a vaccine composition comprising:
(a) a polypeptide which shares at least 85%, 90%, 95%, 96%, 97%, 98%, 99%, or 100% sequence homology with SEQ ID NO:225, wherein the vaccine composition comprises about 10 μg to about 100 μg of the polypeptide;
(b) α-tocopherol, wherein the α-tocopherol comprises about 2.5% (v/v) of the vaccine composition;
(c) squalene, wherein the squalene comprises about 2.5% (v/v) of the vaccine composition; and
(d) polysorbate 80, wherein the polysorbate 80 comprises about 0.9% (v/v) of the vaccine composition;
   wherein the polypeptide, α-tocopherol, squalene, and polysorbate are dissolved or suspended in a phosphate buffered saline (PBS) solution.

In certain embodiments, the disclosure provides a vaccine composition comprising:
(a) a polypeptide which shares at least 85%, 90%, 95%, 96%, 97%, 98%, 99%, or 100% sequence homology with SEQ ID NO:225, wherein the vaccine composition comprises about 10 μg to about 100 μg of the polypeptide;
(b) α-tocopherol, wherein the α-tocopherol comprises about 2.5% (v/v) of the vaccine composition;
(c) squalene, wherein the squalene comprises about 2.5% (v/v) of the vaccine composition;
(d) polysorbate 80, wherein the polysorbate 80 comprises about 0.9% (v/v) of the vaccine composition;
(e) CpG, wherein the vaccine comprises about 100 μg to about 6000 μg of the CpG; wherein the polypeptide, α-tocopherol, squalene, polysorbate, and CpG are dissolved or suspended in a phosphate buffered saline (PBS) solution.

In certain embodiments, the disclosure provides a vaccine composition comprising:
(a) a polypeptide which shares at least 85%, 90%, 95%, 96%, 97%, 98%, 99%, or 100% sequence homology with SEQ ID NO:226, wherein the vaccine composition comprises about 10 μg to about 100 μg of the polypeptide;
(b) α-tocopherol, wherein the α-tocopherol comprises about 2.5% (v/v) of the vaccine composition;
(c) squalene, wherein the squalene comprises about 2.5% (v/v) of the vaccine composition; and
(d) polysorbate 80, wherein the polysorbate 80 comprises about 0.9% (v/v) of the vaccine composition;
wherein the polypeptide, α-tocopherol, squalene, and polysorbate are dissolved or suspended in a phosphate buffered saline (PBS) solution.

In certain embodiments, the disclosure provides a vaccine composition comprising:
(a) a polypeptide which shares at least 85%, 90%, 95%, 96%, 97%, 98%, 99%, or 100% sequence homology with SEQ ID NO:226, wherein the vaccine composition comprises about 10 μg to about 100 μg of the polypeptide;
(b) α-tocopherol, wherein the α-tocopherol comprises about 2.5% (v/v) of the vaccine composition;
(c) squalene, wherein the squalene comprises about 2.5% (v/v) of the vaccine composition;
(d) polysorbate 80, wherein the polysorbate 80 comprises about 0.9% (v/v) of the vaccine composition;
(e) CpG, wherein the vaccine comprises about 100 μg to about 6000 μg of the CpG; wherein the polypeptide, α-tocopherol, squalene, polysorbate, and CpG are dissolved or suspended in a phosphate buffered saline (PBS) solution.

In another aspect, the present disclosure provides a pharmaceutical composition comprising at least one LNP of the present disclosure and a pharmaceutically acceptable excipient.

The invention provides pharmaceutical compositions comprising at least one polypeptide or compound of the invention or a salt or solvate thereof, which are useful to practice methods of the invention. Such a pharmaceutical composition may consist of at least one polypeptide or compound of the invention or a salt or solvate thereof, in a form suitable for administration to a subject, or the pharmaceutical composition may comprise at least one polypeptide or compound of the invention or a salt or solvate thereof, and one or more pharmaceutically acceptable carriers, one or more additional ingredients, or any combinations of these. At least one polypeptide or compound of the invention may be present in the pharmaceutical composition in the form of a physiologically acceptable salt, such as in combination with a physiologically acceptable cation or anion, as is well known in the art.

In certain embodiments, the pharmaceutical compositions useful for practicing the method of the invention may be administered to deliver a dose of between 1 ng/kg/day and 1 mg/kg/day. In other embodiments, the pharmaceutical compositions useful for practicing the invention may be administered to deliver a dose of between 100 ng/kg/day and 1 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for nasal, inhalational, oral, rectal, vaginal, pleural, peritoneal, parenteral, topical, transdermal, pulmonary, intranasal, buccal, ophthalmic, epidural, intrathecal, intravenous, or another route of administration. A composition useful within the methods of the invention may be directly administered to the brain, the brainstem, or any other part of the central nervous system of a mammal or bird. Other contemplated formulations include projected nanoparticles, microspheres, liposomal preparations, coated particles, polymer conjugates, resealed erythrocytes containing the active ingredient, and immunologically-based formulations.

In certain embodiments, the compositions of the invention are part of a pharmaceutical matrix, which allows for manipulation of insoluble materials and improvement of the bioavailability thereof, development of controlled or sustained release products, and generation of homogeneous compositions. By way of example, a pharmaceutical matrix may be prepared using hot melt extrusion, solid solutions, solid dispersions, size reduction technologies, molecular complexes (e.g., cyclodextrins, and others), microparticulate, and particle and formulation coating processes. Amorphous or crystalline phases may be used in such processes.

The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology and pharmaceutics. In general, such preparatory methods include the step of bringing the active ingredient into association with a carrier or one or more other accessory ingredients, and then, if necessary or desirable, shaping or packaging the product into a desired single-dose or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage.

Although the descriptions of pharmaceutical compositions provided herein are principally directed to pharmaceutical compositions suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, dogs, and rodents.

In certain embodiments, the compositions of the invention are formulated using one or more pharmaceutically acceptable excipients or carriers. In certain embodiments, the pharmaceutical compositions of the invention comprise a therapeutically effective amount of at least one polypeptide or compound of the invention and a pharmaceutically acceptable carrier.

Pharmaceutically acceptable carriers, which are useful, include, but are not limited to, glycerol, water, saline, ethanol, recombinant human albumin (e.g., RECOMBUMIN®), solubilized gelatins (e.g., GELOFUSINE®), and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), recombinant human albumin, solubilized gelatins, suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin.

Formulations may be employed in admixtures with conventional excipients, i.e., pharmaceutically acceptable organic or inorganic carrier substances suitable for parenteral, intravenous, subcutaneous, transdermal enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring, and/or fragrance-conferring substances and the like. They may also be combined where desired with other active agents, e.g., other analgesic, anxiolytics or hypnotic agents. As used herein, “additional ingredients” include, but are not limited to, one or more ingredients that may be used as a pharmaceutical carrier.

The composition of the invention may comprise a preservative from about 0.005% to 2.0% by total weight of the composition. The preservative is used to prevent spoilage in the case of exposure to contaminants in the environment. Examples of preservatives useful in accordance with the invention include but are not limited to those selected from the group consisting of benzyl alcohol, sorbic acid, parabens, imidurea and any combinations thereof. One such preservative is a combination of about 0.5% to 2.0% benzyl alcohol and 0.05-0.5% sorbic acid.

The composition may include an antioxidant and a chelating agent that inhibit the degradation of the polypeptide or compound. Antioxidants for some polypeptide or compounds are BHT, BHA, alphα-tocopherol and ascorbic acid in the exemplary range of about 0.01% to 0.3%, or BHT in the range of 0.03% to 0.1% by weight by total weight of the composition. The chelating agent may be present in an amount of from 0.01% to 0.5% by weight by total weight of the composition. Exemplary chelating agents include edetate salts (e.g. disodium edetate) and citric acid in the weight range of about 0.01% to 0.20%, or in the range of 0.02% to 0.10% by weight by total weight of the composition. The chelating agent is useful for chelating metal ions in the composition that may be detrimental to the shelf life of the formulation. While BHT and disodium edetate are exemplary antioxidant and chelating agent, respectively, for some polypeptides or compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethyl cellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, acacia, and ionic or non-ionic surfactants. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, ionic and non-ionic surfactants, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying. Methods for mixing components include physical milling, the use of pellets in solid and suspension formulations and mixing in a transdermal patch, as known to those skilled in the art.

### Administration/Dosing

In clinical settings, delivery systems for the compositions described herein can be introduced into a subject by any of a number of methods, each of which is familiar in the art. For instance, a pharmaceutical formulation of the composition can be administered by intravenous injection.

The regimen of administration may affect what constitutes an effective amount. The therapeutic formulations may be administered to the subject either prior to or after the manifestation of symptoms associated with the disease or condition (i.e., to prevent, treat, and/or ameliorate infection in a subject, or symptoms thereof, and/or to prevent recurrence of infection in a subject, or symptoms thereof).

Administration of the composition of the present invention to a subject, preferably a mammal, more preferably a human and/or laboratory mammal (e.g., rodent), may be carried out using known procedures, at dosages and for periods of time effective to treat, prevent, and/or ameliorate a disease or condition in the subject. An effective amount of the composition necessary to achieve a therapeutic effect may vary according to factors such as the time of administration; the duration of administration; other drugs, polypeptides, compounds or materials used in combination with the composition; the state of the disease or disorder; age, sex, weight, condition, general health and prior medical history of the subject being treated; and like factors well-known in the medical arts. Dosage regimens may be adjusted to provide the optimum therapeutic response. For example, doses may be annually or semi-annually, or the dose may be proportionally reduced as indicated by the exigencies of the therapeutic situation. One of ordinary skill in the art would be able to study the relevant factors and make the determination regarding the effective amount of the composition without undue experimentation. Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the invention include oral, nasal, rectal, intravaginal, parenteral, buccal, sublingual or topical. The polypeptides for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

In certain preferred embodiments, compositions of the present invention are administered intramuscularly.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the polypeptides or compounds of the invention may be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

### Parenteral Administration

For parenteral administration, the polypeptides or compounds of the invention may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Controlled Release Formulations and Drug Delivery Systems

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release that is longer that the same amount of agent administered in bolus form.

For sustained release, the polypeptides or compounds may be formulated with a suitable polymer or hydrophobic material that provides sustained release properties to the polypeptides or compounds. As such, the polypeptides or compounds for use the method of the invention may be administered in the form of microparticles, for example, by injection or in the form of wafers or discs by implantation.

In certain embodiments, the polypeptides or compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration, which may include a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a polypeptide, compound, and/or composition of the present invention depends on the age, sex and weight of the patient, the current medical condition of the patient and the progression of a disease or disorder contemplated herein in the patient being treated. The skilled artisan is able to determine appropriate dosages depending on these and other factors.

A suitable dose of a polypeptide, compound and/or composition of the present invention may be in the range of from about 1 ng to about 1 mg per dosage, such as from about 100 ng to about 1 mg, for example, from about 100 ng to about 500 ng per dosage. In certain embodiments, the suitable dose of a polypeptide, compound and/or composition of the present invention may be in the range of from about 10 μg to about 100 pg. The dose may be administered in a single dosage or in multiple dosages, for example from 1 to 4 or more times. When multiple dosages are used, the amount of each dosage may be the same or different.

In certain embodiments, the composition may be administered to the subject as one to four doses. In certain embodiments, the subject is administered a single dose. In certain embodiments, the subject is administered two doses. In certain embodiments, the subject is administered three doses. In certain embodiments, the subject is administered four doses. In certain embodiments, the subject is administered more than four doses.

In certain embodiments, each dose is administered with an intervening period of about 1 week. In certain embodiments, each dose is administered with an intervening period of about 1 week or less. In certain embodiments, each dose is administered with an intervening period of approximately 1 week or more. In certain embodiments, each dose is administered with an intervening period of about 2 weeks. In certain embodiments, each dose is administered with an intervening period of about 2 weeks or less. In certain embodiments, each dose is administered with an intervening period of approximately 2 weeks or more. In certain embodiments, each dose is administered with an intervening period of about 3 weeks. In certain embodiments, each dose is administered with an intervening period of about 3 weeks or less. In certain embodiments, each dose is administered with an intervening period of approximately 3 weeks or more. In certain embodiments, each dose is administered with an intervening period of about 4 weeks. In certain embodiments, each dose is administered with an intervening period of about 4 weeks or less. In certain embodiments, each dose is administered with an intervening period of approximately 4 weeks or more.

In certain embodiments, the subject is administered two doses, wherein each dose is administered with an intervening period of about 2 weeks between doses. In certain embodiments, the subject is administered two doses, wherein each dose is administered with an intervening period of about 3 weeks between doses. In certain embodiments, the subject is administered two doses, wherein each dose is administered with an intervening period of about 4 weeks between doses. In certain embodiments, the subject is administered two doses, wherein each dose is administered with an intervening period of about 5 weeks between doses. In certain embodiments, the subject is administered two doses, wherein each dose is administered with an intervening period of about 6 weeks between doses. In certain embodiments, the subject is administered two doses, wherein each dose is administered with an intervening period of about 7 weeks between doses. In certain embodiments, the subject is administered two doses, wherein each dose is administered with an intervening period of about 8 weeks between doses.

In certain embodiments, the subject is administered three doses, wherein each dose is administered with an intervening period of about 2 weeks between doses. In certain embodiments, the subject is administered three doses, wherein each dose is administered with an intervening period of about 3 weeks between doses. In certain embodiments, the subject is administered three doses, wherein each dose is administered with an intervening period of about 4 weeks between doses. In certain embodiments, the subject is administered three doses, wherein each dose is administered with an intervening period of about 5 weeks between doses. In certain embodiments, the subject is administered three doses, wherein each dose is administered with an intervening period of about 6 weeks between doses. In certain embodiments, the subject is administered three doses, wherein each dose is administered with an intervening period of about 7 weeks between doses. In certain embodiments, the subject is administered three doses, wherein each dose is administered with an intervening period of about 8 weeks between doses.

In certain embodiments, the subject is administered four doses, wherein each dose is administered with an intervening period of about 2 weeks between doses. In certain embodiments, the subject is administered four doses, wherein each dose is administered with an intervening period of about 3 weeks between doses. In certain embodiments, the subject is administered four doses, wherein each dose is administered with an intervening period of about 4 weeks between doses. In certain embodiments, the subject is administered four doses, wherein each dose is administered with an intervening period of about 5 weeks between doses. In certain embodiments, the subject is administered four doses, wherein each dose is administered with an intervening period of about 6 weeks between doses. In certain embodiments, the subject is administered four doses, wherein each dose is administered with an intervening period of about 7 weeks between doses. In certain embodiments, the subject is administered four doses, wherein each dose is administered with an intervening period of about 8 weeks between doses.

In certain preferred, non-limiting embodiments, the subject is administered four doses with an intervening period of about 2 weeks between doses (i.e., first dose on day 0, second dose on about day 14, third dose on about day 28, and fourth dose on about day 42).

In certain preferred, non-limiting embodiments, the subject is administered two doses with an intervening period of about 3 weeks between doses (i.e., first dose on day 0 and second dose on about day 21). In certain preferred, non-limiting embodiments, the subject is administered two doses with an intervening period of about 4 weeks between doses (i.e., first dose on day 0 and second dose on about day 28). In certain preferred, non-limiting embodiments, the subject is administered two doses with an intervening period of about 8 weeks between doses (i.e., first dose on day 0 and second dose on about day 56).

Actual dosage levels of the cells in the pharmaceutical formulations of this invention may be varied so as to obtain an amount of the composition that are effective to achieve the desired therapeutic response for a particular subject, composition, and mode of administration, without being toxic to the subject.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such polypeptides, compounds and/or compositions lies preferably within a range of circulating concentrations that include the ED₅₀ with minimal toxicity. The dosage optionally varies within this range depending upon the dosage form employed and the route of administration utilized.

### EXAMPLES

Various embodiments of the present application can be better understood by reference to the following Examples which are offered by way of illustration. The scope of the present application is not limited to the Examples given herein.

### Preparation of Antigens

In certain embodiments, the polypeptides described herein were prepared utilizing one or more bacterial expression vectors (e.g., E. coli), according to methods known to those skilled in the art (Front. Microbiol. 2014, 5:172). In one aspect, the construction of such recombination DNA molecules comprises the steps of: (a) producing a single-stranded DNA copy (cDNA) of a purified messenger RNA (mRNA) as a template for the desired protein; (b) converting the cDNA into double-stranded DNA; (c) binding the DNA to a suitable point in a suitable clone carrier to form a recombination DNA molecule; and (d) transforming a suitable host with this recombination DNA molecule. Such a transformation allows the host to produce the desired protein.

An expression vector has features that any vector may have, such as an origin of replication, a selectable marker, and a suitable site for the insertion of a gene like the multiple cloning site. The cloned gene may be transferred from a specialized cloning vector to an expression vector, although it is possible to clone directly into an expression vector. The cloning process can be performed in E. coli. Vectors used for protein production in organisms other than 15 E. coli may have, in addition to a suitable origin of replication for its propagation in E. coli, elements that allow them to be maintained in another organism (i.e., shuttle vectors).

### HLA-DR4 Mice

HLA-DR4 allele is associated with the development of autoimmune diseases such as rheumatoid arthritis and multiple sclerosis. In an attempt to provide a mouse model for these diseases, a hybrid MHC class II molecule between the peptide binding domains of human HLA-DRA and HLA-DRB*0401 and the membrane proximal domains of mouse I-E (H2-E) was engineered and co-injected into C57BL/6 fertilized eggs. The transgenic offspring were bred to a mouse incapable of expressing other MHC class II molecules (Abb knockout on B⁶ background). By preserving the alpha 2 and beta 2 domains of mouse MHC class II, interactions with CD4 co-receptors on T cells were preserved. This mouse is healthy and breeds normally. Immunization with a peptide from a proteolipid protein known to bind to HLA-DR4, provoked a strong T cell proliferative response, caused inflammatory lesions in CNS white matter, and symptoms of experimental allergic encephalomyelitis (Ito K, et al. J. Exp. Med 1996, 183(6):2635-2644).

### Immunization of HLA DR4 Mice

HLA-DR4 transgenic mice, each of an age within one week of all other mice within the study, were used in the experiments described herein. HLA-DR4 mice were divided into treatment groups and control groups (e.g., naïve and/or placebo). Mice were immunized twice via the route of administration indicated elsewhere herein (e.g., subcutaneously or intramuscularly) on the specified days with vaccine compositions having a total volume of 50 μL or 100 μL, wherein the vaccine compositions comprise 100 μg of antigen (e.g., SEQ ID NO:127, SEQ ID NO:128, SEQ ID N:132, SEQ ID NO:133, or SEQ ID NO:135) and an adjuvant (e.g., AlOH, dmLT, CpG, and/or AddaS03™), and diluted to the total volume with a phosphate buffered saline (PBS) solution. In certain embodiments, HLA-DR4 mice were immunized as described in Tables 1-7 (i.e., Experiment Nos. 1-7). Sera and urine samples were obtained from the mice at specified timepoints, as described elsewhere herein.

TABLE 1
Immunization of HLA-DR4 mice^{a} (Exp. No. 1)

| Group | Antigen | Adjuvant |
|---|---|---|
| 1 | —^{b} | —^{b} |
| 2 | SEQ ID NO: 127 | 0.4% AlOH^{c} + 2 μg dmLT^{d} |
| 3 | SEQ ID NO: 128 | 0.4% AlOH^{c} + 2 μg dmLT^{d} |
| 4 | SEQ ID NO: 127 | 0.4% AlOH^{c} + 50 μg CpG^{e} |
| 5 | SEQ ID NO: 128 | 0.4% AlOH^{c} + 50 μg CpG^{e} |

^{a}n = 3 mice per group, 100 μL total volume, PBS diluent, 100 μg antigen, administered twice subcutaneously (approximately 3 weeks between vaccinations);
^{b}naïve control group;
^{c}aluminum hydroxide;
^{d}double mutant heat labile toxin (dmLT);
^{e}CpG ODN 2395.

TABLE 2
Immunization of HLA-DR4 mice^{a} (Exp. No. 2)

| Group | Antigen | Adjuvant |
|---|---|---|
| A | —^{b} | 0.4% AlOH^{c} + 10 μg CpG^{d} |
| B | SEQ ID NO: 127 | 0.4% AlOH^{c} + 10 μg CpG^{d} |
| C | SEQ ID NO: 128 | 0.4% AlOH^{c} + 10 μg CpG^{d} |
| D | SEQ ID NO: 132 | 0.4% AlOH^{c} + 10 μg CpG^{d} |
| E | SEQ ID NO: 133 | 0.4% AlOH^{c} + 10 μg CpG^{d} |
| F | SEQ ID NO: 135 | 0.4% AlOH^{c} + 10 μg CpG^{d} |

^{a}n = 5 mice per group, 100 μL total volume, PBS diluent, 100 μg antigen, administered twice subcutaneously, and mice aged 9-10 months at first vaccination (approximately 2 weeks between vaccinations);
^{b}placebo control group;
^{c}aluminum hydroxide;
^{d}CpG ODN 2395.

TABLE 3
Immunization of HLA-DR4 mice^{a} (Exp. No. 3)

| Group | Antigen | Adjuvant |
|---|---|---|
| A | SEQ ID NO: 132 | 50% AddaS03 ™ + 10 μg CpG^{b} |

^{a}n = 5 mice per group, 50 μL total volume, PBS diluent, 100 μg antigen, administered twice intramuscularly, and mice aged 8-10 weeks at first vaccination (approximately 3 weeks between vaccinations);
^{b}CpG ODN 2395.

TABLE 4
Immunization of HLA-DR4 mice^{a} (Exp. No. 4)

| Group | Antigen | Adjuvant |
|---|---|---|
| A | —^{b} | 50% AddaS03 ™ + 10 μg CpG^{c} |
| B | SEQ ID NO: 136 | 50% AddaS03 ™ + 10 μg CpG^{c} |
| C | SEQ ID NO: 137 | 50% AddaS03 ™ + 10 μg CpG^{c} |
| D | SEQ ID NO: 138 | 50% AddaS03 ™ + 10 μg CpG^{c} |
| E | SEQ ID NO: 139 | 50% AddaS03 ™ + 10 μg CpG^{c} |
| F | SEQ ID NO: 140 | 50% AddaS03 ™ + 10 μg CpG^{c} |

^{a}n = 5 mice per group for (A), n = 4 mice per group for (B-F), 50 μL total volume, PBS diluent, 100 μg antigen, administered twice intramuscularly, and mice aged 6-7 weeks at first vaccination (approximately 3 weeks between vaccinations);
^{b}placebo control group;
^{c}CpG ODN 2395.

TABLE 5
Immunization of HLA-DR4 mice^{a} (Exp. No. 5)

| Group | Antigen | Adjuvant |
|---|---|---|
| A | —^{b} | 0.4% AlOH^{c} + 10 μg CpG^{d} |
| B | SEQ ID NO: 136 | 0.4% AlOH^{c} + 10 μg CpG^{d} |
| C | SEQ ID NO: 138 | 0.4% AlOH^{c} + 10 μg CpG^{d} |
| D | —^{b} | 0.4% AlOH^{c} |
| E | SEQ ID NO: 136 | 0.4% AlOH^{c} |
| F | SEQ ID NO: 138 | 0.4% AlOH^{c} |

^{a}n = 5 mice per group, 50 μL total volume, PBS diluent, 100 μg antigen, administered twice intramuscularly, and mice aged 9-10 weeks at first vaccination (approximately 3 weeks between vaccinations);
^{b}placebo control group;
^{c}aluminum hydroxide;
^{d}CpG ODN 2395.

TABLE 6
Immunization of HLA-DR4 mice^{a} (Exp. No. 6)

| Group | Antigen | Adjuvant |
|---|---|---|
| A | —^{b} | 50% AddaS03 ™ + 10 μg CpG^{c} |
| B | SEQ ID NO: 141 | 50% AddaS03 ™ + 10 μg CpG^{c} |
| C | SEQ ID NO: 142 | 50% AddaS03 ™ + 10 μg CpG^{c} |
| D | SEQ ID NO: 143 | 50% AddaS03 ™ + 10 μg CpG^{c} |
| E | SEQ ID NO: 144 | 50% AddaS03 ™ + 10 μg CpG^{c} |
| F | SEQ ID NO: 145 | 50% AddaS03 ™ + 10 μg CpG^{c} |

^{a}n = 3 mice per group, 50 μL total volume, PBS diluent, 100 μg antigen, administered twice intramuscularly, and mice aged 6-8 weeks at first vaccination (approximately 3 weeks between vaccinations);
^{b}placebo control group;
^{c}CpG ODN 2395.

TABLE 7
Immunization of HLA-DR4 mice^{a} (Exp. No. 7)

| Group | Antigen | Adjuvant |
|---|---|---|
| A | —^{b} | 50% AddaS03 ™ + 10 μg CpG^{c} |
| B | SEQ ID NO: 138^{e} | 50% AddaS03 ™ + 10 μg CpG^{c} |
| C | SEQ ID NO: 138^{f} | 50% AddaS03 ™ + 10 μg CpG^{c} |
| D | —^{b} | 0.4% AlOH^{d} + 10 μg CpG^{c} |
| E | SEQ ID NO: 138^{f} | 0.4% AlOH^{d} + 10 μg CpG^{c} |
| F | —^{b} | 0.4% AlOH^{d} |
| G | SEQ ID NO: 138^{f} | 0.4% AlOH^{d} |

^{a}n = 3 mice per group (A-B and D-G), n = 4 mice per group (C), 50 μL total volume, PBS diluent, 100 μg antigen, administered twice intramuscularly (approximately 3 weeks between vaccinations), mice aged 7-9 weeks at first vaccination, and all mice received 1 challenge with UPEC25;
^{b}placebo control group;
^{c}CpG ODN 2395;
^{d}aluminum hydroxide;
^{e}buffer conditions (20 mM Na₂PO₄, 300 mM urea, 300 mM NaCl, pH 9.5);
^{f}buffer conditions (20 mM Na₂PO, 300 mM NaCl, pH 9.5).

### CD1 Mice

CD1 IGS (i.e., Crl:CD1(ICR)) is an albino outbred strain of mouse model frequently used in toxicology and pharmacological research. A notable feature of CD1 IGS mice is their large genetic diversity, which is similar to that found within and between human populations. CD 1 mice were immunized in a manner analogous to that which is described for HLA-DR4 mice. A detailed description of experiments performed utilizing CD1 mice is provided herein (Table 8).

TABLE 8
Immunization of CD1 mice^{a} (Exp. No. 8)

| Group | Antigen | Adjuvant |
|---|---|---|
| A^{b} | —^{d} | 0.4% AlOH^{e} |
| B^{b} | SEQ ID NO: 132 | 0.4% AlOH^{e} |
| C^{c} | SEQ ID NO: 135 | 0.4% AlOH^{e} |

^{a}100 μL total volume, PBS diluent, 100 μg antigen, administered twice subcutaneously, and mice aged 11 weeks at first vaccination (approximately 2 weeks between vaccinations);
^{b}n = 3 mice;
^{c}n = 4 mice;
^{d}placebo control group;
^{e}aluminum hydroxide.

### C57BL/6 Mice

C57BL/6 mice are the most used inbred strain in research and are commonly used as models of human disease. C57BU/6 mice were immunized in a manner analogous to that which is described for HLA-DR4 mice. A detailed description of experiments performed utilizing C57BU/6 mice is provided herein (Tables 9-11).

TABLE 9
Immunization of C57BL/6^{a} (Exp. No. 9)

| Group | Antigen | Adjuvant |
|---|---|---|
| A | —^{b} | 50% AddaS03 ™ + 10 μg CpG^{c} |
| B | SEQ ID NO: 136 | 50% AddaS03 ™ + 10 μg CpG^{c} |
| C | SEQ ID NO: 137 | 50% AddaS03 ™ + 10 μg CpG^{c} |
| D | SEQ ID NO: 138 | 50% AddaS03 ™ + 10 μg CpG^{c} |
| E | SEQ ID NO: 139 | 50% AddaS03 ™ + 10 μg CpG^{c} |

^{a}n = 15 mice per group, 50 μL total volume, PBS diluent, 100 μg antigen, administered twice intramuscularly, and mice aged 6-7 weeks at first vaccination (approximately 3 weeks between vaccinations), single challenge with UPEC25, immunological samples harvested at approximately 7 days post injection;
^{b}placebo control group;
^{c}CpG ODN 2395.

TABLE 10
Immunization of C57BL/6 mice^{a} (Exp. No. 10)

| Group | Antigen | Adjuvant |
|---|---|---|
| A | —^{b} | 50% AddaS03 ™ + 10 μg CpG^{c} |
| B | SEQ ID NO: 225 | 50% AddaS03 ™ + 10 μg CpG^{c} |
| C | —^{b} | 0.4% AlOH^{d} |
| D | SEQ ID NO: 225 | 0.4% AlOH^{d} |

^{a}n = 20 mice per group, 50 μL total volume, PBS diluent, 100 μg antigen, administered twice intramuscularly, and mice aged 6-7 weeks at first vaccination (approximately 3 weeks between vaccinations), immunological samples harvested at approximately 14 days post second vaccination;
^{b}placebo control group;
^{c}CpG ODN 2395;
^{d}aluminum hydroxide.

TABLE 11
Immunization of C57BL/6 mice^{a} (Exp. No. 11)

| Group | Antigen | Adjuvant |
|---|---|---|
| A | SEQ ID NO: 225 | — |
| B | —^{b} | 50% AddaS03 ™ |
| C | SEQ ID NO: 225 | 50% AddaS03 ™ |
| D | —^{b} | 50% AddaS03 ™ + 10 μg CpG^{c} |
| E | SEQ ID NO: 225 | 50% AddaS03 ™ + 10 μg CpG^{c} |
| F | —^{b} | 0.4% AlOH^{d} |
| G | SEQ ID NO: 225 | 0.4% AlOH^{d} |
| H | —^{b} | 0.4% AlOH^{d} + 10 μg CpG^{c} |
| I | SEQ ID NO: 225 | 0.4% AlOH^{d} + 10 μg CpG^{c} |

^{a}n = 4 mice per group, 50 μL total volume, PBS diluent, 100 μg antigen, administered twice intramuscularly, and mice aged 6 weeks at first vaccination (approximately 3 weeks between vaccinations), immunological samples harvested at approximately 12 days post second vaccination;
^{b}placebo control group;
^{c}CpG ODN 2395;
^{d}aluminum hydroxide.

### C3H/HeN Mice

C3H/HeN mice are homozygous for the Pde6brd1 allele and have an early onset severe retinal degeneration that causes blindness at weaning age. Despite an atherogenic diet, C3H/HeN mice do not develop aortic atherosclerosis, in contrast to the C57BU/6J. There is a genetically mediated difference between the C3H/HeN and the C3H/HeJ regarding their response to bacterial endotoxin (LPS). This response is linked to the TL4 protein (Toll-like receptor 4). C3H/HeN are Tlr4lps-n (toll-like receptor 4; normal LPS response). This strain has a normal response to an LPS challenge and is endotoxin-sensitive. Per contra, the C3H/HeJ strain is Tlr4lps-d, and said to be endotoxin-resistant. C3H/HeN mice were immunized in a manner analogous to that which is described for HLA-DR4 mice. A detailed description of experiments performed utilizing C3H/HeN mice is provided herein (Table 12).

TABLE 12
Immunization of C3H/HeN mice^{a} (Exp. No. 12)

| Group | Antigen | Adjuvant |
|---|---|---|
| A | —^{b} | 50% AddaS03 ™ + 10 μg CpG^{c} |
| B | SEQ ID NO: 225 | 50% AddaS03 ™ + 10 μg CpG^{c} |
| C | —^{b} | 0.4% AlOH^{d} |
| D | SEQ ID NO: 225 | 0.4% AlOH^{d} |

^{a}n = 10 mice per group, 50 μL total volume, PBS diluent, 100 μg antigen, administered twice intramuscularly, and mice aged 6-7 weeks at first vaccination (approximately 12 days between vaccinations), immunological samples harvested at approximately 14 days post second vaccination;
^{b}placebo control group;
^{c}CpG ODN 2395;
^{d}aluminum hydroxide.

### Cytometric Bead Array (CBA)

Cytometric Bead Array (CBA) assay (BD Biosciences) provides a method of capturing a soluble analyte or set of analytes with beads of known size and fluorescence, enabling detection of analytes using flow cytometry. Each capture bead in the CBA kit has a distinct fluorescence and is coated with an antibody specific for a soluble protein. The detection reagent is a mixture of phycoerythrin (PE)-conjugated antibodies, which provides a fluorescent signal in proportion to the amount of bound analyte.

When capture beads and detection reagent are incubated with standards or unknown samples containing recognized analytes, sandwich complexes are formed (e.g., capture bead+analyte+detection reagent). These complexes are measured using flow cytometry to identify particles with fluorescence characteristics of both the bead and the detector, enabling identification of analyte.

The immunological response to the compositions described herein was determined by measuring cytokine levels (e.g., IL-6, IL-17A, IL-2, TNF-α, IFN-γ, IL-4, and/or IL-10) by CBA.

### Enzyme-Linked Immunoassay (EUSA)

The immunological response to the compositions described herein was determined by measuring sample optical density and/or antibody, antigen, protein, and/or glycoprotein titers (e.g., IgG1, IgG2b, IgA, and/or GTxMS IgG) by standard ELISA and/or peptide-based ELISA. Experimental protocols for standard ELISA and/or peptide ELISA are known to those skilled in the art. The following example is offered by way of illustration. It is understood that the scope of the present application is not limited to the example provided herein.

In brief, 100 μL of polypeptide at 2 μg/mL, solubilized in 5 M urea, was added to each well of a 96-well EIA/RIA plate (Coming/Costar 3590) and incubated overnight at 4° C. All remaining steps were performed at room temperature. The plate was washed three times with PBS wash buffer (PBS containing 0.05% Tween 20) followed by the addition of 200 μL/well sample buffer consisting of PBS containing 0.05% Tween 20 and 1% bovine serum albumin. After 90 minutes, the sample buffer was replaced with 100 μL/well PBS sample buffer. Serial 1:3 dilutions of the primary antisera were performed in the plate by the addition of 50 μL to the first row, mixing 10 times, and transfer of 50 μL to the next row. The plate was incubated for 90 minutes followed by three washes and addition of 100 μL/well of the antibody, antigen, protein, and/or glycoprotein response to be measured. After a 90 minute incubation period the plate was washed four times followed by the addition of 100 pl TMB (BioFx; Surmodics, Eden Prairie, MN)/well.

Color was allowed to develop for 30 minutes, and the reaction was stopped by the addition of 100 pl stop reagent (BioFx). The absorbance was measured at a wavelength of 450 nm, and the titer was calculated as the inverse of the dilution corresponding to an absorbance of 1.0. Controls included a standardized primary serum included on each plate to monitor assay variability and wells that were uncoated to subtract background. The limit of detection for the assay was the inverse of the initial serum dilution.

### Evaluation of Secreted Cytokines by MILLIPLEX® Multiplex Immunoassay

The MILLIPLEX® multiplex immunoassay (Millipore Sigma), based on the Luminex® xMAP® bead-based multiplex assay platform, provides a method of capturing a soluble analyte or set of analytes with magnetic microspheres of known size and fluorescence, enabling detection of analytes using a Luminex® Detection System. Briefly, each magnetic MagPlex® microsphere bead is fluorescently coded with one of 500 specific ratios of two fluorophores and is coated with analyte-specific capture antibodies. These microspheres are incubated with sample, washed, and are then incubated with a detection reagent comprised of a mixture of a biotinylated analyte-specific detection antibody and a R-Phycoerythrin-conjugated streptavidin reagent which provides a fluorescent signal in proportion to the amount of bound analyte. When capture microspheres and detection reagent are incubated with standards or unknown samples containing recognized analytes, sandwich complexes are formed (e.g., capture microsphere+analyte+detection reagent). These complexes are measured using a Luminex MAGPIX® detection system to identify particles with fluorescence characteristics of both the bead and the detector, enabling identification of analyte.

The immunological response to the compositions described herein was determined by measuring cytokine levels (e.g., IL-6, IL-17A, IL-2, TNF-α, IFN-γ, IL-4, IL-22, and/or IL-10) by MILLIPLEX® multiplex immunoassay.

For the assays described herein, splenic lymphocytes (1×10⁶ cells in 0.2 mL) and bladder lymphocytes (2×10⁵ cells in 0.2 mL) were cultured with 12.5 pg of antigen at 37° C. for 48 hours. At the end of the culture period, culture plates were briefly centrifuged to pellet the cells after which the culture supernatants were removed. Samples were acquired on a Luminex MAGPIX® analyzer using Luminex® acquisition software, and data were analyzed by Belysa® Immunoassay Curve Fitting software.

### Splenocyte Studies

Mice used for T cell studies were euthanized, and their spleens were removed and processed to lyse red blood cells. Splenic lymphocytes (1×10⁶ cells in 0.2 mL RPMI-1640 containing 10% fetal bovine serum) were cultured with 12.5 pg of antigen for 48 hours, and the release of cytokines into cell supernatants was measured according to the manufacturer's protocol (BD Cytometric Bead Array (CBA), BD Biosciences, San Jose, CA).

### Dissociation of Bladder and Spleen Tissue

Bladders were dissected, pooled by group (3-5 bladders per group), cut into small pieces, and digested using the Multi Tissue Dissociation Kit 1 (Miltenyi) per the manufacturer's protocol at 37° C. for 1 hour with robust manual agitation every 15 min. Digestion was stopped by adding an equal volume of RPMI-1640 containing 10% fetal bovine serum. Remaining tissue was disrupted using the plunger from a 3cc syringe and was passed through 70 μm filters (Greiner Bio-One). Cells obtained from pooled bladders preparations were resuspended at a final concentration of 2×10⁶ cells/mL of RPMI-1640 containing 10% fetal bovine serum.

Individual spleens were dissected, disrupted using the plunger from a 3cc syringe and were passed through 70 sm filters before being processed to remove red blood cells and being resuspended in RPMI-1640 containing 10% fetal bovine serum. Cells obtained from individual spleen preparations were resuspended at a final concentration of 1×10⁷ cells/mL of RPMI-1640 containing 10% fetal bovine serum.

### Evaluation of Activation Induced Markers (AIM) on Restimulated Lymphocytes by Flow Cytometry

Activation induced marker (AIM) proteins are a group of proteins that become expressed on immune cells when those immune cells have contacted and/or bound their cognate antigen and have become activated. AIM proteins are not expressed, or are expressed only at low levels, on resting, inactivated immune cells. In the experiments described herein, the combination of CD49d and CD11a was used to identify effector/memory CD4⁺ and CD8⁺ T cells induced by vaccination. The combination of CD49d and CD80 was used to identify effector/memory B cells induced by vaccination. The combination of the AIM proteins OX40 and PD-L1 was subsequently used to identify CD4⁺ T cells activated in vitro by protein antigen; the combination of the AIM proteins CD69 and PD-L1 was subsequently used to identify CD8+ T cells activated in vitro by protein antigen; combination of the AIM proteins CD69 and PD-L1 or CD69 and CD86 was subsequently used to identify B cells activated in vitro by protein antigen.

Splenic lymphocytes (1×10⁶ cells in 0.2 mL) and bladder lymphocytes (2×10⁵ cells in 0.2 mL) were cultured with 12.5 pg of antigen at 37° C. for 24-48 hours. At the end of the culture period, cells were washed in PBS and resuspended in an amine-reactive Live/Dead dye solution to exclude dead cells and with anti-mouse CD16/CD32 antibodies to block Fc receptors. Cells were incubated at 4° C. for 25 to 30 min protected from light. Cells were then washed in FACS buffer (PBS supplemented with 2% fetal bovine serum and 0.095% Sodium Azide) and stained with combinations of fluorescent antibodies to identify T cells (CD3, CD4, CD8), B cells (CD19, HLA-DR) and to determine the expression of activation induced marker (AIM) proteins (CD11a, CD25, CD49d, OX40, PD-L1, CD69 CD80 and CD86) on these cells following restimulation with protein antigen. Antibodies were purchased from BD, BioLegend, and ThermoFisher and optimal staining concentrations were determined empirically based on the manufacturers recommended concentrations. Cells were stained with fluorescent antibodies for 25 to 30 min at 4° C. Samples were acquired on an Agilent NovoCyte Quanteon Flow Cytometer using NovoExpress software, and data were analyzed by FlowJo (Treestar) software. Expression of AIM proteins was evaluated on live, single cells.

### Example 1: Immunized Mice Demonstrate an Innate Immune Response

Exp. No. I

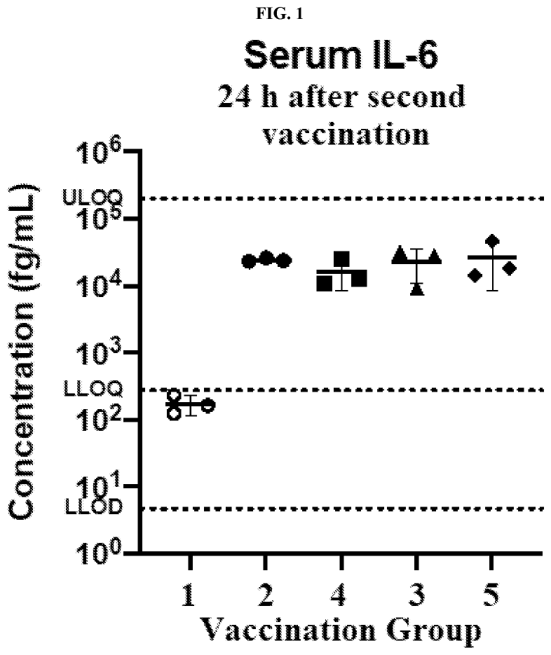

HLA-DR4 mice were immunized as described herein using the vaccine compositions indicated in Table 1. Serum samples were obtained from mice 24 h after the second vaccination and analyzed for serum IL-6 concentration. Comparable levels of IL-6 were observed across all treatment groups (i.e., groups 2-5 of Table 1), whereas serum IL-6 levels of naïve mice were below the lower limit of quantification (FIG. 1). Thus, the data indicates that immunization elicits an innate immune response.

### Example 2: Immunized Mice Demonstrate an Adaptive Immune Response (B Cell Mediated Antibody Production)

Exp. No. 1

HLA-DR4 mice were immunized as described herein using the vaccine compositions indicated in Table 1.

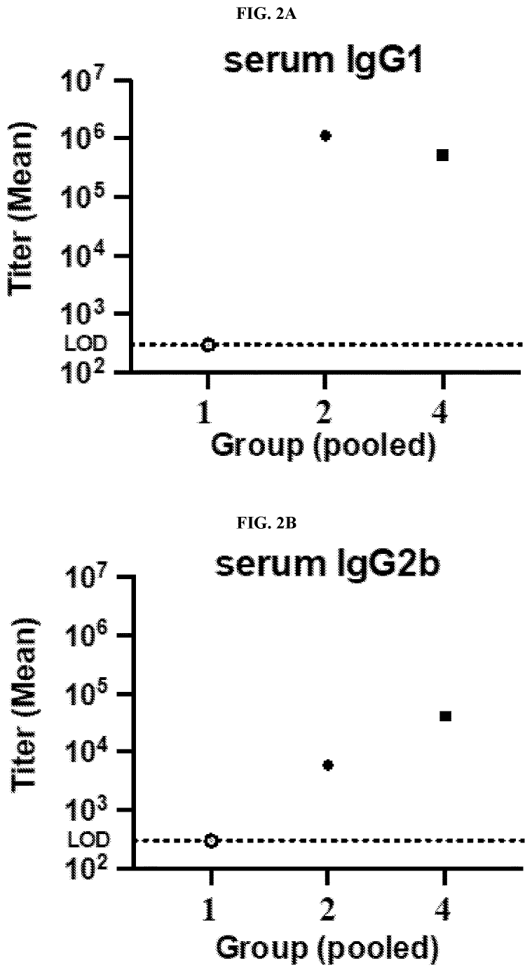

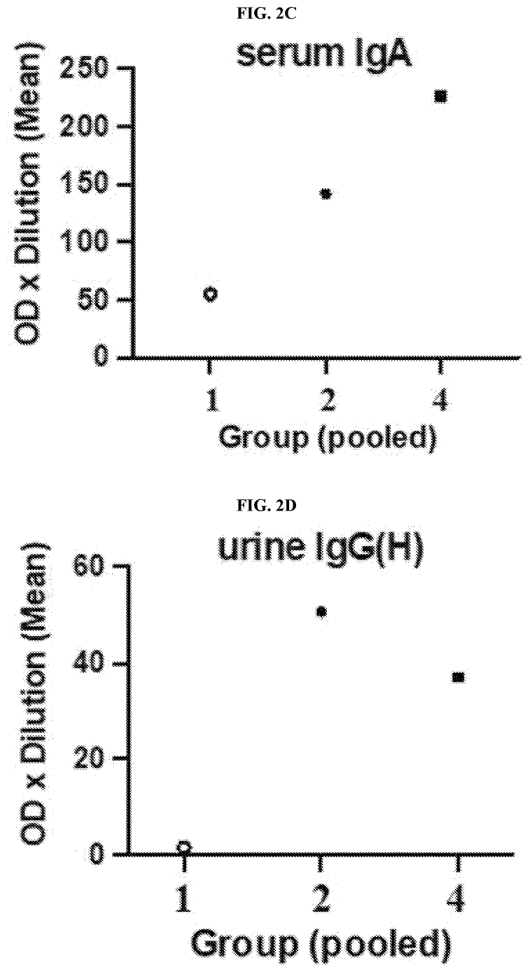

Serum samples were obtained from mice of groups 1-2 and 4 (i.e., SEQ ID NO:127 with either AlOH+dmLT or AlOH+CpG adjuvants) approximately 14 days after the second vaccination (i.e., approximately day 35) and analyzed by ELISA for serum IgG1 (FIG. 2A), serum IgG2b (FIG. 2B), serum IgA (FIG. 2C), and urine IgG(H) (FIG. 2D). The results indicate that SEQ ID NO:127 immunization induces both IgG1 and IgG2b production in HLA-DR4 mice.

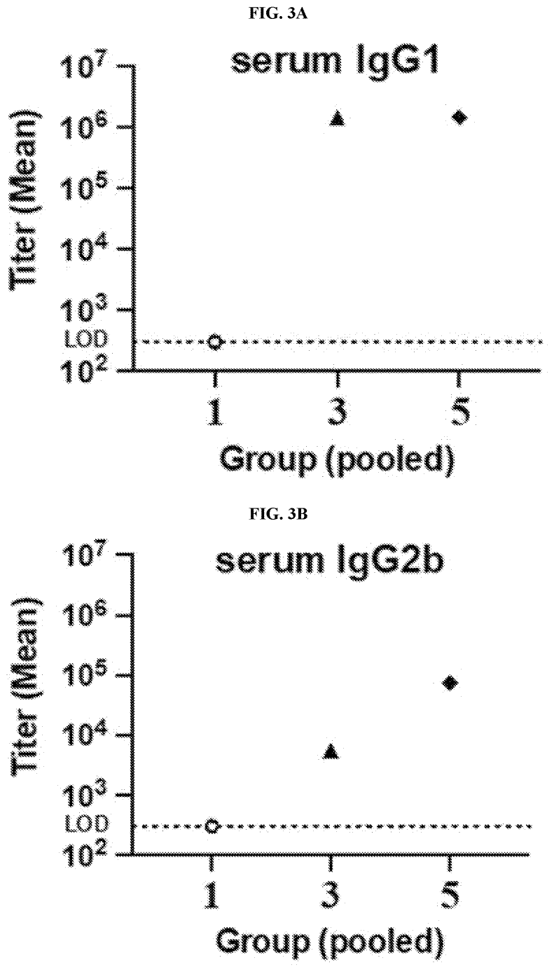

Serum samples were obtained from mice from of groups 1, 3, and 5 (i.e., SEQ ID NO:128 with either AlOH+dmLT or AlOH+CpG adjuvants) approximately 14 days after the second vaccination (i.e., day 35) and analyzed by ELISA for serum IgG1 (FIG. 3A) and serum IgG2b (FIG. 3B). The results indicate that SEQ ID NO:128 immunization induces both IgG1 and IgG2b production in HLA-DR4 mice.

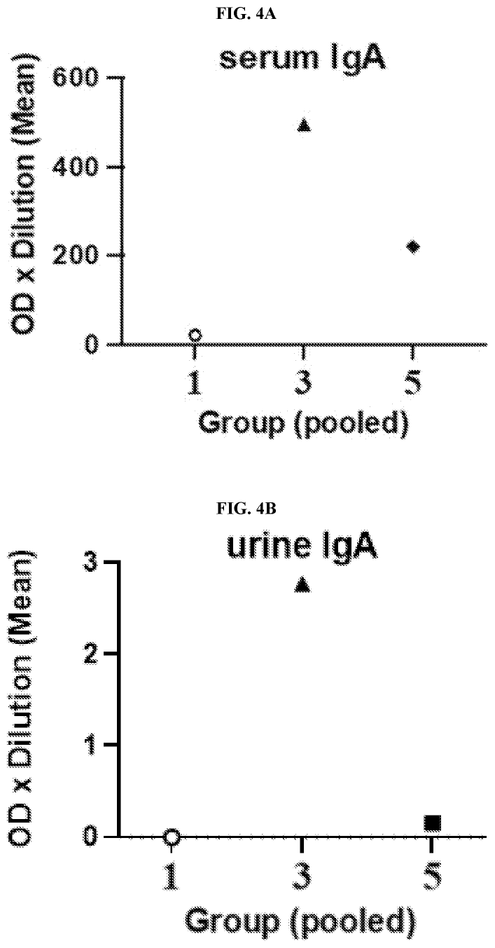

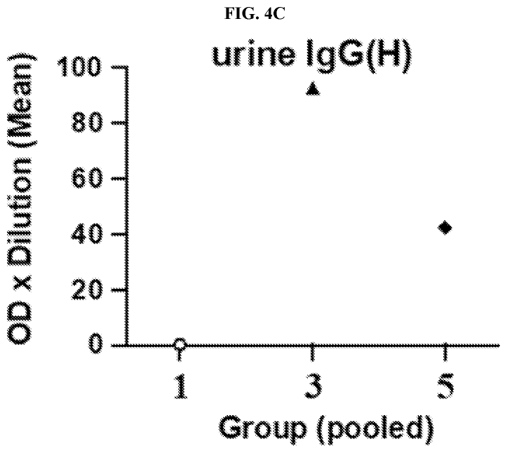

Serum and urine samples were obtained from mice from of groups 1, 3, and 5 (i.e., SEQ ID NO:128 with either AlOH+dmLT or AlOH+CpG adjuvants) approximately 14 days after the second vaccination (i.e., day 35) and analyzed by ELISA for serum IgA (FIG. 4A), urine IgA (FIG. 4B), and urine IgG(H) (FIG. 4C). The results indicate that SEQ ID NO:128 immunization induces IgA and IgG(H) production in HLA-DR4 mice, as evidenced by urine and/or serum samples.

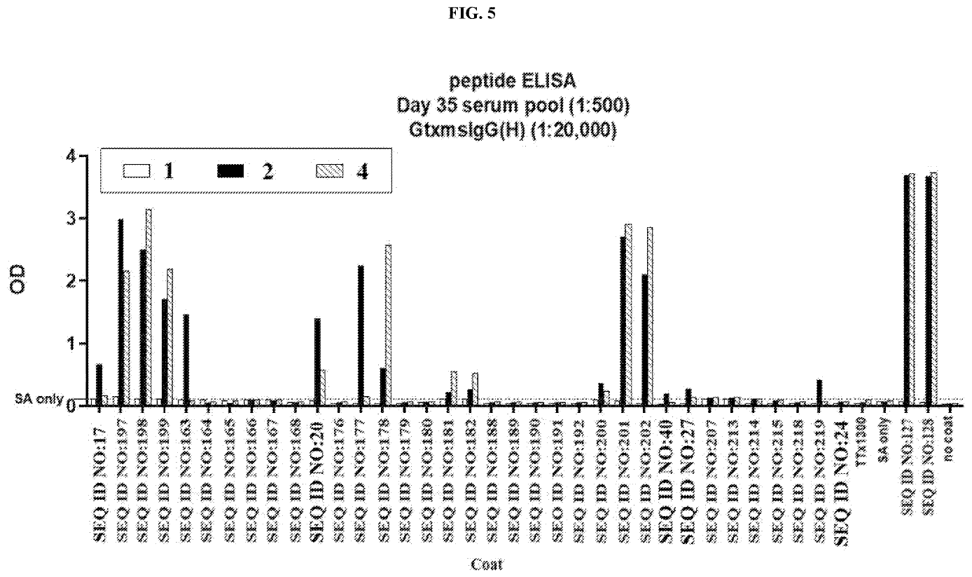

Serum samples were obtained from mice from groups 1, 2, and 4 (i.e., SEQ ID NO:127 with either AlOH+dmLT or AlOH+CpG adjuvants) approximately 14 days after the second vaccination (i.e., day 35) and analyzed by peptide ELISA, wherein wells were coated with certain polypeptides and/or antigen fragments (e.g., SEQ ID NOs: 17, 20, 24, 40, 197-199, 163-168, 176-182, 188-192, 200-202, 206-207, 213-215, and 218-219); antigens (e.g., SEQ ID NO:127 and SEQ ID NO:128), and controls (i.e., tetanus toxin, serum albumin only and no coat). Antibody production was not substantially observed in unvaccinated subjects (i.e., signal below or minimally over serum albumin only control in Group 1), whereas significant peptide specific antibody production was observed for many of the antigens, or antigen fragments thereof, evaluated in this study (FIG. 5).

Thus, in certain embodiments, the antigens and/or antigen fragments of the present disclosure elicit an adaptive immune response mediated by B cell activation, as indicated by elevated levels of several antibodies, non-limiting examples including IgG1, IgG2b, IgA, and IgG(H).

### Exp No. 2

HLA-DR4 mice were immunized as described herein using the vaccine compositions indicated in Table 2.

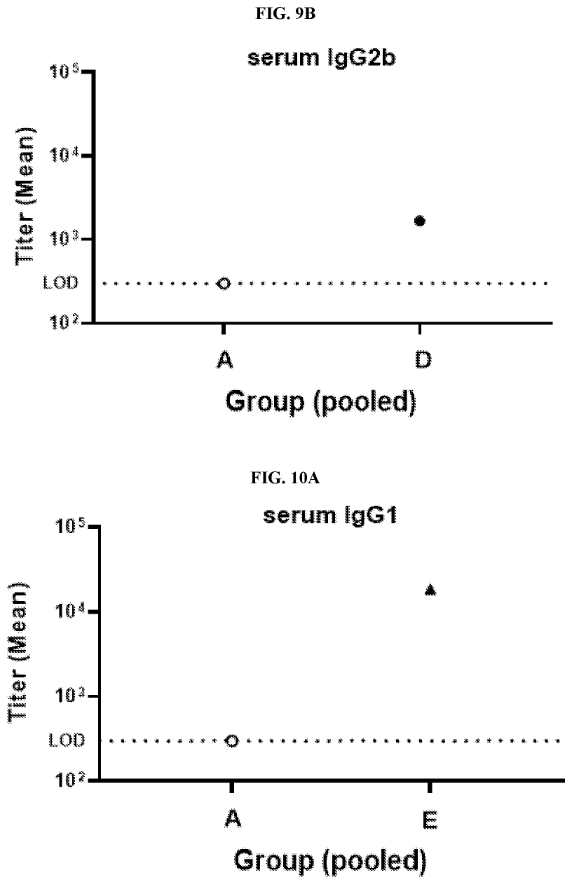

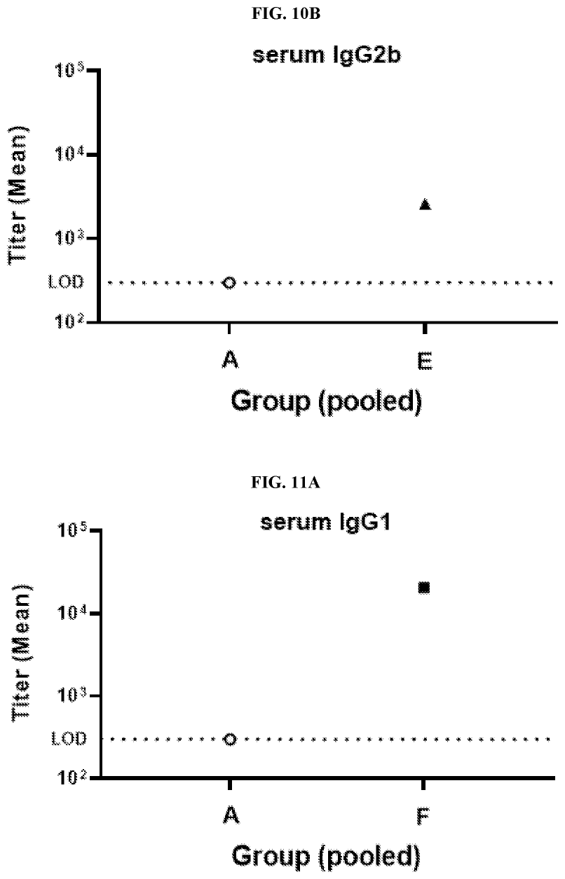

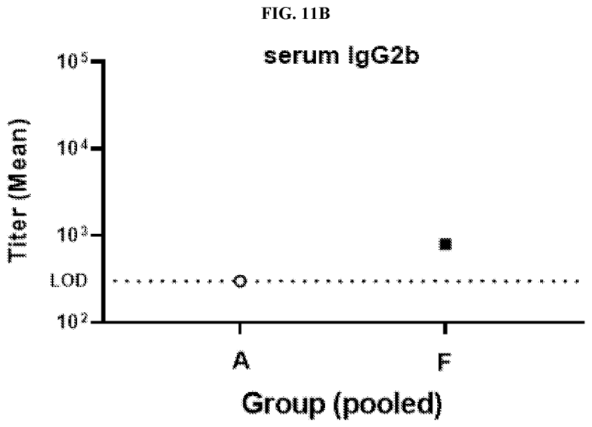

Serum samples were obtained from mice from each of groups A and D-F approximately 14 days after the second vaccination (i.e., day 28) and analyzed by ELISA for serum IgG1 (FIG. 9A, FIG. 10A, FIG. 11A) and serum IgG2b (FIG. 9B, FIG. 10B, and FIG. 11B). The results indicate that immunization with each of SEQ ID NO:132, SEQ ID NO:133, or SEQ ID NO:135 induces both IgG1 and IgG2b production in HLA-DR4 mice.

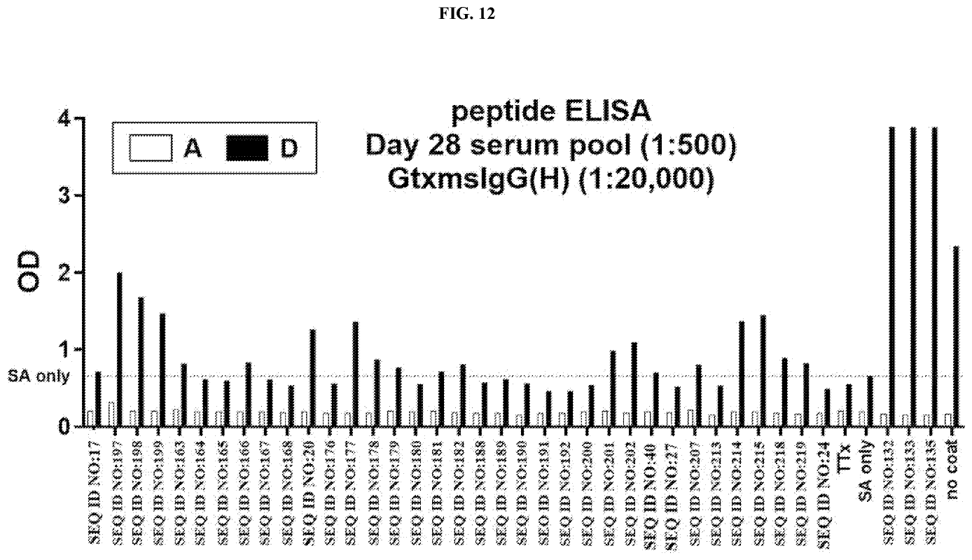

Serum samples were obtained from mice from groups A (i.e., placebo) and D (i.e., SEQ ID NO:132) approximately 14 days after the second vaccination (i.e., day 28) and analyzed by peptide ELISA, wherein wells were coated with certain polypeptides and/or antigen fragments (e.g., SEQ ID NOs: 17, 20, 24, 40, 197-199, 163-168, 176-182, 188-192, 200-202, 206-207, 213-215, and 218-219), antigens (i.e., SEQ ID NO:132, SEQ ID NO:133, and SEQ ID NO:135), and controls (i.e., tetanus toxin, serum albumin only and no coat). Antibody production was not substantially observed in subjects administered placebo (i.e., signal below or minimally over serum albumin only control in Group A), whereas significant peptide specific antibody production was observed for many of the antigens, or antigen fragments thereof, evaluated in this study (FIG. 12).

Thus, in certain embodiments, the antigens and/or antigen fragments of the present disclosure elicit an adaptive immune response mediated by B cell activation, as indicated by elevated levels of several antibodies, non-limiting examples including IgG1 and IgG2b.

Exp. No. 3

HLA-DR4 mice were immunized as described herein using the vaccine compositions indicated in Table 3.

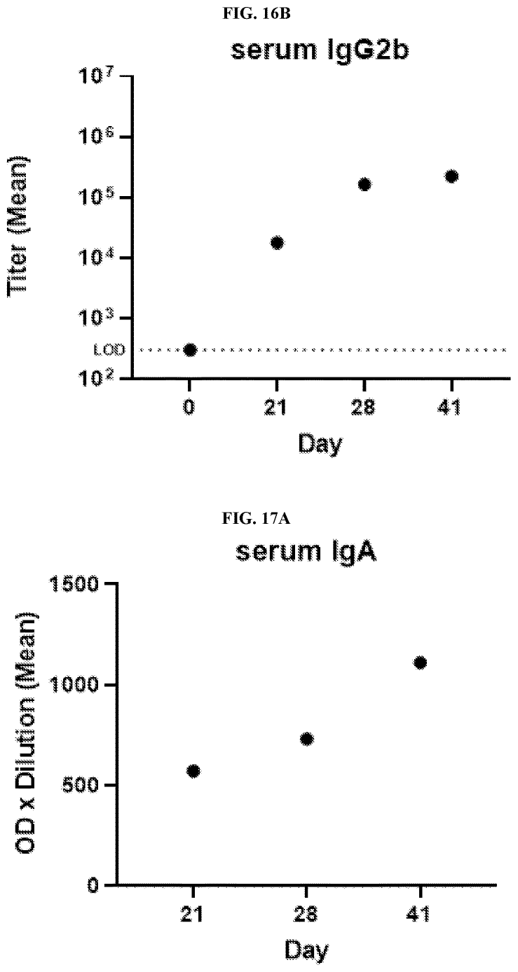

Serum samples were obtained from mice from the treatment group (i.e., group A) on days 0 (i.e., day of first vaccination; sample taken before vaccination), 21 (i.e., day of second vaccination), 28, and 41 (i.e., approximately 20 days after second vaccination) and analyzed by ELISA for serum IgG1 (FIG. 16A) and IgG2b (FIG. 16B). The results indicate that immunization with SEQ ID NO:132 induces both IgG1 and IgG2b production in HLA-DR4 mice.

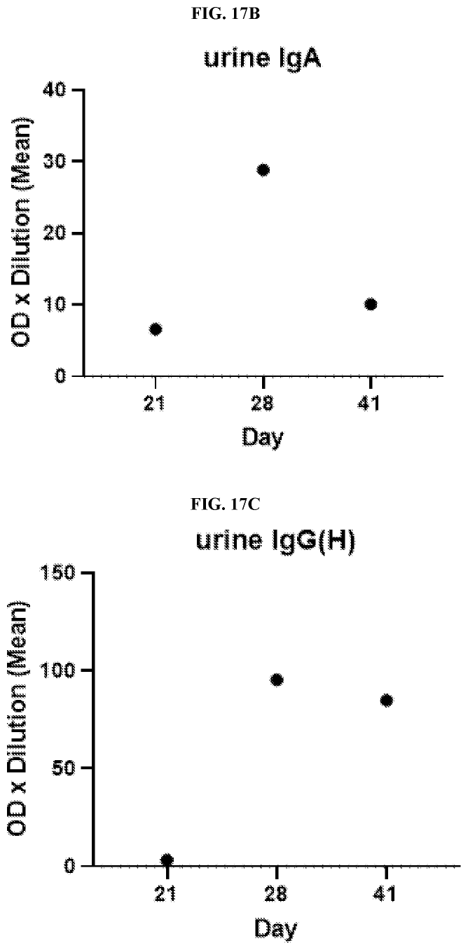

Serum and urine samples were obtained from mice from the treatment group (i.e., group A) on day 0 (i.e., day of first vaccination; sample taken before vaccination), and approximately each of days 21 (i.e., day of second vaccination), 28, and 41 (i.e., 20 days after second vaccination) and analyzed by ELISA for serum IgA (FIG. 17A), urine IgA (FIG. 17B), and urine IgG(H) (FIG. 17C). The results indicate that SEQ ID NO:132 immunization induces IgA and IgG(H) production in HLA-DR4 mice, as evidenced by urine and/or serum samples.

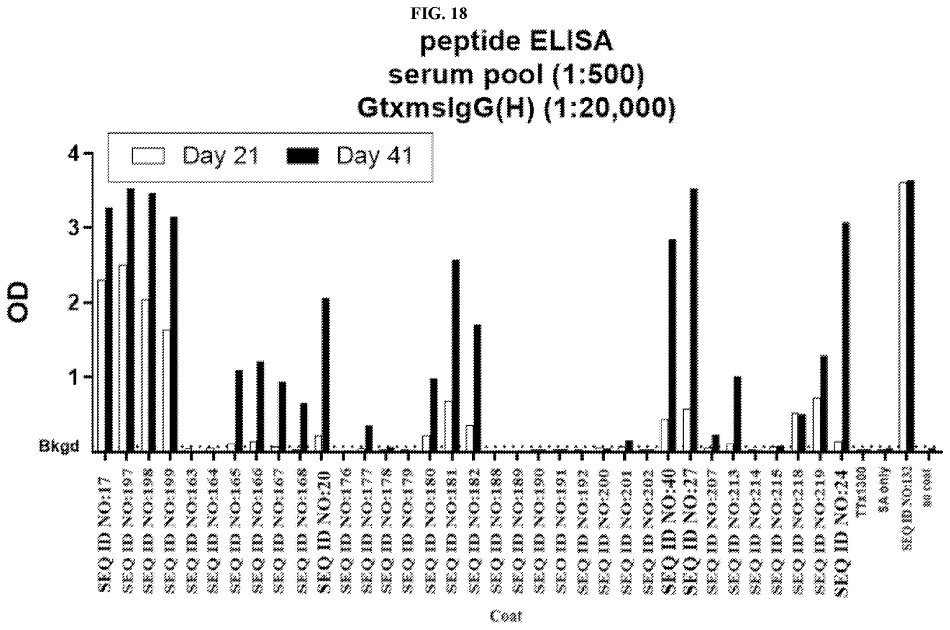

Serum samples were obtained from mice from the treatment group (i.e., group A) approximately on days 21 and 41 analyzed by peptide ELISA, wherein wells were coated with certain polypeptides and/or antigen fragments (e.g., SEQ ID NOs: 17, 20, 24, 40, 197-199, 163-168, 176-182, 188-192, 200-202, 206-207, 213-215, and 218-219), antigens (i.e., SEQ ID NO:132), and controls (i.e., tetanus toxin, serum albumin only and no coat). Generally, antibody production was greater on approximately day 41 than on approximately day 21 (FIG. 18).

Thus, in certain embodiments, the antigens and/or antigen fragments of the present disclosure elicit an adaptive immune response mediated by B cell activation, as indicated by elevated levels of several antibodies, non-limiting examples including IgG1, IgG2b, IgA, and IgG(H).

Exp. No. 4

HLA-DR4 mice were immunized as described herein using the vaccine compositions indicated in Table 4.

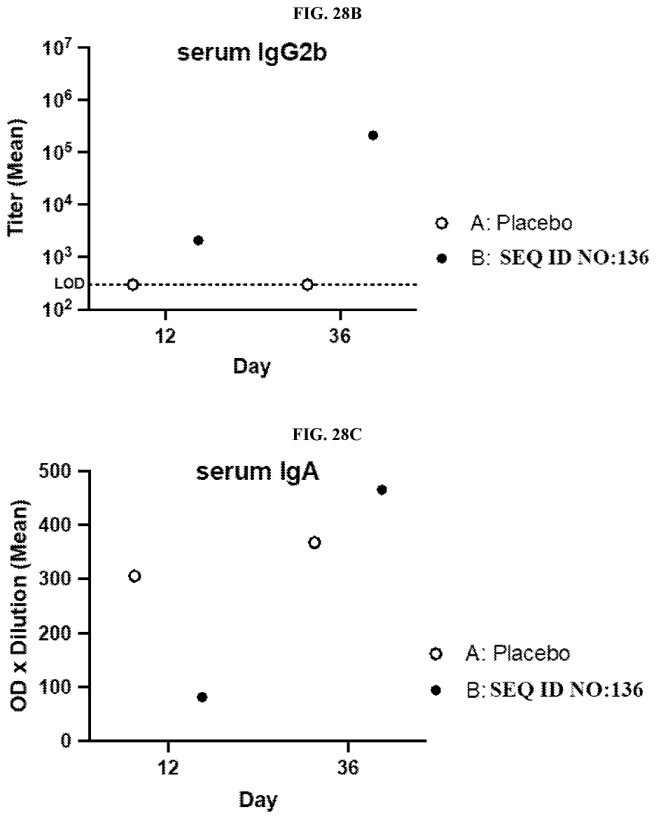

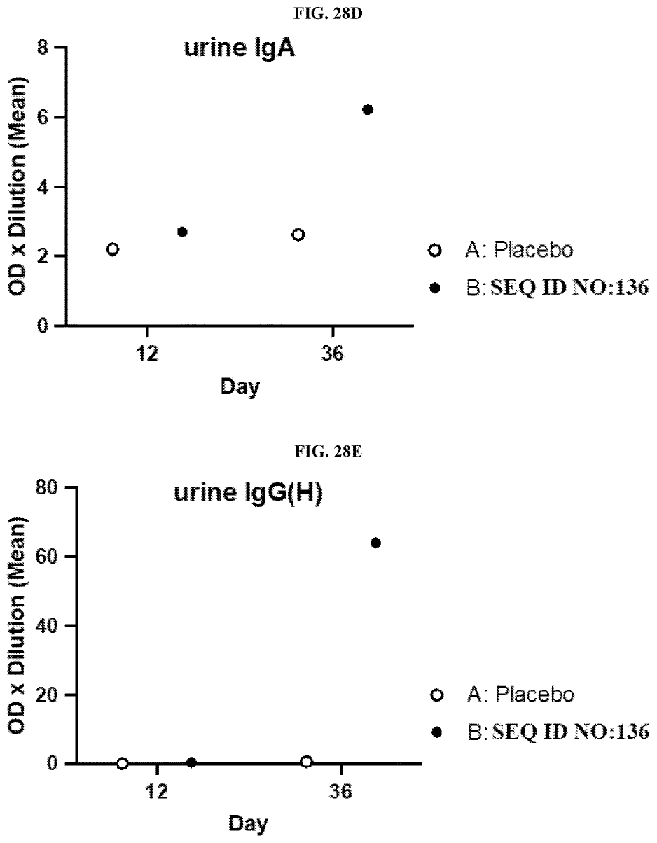

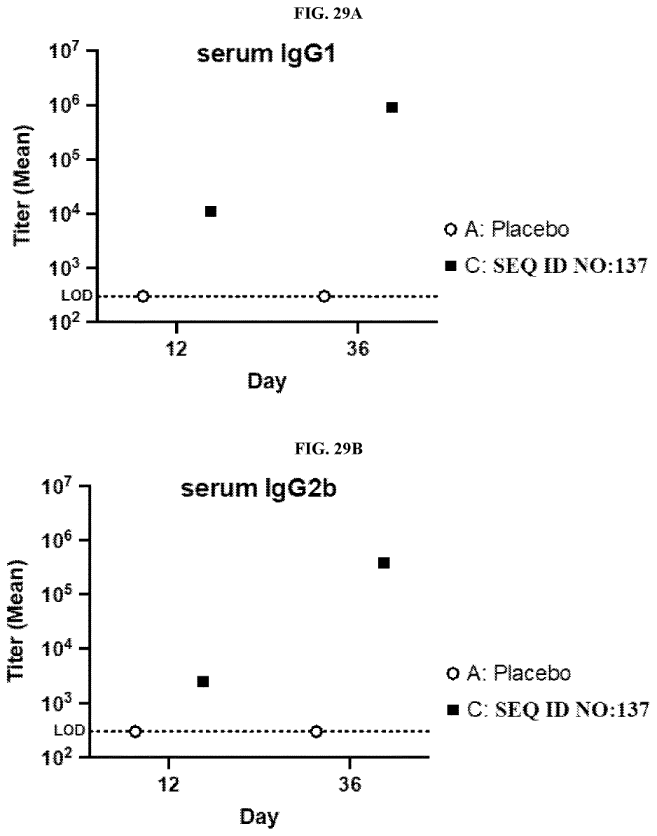

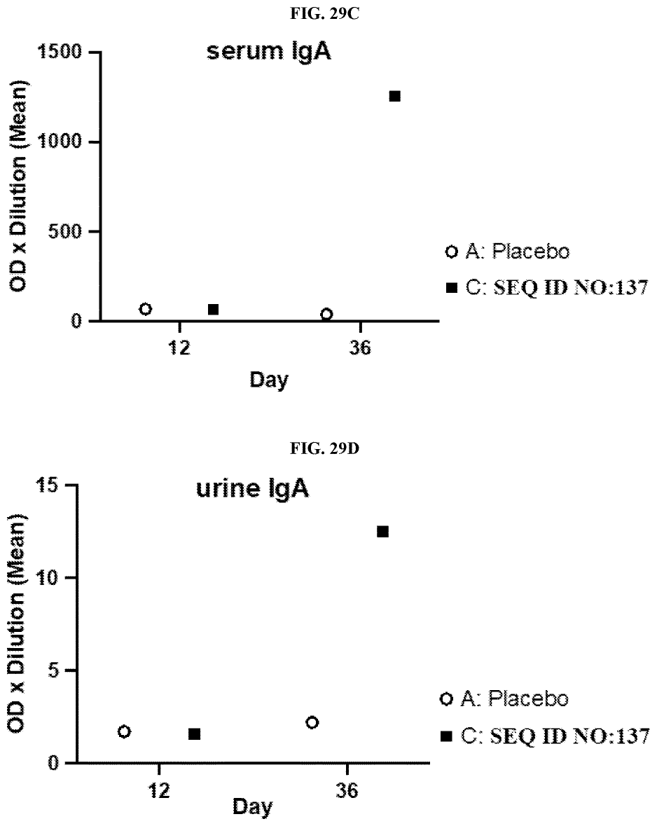

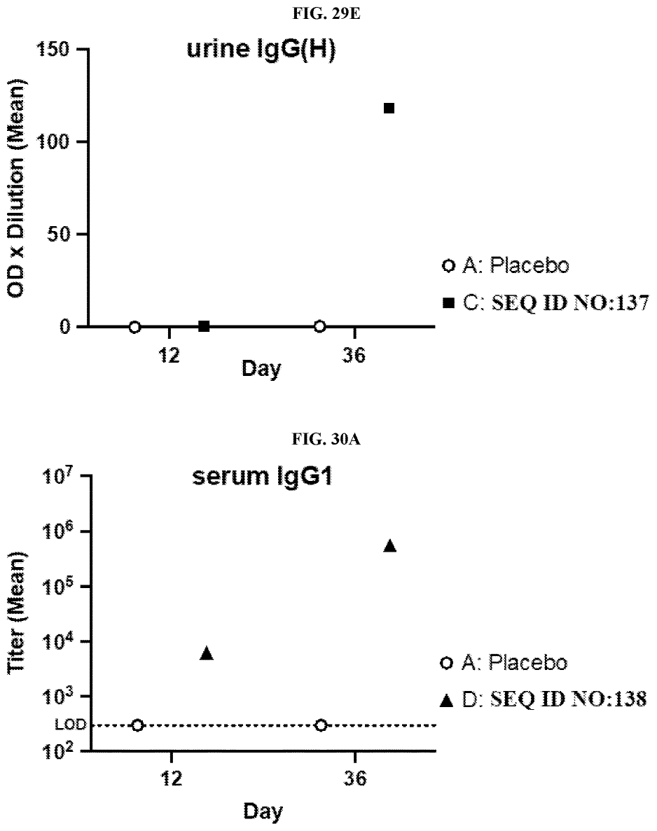

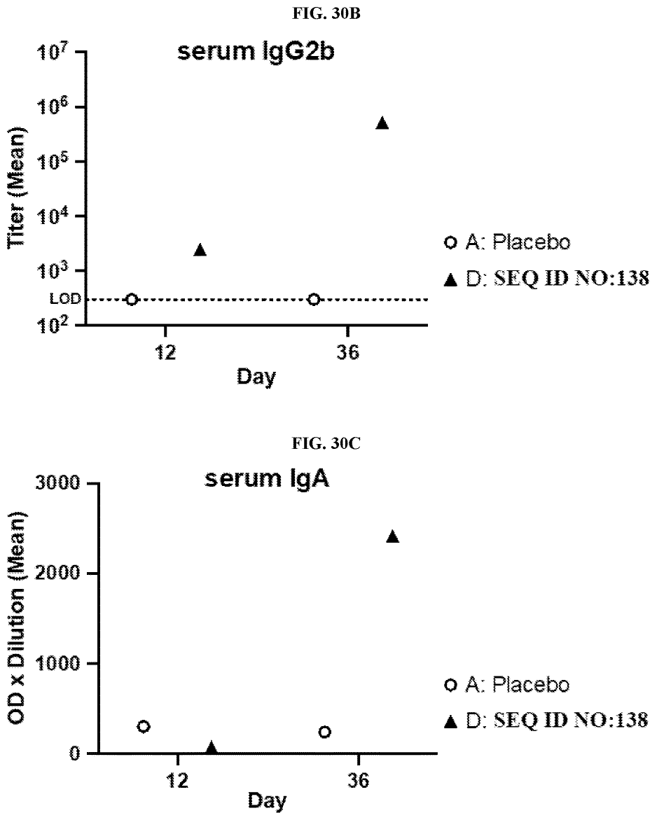

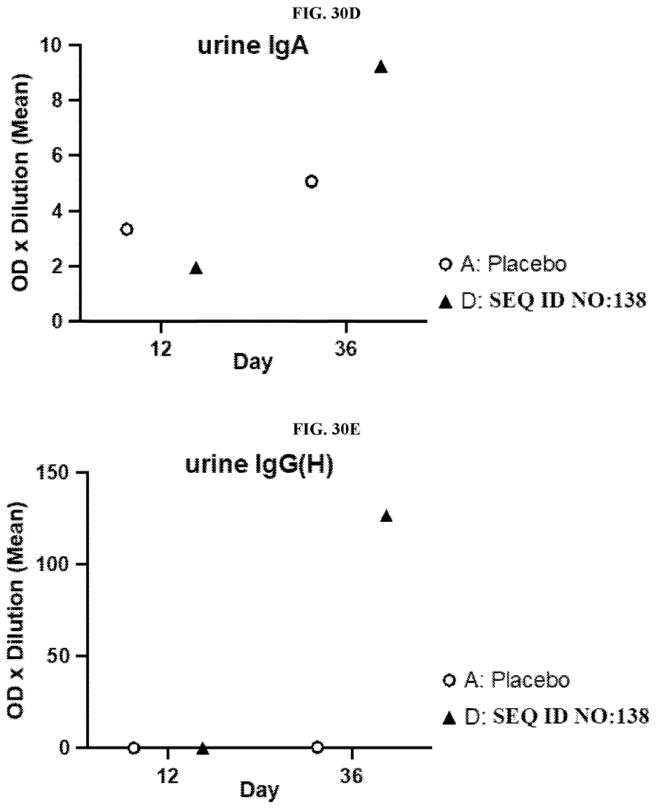

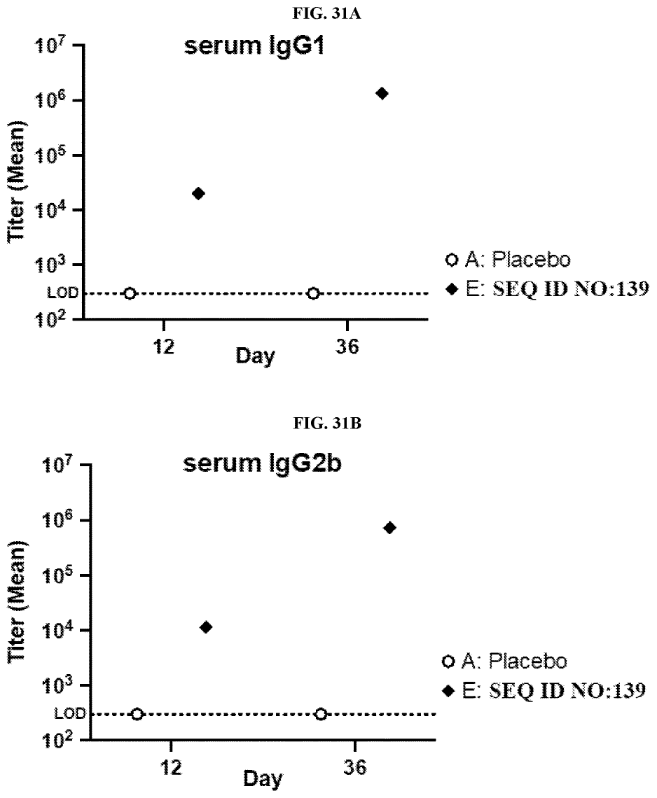

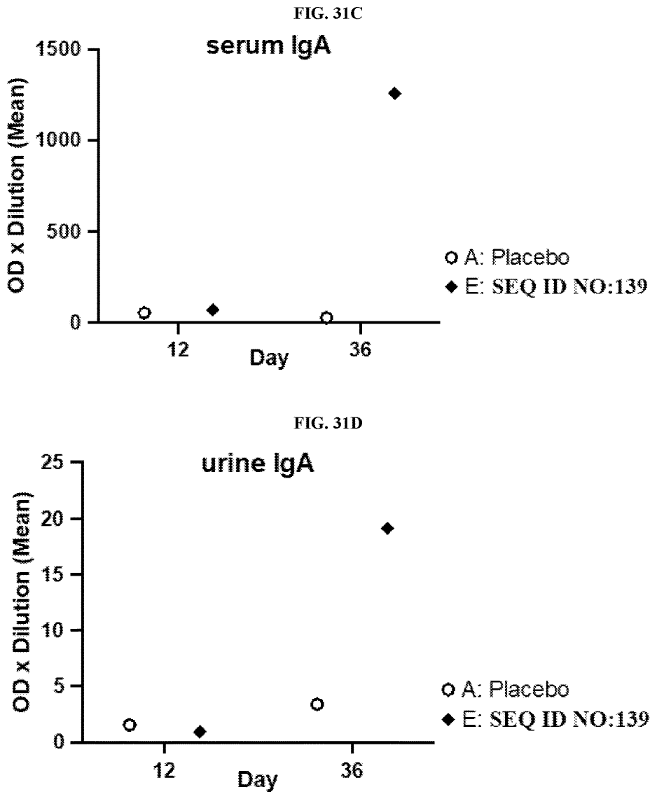

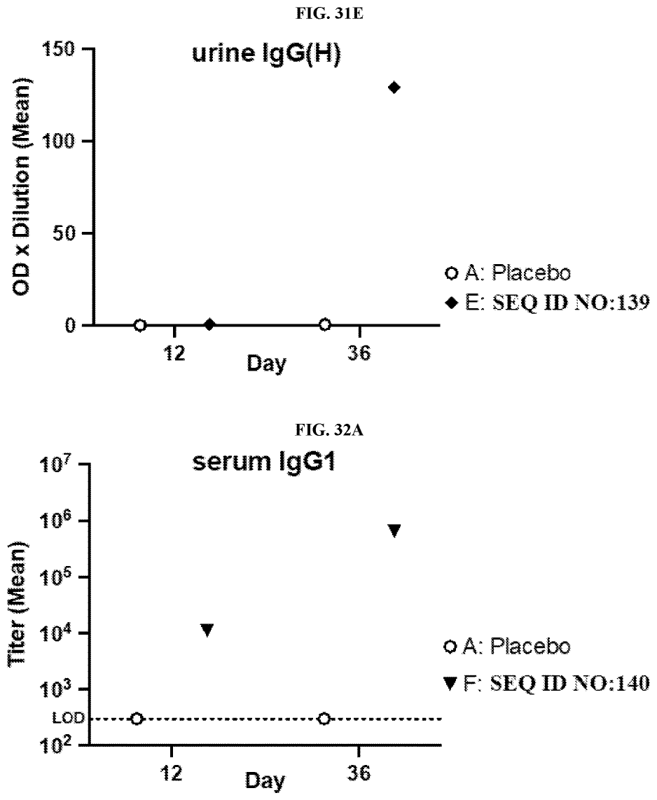

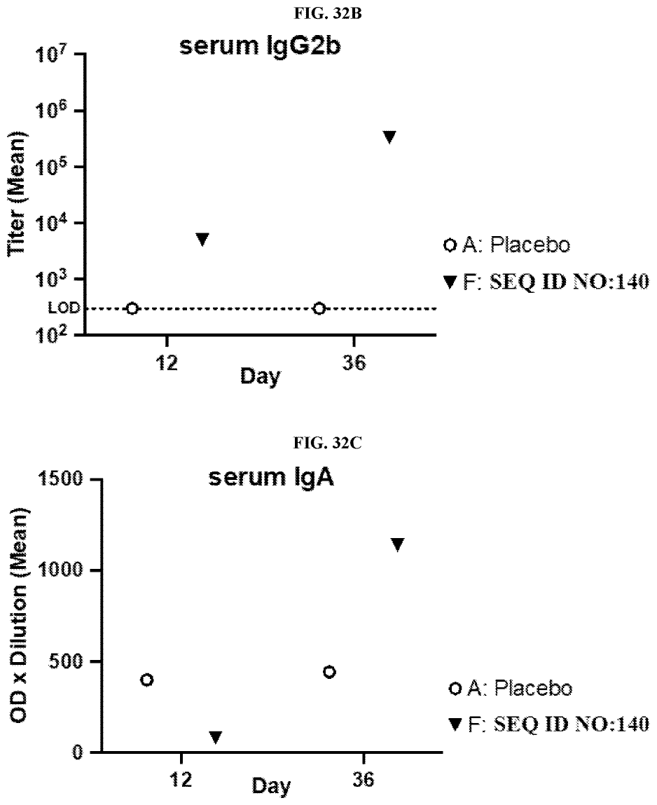

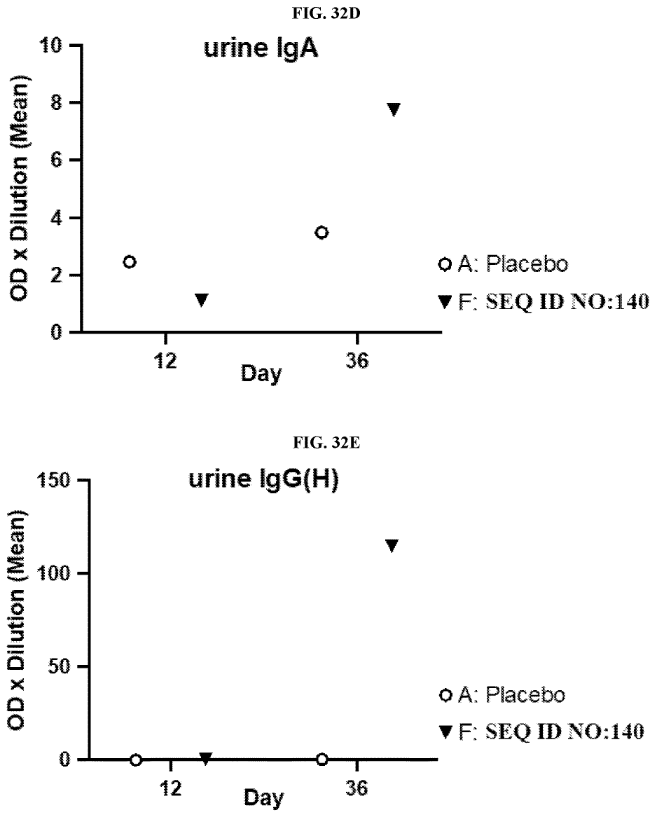

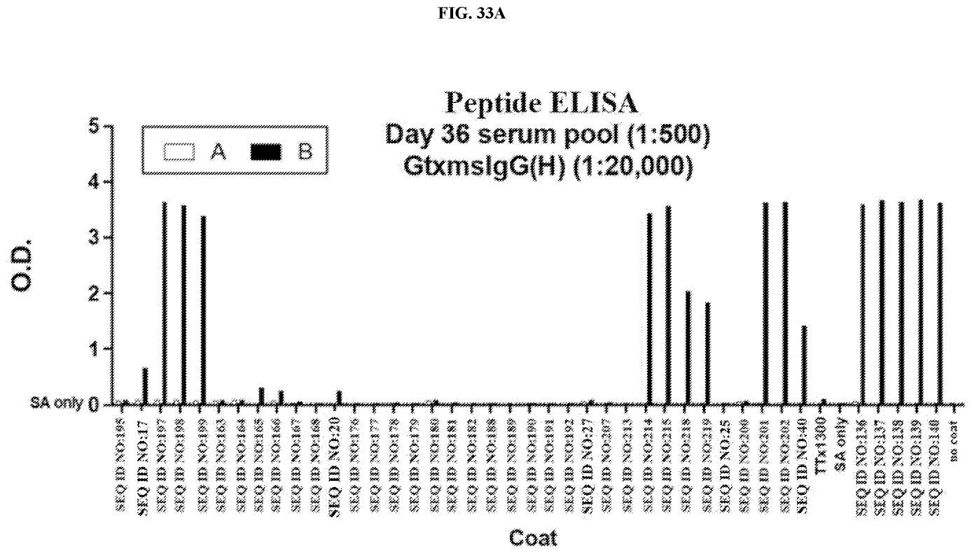

Serum and urine samples were obtained from mice from each treatment group (i.e., Groups B-F) and a control group (i.e., Group A) at several time points after initial vaccination and analyzed by ELISA for serum IgG1, serum IgG2b, serum IgA, urine IgA, and urine IgG(H) in wells coated with SEQ ID NO:136 (FIGS. 28A-28E), SEQ ID NO:137 (FIGS. 29A-29E), SEQ ID NO:138 (FIGS. 30A-30E), SEQ ID NO:139 (FIGS. 31A-31E), and SEQ ID NO:140 (FIGS. 32A-32E).

In certain embodiments, the results indicate that all evaluated immunizations induce IgG1, IgG2b, and IgA production in HLA-DR4 mice sera approximately 5 weeks after initial vaccination, as compared to the control. In certain embodiments, the results indicate that most evaluated immunizations induce IgG1, IgG2b, and IgA production in HLA-DR4 mice sera approximately 2 weeks after second vaccination, as compared to the control. In certain embodiments, the results indicate that most evaluated immunizations induce IgG1, IgG2b, and IgA production in HLA-DR4 mice sera approximately 2 weeks after initial vaccination, as compared to the control.

In certain embodiments, the results indicate that all evaluated immunizations induce IgA and IgG(H) production in the urine of HLA-DR4 mice approximately 5 weeks after initial vaccination, as compared to the control.

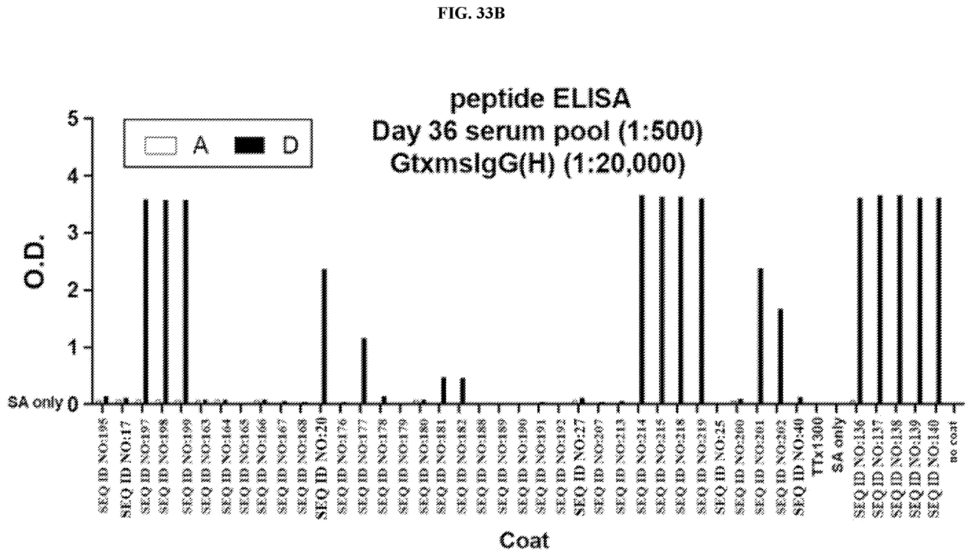

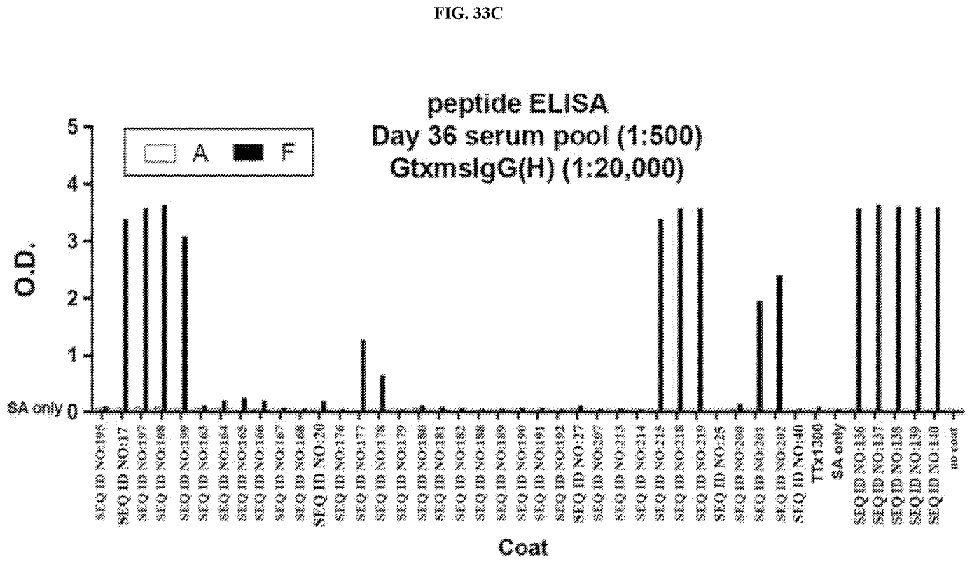

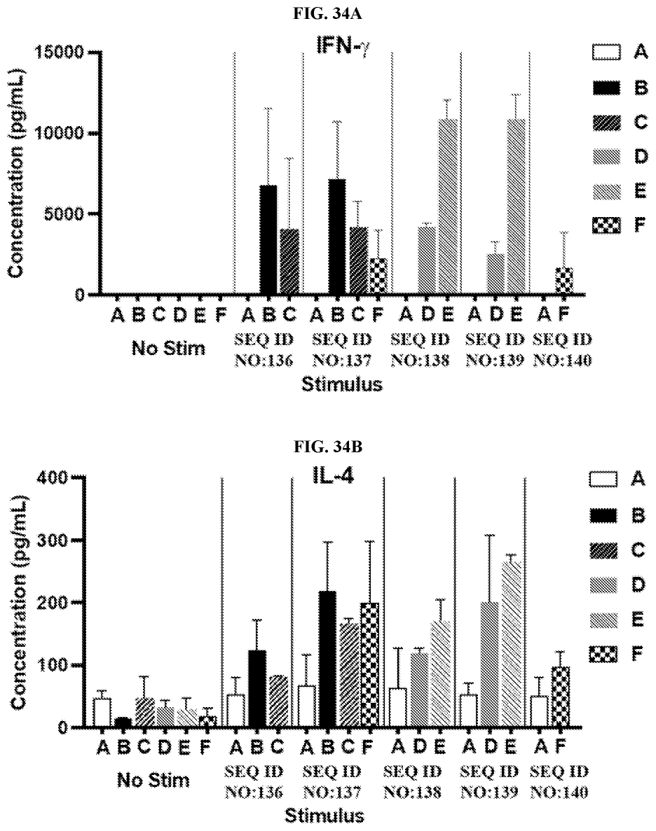

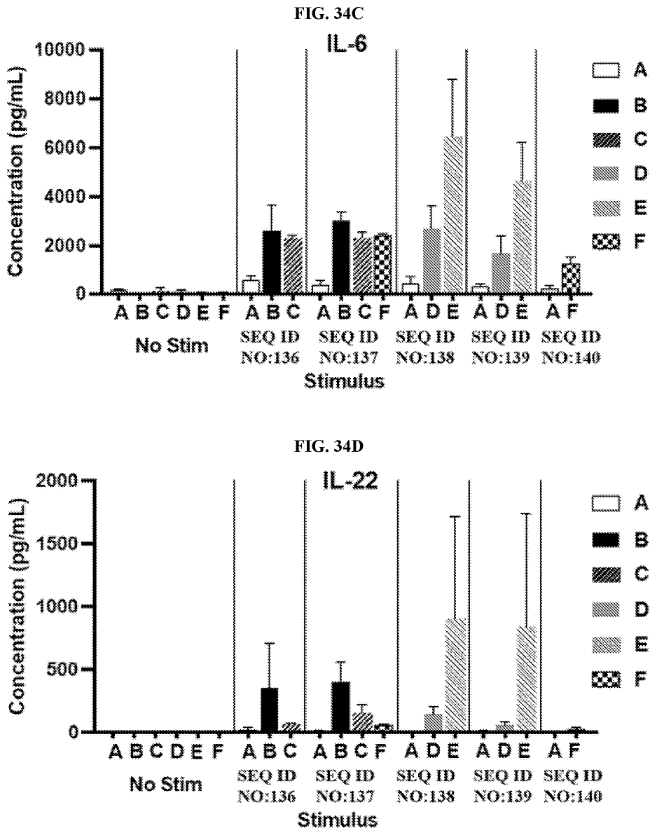

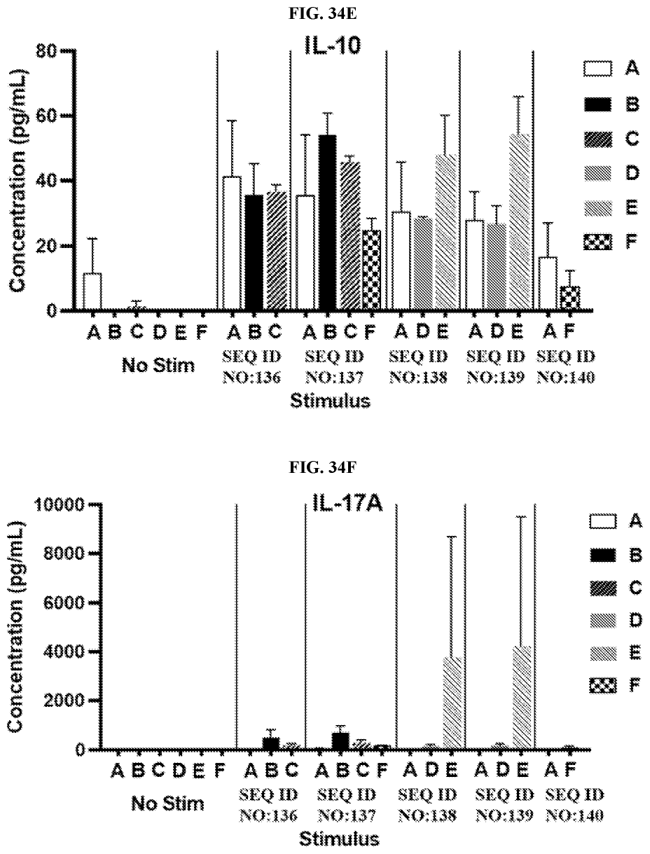

Serum samples were obtained from mice from the control group (i.e., Group A) and each of the treatment groups B (FIG. 33A), D (FIG. 33B), and F (FIG. 33C) on day 36 after first vaccination (approximately 2 weeks after second vaccination) and analyzed by peptide ELISA, wherein wells were coated with certain polypeptides and/or antigen fragments (e.g., SEQ ID NOs: 17, 20, 25, 27, 195, 197-199, 163-168, 176-182, 188-192, 207, 213-215, 218-219, 200-203, and 136-140) and controls (i.e., tetanus toxin, serum albumin only and no coat). The data depict antibody production in wells containing each antigen utilized in Groups B-F and certain exemplary antigenic fragments.

Thus, in certain embodiments, the antigens and/or antigen fragments of the present disclosure elicit an adaptive immune response mediated by B cell activation, as indicated by elevated levels of certain antibodies, non-limiting examples including IgG1, IgG2b, IgA, and IgG(H).

Exp. No. 5

HLA-DR4 mice were immunized as described herein using the vaccine compositions indicated in Table 5.

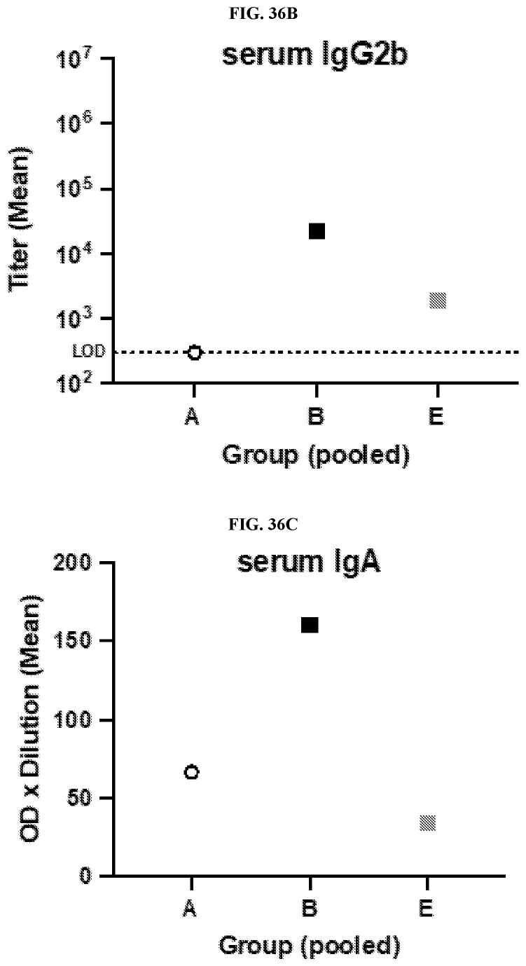

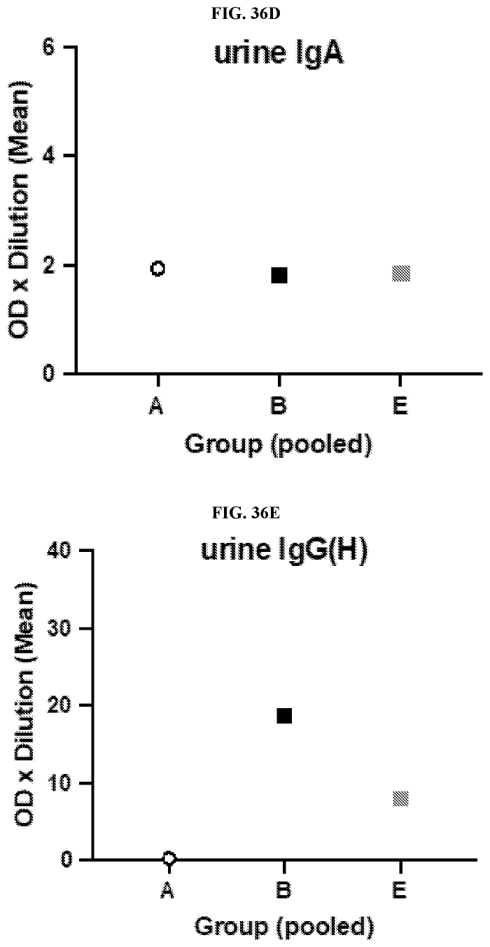

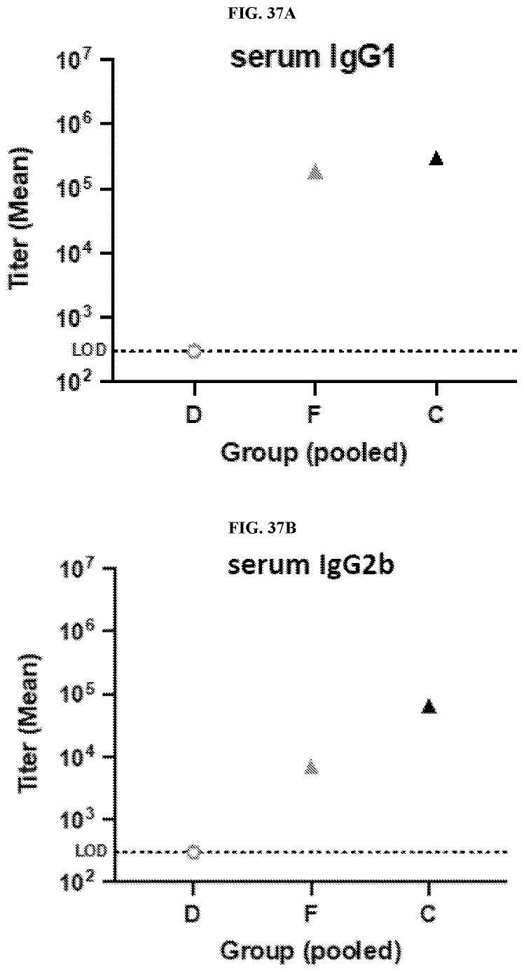

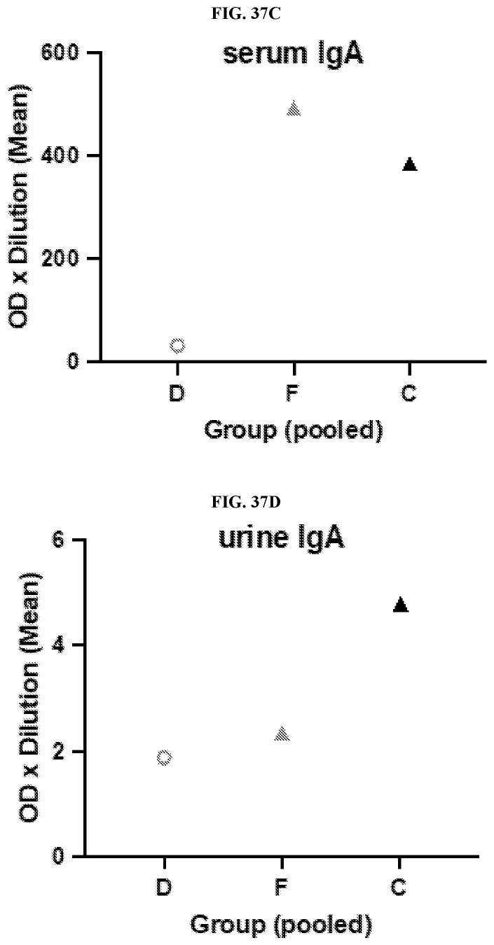

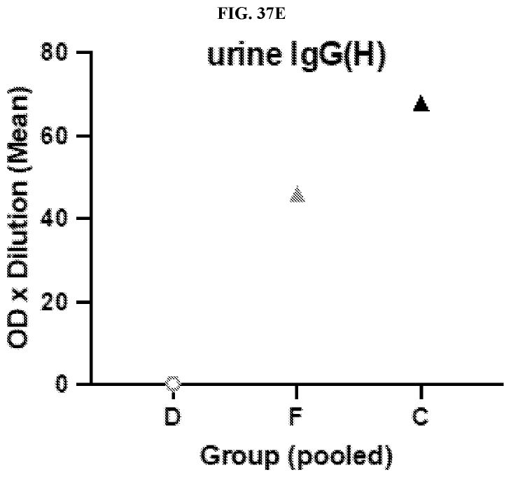

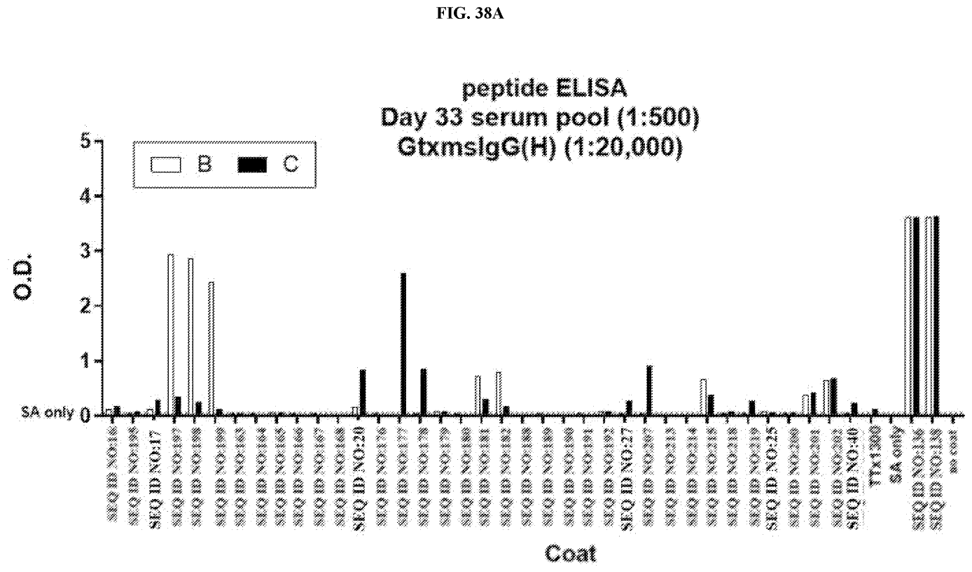

Serum and urine samples were obtained from mice from each treatment group (i.e., Groups B-C and E-F) and a control group (i.e., Groups A and D) 33 days after initial vaccination (approximately 2 weeks after second vaccination) and analyzed by ELISA for serum IgG1, serum IgG2b, serum IgA, urine IgA, and urine IgG(H) in wells coated with SEQ ID NO:136 (FIG. 36A-36E) or SEQ ID NO:138 (FIG. 37A-37E).

In certain embodiments, the results indicate that all evaluated immunizations induce IgG1, IgG2b, and IgA production in HLA-DR4 mice sera 33 days after initial vaccination (approximately 2 weeks after second vaccination), as compared to the control. In certain embodiments, the results indicate that most evaluated immunizations induce IgA and IgG(H) production in the urine of HLA-DR4 mice 33 days after initial vaccination (approximately 2 weeks after second vaccination), as compared to the control.

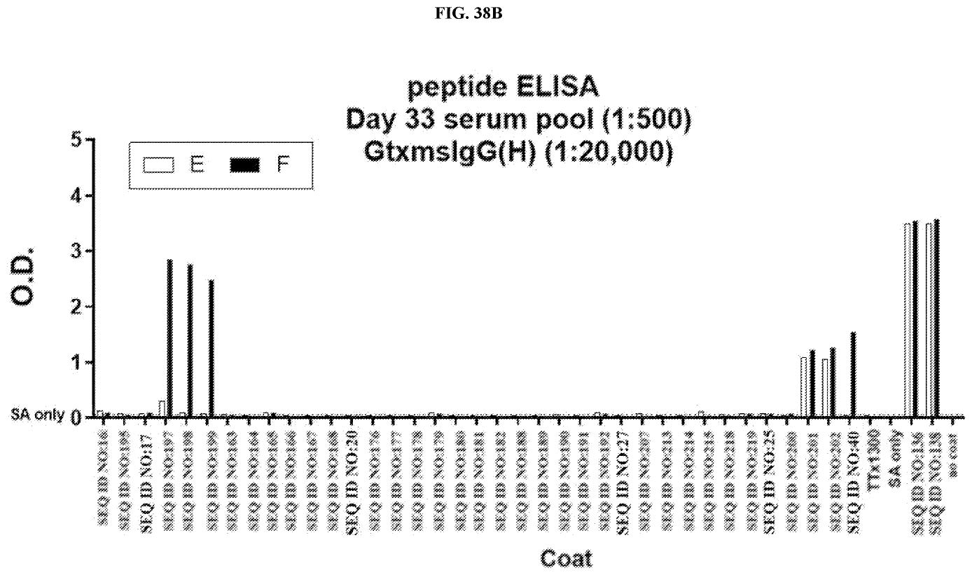

Serum samples were obtained from mice from the treatment groups B and D (FIG. 38A) and treatment groups E and F (FIG. 38B) on day 33 after first vaccination (approximately 2 weeks after second vaccination) and analyzed by peptide ELISA, wherein wells were coated with certain polypeptides and/or antigen fragments (e.g., SEQ ID NOs: 16-17, 20, 25, 27, 195, 197-199, 163-168, 176-182, 188-192, 207, 213-215, 218-219, 200-203, 136 and 138) and controls (i.e., tetanus toxin, serum albumin only and no coat). The data depict antibody production in wells containing each antigen utilized in exemplary vaccine antigens and/or fragments thereof.

Thus, in certain embodiments, the antigens and/or antigen fragments of the present disclosure elicit an adaptive immune response mediated by B cell activation, as indicated by elevated levels of certain antibodies, non-limiting examples including IgG1, IgG2b, IgA, and IgG(H).

Exp. No. 6 HLA-DR4 mice were immunized as described herein using the vaccine compositions indicated in Table 6.

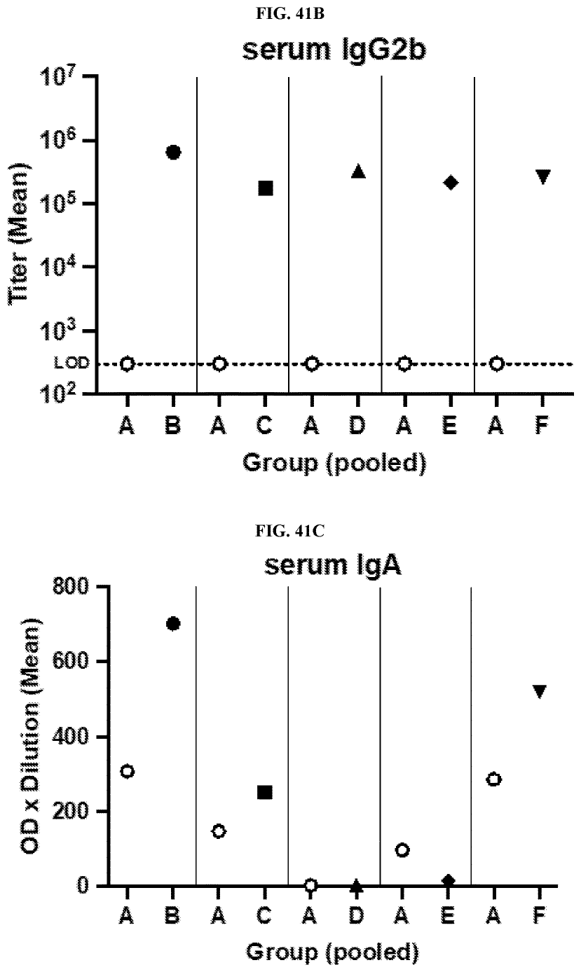

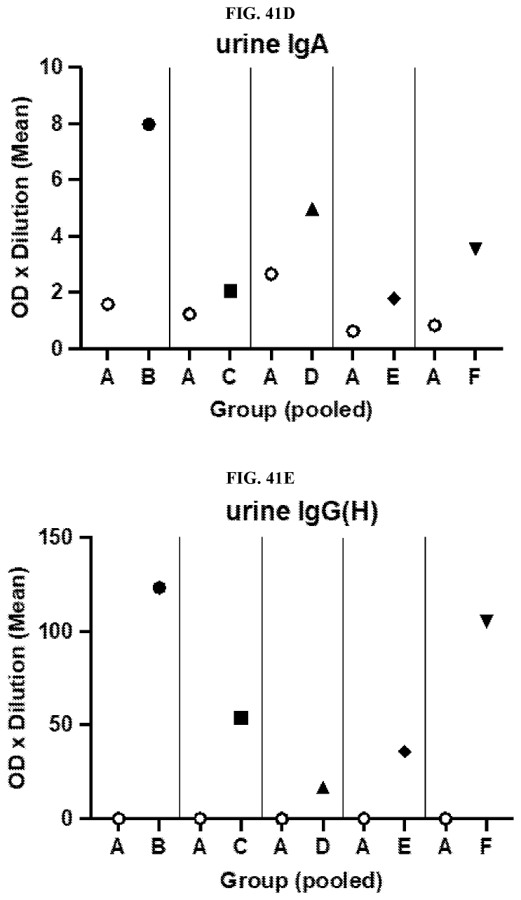

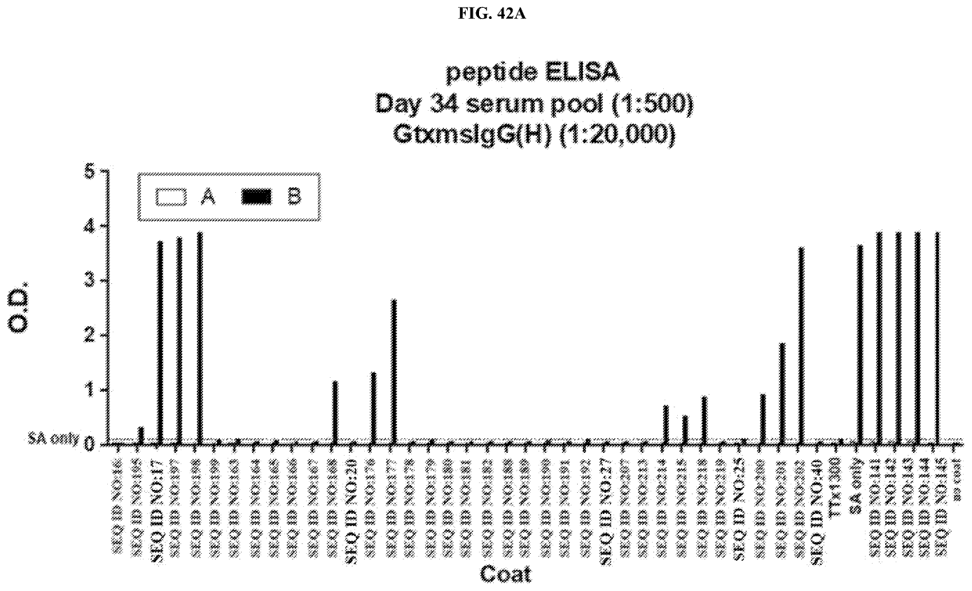

Serum and urine samples were obtained from mice from each treatment group (i.e., Groups B-F) and a control group (i.e., Group A) 34 days after initial vaccination (approximately 2 weeks after second vaccination) and analyzed by ELISA for serum IgG1, serum IgG2b, serum IgA, urine IgA, and urine IgG(H) (FIG. 41A-41E).

In certain embodiments, the results indicate that the evaluated immunizations induce IgG1, IgG2b, and IgA production in HLA-DR4 mice sera 34 days after initial vaccination (approximately 2 weeks after second vaccination), as compared to the control. In certain embodiments, the results indicate that the evaluated immunizations induce IgA and IgG(H) production in the urine of HLA-DR4 mice 34 days after initial vaccination (approximately 2 weeks after second vaccination), as compared to the control.

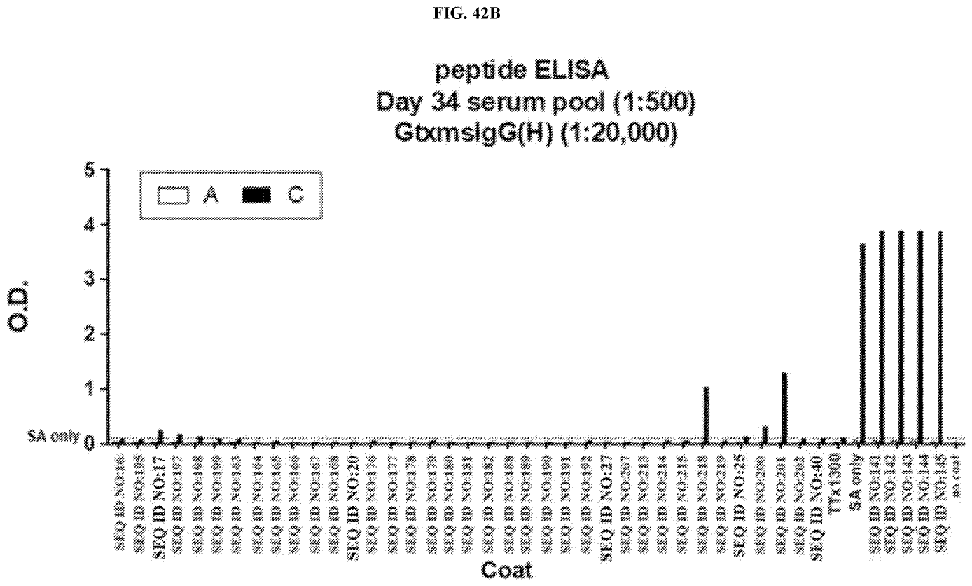

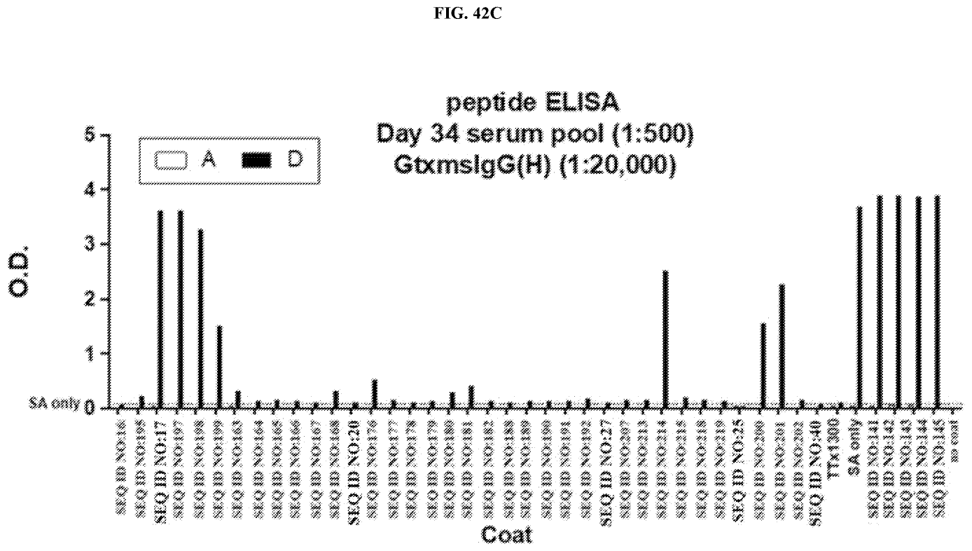

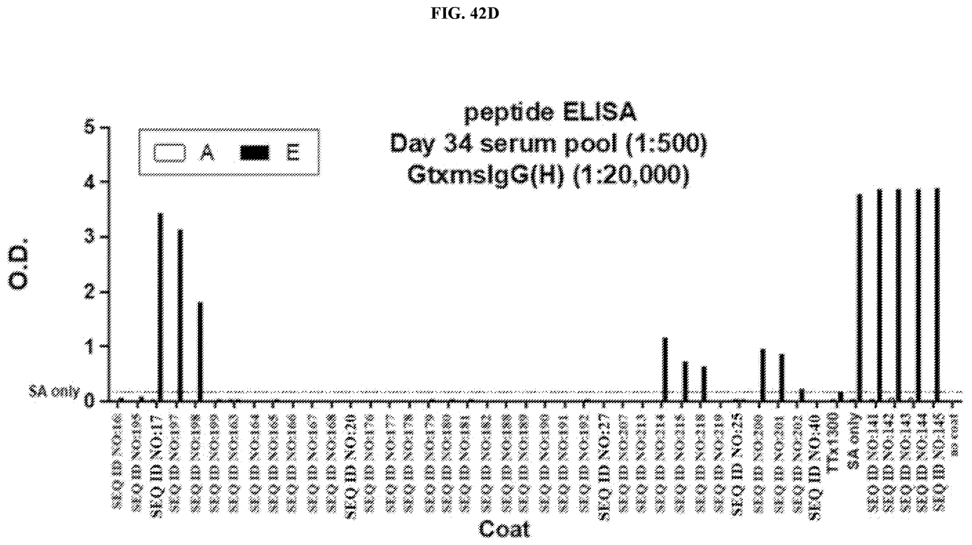

Serum samples were obtained from mice from the control (Group A) and each of treatment groups B (FIG. 42A), C (FIG. 42B), D (FIG. 42C), and E (FIG. 42D) on day 34 after first vaccination (approximately 2 weeks after second vaccination) and analyzed by peptide ELISA, wherein wells were coated with certain polypeptides and/or antigen fragments (e.g., SEQ ID Nos: 16-17, 20, 25, 27, 195, 197-199, 163-168, 176-182, 188-192, 207, 213-215, 218-219, 200-203, and 141-145) and controls (i.e., tetanus toxin, serum albumin only and no coat). The data depict antibody production in wells containing each antigen utilized in exemplary vaccine antigens and/or fragments thereof.

Thus, in certain embodiments, the antigens and/or antigen fragments of the present disclosure elicit an adaptive immune response mediated by B cell activation, as indicated by elevated levels of certain antibodies, non-limiting examples including IgG1, IgG2b, IgA, and IgG(H).

Exp. No. 7

HLA-DR4 mice were immunized as described herein using the vaccine compositions indicated in Table 7.

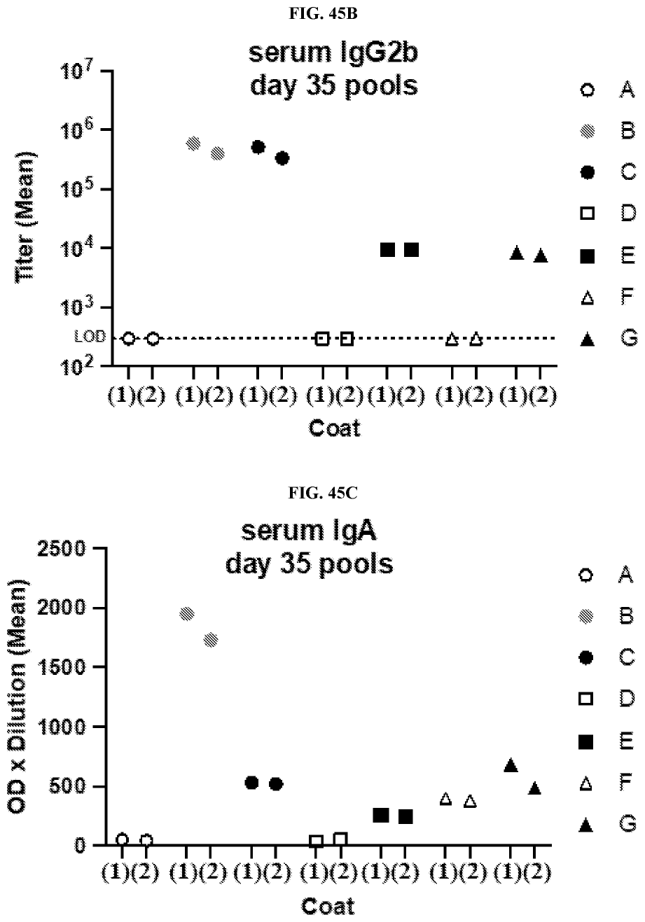

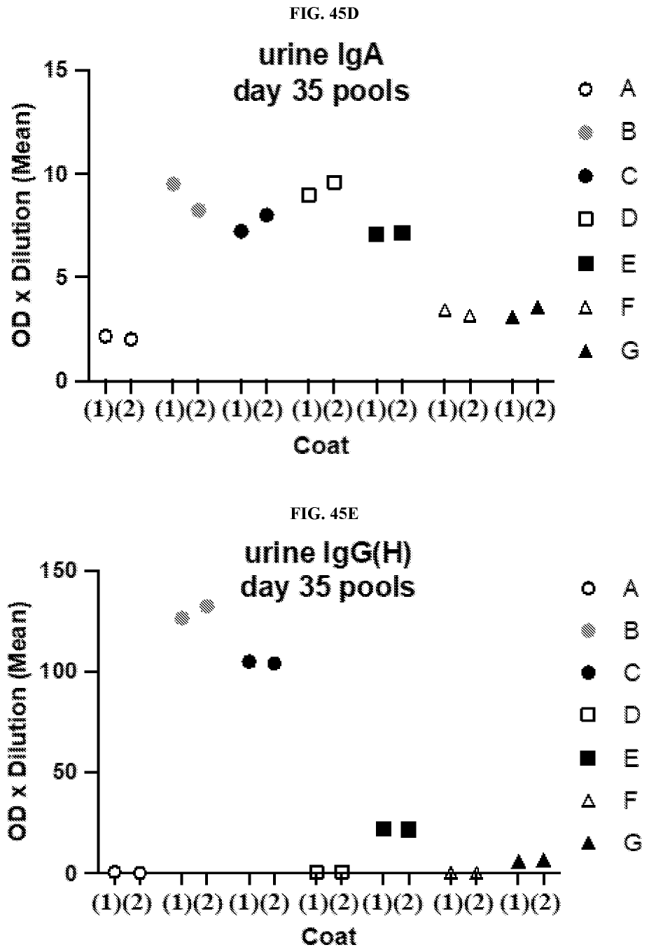

Serum and urine samples were obtained from mice from each treatment group (i.e., Groups B-C, E, and G) and each control group (i.e., Groups A, D, and F) 35 days after initial vaccination (approximately 2 weeks after second vaccination) and analyzed by ELISA for serum IgG1, serum IgG2b, serum IgA, urine IgA, and urine IgG(H) in wells coated with SEQ ID NO:138 (1) or (2), wherein (1) and (2) vary with respect to buffer conditions, as described in Table 7 (FIGS. 45A-45E).

In certain embodiments, the results indicate that certain evaluated immunizations induce IgG1, IgG2b, and IgA production in HLA-DR4 mice sera 35 days after initial vaccination (approximately 2 weeks after second vaccination), as compared to the control. In certain embodiments, the results indicate that certain evaluated immunizations induce IgA and IgG(H) production in the urine of HLA-DR4 mice 35 days after initial vaccination (approximately 2 weeks after second vaccination).

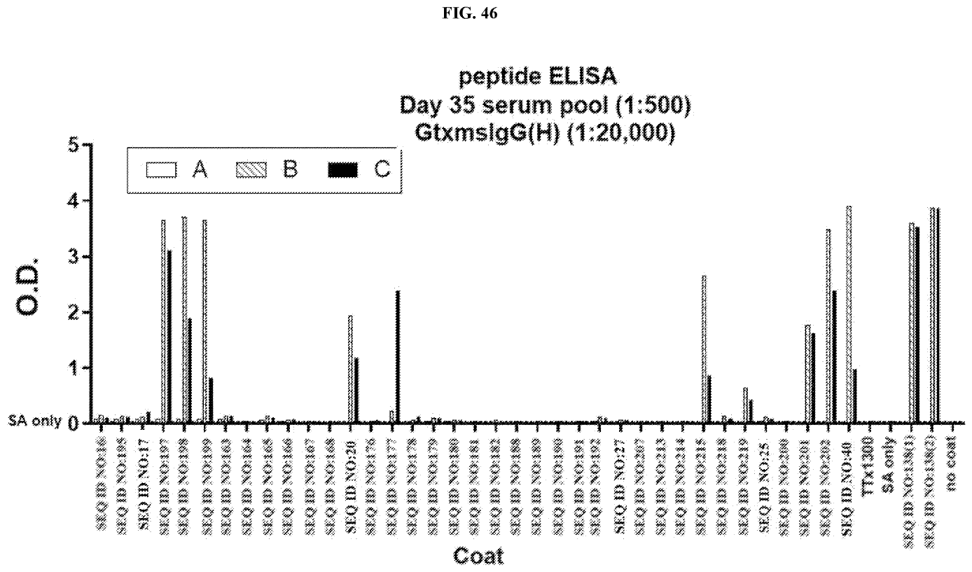

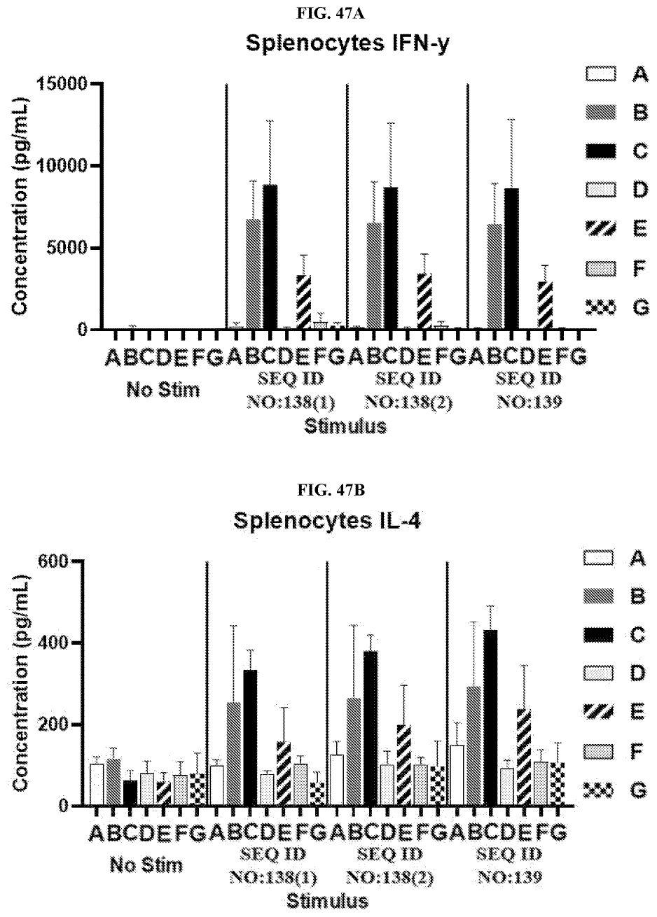

Serum samples were obtained from mice from the control (Group A) and each of treatment groups B-C(FIG. 46) on day 35 after first vaccination (approximately 2 weeks after second vaccination) and analyzed by peptide ELISA, wherein wells were coated with certain polypeptides and/or antigen fragments (e.g., SEQ ID NOs: 16-17, 20, 25, 27, 195, 197-199, 163-168, 176-182, 188-192, 207, 213-215, 218-219, 200-203, 138(1), and 138(2), wherein (1) and (2) vary with respect to buffer conditions, as described in Table 7) and controls (i.e., tetanus toxin, serum albumin only and no coat). The data depict antibody production in wells containing each antigen utilized in exemplary vaccine antigens and/or fragments thereof.

Thus, in certain embodiments, the antigens and/or antigen fragments of the present disclosure elicit an adaptive immune response mediated by B cell activation, as indicated by elevated levels of certain antibodies, non-limiting examples including IgG1, IgG2b, IgA, and IgG(H).

Exp. No. 8

CD1 mice were immunized as described herein using the vaccine compositions indicated in Table 8.

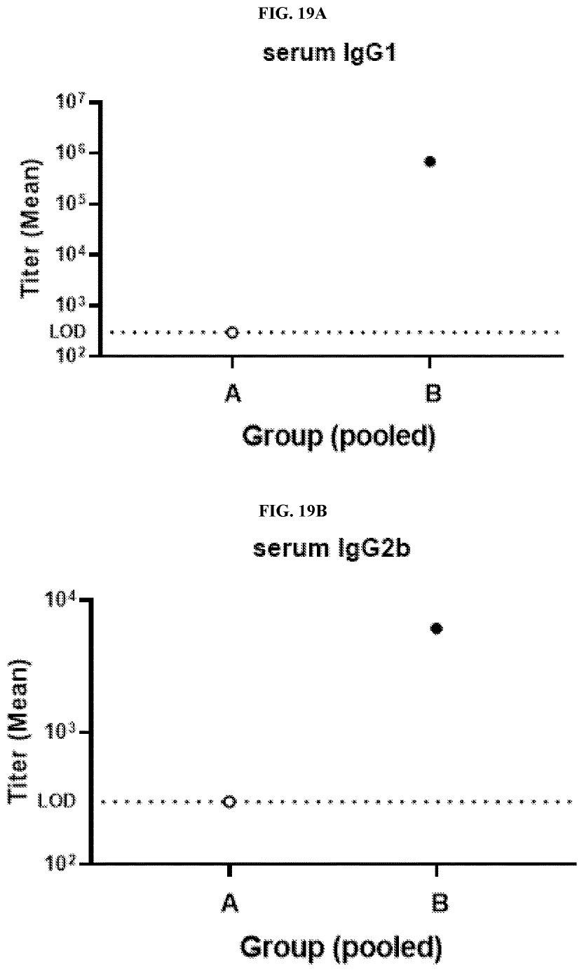

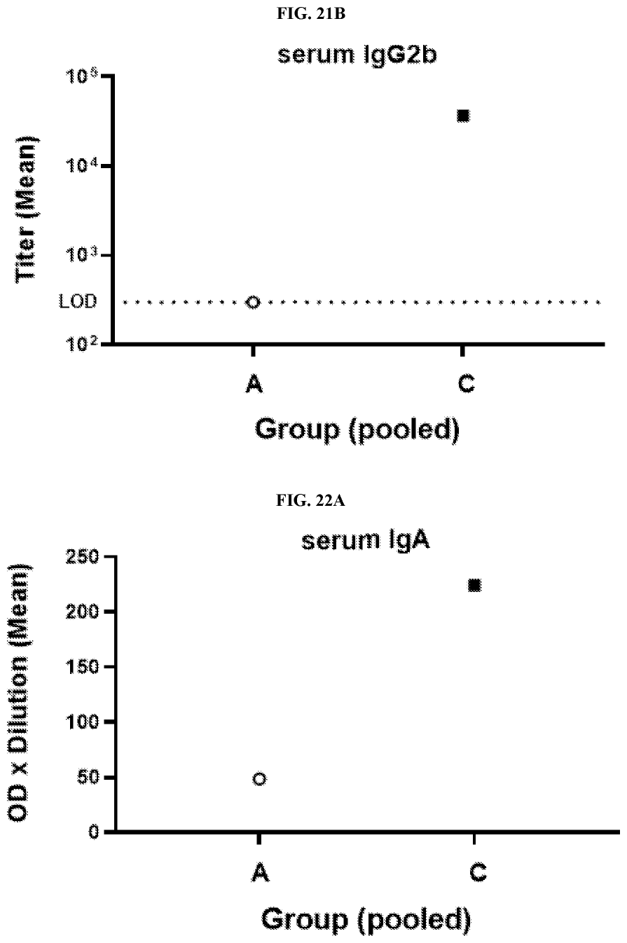

Serum samples were obtained from mice from each of groups A-C approximately 14 days after the second vaccination (i.e., approximately day 28) and analyzed by ELISA for serum IgG1 (FIG. 19A and FIG. 21A) and serum IgG2b (FIG. 19B and FIG. 21B). The results indicate that immunization with each of SEQ ID NO:132 and SEQ ID NO:135 induces both IgG1 and IgG2b production in CB1 mice.

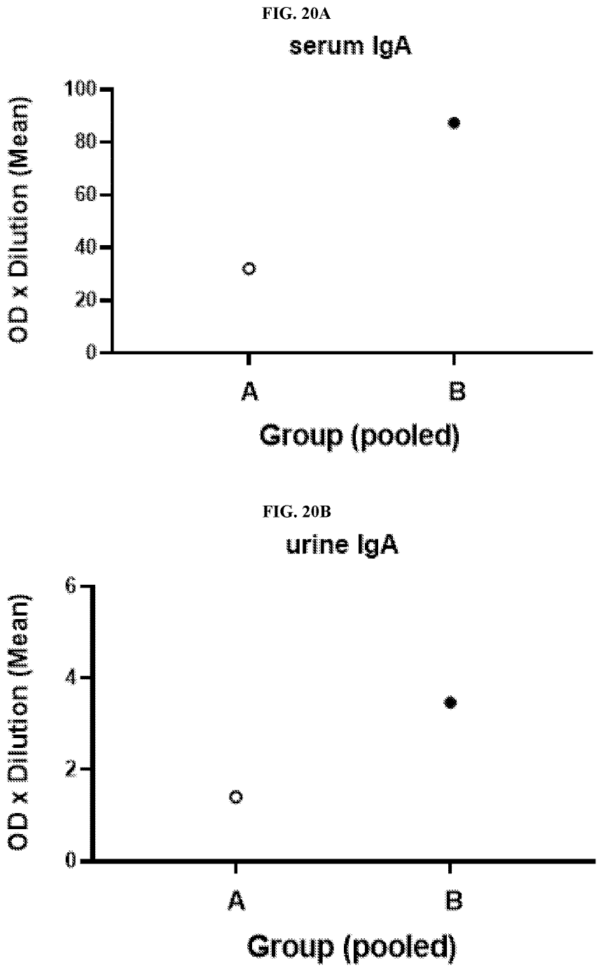

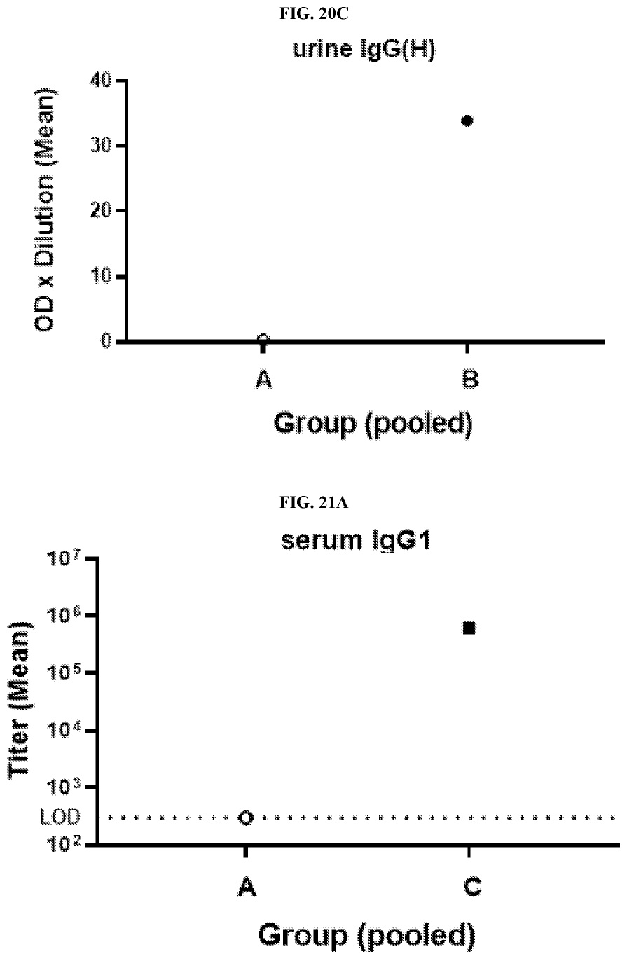

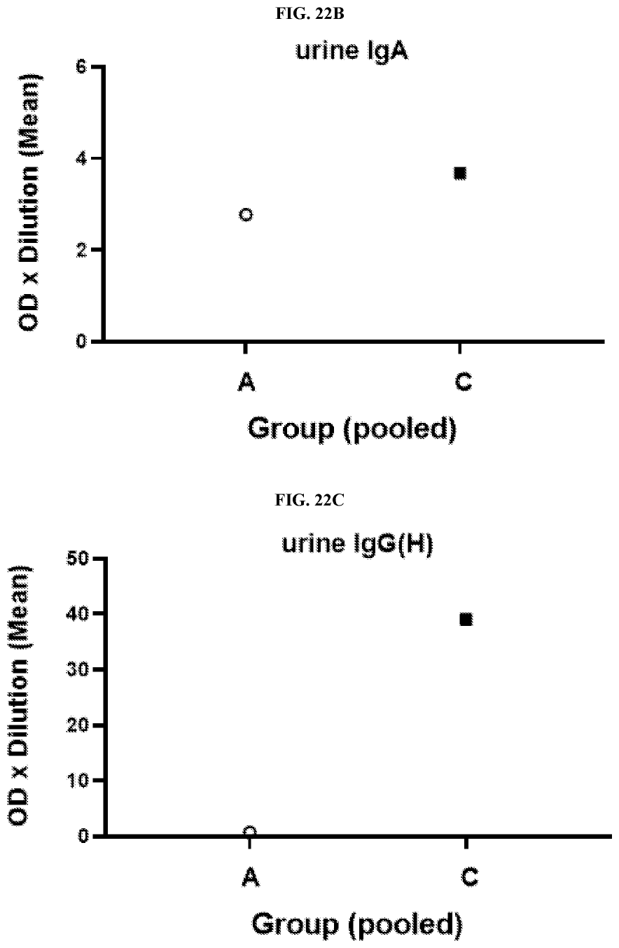

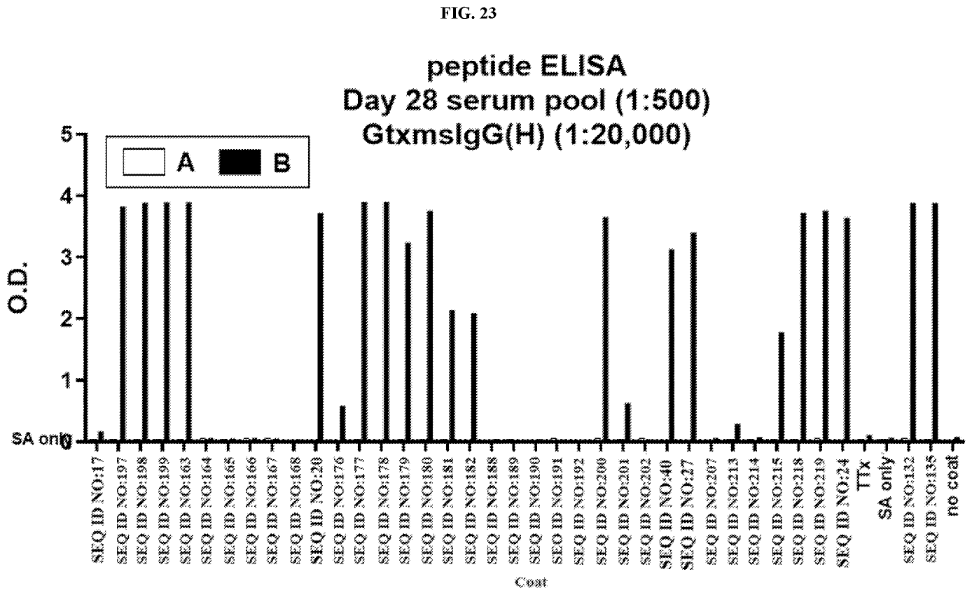

Serum samples were obtained from mice from each of groups A-C approximately 14 days after the second vaccination (i.e., approximately day 28) and analyzed by ELISA for serum IgA (FIG. 20A and FIG. 22A), urine IgA (FIG. 20B and FIG. 22B), and urine IgG(H) (FIG. 20C and FIG. 22C). The results indicate that SEQ ID NO:132 immunization induces IgA and IgG(H) production in CB1 mice, as evidenced by urine and/or serum samples.

Serum samples were obtained from mice from groups A (i.e., placebo) and B (i.e., SEQ ID NO:132) on day 28 (i.e., approximately 14 days after the second vaccination) and analyzed by peptide ELISA, wherein wells were coated with certain polypeptides and/or antigen fragments (e.g., SEQ ID NOs:17-20, 23-25, 27, 32, 35, 37, 39-40), antigens (i.e., SEQ ID NO:132 and SEQ ID NO:135), and controls (i.e., tetanus toxin, serum albumin only and no coat). Antibody production was not substantially observed in subjects administered placebo (i.e., signal below or minimally over serum albumin only control in Group A), whereas significant peptide specific antibody production was observed for many of the antigens, or antigen fragments thereof, evaluated in this study (FIG. 23).

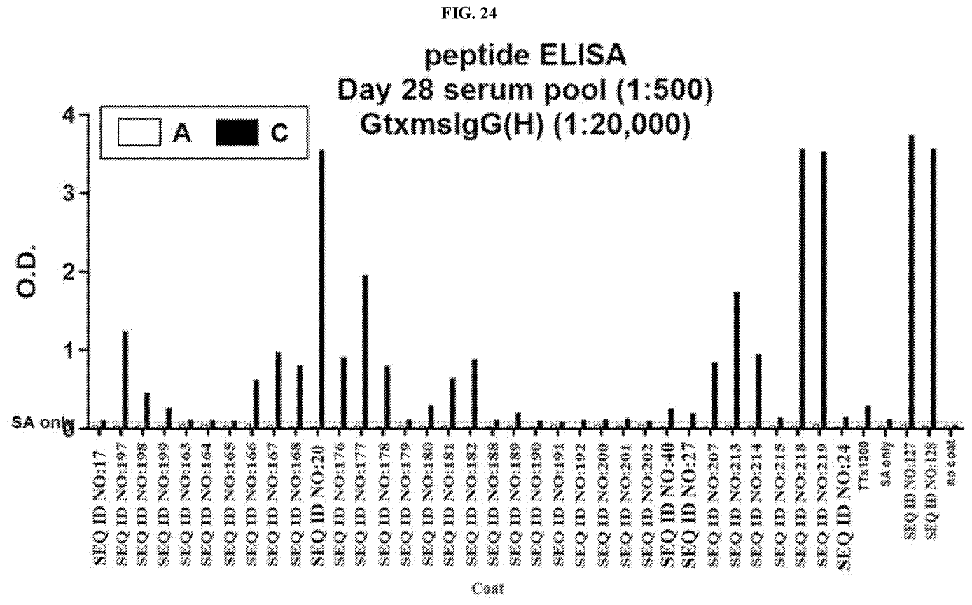

Serum samples were obtained from mice from groups A (i.e., placebo) and C (i.e., SEQ ID NO:135) on day 28 (i.e., approximately 14 days after the second vaccination) and analyzed by peptide ELISA, wherein wells were coated with certain polypeptides and/or antigen fragments (e.g., SEQ ID NOs:17-20, 23-25, 27, 32, 35, 37, 39-40), antigens (i.e., SEQ ID NO:132 and SEQ ID NO:135), and controls (i.e., tetanus toxin, serum albumin only and no coat). Antibody production was not substantially observed in subjects administered placebo (i.e., signal below or minimally over serum albumin only control in Group A), whereas significant peptide specific antibody production was observed for many of the antigens, or antigen fragments thereof, evaluated in this study (FIG. 24).

Exp. No. 9

C57BL/6 mice were immunized as described herein using the vaccine compositions indicated in Table 9.

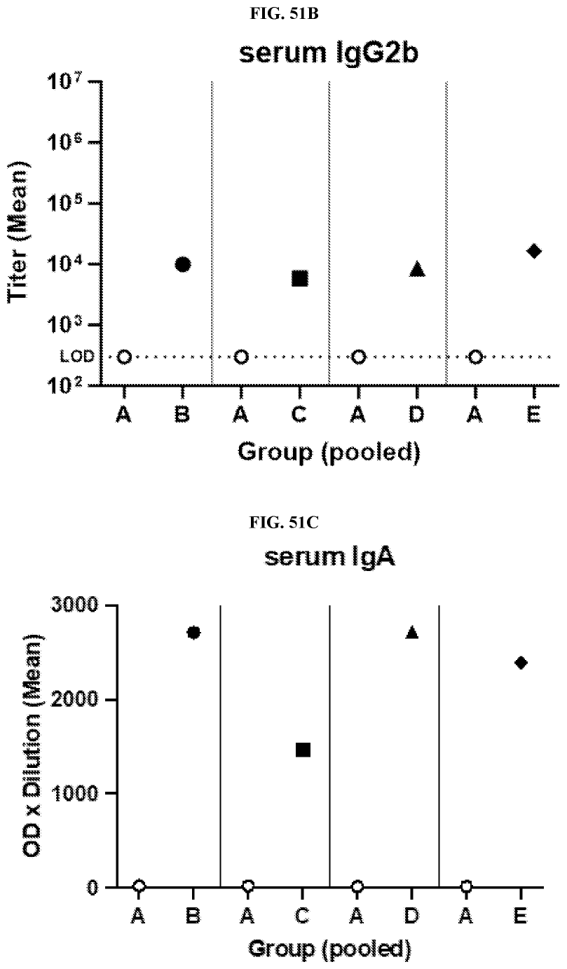

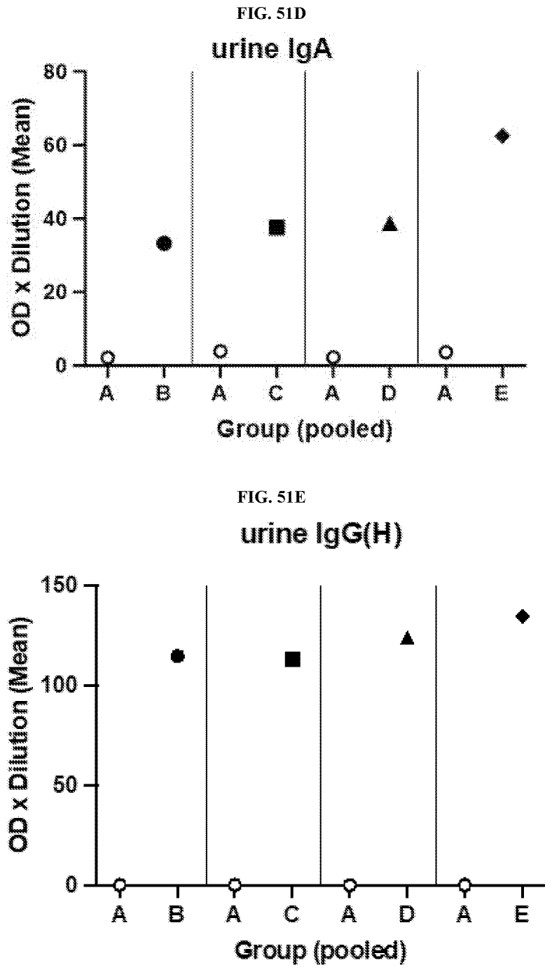

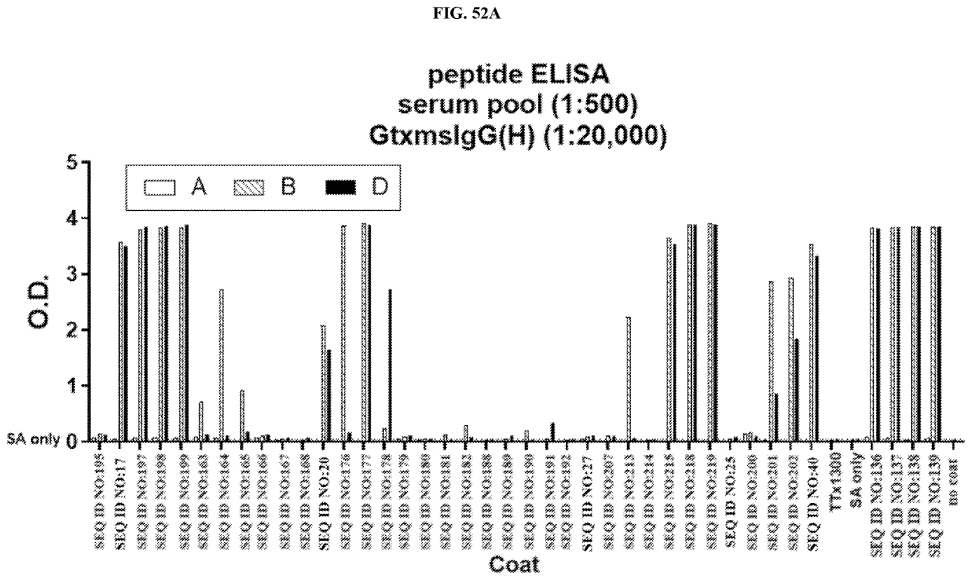

Serum and urine samples were obtained from mice from each treatment group (i.e., Groups B-E) and the control group (i.e., Groups A) 34 days after initial vaccination (approximately 2 weeks after second vaccination) and analyzed by ELISA for serum IgG1, serum IgG2b, serum IgA, urine IgA, and urine IgG(H) (FIGS. 51A-51E).

In certain embodiments, the results indicate that certain evaluated immunizations induce IgG1, IgG2b, and IgA production in C57BL/6 mice sera 34 days after initial vaccination (approximately 2 weeks after second vaccination), as compared to the control. In certain embodiments, the results indicate that certain evaluated immunizations induce IgA and IgG(H) production in the urine of HLA-DR4 mice 34 days after initial vaccination (approximately 2 weeks after second vaccination).

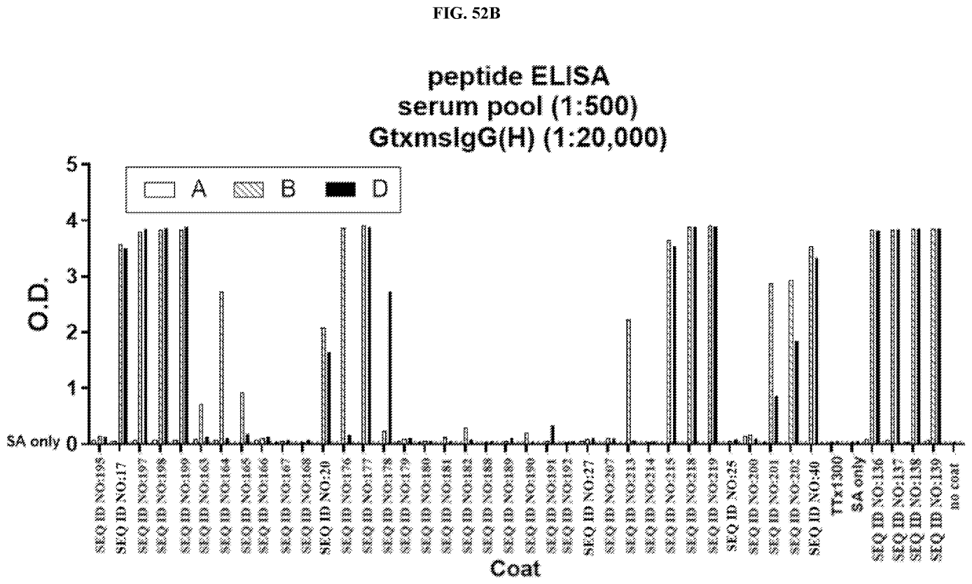

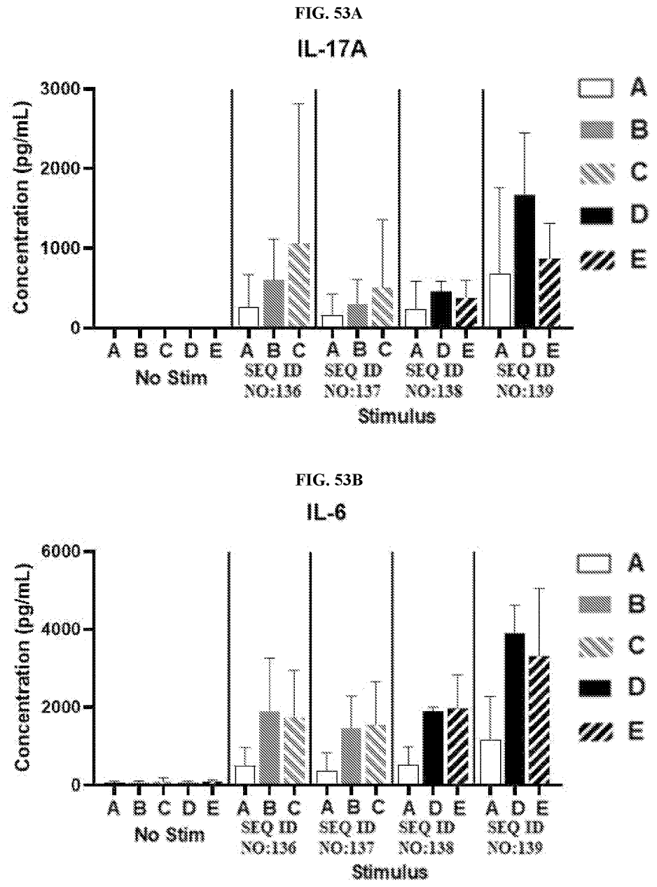

Serum samples were obtained from mice from the control (Group A) and each of treatment groups B and D (FIG. 52A) and treatment groups C and E (FIG. 52B) on day 34 after first vaccination (approximately 2 weeks after second vaccination) and analyzed by peptide ELISA, wherein wells were coated with certain polypeptides and/or antigen fragments (e.g., SEQ ID NOs: 17, 20, 25, 27, 195, 197-199, 163-168, 176-182, 188-192, 207, 213-215, 218-219, 200-203, and 136-139) and controls (i.e., tetanus toxin, serum albumin only and no coat). The data depict antibody production in wells containing each antigen utilized in exemplary vaccine antigens and/or fragments thereof.

Thus, in certain embodiments, the antigens and/or antigen fragments of the present disclosure elicit an adaptive immune response mediated by B cell activation, as indicated by elevated levels of certain antibodies, non-limiting examples including IgG1, IgG2b, IgA, and IgG(H).

Exp. No. 10

C57BL/6 mice were immunized as described herein using the vaccine compositions indicated in Table 10.

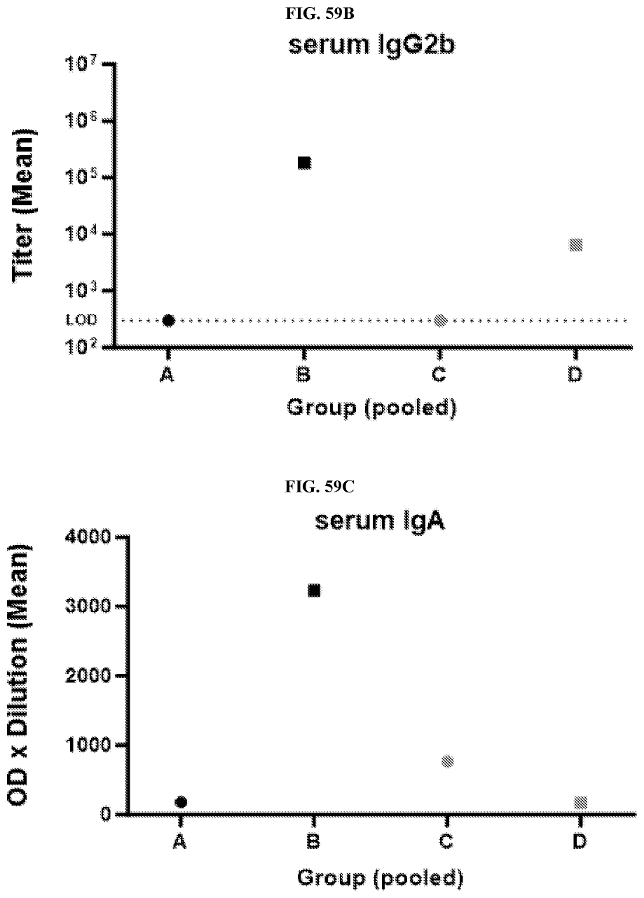

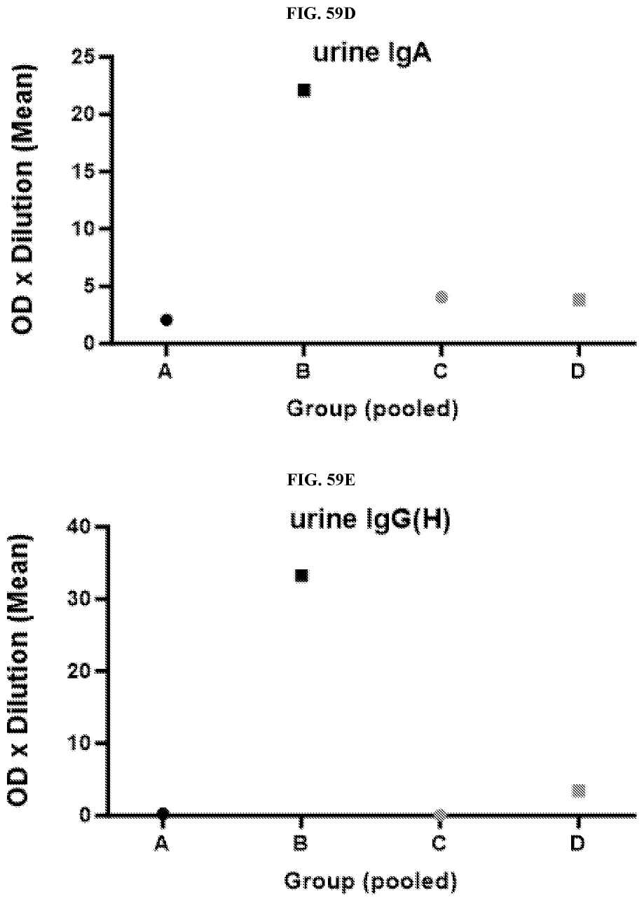

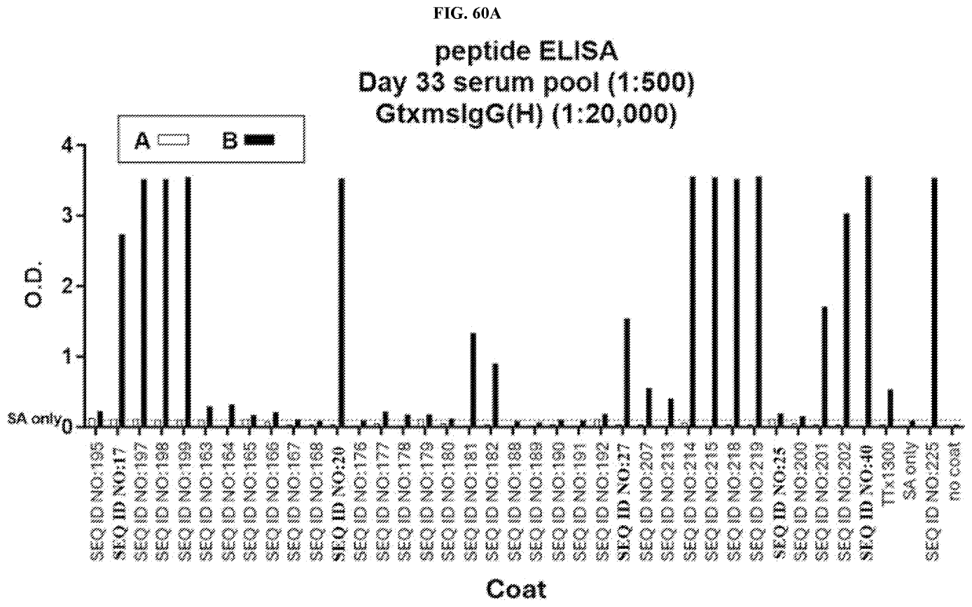

Serum and urine samples were obtained from mice from each treatment group (i.e., Groups B and D) and the control group (i.e., Groups A and C) 33 days after initial vaccination (approximately 12 days after second vaccination) and analyzed by ELISA for serum IgG1, serum IgG2b, serum IgA, urine IgA, and urine IgG(H) (FIGS. 59A-59E).

In certain embodiments, the results indicate that certain evaluated immunizations induce IgG1, IgG2b, and IgA production in C57BL/6 mice sera 33 days after initial vaccination (approximately 12 days after second vaccination), as compared to the control. In certain embodiments, the results indicate that certain evaluated immunizations induce IgA and IgG(H) production in the urine of HLA-DR4 mice 33 days after initial vaccination (approximately 2 weeks after second vaccination).

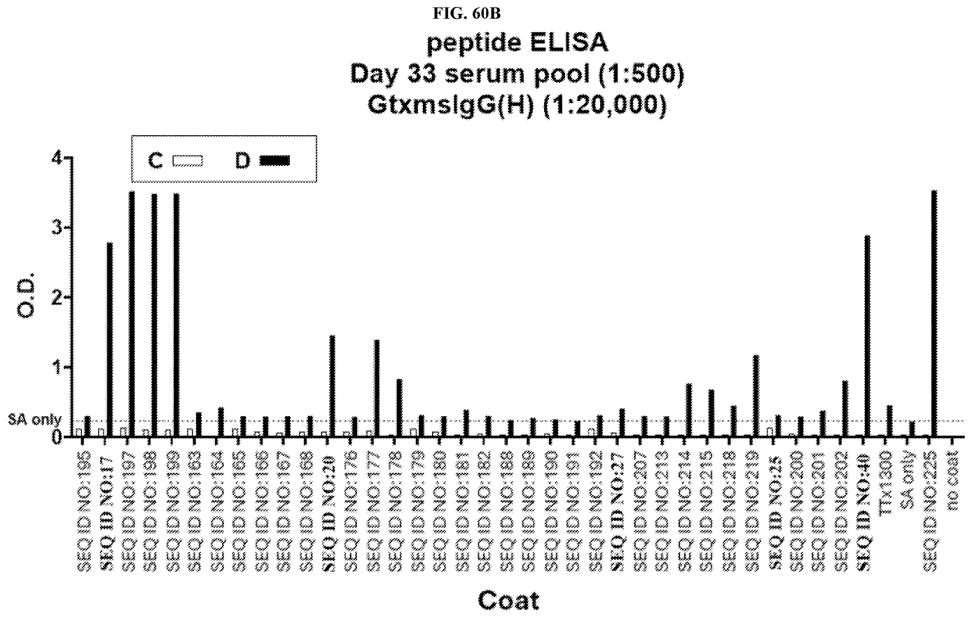

Serum samples were obtained from mice from the control Group A and treatment Group B (FIG. 60A), and control Group C and treatment Group D (FIG. 60B) on day 33 after first vaccination (approximately 12 days after second vaccination) and analyzed by peptide ELISA, wherein wells were coated with certain polypeptides and/or antigen fragments (e.g., SEQ ID NOs: 17, 20, 25, 27, 40, 163-168, 176-182, 188-192, 195, 197-202, 207, 213-215, 218-219, and 225) and controls (i.e., tetanus toxin, serum albumin only and no coat). The data depict antibody production in wells containing each antigen utilized in exemplary vaccine antigens and/or fragments thereof.

Thus, in certain embodiments, the antigens and/or antigen fragments of the present disclosure elicit an adaptive immune response mediated by B cell activation, as indicated by elevated levels of certain antibodies, non-limiting examples including IgG1, IgG2b, IgA, and IgG(H).

Exp. No. 11

C57BL/6 mice were immunized as described herein using the vaccine compositions indicated in Table 11.

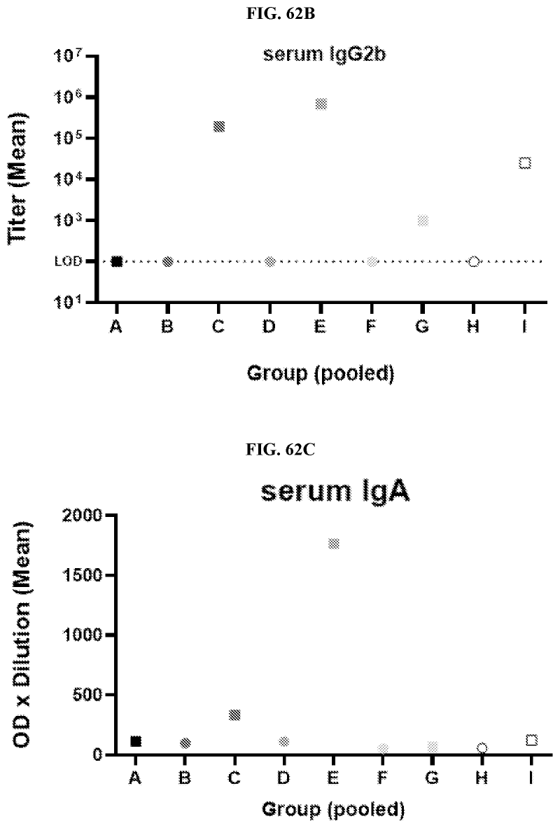

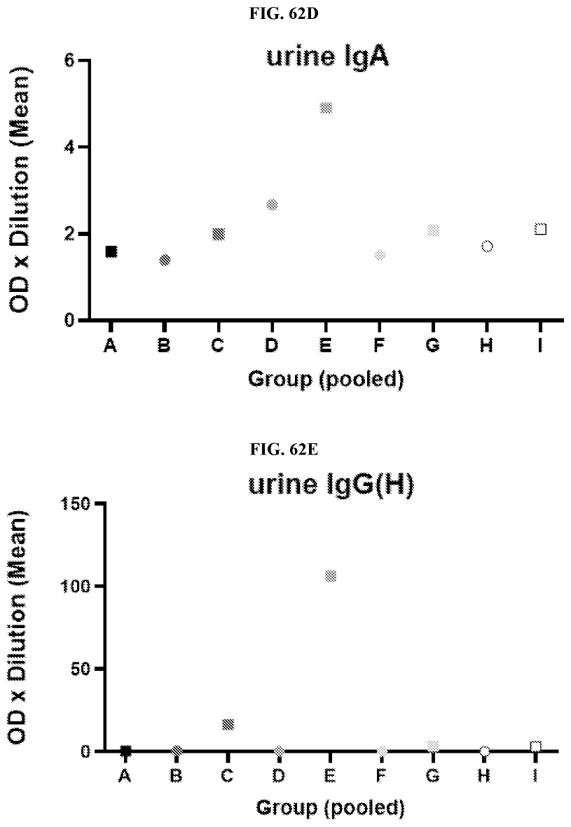

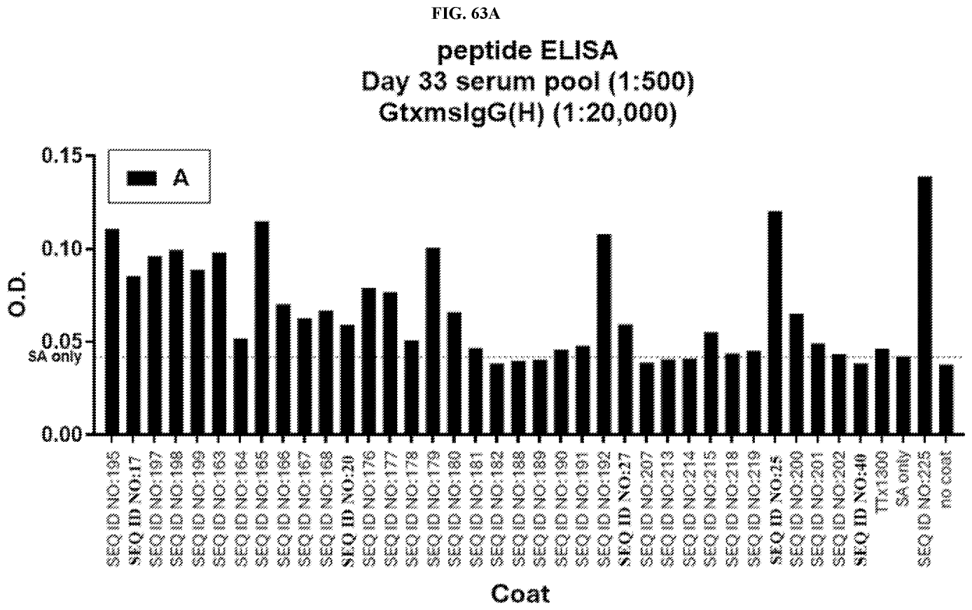

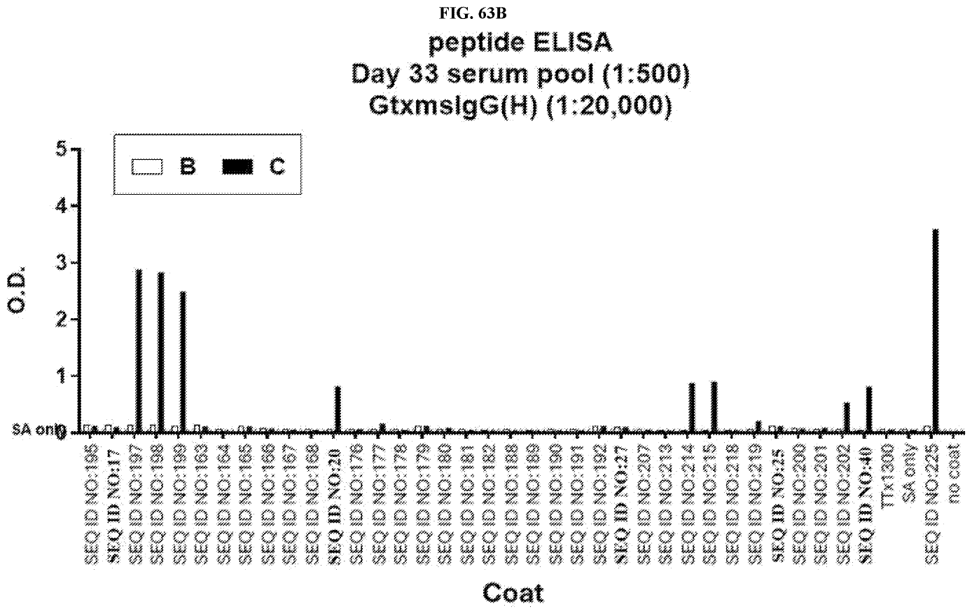

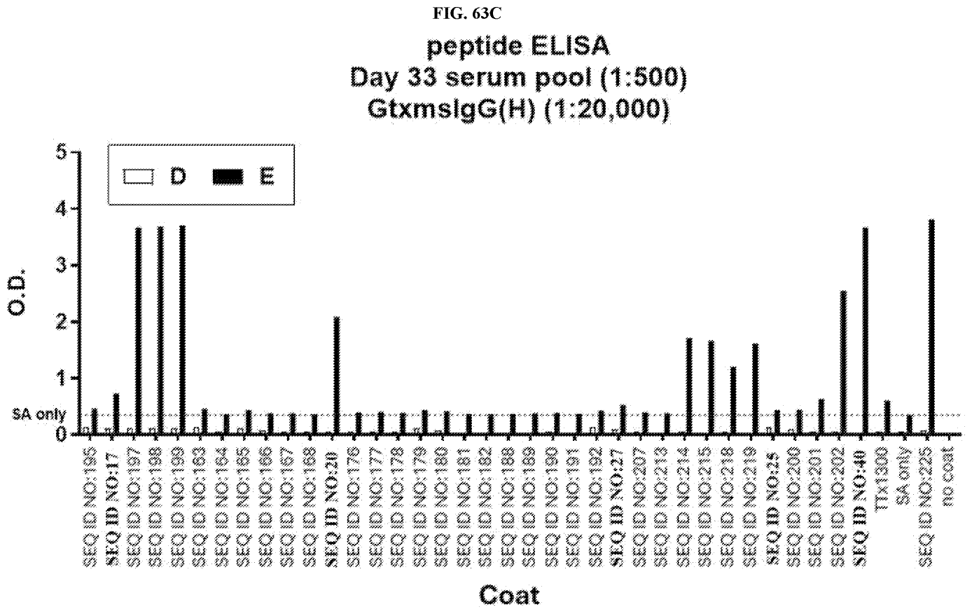

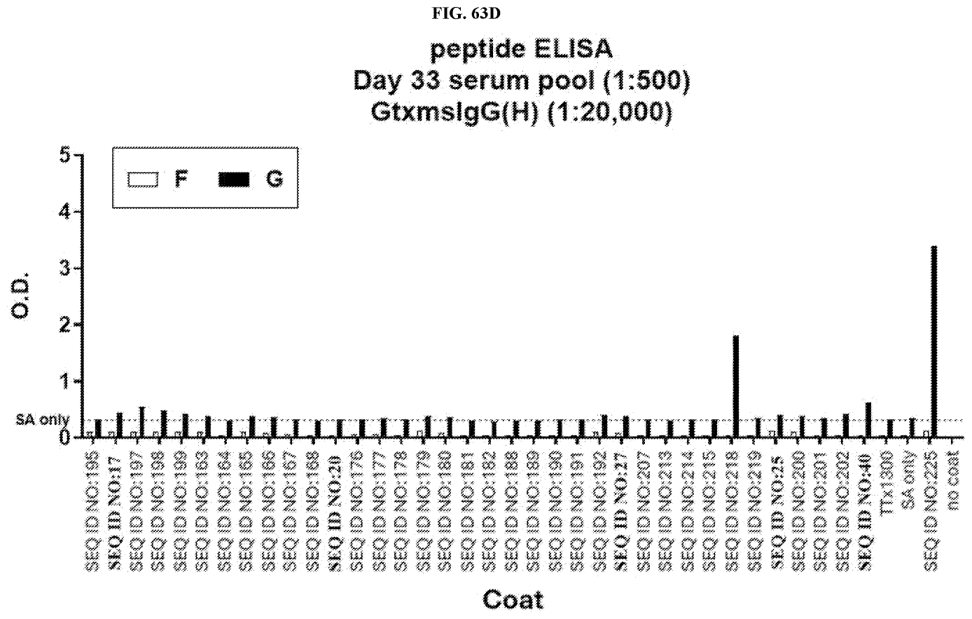

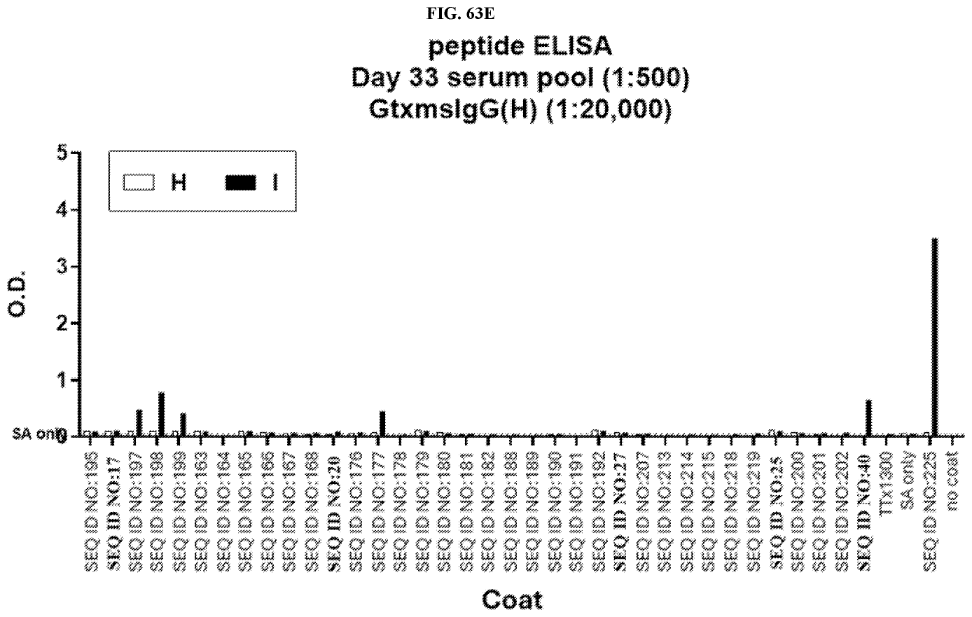

Serum and urine samples were obtained from mice from each treatment group (i.e., Groups A, C, E, G, and I) and control/placebo groups (i.e., Groups B, D, F, and H) 12 days after second vaccination (i.e., 33 days post first vaccination) and analyzed by ELISA for serum IgG1, serum IgG2b, serum IgA, urine IgA, and urine IgG(H) (FIGS. 62A-62E).

In certain embodiments, the results indicate that the evaluated immunizations induce IgG1, IgG2b, and IgA production in C57BL/6 mice sera 12 days after second vaccination, as compared to the control. In certain embodiments, the results indicate that the evaluated immunizations induce IgA and IgG(H) production in the urine of C57BL/6 mice 12 days after initial vaccination, as compared to the control. In certain embodiments, the results indicate the use of an adjuvant, when administering the antigen parenterally (e.g., intramuscularly), is critical for stimulating IgG1, IgG2b, IgA, and/or IgG(H) production (cf. Group A and any of groups C, E, G, and I).

Serum samples were obtained from C57BL/6 mice from each treatment group (i.e., Groups A, C, E, G, and I) and control/placebo groups (i.e., Groups B, D, F, and H) 12 days after second vaccination (i.e., 33 days post first vaccination) and analyzed by peptide ELISA, wherein wells were coated with certain polypeptides and/or antigen fragments (i.e., SEQ ID NOs: 17, 20, 25, 27, 40, 163-168, 176-182, 188-192, 195, 197-202, 207, 213-215, 218-219, and 225) and controls (i.e., tetanus toxin, serum albumin only, and no coat). The data depict antibody production in wells containing each antigen and/or fragments thereof utilized in exemplary vaccine compositions of the disclosure.

Thus, in certain embodiments, the antigens and/or antigen fragments of the present disclosure elicit an adaptive immune response mediated by B cell activation, as indicated by elevated levels of certain antibodies, non-limiting examples including IgG1, IgG2b, IgA, and IgG(H).

Exp. No. 12

C3H/HeN mice were immunized as described herein using the vaccine compositions indicated in Table 12.

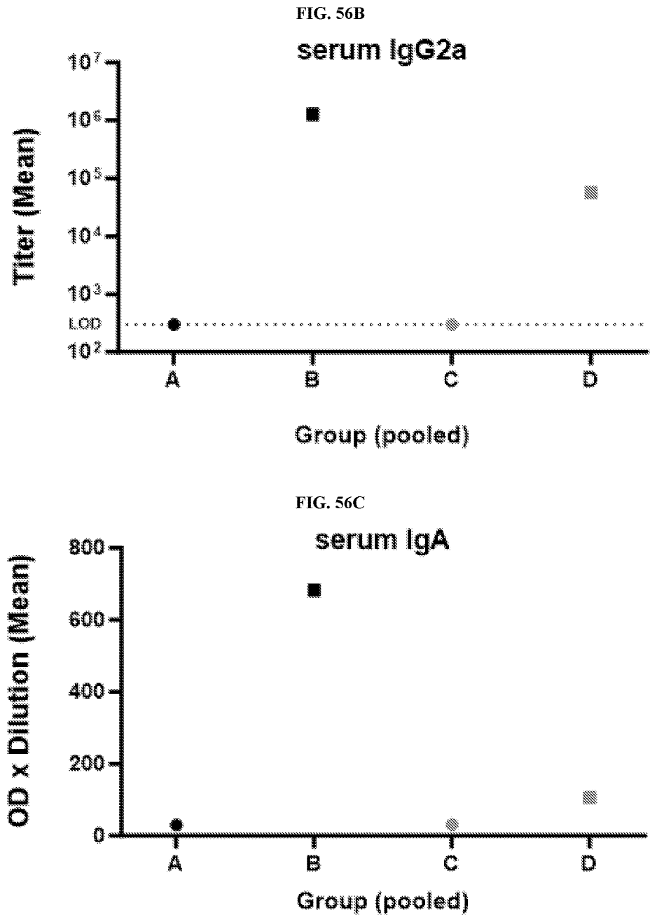

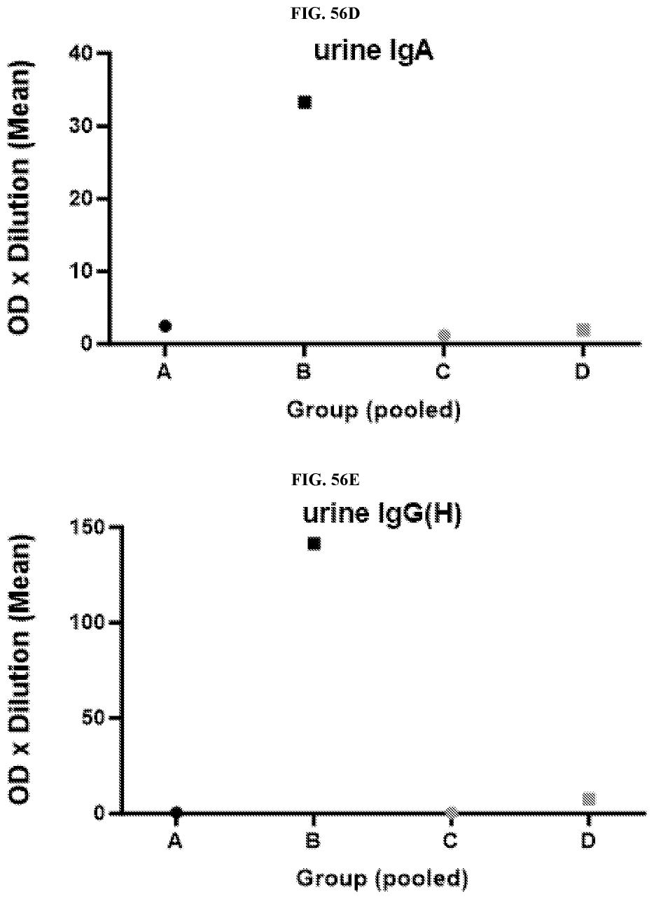

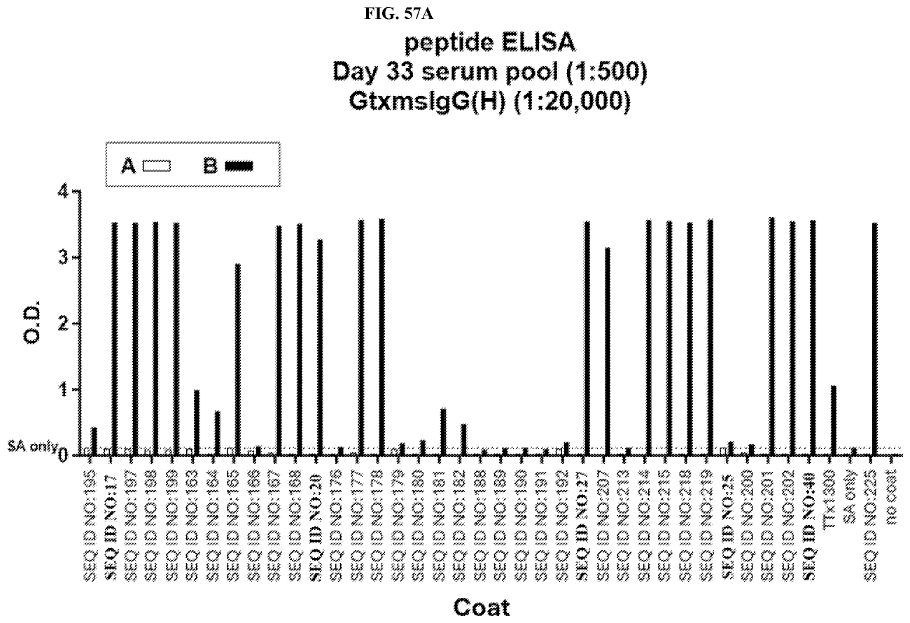

Serum and urine samples were obtained from mice from each treatment group (i.e., Groups B and D) and the control group (i.e., Groups A and C) 33 days after initial vaccination (approximately 12 days after second vaccination) and analyzed by ELISA for serum IgG1, serum IgG2a, serum IgA, urine IgA, and urine IgG(H) (FIGS. 56A-56E).

In certain embodiments, the results indicate that certain evaluated immunizations induce IgG1, IgG2a, and IgA production in C3H/HeN mice sera 33 days after initial vaccination (approximately 12 days after second vaccination), as compared to the control. In certain embodiments, the results indicate that certain evaluated immunizations induce IgA and IgG(H) production in the urine of HLA-DR4 mice 33 days after initial vaccination (approximately 2 weeks after second vaccination).

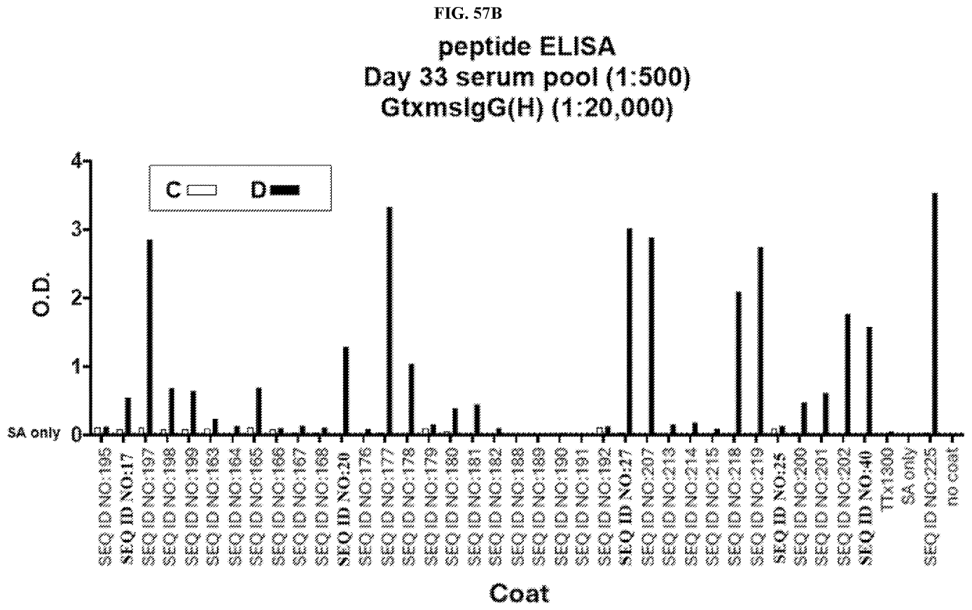

Serum samples were obtained from mice from the control Group A and treatment Group B (FIG. 57A), and control Group C and treatment Group D (FIG. 57B) on day 33 after first vaccination (approximately 12 days after second vaccination) and analyzed by peptide ELISA, wherein wells were coated with certain polypeptides and/or antigen fragments (e.g., SEQ ID NOs: 17, 20, 25, 27, 40, 163-168, 176-182, 188-192, 195, 197-202, 207, 213-215, 218-219, and 225) and controls (i.e., tetanus toxin, serum albumin only and no coat). The data depict antibody production in wells containing each antigen utilized in exemplary vaccine antigens and/or fragments thereof.

Thus, in certain embodiments, the antigens and/or antigen fragments of the present disclosure elicit an adaptive immune response mediated by B cell activation, as indicated by elevated levels of certain antibodies, non-limiting examples including IgG1, IgG2a, IgA, and IgG(H).

### Example 3: Immunized Mice Demonstrate an Adaptive Immune Response (T Cell Mediated Antibody Production)

Exp. No. 1

Assessment of adaptive immune response mediated by T cells was performed by analysis of splenocytes obtained from HLA-DR4 mice immunized as described elsewhere herein using the vaccine compositions indicated in Table 1.

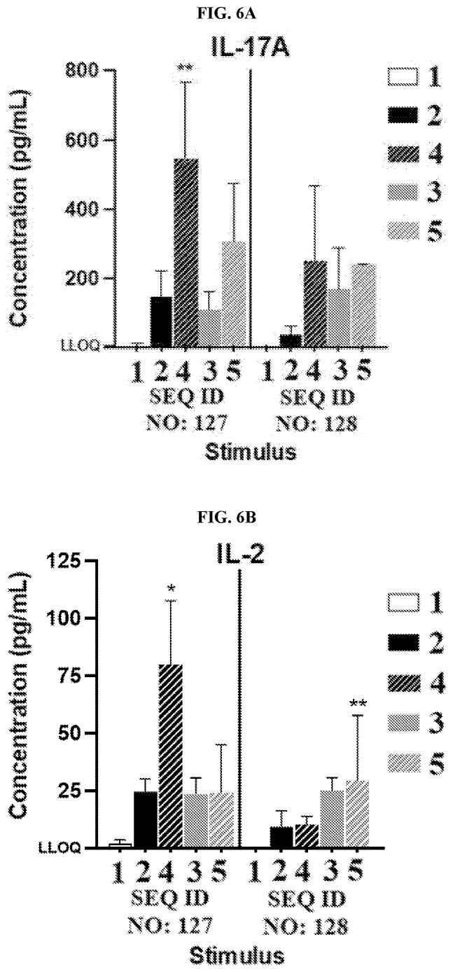

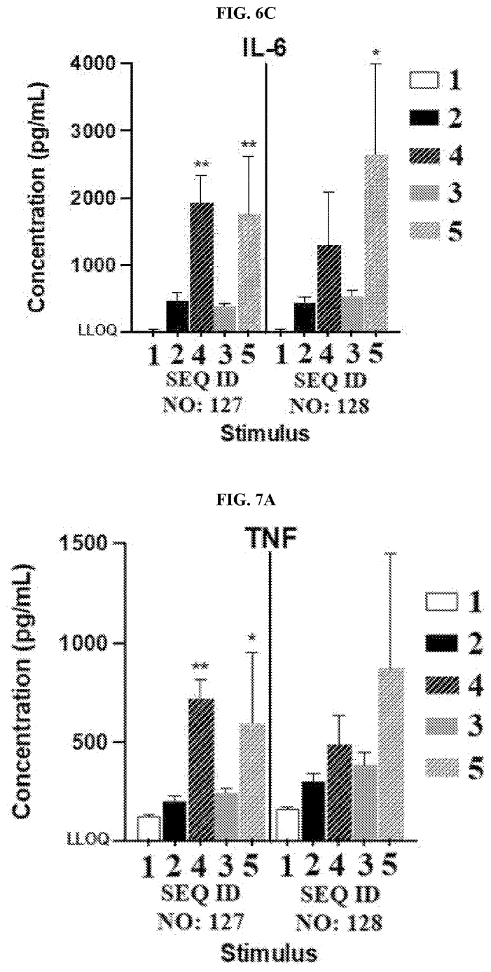

Splenocytes were obtained from mice of groups 1-5 approximately 22 days after second vaccination and stimulated with SEQ ID NO:127, SEQ ID NO:128, and analyzed for production of Th1/17/pro-inflammatory cytokines IL-17A (FIG. 6A), IL-2 (FIG. 6B), and IL-6 (FIG. 6C) by Cytometric Bead Array (CBA) using a Th1/2/17 kit (BD Biosciences). Results indicate that a strong response (i.e., high concentration of antibody) was observed in response to stimulation with antigens SEQ ID NO:127 and SEQ ID NO:128 in splenocytes derived from treatment group mice (i.e., groups 2-5).

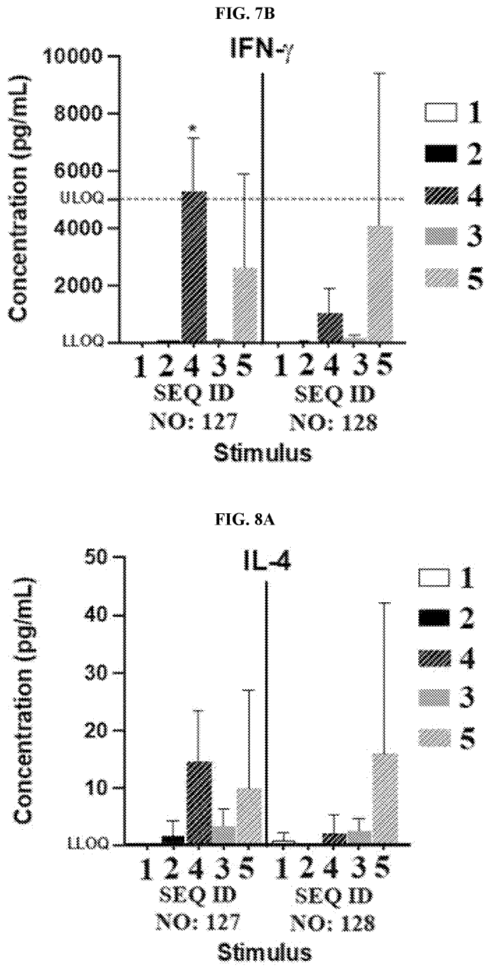

Splenocytes were obtained from mice of groups 1-5 approximately 22 days after second vaccination and stimulated with SEQ ID NO:127 and SEQ ID NO:128, and analyzed for production of Th1/pro-inflammatory cytokines TNF-α (FIG. 7A) and IFN-γ (FIG. 7B) by Cytometric Bead Array (CBA) using a Th1/2/17 kit (BD Biosciences). Results indicate that TNF-α production was observed in response to stimulation with antigens SEQ ID NO:127 and SEQ ID NO:128 in splenocytes derived from treatment group mice (i.e., groups 2-5).

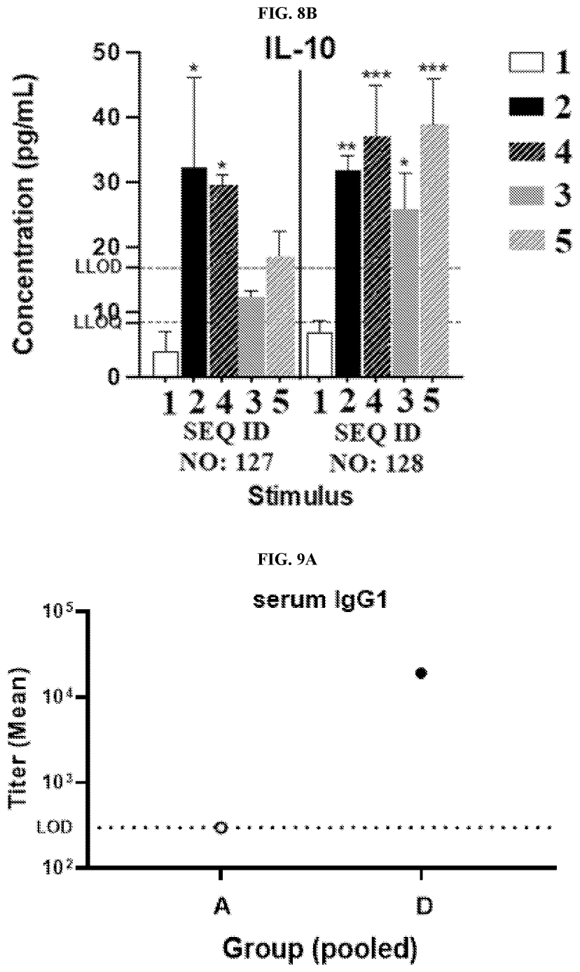

Splenocytes were obtained from mice of groups 1-5 approximately 22 days after second vaccination and stimulated with SEQ ID NO:127 and SEQ ID NO:128, and analyzed for production of Th2/pro-inflammatory cytokines IL-4 (FIG. 8A) and IL-10 (FIG. 8B) by Cytometric Bead Array (CBA) using a Th1/2/17 kit (BD Biosciences). Generally, the results indicate that IL-4 production was observed in response to stimulation with antigens SEQ ID NO:127 and SEQ ID NO:128 in splenocytes derived from treatment group mice (i.e., groups 2-5).

Thus, in certain embodiments, the antigens and/or antigen fragments of the present disclosure elicit an adaptive immune response mediated by T cell activation, as indicated by elevated levels of several antibodies and/or cytokines, non-limiting examples including IL-17A, IL-2, IL-6, TNF-α, IFN-γ, IL-4, and IL-10.

Exp. No. 2

Assessment of adaptive immune response mediated by T cells was performed by analysis of splenocytes obtained from HLA-DR4 mice immunized as described elsewhere herein using the vaccine compositions indicated in Table 2.

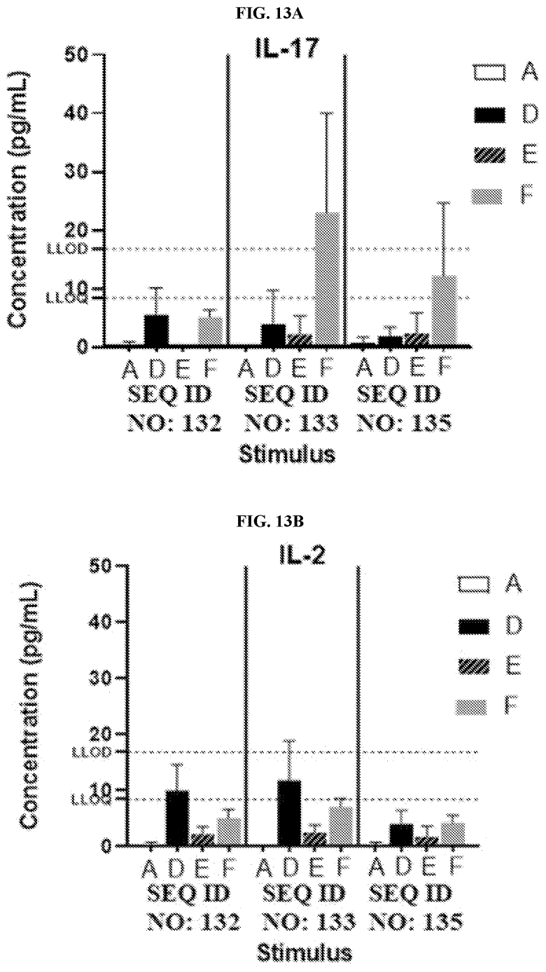

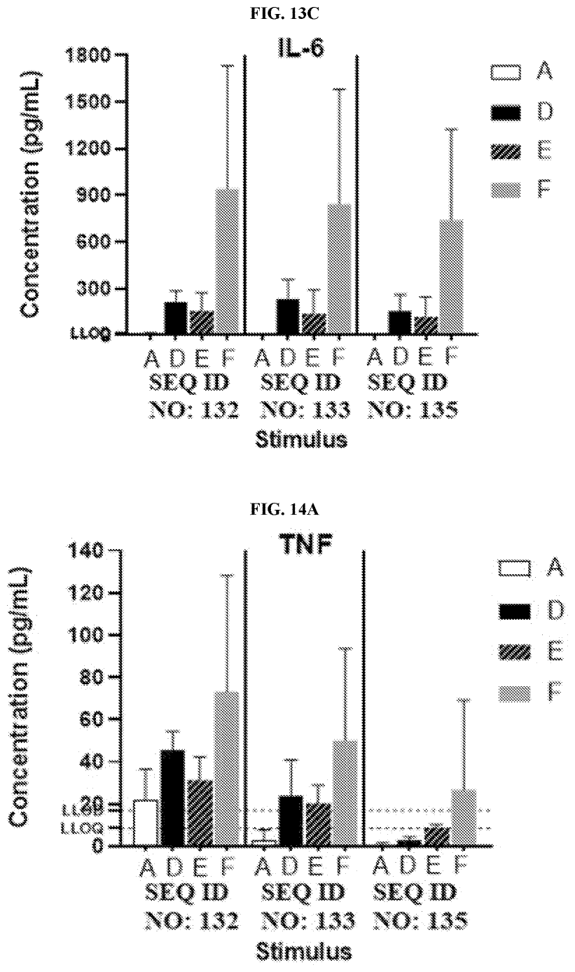

Splenocytes were obtained from mice of groups A-F approximately 14 days after second vaccination and stimulated with SEQ ID NO:132, SEQ ID NO:133, or SEQ ID NO:135, and analyzed for production of Th1/Th17/pro-inflammatory cytokines IL-17A (FIG. 13A), IL-2 (FIG. 13B), and IL-6 (FIG. 13C) by Cytometric Bead Array (CBA) using a Th1/2/17 kit (BD Biosciences). Results indicate that a response was observed in response to stimulation with antigens SEQ ID NO:132, SEQ ID NO:133, or SEQ ID NO:135 in splenocytes derived from treatment group mice.

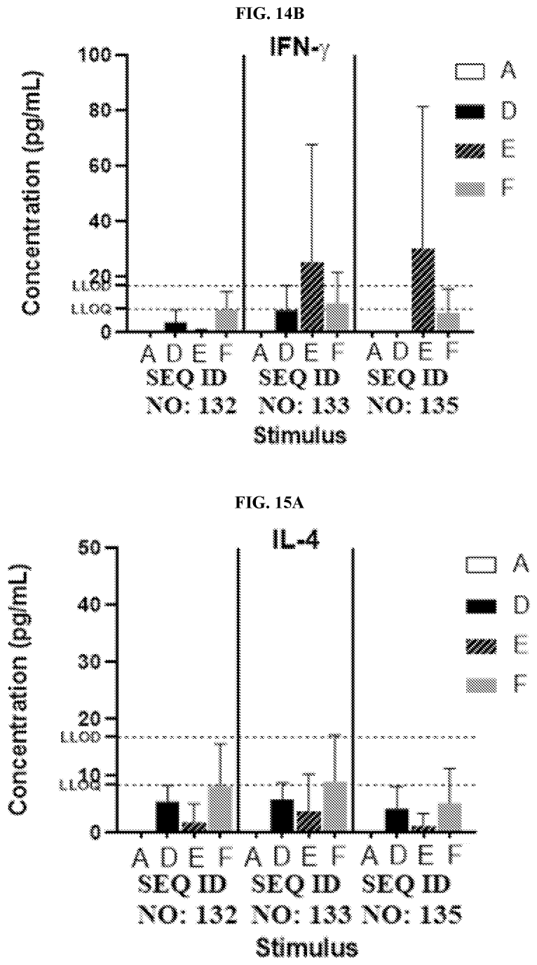

Splenocytes were obtained from mice of groups A and D-F approximately 14 days after second vaccination and stimulated with SEQ ID NO:132, SEQ ID NO:133, or SEQ ID NO:135, and analyzed for production of Th1/pro-inflammatory cytokines TNF-α (FIG. 14A) and IFN-γ (FIG. 14B) by Cytometric Bead Array (CBA) using a Th1/2/17 kit (BD Biosciences). Results indicate that TNF-α and IFN-γ production was observed in response to stimulation with antigens SEQ ID NO:132, SEQ ID NO:133, or SEQ ID NO:135 in splenocytes derived from treatment group mice (i.e., groups D-F).

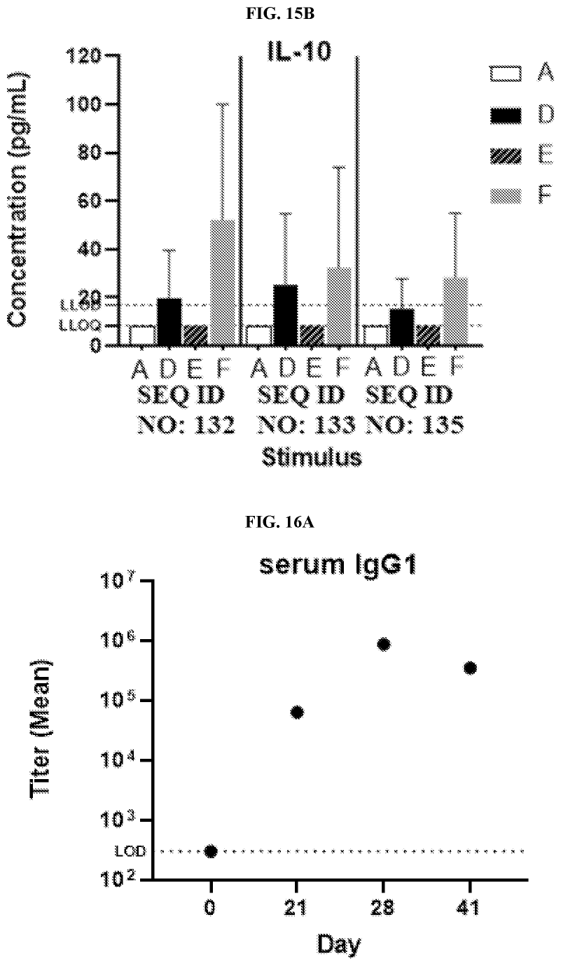

Splenocytes were obtained from mice of groups A and D-F 14 approximately days after second vaccination and stimulated with SEQ ID NO:132, SEQ ID NO:133, or SEQ ID NO:135, and analyzed for production of Th2/anti-inflammatory cytokines IL-4 (FIG. 15A) and IL-10 (FIG. 15B) by Cytometric Bead Array (CBA) using a Th1/2/17 kit (BD Biosciences). Generally, the results indicate that IL-10 production was observed in response to stimulation with antigens SEQ ID NO:132, SEQ ID NO:133, or SEQ ID NO:135 in splenocytes derived from treatment group mice (i.e., groups D-F).

Thus, in certain embodiments, the antigens and/or antigen fragments of the present disclosure elicit an adaptive immune response mediated by T cell activation, as indicated by elevated levels of several antibodies and/or cytokines, non-limiting examples including IL-17A, IL-2, IL-6, TNF-α, IFN-γ, IL-4, and IL-10.

Exp. No. 4

Assessment of adaptive immune response mediated by T cells was performed by analysis of splenocytes obtained from HLA-DR4 mice immunized as described elsewhere herein using the vaccine compositions indicated in Table 4.

Splenocytes were obtained from mice of groups A-F 49 days after first vaccination (approximately 30 days after second vaccination), and approximately 3 days after UPEC25 challenge, and stimulated with SEQ ID NO:136, SEQ ID NO:137, SEQ ID NO:138, SEQ ID NO:139, SEQ ID NO:140, or no stimulation, and cytokine levels measured using a MagPix® system. Results indicate that a cytokine response was observed in response to stimulation with certain antigens in mice from treatment groups, with the strongest responses observed for IFN-γ, IL-4, IL-6, IL-22, IL-17a, and IL-10.

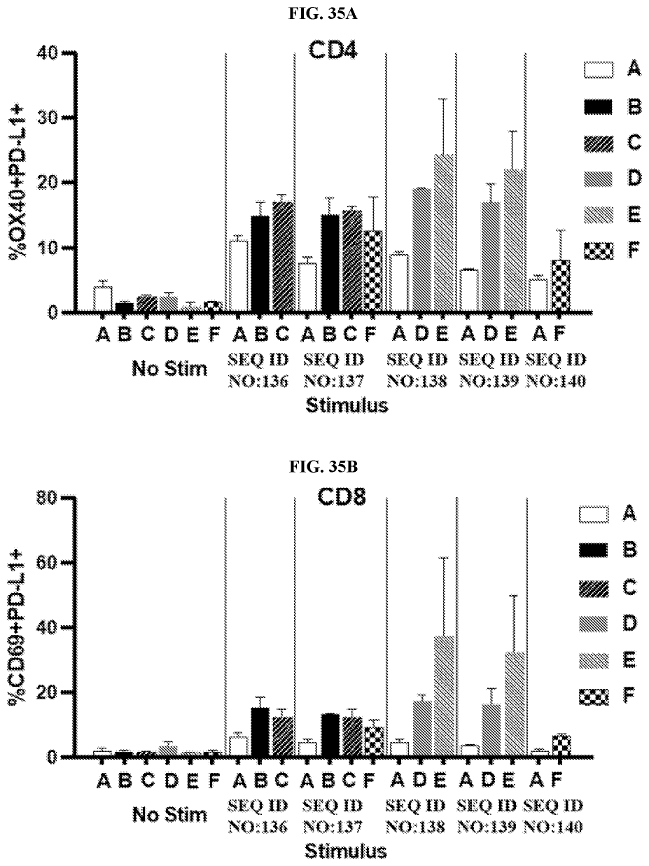

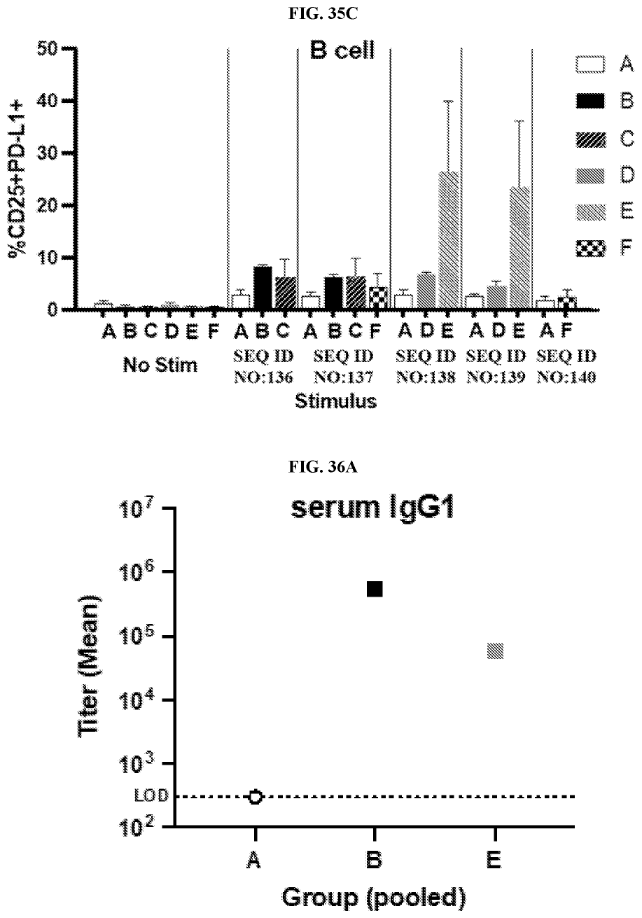

The combination of the AIM protein markers OX40 and PD-L1, CD69 and PD-L1, and CD25 and PD-L1 were used to identify antigen specific CD4⁺ T cells, CD8⁺ T cells, and B cells, respectively, by flow cytometry of splenocytes obtained from HLA-DR4 mice in all treatment (Groups B-F) and control (Group A) groups, which were activated in vitro by exemplary antigens: SEQ ID NO:136; SEQ ID NO:137; SEQ ID NO:138; SEQ ID NO:139; and SEQ ID NO:140; or no stimulation (FIG. 35A-35C).

Thus, in certain embodiments, the antigens of the present disclosure elicit an adaptive immune response mediated by T cell activation, as indicated by elevated levels of certain exemplary cytokines and/or certain exemplary T cells.

Exp. No. 5

Assessment of adaptive immune response mediated by T cells was performed by analysis of splenocytes obtained from HLA-DR4 mice immunized as described elsewhere herein using the vaccine compositions indicated in Table 5.

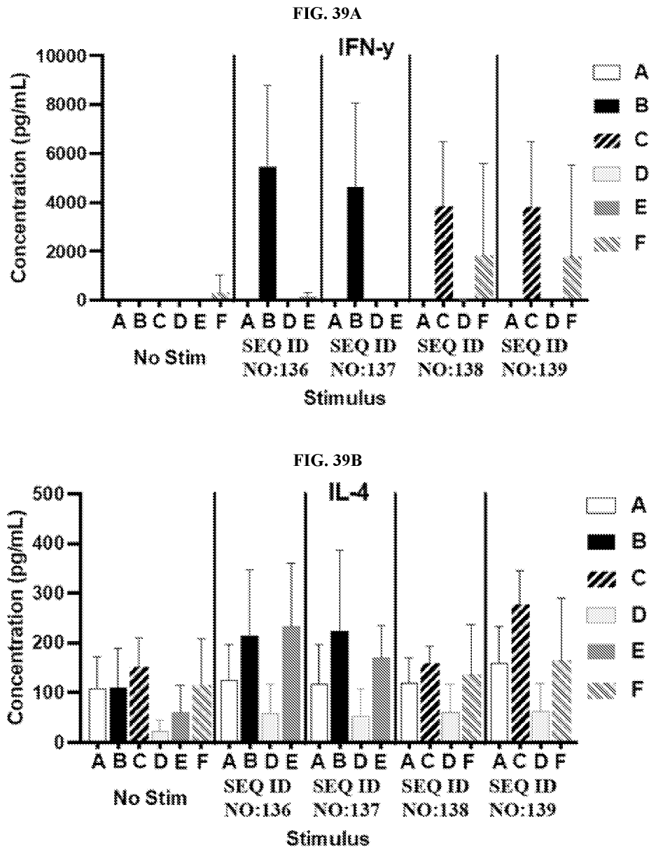

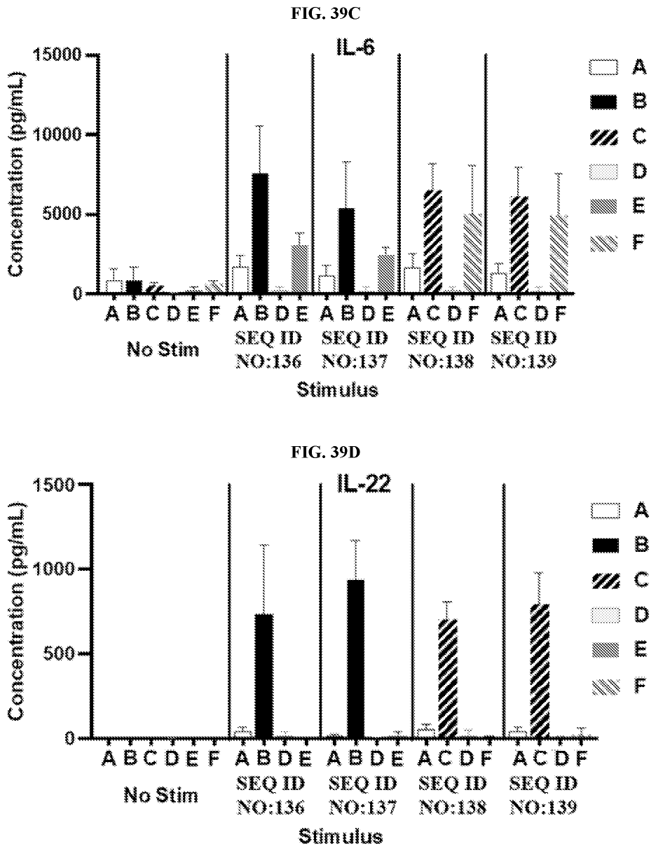

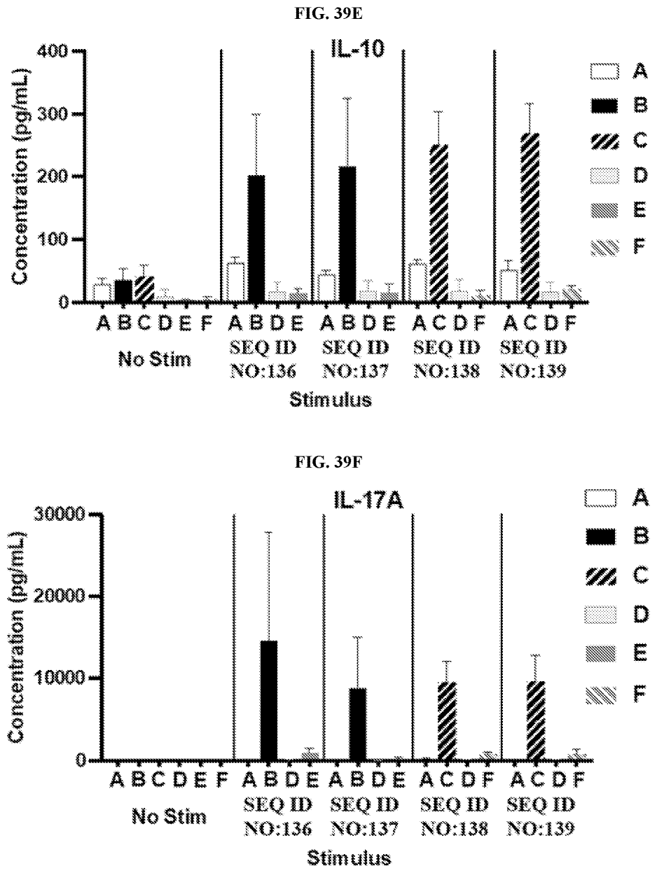

Splenocytes were obtained from mice of groups A-F 54 days after first vaccination (approximately 30 days after second vaccination), and approximately 7 days after UPEC25 challenge, and stimulated with SEQ ID NO:136, SEQ ID NO:137, SEQ ID NO:138, SEQ ID NO:139, SEQ ID NO:140, or no stimulation, and cytokine levels measured using a MagPix® system. Results indicate that a response was observed in response to stimulation with certain antigens in mice from treatment groups (FIGS. 39A-39F).

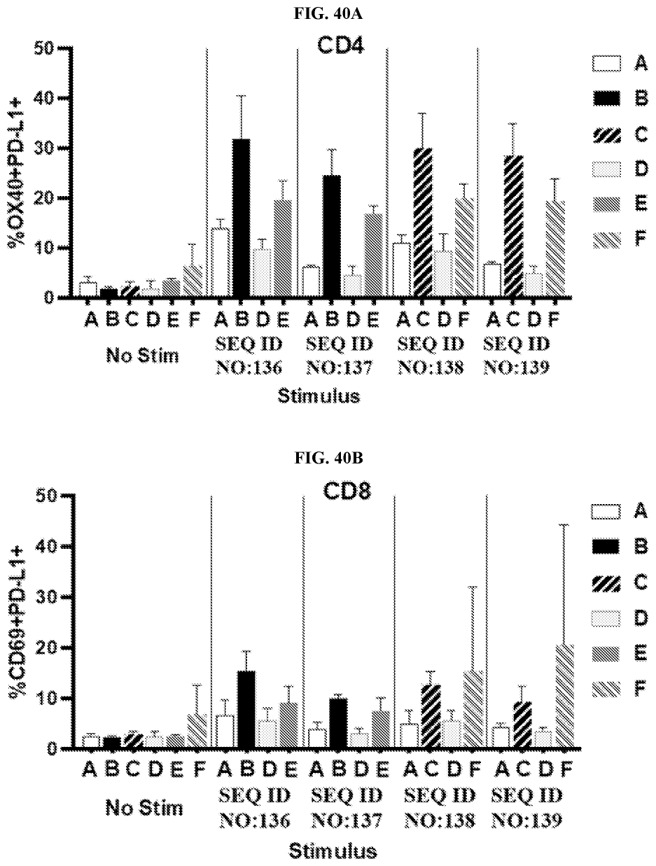

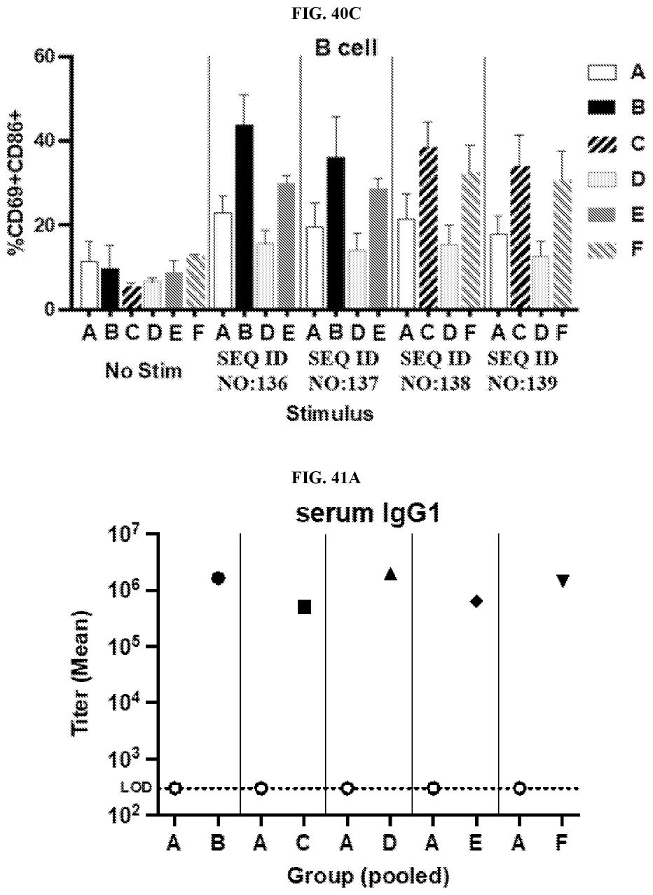

The combination of the AIM protein markers OX40 and PD-L1, CD69 and PD-L1, and CD69 and CD86 were used to identify antigen specific CD4⁺ T cells, CD8⁺ T cells, and B cells, respectively, by flow cytometry of splenocytes obtained from HLA-DR4 mice in all treatment (Groups B-F) and control (Group A) groups, which were activated in vitro by exemplary antigens: SEQ ID NO:136; SEQ ID NO:137; SEQ ID NO:138; SEQ ID NO:139; and SEQ ID NO:140; or no stimulation (FIG. 40A-40C).

Thus, in certain embodiments, the antigens of the present disclosure elicit an adaptive immune response mediated by antigen specific T cell activation, as indicated by elevated levels of certain exemplary cytokines and/or certain exemplary T cells.

Exp. No. 6

Assessment of adaptive immune response mediated by T cells was performed by analysis of splenocytes obtained from HLA-DR4 mice immunized as described elsewhere herein using the vaccine compositions indicated in Table 6.

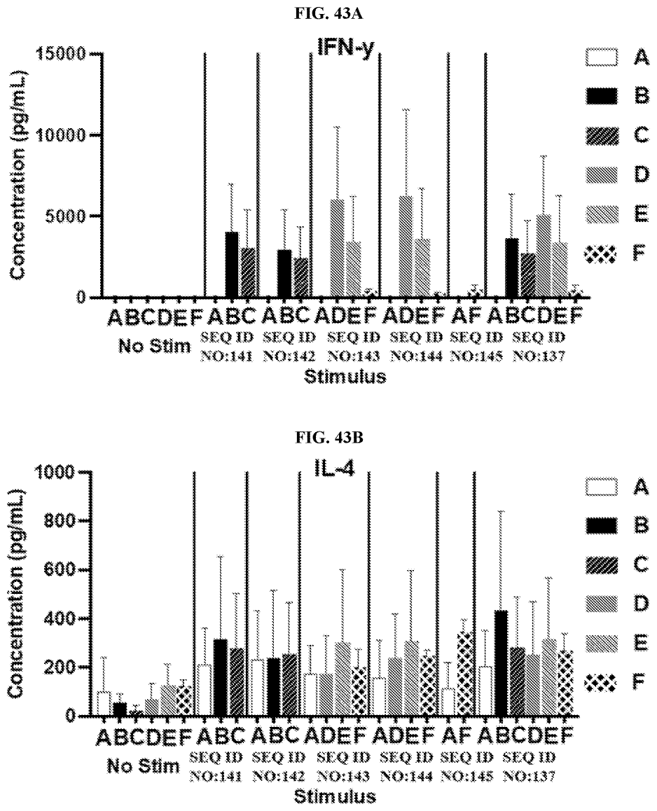

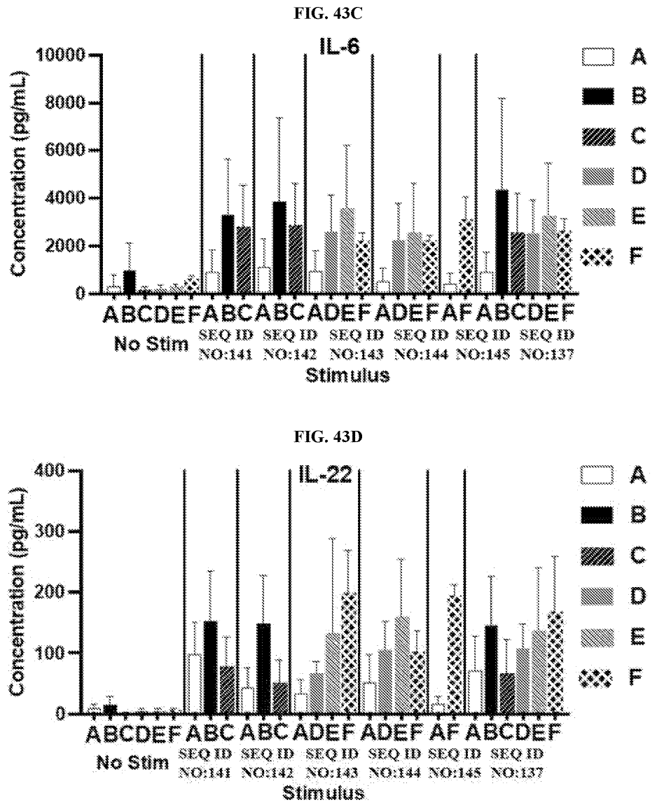

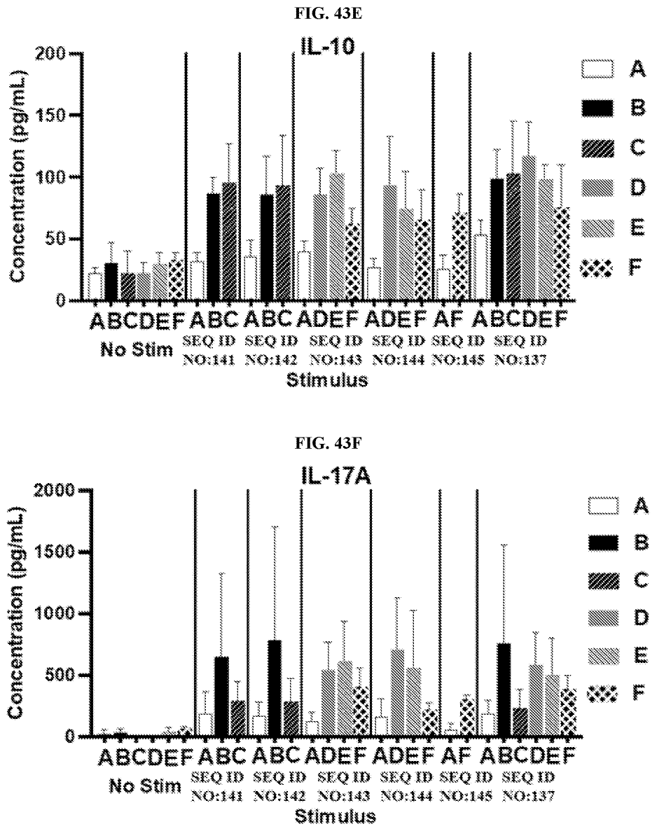

Splenocytes were obtained from mice of groups A-F 47 days after first vaccination (approximately 30 days after second vaccination), and approximately 7 days after UPEC25 challenge, and stimulated with SEQ ID NO:141, SEQ ID NO:142, SEQ ID NO:143, SEQ ID NO:144, SEQ ID NO:145, and SEQ ID NO:137, and cytokine levels measured using a MagPix® system. Results indicate that a cytokine response was observed in response to stimulation with certain antigens in mice from treatment groups (FIGS. 43A-43F).

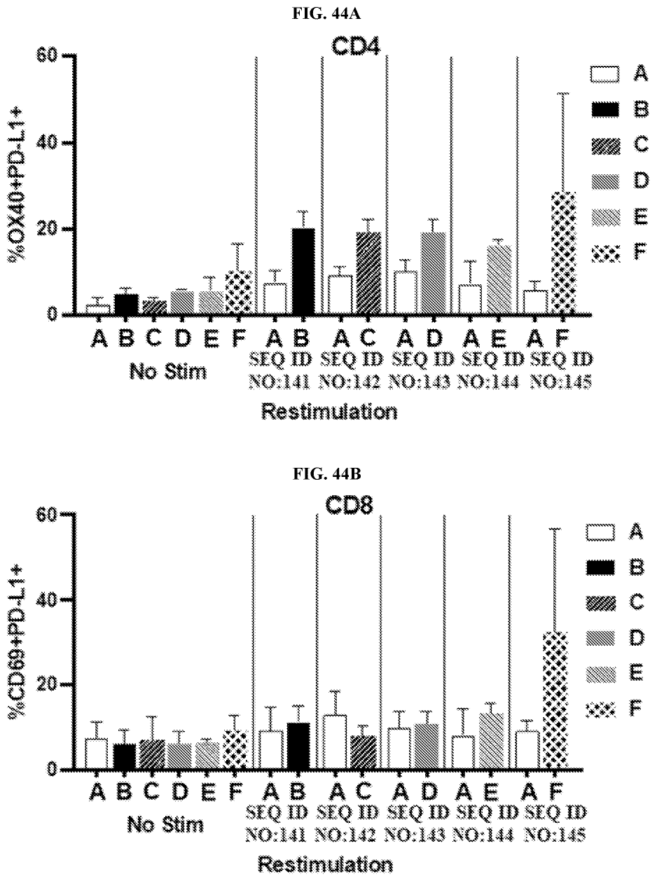

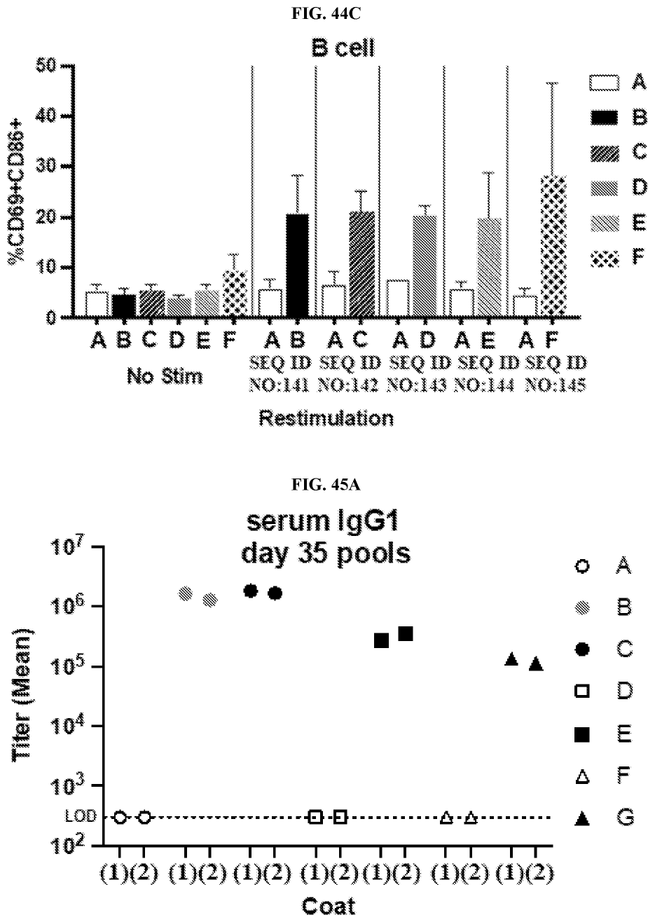

The combination of the AIM protein markers OX40 and PD-L1, CD69 and PD-L1, and CD69 and CD86 were used to identify antigen specific CD4⁺ T cells, CD8⁺ T cells, and B cells, respectively, by flow cytometry of splenocytes obtained from HLA-DR4 mice in all treatment (Groups B-F) and control (Group A) groups, which were activated in vitro by exemplary antigens: SEQ ID NO:141, SEQ ID NO:142, SEQ ID NO:143, SEQ ID NO:144, and SEQ ID NO:145, or no stimulation (FIG. 44A-44C).

Thus, in certain embodiments, the antigens of the present disclosure elicit an adaptive immune response mediated by antigen specific T cell activation, as indicated by elevated levels of certain exemplary cytokines and/or certain exemplary T cells.

Exp. No. 7

Assessment of adaptive immune response mediated by T cells was performed by analysis of splenocytes and spleen cells obtained from HLA-DR4 mice immunized as described elsewhere herein using the vaccine compositions indicated in Table 7.

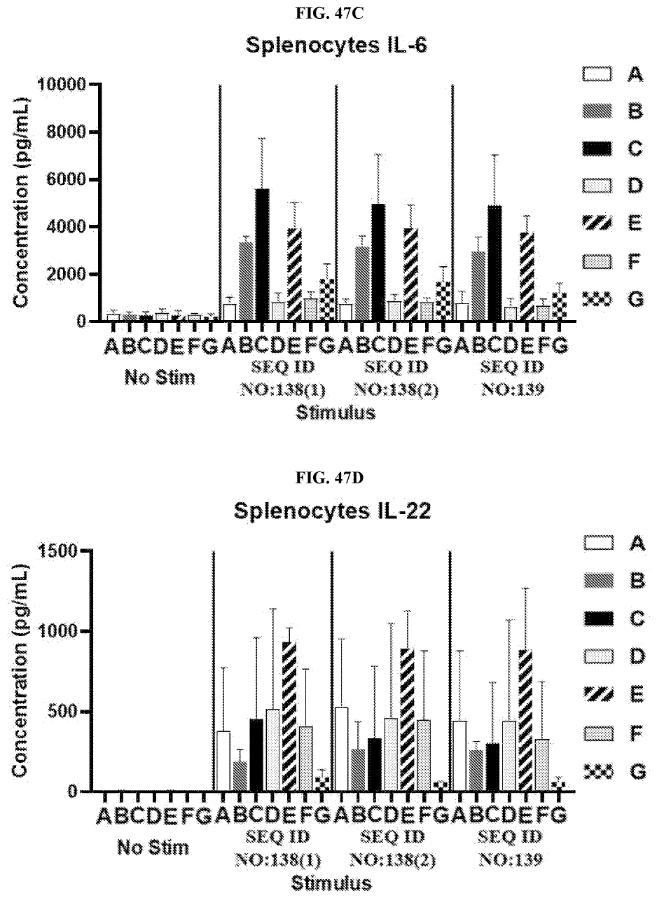

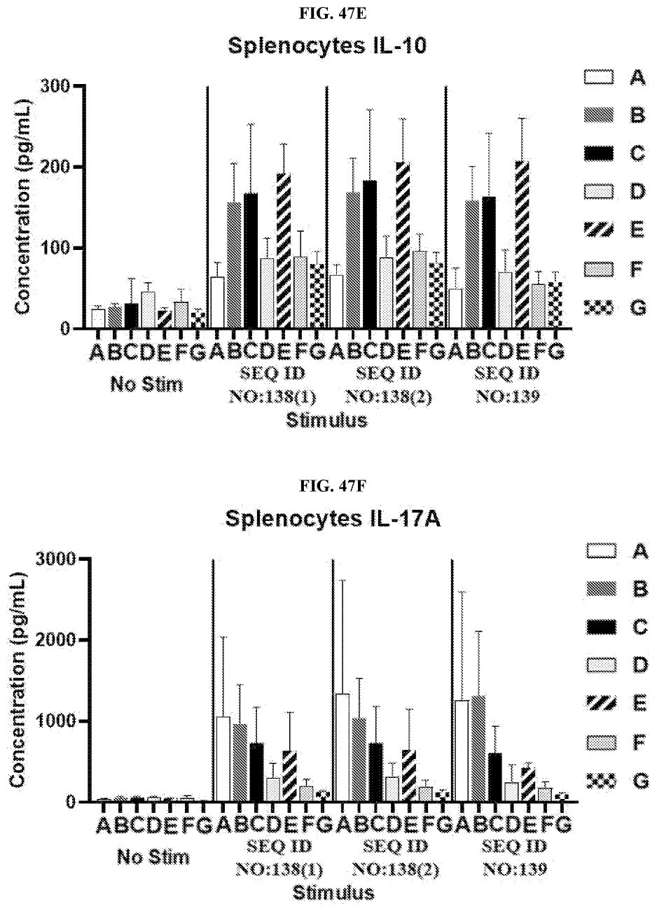

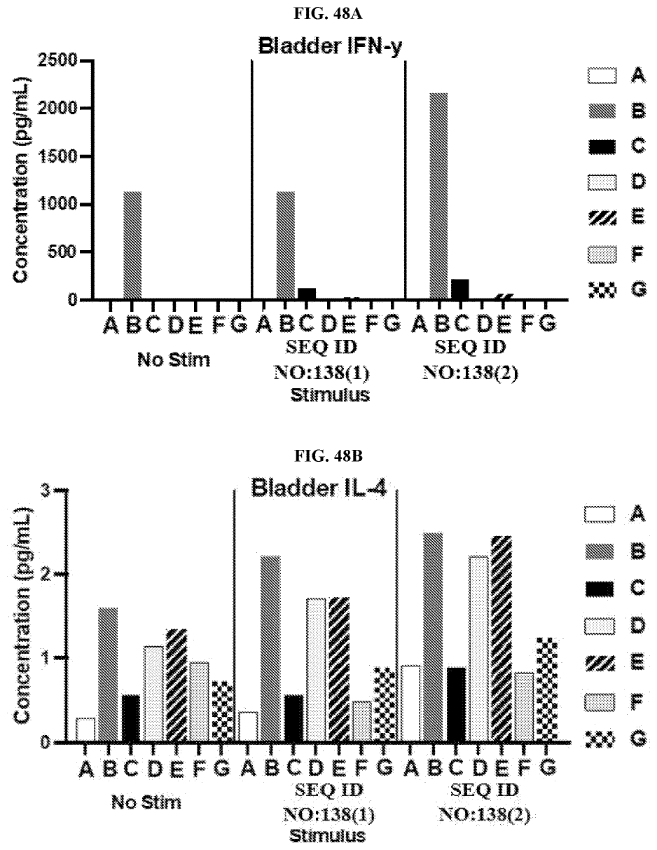

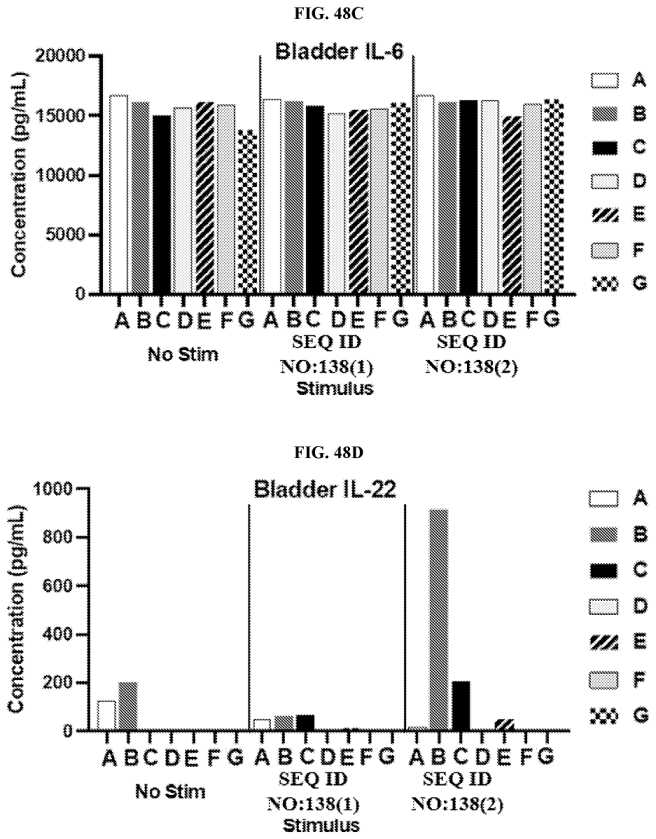

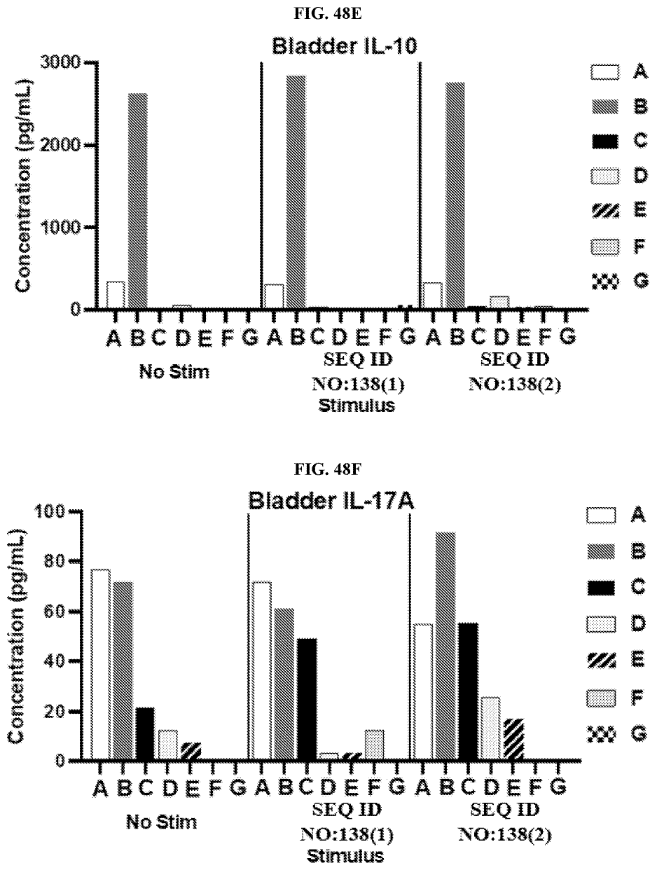

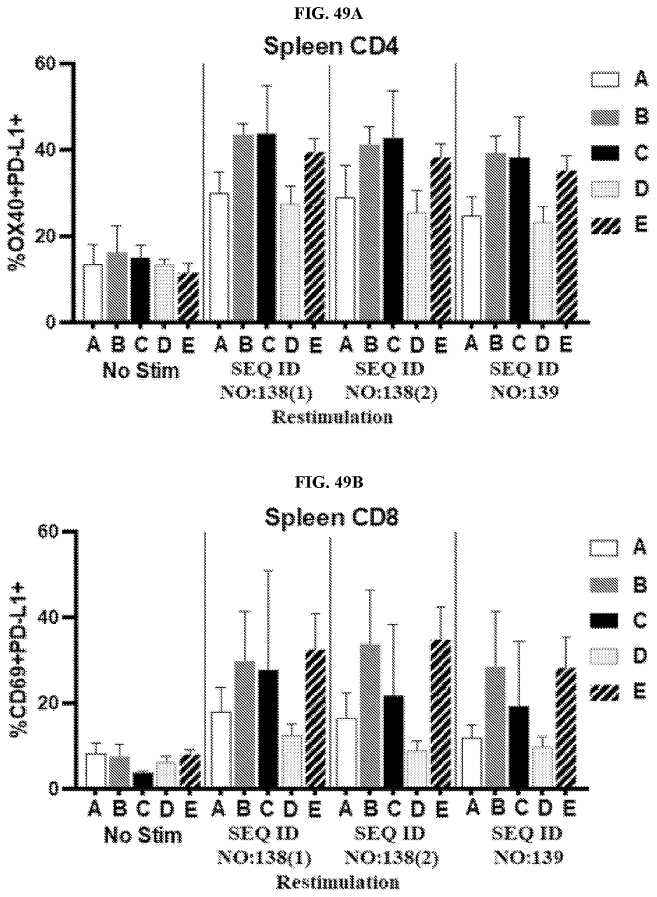

Splenocytes (FIGS. 47A-47F) and spleen cells (FIGS. 48A-48F) were obtained from mice of groups A-G 55 days after first vaccination (approximately 30 days after second vaccination), and approximately 7 days after UPEC25 challenge, and stimulated with SEQ ID NO:138(1), SEQ ID NO:138(2), and SEQ ID NO:139, wherein (1) and (2) vary with respect to buffer conditions, as described in Table 7, and cytokine levels measured using a MagPix® system. Results indicate that a cytokine response was observed in response to stimulation with certain antigens in mice from treatment groups.

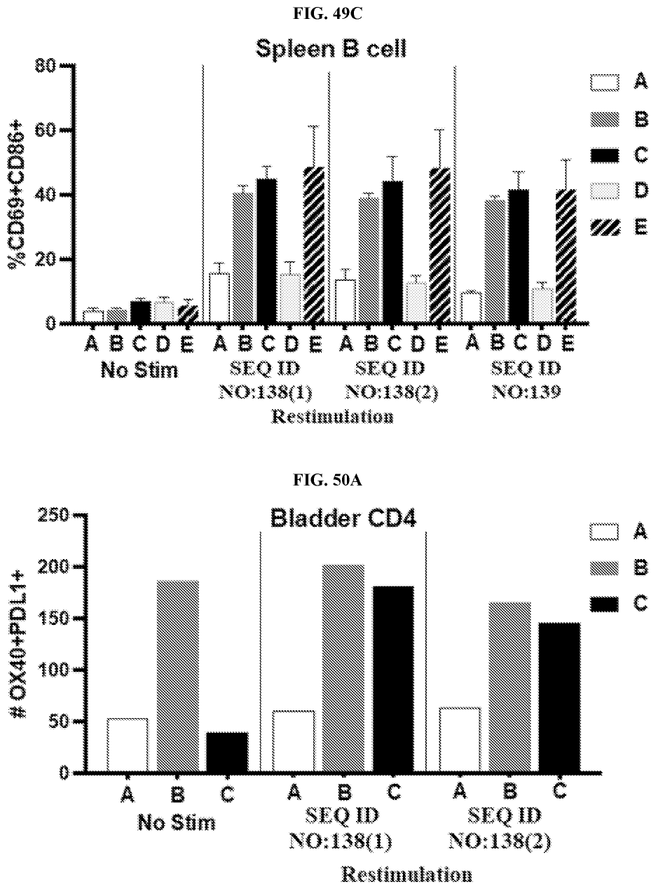

The combination of the AIM protein markers OX40 and PD-L1, CD69 and PD-L1, and CD69 and CD86 were used to identify antigen specific CD4⁺ T cells, CD8⁺ T cells, and B cells, respectively, by flow cytometry of splenocytes obtained from HLA-DR4 mice in all treatment (Groups B-E) and control (Group A) groups, which were activated in vitro by exemplary antigens: SEQ ID NO:138(1), SEQ ID NO:138(2), and SEQ ID NO:139, wherein (1) and (2) vary with respect to buffer conditions, as described in Table 7, or no stimulation (FIG. 49A-49C).

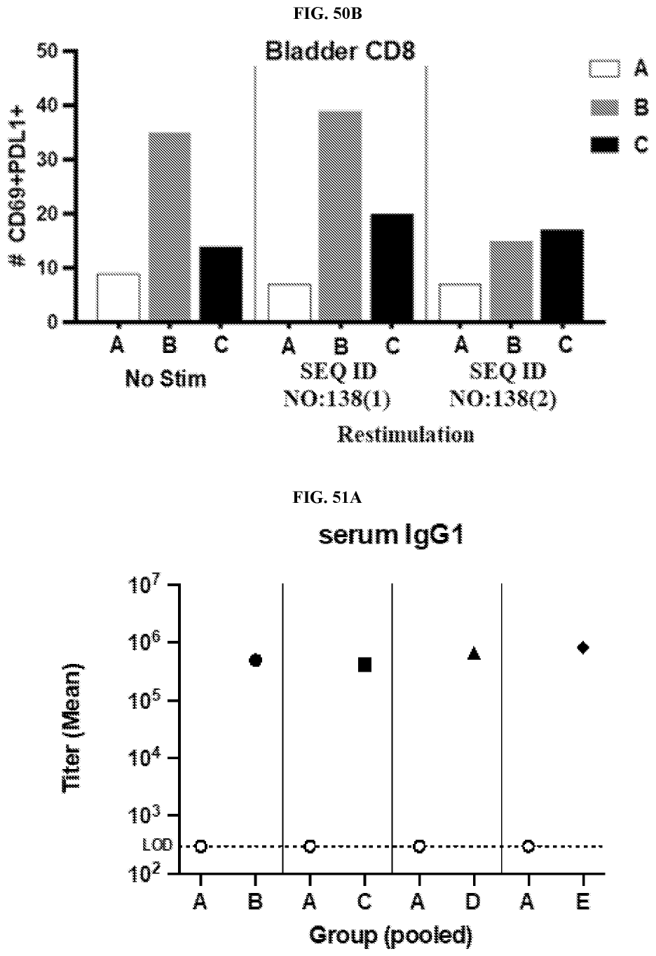

The combination of the AIM protein markers OX40 and PD-L1, and CD69 and PD-L1, were used to identify antigen specific CD4⁺ T cells and CD8⁺ T cells, respectively, by flow cytometry of bladder cells obtained from HLA-DR4 mice in all treatment (Groups B-C) and control (Group A) groups, which were activated in vitro by exemplary antigens: SEQ ID NO:138(1), SEQ ID NO:138(2), wherein (1) and (2) vary with respect to buffer conditions, as described in Table 7, or no stimulation (FIG. 50A-50B).

Thus, in certain embodiments, the antigens of the present disclosure elicit an adaptive immune response mediated by antigen specific T cell activation, as indicated by elevated levels of certain exemplary cytokines and/or certain exemplary T cells.

Exp. No. 8

Assessment of adaptive immune response mediated by T cells was performed by analysis of splenocytes obtained from CB1 mice immunized as described elsewhere herein using the vaccine compositions indicated in Table 8.

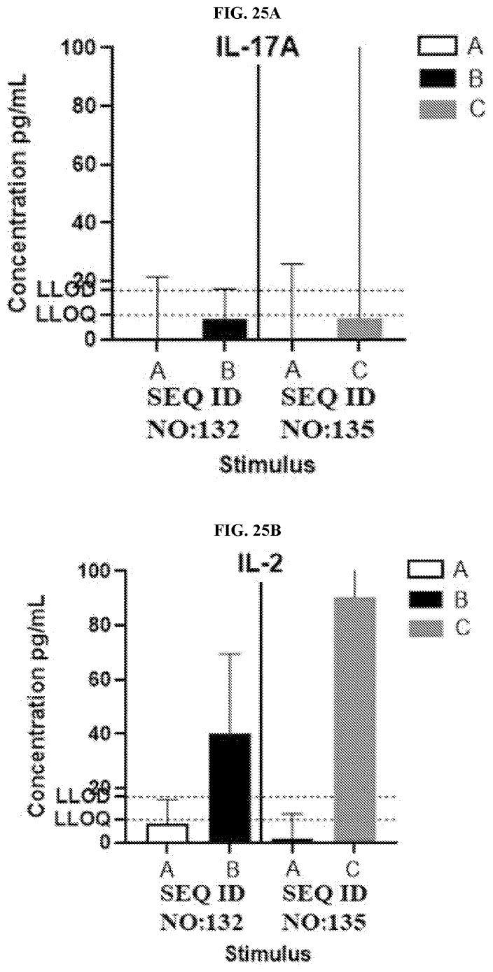

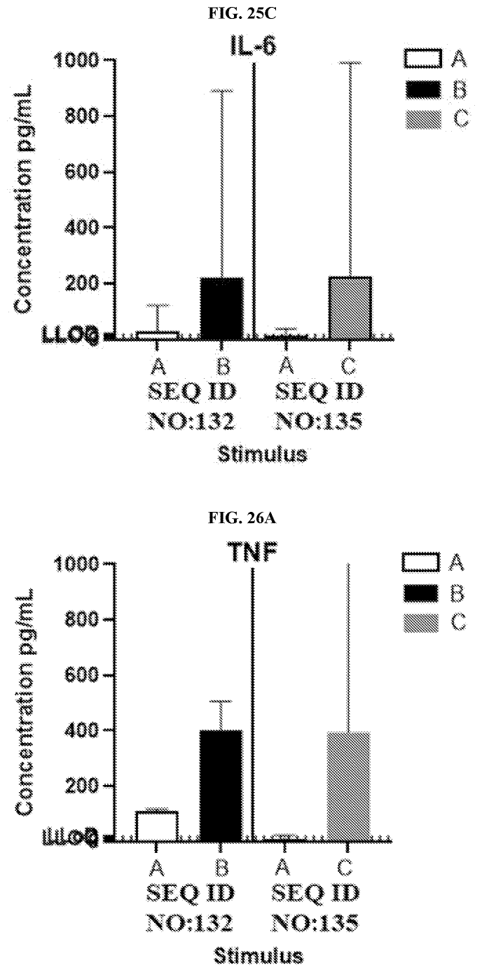

Splenocytes were obtained from mice of groups A-C approximately 14 days after second vaccination and stimulated with SEQ ID NO:132 or SEQ ID NO:135, and analyzed for production of Th1/Th17/pro-inflammatory cytokines IL-17A (FIG. 25A), IL-2 (FIG. 25B), and IL-6 (FIG. 25C) by Cytometric Bead Array (CBA) using a Th1/2/17 kit (BD Biosciences). Results indicate that an IL-2 and IL-6 response was observed in response to stimulation with antigens SEQ ID NO:132 or SEQ ID NO:135 in splenocytes derived from treatment group mice.

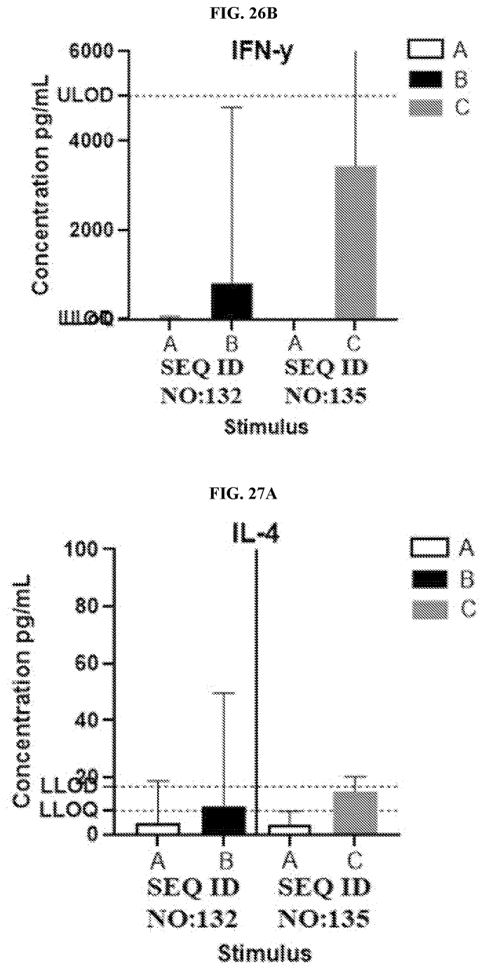

Splenocytes were obtained from mice of groups A-C approximately 14 days after second vaccination and stimulated with SEQ ID NO:132 or SEQ ID NO:135, and analyzed for production of Th1/pro-inflammatory cytokines TNF-α (FIG. 26A) and IFN-γ (FIG. 26B) by Cytometric Bead Array (CBA) using a Th1/2/17 kit (BD Biosciences). Results indicate that TNF-α and IFN-γ production was observed in response to stimulation with antigens SEQ ID NO:132 or SEQ ID NO:135 in splenocytes derived from treatment group mice.

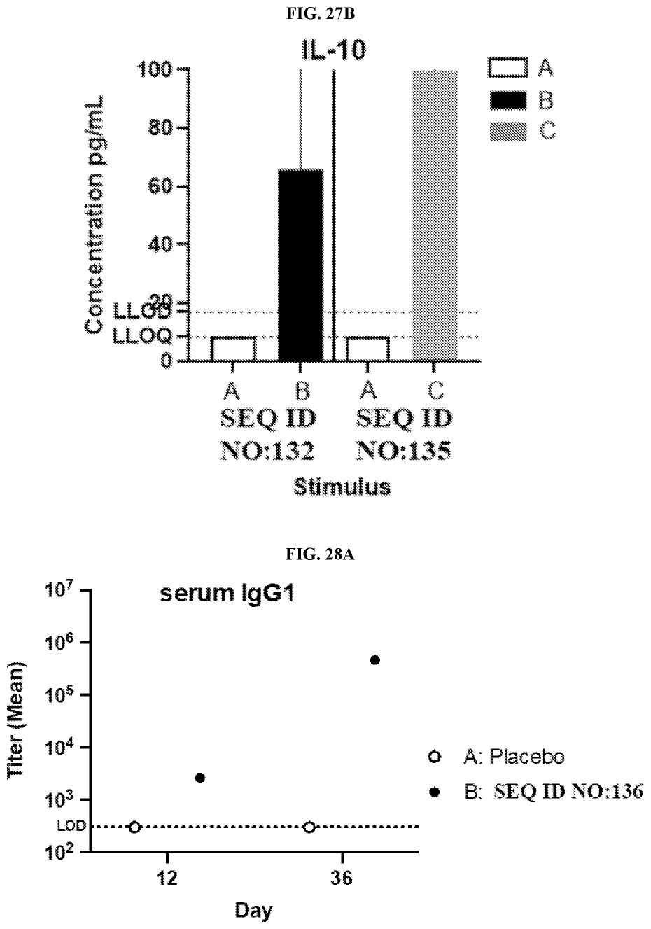

Splenocytes were obtained from mice of groups A-C approximately 14 days after second vaccination and stimulated with SEQ ID NO:132 or SEQ ID NO:135, and analyzed for production of Th2/anti-inflammatory cytokines IL-4 (FIG. 27A) and IL-10 (FIG. 27B) by Cytometric Bead Array (CBA) using a Th1/2/17 kit (BD Biosciences). Generally, the results indicate that IL-10 production was observed in response to stimulation with antigens SEQ ID NO:132 or SEQ ID NO:135 in splenocytes derived from treatment group mice (i.e., groups D-F).

Thus, in certain embodiments, the antigens and/or antigen fragments of the present disclosure elicit an adaptive immune response mediated by T cell activation, as indicated by elevated levels of several antibodies and/or cytokines, non-limiting examples including IL-17A, IL-2, IL-6, TNF-α, IFN-γ, IL-4, and IL-10.

Exp. No. 9

Assessment of adaptive immune response mediated by T cells was performed by analysis of splenocytes and spleen cells obtained from C57BL/6 mice immunized as described elsewhere herein using the vaccine compositions indicated in Table 9.

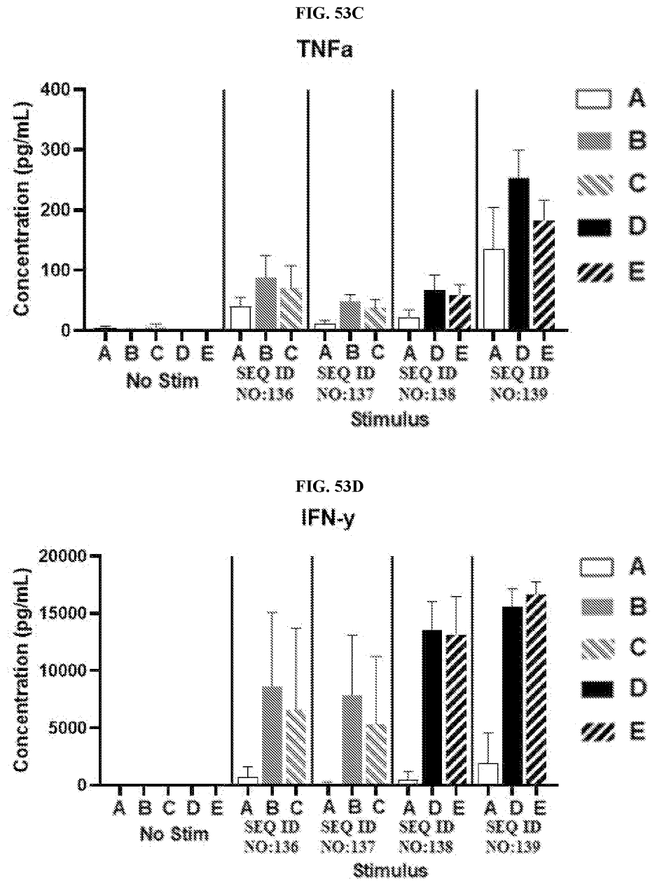

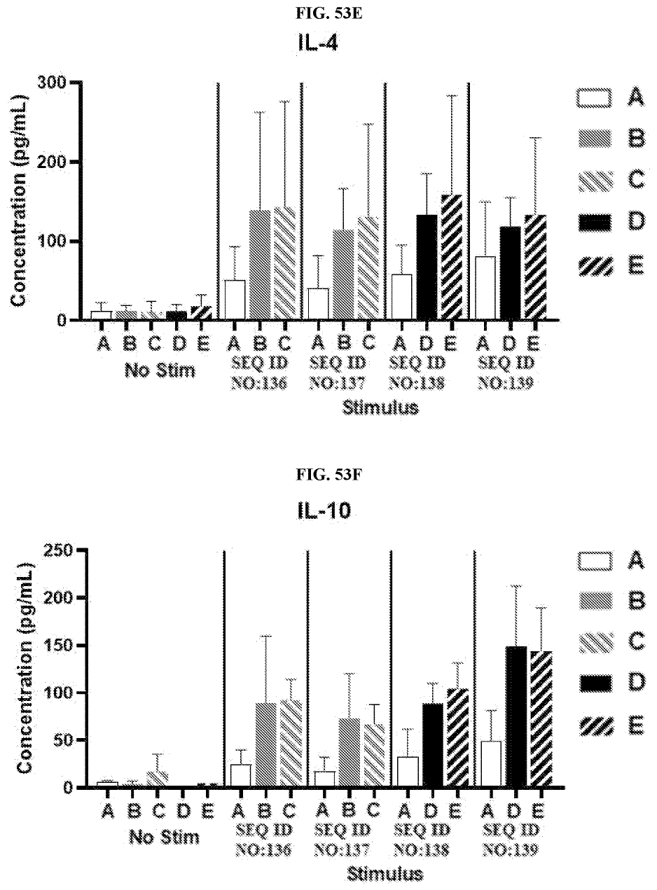

Splenocytes were obtained from mice of groups A-E 52 days after first vaccination (approximately 30 days after second vaccination), and approximately 7 days after UPEC25 challenge, and stimulated with SEQ ID NO:136, SEQ ID NO:137, SEQ ID NO:138, SEQ ID NO:139, or no stimulation, and cytokine levels measured using a MagPix® system. Results indicate that a response was observed in response to stimulation with certain antigens in mice from treatment groups (FIGS. 53A-53F).

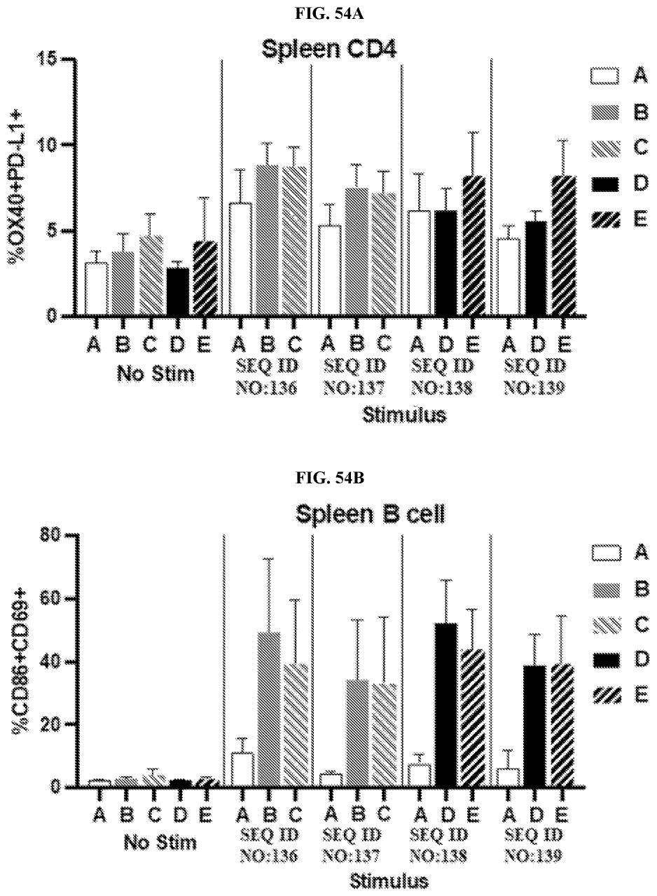

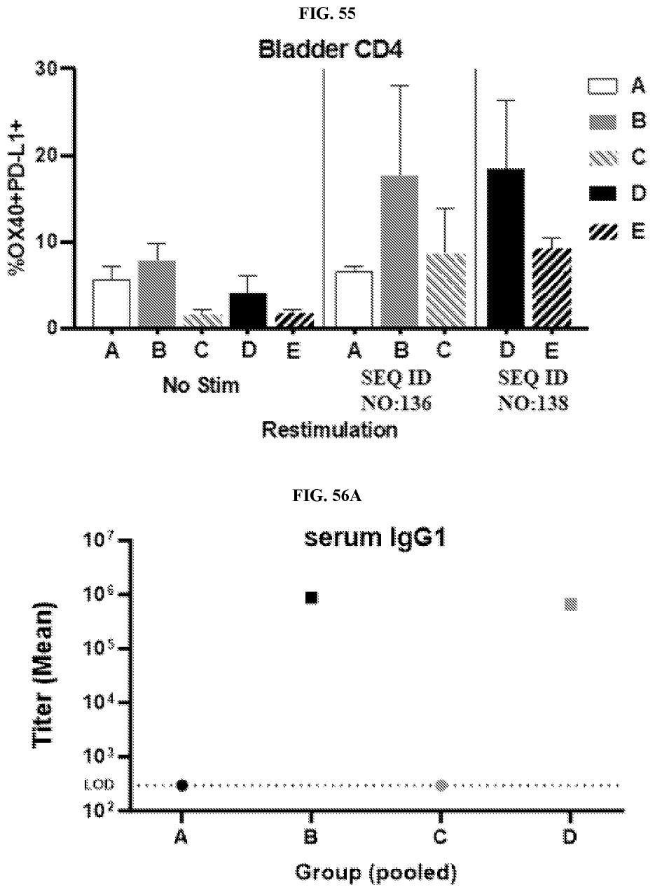

The combination of the AIM protein markers OX40 and PD-L1, and CD86 and CD69, were used to identify antigen specific CD4⁺ T cells and B cells, respectively, by flow cytometry of splenocytes obtained from C57BL/6 mice in all treatment (Groups B-E) and control (Group A) groups, which were activated in vitro by exemplary antigens SEQ ID NO:136, SEQ ID NO:137, SEQ ID NO:138, SEQ ID NO:139, or no stimulation (FIGS. 54A-54B).

The combination of the AIM protein markers OX40 and PD-L1 was used to identify antigen specific CD4⁺ T cells by flow cytometry bladder cells obtained from C57BIU6 mice in all treatment (Groups B-E) and control (Group A) groups, which were activated in vitro by exemplary antigens SEQ ID NO:136, SEQ ID NO:137, SEQ ID NO:138, SEQ ID NO:139, or no stimulation (FIG. 55).

Thus, in certain embodiments, the antigens of the present disclosure elicit an adaptive immune response mediated by antigen specific T cell activation, as indicated by elevated levels of certain exemplary cytokines and/or certain exemplary T cells.

Exp. No. 10

Assessment of adaptive immune response mediated by T cells was performed by analysis of cells obtained from the spleen (individual), lymph node (pooled), and bladder (pooled) of C57BL/6 mice immunized as described elsewhere herein using the vaccine compositions indicated in Table 10.

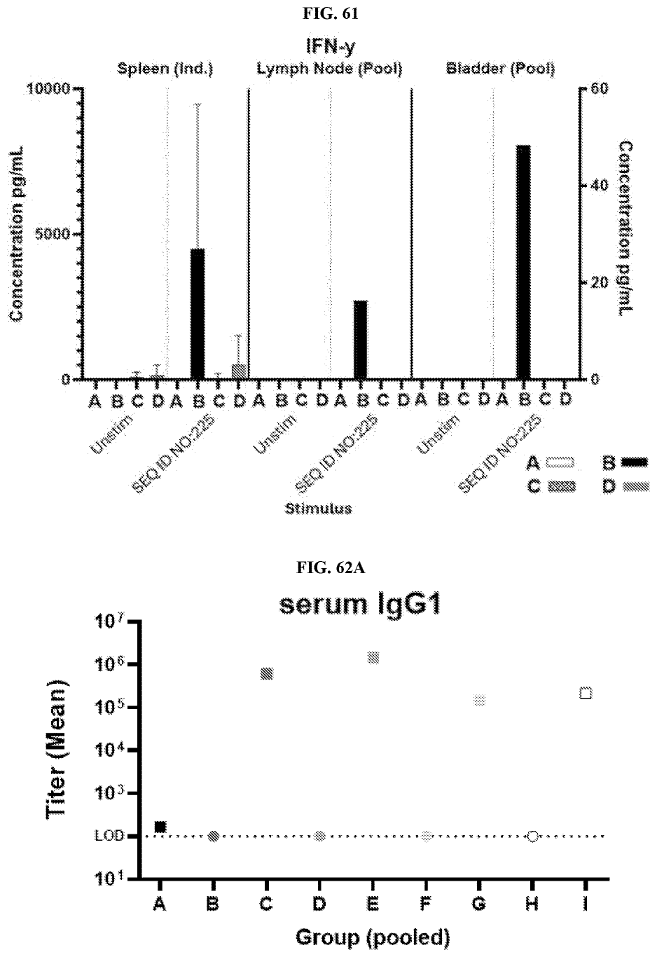

Spleen, lymph node, and bladder cells were obtained from mice groups A-D 33 days after first vaccination (approximately 12 days after second vaccination) and subjected to stimulation with SEQ ID NO:225 or no stimulation, and IFN-γ concentration was measured using a MagPix® system. Results indicate that a response was observed in response to stimulation with certain antigens in mice from treatment groups (FIG. 61).

Thus, in certain embodiments, the antigens of the present disclosure elicit an adaptive immune response mediated by antigen specific T cell activation, as indicated by elevated levels of IFN-γ.

Exp. No. 11

Assessment of adaptive immune response mediated by T cells was performed by analysis of splenocytes obtained from C57BL/6 mice immunized as described elsewhere herein using the vaccine compositions indicated in Table 11.

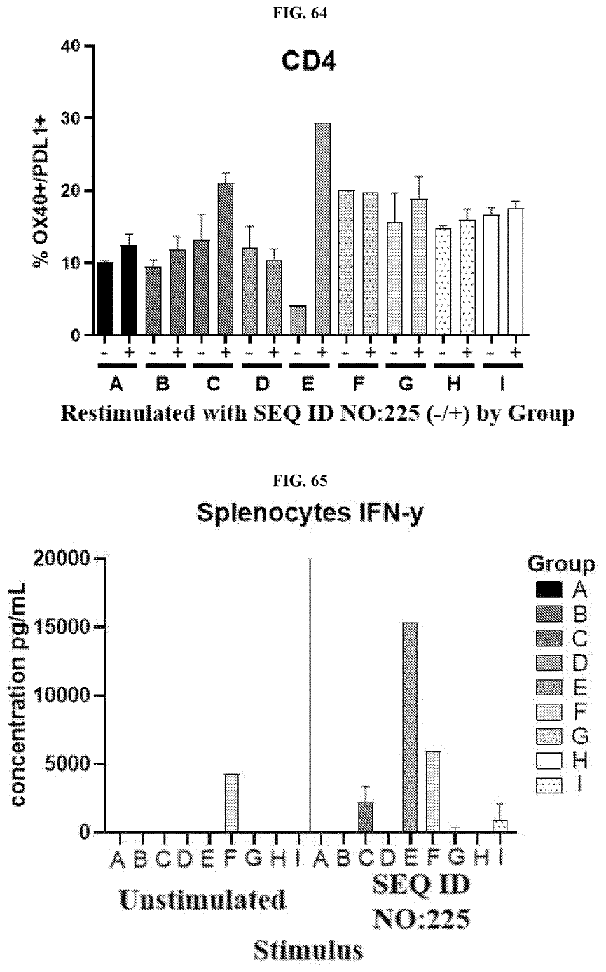

The combination of AIM protein markers OX40 and PD-L1 was used to identify specific CD4⁺ T cells by flow cytometry of splenocytes obtained from C57BL/6 mice in all treatment (i.e., Groups A, C, E, G, and I) and control or placebo (i.e., Groups B, D, F, and H) groups, which were activated in vitro by exemplary antigen SEQ ID NO:225, or unstimulated (FIG. 64).

Splenocytes were obtained from mice of groups A-I 12 days post second vaccination (approximately 33 days post first vaccination) and either stimulated with SEQ ID NO:225 or unstimulated (i.e., “Unstim”), and cytokine levels were measured using a MagPix® system. Results indicate that a cytokine response was observed in response to stimulation with SEQ ID NO:225 (FIG. 65).

Thus, in certain embodiments, the antigens of the present disclosure elicit an adaptive immune response mediated by antigen specific T cell activation, as indicated by elevated levels of certain exemplary cytokines and/or certain exemplary T cells. In certain embodiments, the results indicate the use of an adjuvant, when administering the antigen parenterally (e.g., intramuscularly), is critical for stimulating certain exemplary cytokines and/or certain exemplary T cells (cf Group A and any of Groups C, E, G, and I).

Exp. No. 12

Assessment of adaptive immune response mediated by T cells was performed by analysis of cells obtained from the spleen (individual), lymph node (pooled), and bladder (pooled) of C3H/HeN mice immunized as described elsewhere herein using the vaccine compositions indicated in Table 12.

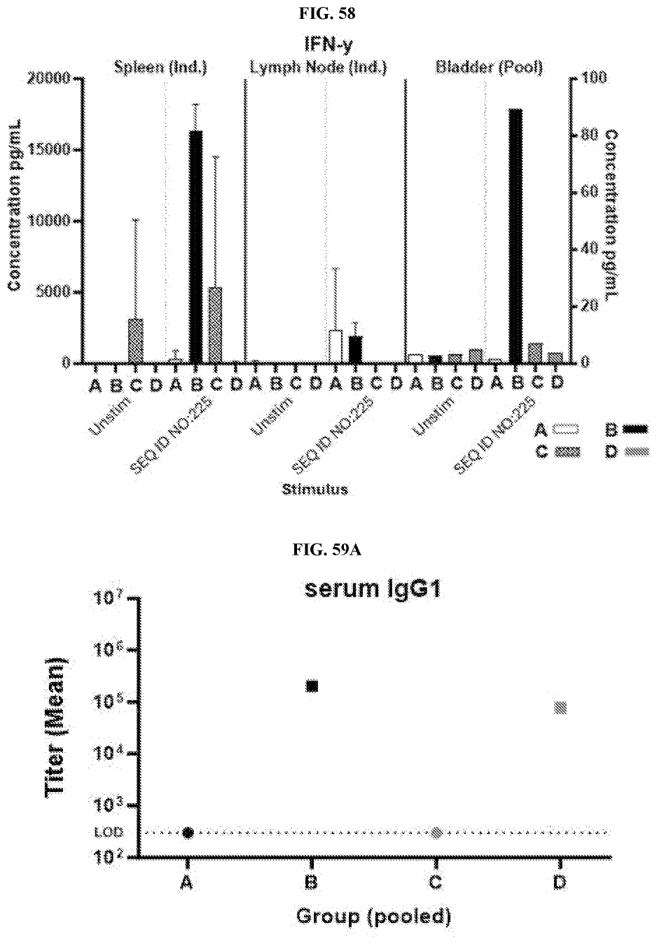

Spleen, lymph node, and bladder cells were obtained from mice groups A-D 33 days after first vaccination (approximately 12 days after second vaccination) and subjected to stimulation with SEQ ID NO:225 or no stimulation, and IFN-γ concentration was measured using a MagPix® system. Results indicate that a response was observed in response to stimulation with certain antigens in mice from treatment groups (FIG. 58).

Thus, in certain embodiments, the antigens of the present disclosure elicit an adaptive immune response mediated by antigen specific T cell activation, as indicated by elevated levels of IFN-γ.

Sequence Listing
SEQ ID NO: 1
MKRVITLFAVLLMGWSVNAWSFACKTANGTAIPIGGGSANVYVNLAPAVNVGQNLVVDLSTQIF
CHNDYPETITDYVTLQRGSAYGGVLSSFSGTVKYNGSSYPFPTTSETPRVVYNSRTDKPWPVAL
YLTPVSSAGGVAIKAGSLIAVLILRQTNNYNSDDFQFVWNIYANNDVVVPTGGCDASARDVTVT
LPDYPGSVPIPLTVYCAKSQNLGYYLSGTTADAGNSIFTNTASFSPAQGVGVQLTRNGTIIPAN
NTVSLGAVGTSAVSLGLTANYARTGGQVTAGNVQSIIGVTFVYQ
SEQ ID NO: 2
MKKIIPLFTTLLLLGWSMNAWSFACKTATGATIPIGGGSANVYVNLTPAVNVGQNLVVDLSTQI
FCHNDYPETITDYVTLQRGSAYGGVLSSFSGTVKYNGTSYPFPTTTETARVIYDSRTDKPWPAV
LYLTPVSTAGGVAITAGSLIAVLILHQTNNYNSDSFQFIWNIYANNDVVVPTGGCDVSARDVTV
TLPDYPGSMAVPLTVHCAQSQQLGYYLSGTTADSANAIFTNTASASPAQGIGVQLTRNGSAVPA
NSTVSLGTVGTSPVNLGLTATYARTTGQVTAGNVQSIIGITEVYQ
SEQ ID NO: 3
MKLLKVAAIAAIVFSGSALAGVVPQYGGGGGNHGGGGNNSGPNSELNIYQYGGGNSALAQQADA
RNSDLTITQHGGGNGADVGQGSDDSSIDLTQRGFGNSATLDQWNGKDSTMTVKQFGGGNGAAVD
QTASNSSVNVTQVGFGNNATAHQY
SEQ ID NO: 4
MHLRHLFSSRLRGSLLLGSLLVASSFSTQAAEEMLRKAVGKGAYEMAYSQQENALWLATSQSRK
LDKGGVVYRLDPVTLEVTQAIHNDLKPFGATINNTTQTLWFGNTVNSAVTAIDAKTGEVKGRLV
LDDRKRTEEVRPLQPRELVADDATNTVYISGIGKESVIWVVDGENIKLKTAIQNTGKMSTGLAL
DSKGKRLYTTNADGELITIDTADNKILSRKKLLDDGKEHFFINISLDTTRQRAFITDSKAAEVL
VVDTRNGNILAKVAAPESLAVLFNPARNEAYVTHRQAGKVSVIDAKSYKVVKTFDTPTHPNSLA
LSADGKTLYVSVKQKSTKQQEATQPDDVIRIAL
SEQ ID NO: 5
MSLRHFAAPRLRHSLLVTSLLLAGSFSAHAAEEMLRKAVGKGAYEMAYSQQENALWVATSQSRS
LDKGGVVYRLDPTTLDVTQIIHNDLKPFGATINHATGTLWFGNTVDSTVTAIDAKTGAVKGRLV
LDERQRSETVRPLQPRELAVNEQTNTVYITGLGKESVIWVVDGATLKLKTTITGTGAMATGLAI
DPQAKRLYTTNADGELLTIDSESNTIASRKKLQDDGKAHFYLNLSLDTAGHRAFITDSKQPEVL
VVDTRDGKVLEKIAAPESLAVLENPARNEAYVTHRKAGEVSVIDGKSYKVVKTFKTPTHPNSLA
LSEDGKTLYVSVKQASSREKEATAPDDVIRIAL
SEQ ID NO: 6
MKKWFPALLFSLCVSGESSAWNNIVFYSLGNVNSYQGGNVVITQRPQFITSWRPGIATVTWNQC
NGPEFADGSWAYYREYIAWVVFPKKVMTKNGYPLFIEVHNKGSWSEENTGDNDSYFFLKGYKWD
ERAFDAGNLCQKPGETTRLTEKENDIIFKVALPADLPLGDYSVTIPYTSGIQRHFASYLGARFK
IPYNVAKTLPRENEMLFLFKNIGGCRPSAQSLEIKHGDLSINSANNHYAAQTLSVSCDVPANIR
FMLLRNTTPTYSHGKKFSVGLGHGWDSIVSVNGVDTGETTMRWYKAGTQNLTIGSRLYGESSKI
QPGVLSGSATLLMILP*
SEQ ID NO: 7
MKKWLPAFLFLSLSGCNDALAANQSTMFYSENDNIYRPQLSVKVTDIVQFIVDINSASSTATLS
YVACNGFTWIHGLYWSEYFAWLVVPKHVSYNGYNIYLELQSRGSFSLDAEDNDNYYLTKGFAWD
EVNTSGQTCFNIGEKRSLAWSFGGVTLNARLPVDLPKGDYTFPVKFLRGIQRNNYDYIGGRYKI
PSSLMKTFPFNGTLNFSIKNTGGCRPSAQSLEINHGDLSINSANNHYAAQTLSVSCDVPTNIRF
FLLSNTNPAYSHGQQFSVGLGHGWDSIISINGVDTGETTMRWYRAGTQNLTIGSRLYGESSKIQ
PGVLSGSATLLMILP
SEQ ID NO: 8
MSRYKTDNKQPRFRYSVLARCVAWANISVQVLFPLAVTFTPVMAARAQHAVQPRLSMENTTVTA
DNNVEKNVASLAANAGTFLSSQPDSDATRNFITGMATAKANQEIQEWLGKYGTARVKLNVDKKF
SLKDSSLEMLYPIYDTPTNMLFTQGAIHRTDDRTQSNIGFGWRHFSENDWMAGVNTFIDHDLSR
SHTRIGVGAEYWRDYLKLSANGYIRASGWKKSPDVEDYQERPANGWDIRAEGYLPAWPQLGASL
MYEQYYGDEVGLFGKDKRQKDPHAITAEVNYTPVPLLTLSAGHKQGKSGENDTREGLEVNYRIG
EPLEKQLDTDSIRERRMLAGSRYDLVERNNNIVLEYRKSEVIRIALPERIEGKGGQTVSLGLVV
SKATHGLKNVQWEAPSLLAAGGKITGQGNQWQVTLPAYQAGKDNYYAISAIAYDNKGNASKRVQ
TEVVISGAGMSADRTALTLDGQSRIQMLANGNEQKPLVLSLRDAEGQPVTGMKDQIKTELTEKP
AGNIVTRTLKATKSQAKPTLGEFTETEAGVYQSVFTTGTQSGEATITVSVDDMSKTVTAELRAT
MMDVANSTLSANEPSGDVVADGQQAYTLTLTAVDSEGNPVTGEASRLRLVPQDINGVTVGAISE
IKPGVYSATVSSTRAGNVVVRAFSEQYQLGTLQQTLKFVAGPLDAAHSSITLNPDKPVVGGTVT
AIWTAKDANDNPVTGLNPDAPSLSGAAAAGSTASGWTDNGDGTWTAQISLGTTAGELDVMPKLN
GQDAAANAAKVTVVADALSSNQSKVSVAEDHVKAGESTTVTLVAKDAHGNAISGLSLSASLTGT
ASEGATVSSWTEKGDGSYVATLTTGGKTGELRVMPLENGQPAATEAAQLTVIAGEMSSANSTLV
ADNKTPTVKTTTELTFTVKDAYGNPVTGLKPDAPVESGAASTGSERPSAGNWTEKGNGVYVSTL
TLGSAAGQLSVMPRVNGQNAVAQPLVLNVAGDASKAEIRDMTVKVNNQLANGQSANQITLTVVD
SYGNPLQGQEVTLTLPQGVTSKTGNTVTTNAAGKVDIELMSTVAGELEIEASVKNSQKTVKVKF
KADFSTGQASLEVDAAAQKVANGKDAFTLTATVKDQYGNLLPGAVVVENLPRGVKPLADGNIMV
NADKEGKAELKVVSVTAGTYEITASAGNDQPSNAQSVTFVADKTTATISSIEVIGNRAVADGKT
KQTYKVTVTDANNNLLKDSEVTLTASPENLVLTPNGTATTNEQGQAIFTATTTVAATYTLTAKV
EQADGQESTKTAESKFVADDKNAVLAASPERVDSLVADGKTTATLTVTLMSGVNPVGGTMWVDI
EAPEGVTEADYQFLPSKNDHFASGKITRTFSTNKPGTYTFTENSLTYGGYEMKPVTVTINAVPA
DTEGAEEK
SEQ ID NO: 9
MFIFKRFPAIFILIAGLFSAPAMASIFSYITESTGTPSNATYTYVIERWDPETSGILNPCYGWP
VCYVTVNHKHTVNGTGGNPAFQIARIEKLRTLAEVRDVVLKNRSFPIEGQTTHRGPSLNSNQEC
VGLFYQPNSSGISPRGKLLPGSLCGIAPPPVGACKISEGAVNLNYGDIDEASLSGAKRSETINV
TCNLAMKVLVIASGSDSGRVPLRADKSLYADLYLNNYPGEKGVTVNVPAGGSAPVSVSSTLRTN
GRVAPGRESGSGSIILTMP
SEQ ID NO: 10
MRKKYMPRALGPLLLVVLSPAVAQQNDDNEIIVSASRSNRTVAEMAQTTWVIENAELEQQIQGG
KELKDALAQLIPGLDVSSQSRTNYGMNMRGRPLVVLIDGVRLNSSRSDSRQLDSVDPENIDHIE
VISGATALYGGGSTGGLINIVTKKGQPETMMEFEAGTKSGENSSKDHDERIAGAVSGGNDHISG
RLSVAYQKFGGWFDGNGDATLLDNTQTGLQHSNRLDIMGTGTLNIDESRQLQLITQYYKSQGDD
NYGLNLGKGFSAISGSSTPYVSKGLNSDRIPGTERHLISLQYSDSDELRQELVGQVYYRDESLR
FYPFPTVNANKQATAFSSSQQDTDQYGMKLTLNSQLMDGWQITWGLDAEHERFTSNQMFFDLAQ
ASASGGLNNHKIYTTGRYPSYDITNLAAFLQSSYDINDIFTVSGGVRYQYTENRVDDFIDYTQQ
QKIAAGKAISADAIPGGSVDYDNFLFNAGLLMHITERQQAWFNFSQGVALPDPGKYYGRGIYGA
AVNGHLPLTKSVNVSDSKLEGVKVDSYELGWRFTGDNLRTQIAAYYSLSNKSVERNKDLTISVK
DDRRRIYGVEGAVDYLIPDTDWSTGVNFNVLKTESKVNGQWQKYDVKESSPSKATAYINWAPEP
WSLRVQSTTSFDVSDAEGNDINGYTTVDFISSWQLPVGTLSFSVENLFDRDYTTVWGQRAPLYY
SPGYGPASLYDYKGRGRTFGLNYSVLF
SEQ ID NO: 11
MKKRLWVLHPLLLASTLPALAAQSDEDSIIVSANRTHRTVAEMAQTTWVIEGQEIEQQVQGGKE
FKDVLAQLIPGIDVSSQGRTNYGMNMRGRAIVVLIDGVRLNSSRTDSRQLDAIDPFNIEHIEVI
SGATSLYGGGSTGGLINIVTKKGQQDRQVDLEVGSKSGFANSNDHDERVAAAVSGGTDHASGRL
SVAYQRFGGWYDGNNDALILDNTQTGLQHSDRLDVMGTGTIEIDDNRQLQLVTQYYKSQGDDDY
GLWLGKNMSAVTSGGKAYTTDGLNSDRIPGTERHLISLQYSDADFFGQNLVSQVYYRDESLTFY
PFPTLTKGQVSSFSSSQQDTDQYGAKLTLNSQPLAGWDLTWGLDADHETFNANQMFFDLPQSMA
SGGLHNESIYTTGRYPGYSISNVAPFLQSSYDLNDIFTVSGGVRYQWTENRVDDFVGYAQQQDI
ANGKARSADAIKGGKTDYDNFLFNAGIVAHLTERQQTWFNFSQGVELPDPGKYYGIGKYGAAVN
GHLPLISSVNVDDSPLQGIKVNSYELGWRYTGDNLRTQLAAYYSTSDKTIVVNRTDMTIDVQSD
KRRIYGVEGAVDYFIPDSDWSVGGNFNVLKSQVQTDGRWQKWDVTLASPSKATAWVGWAPDPWS
LRVQSQQVFDLSDAAGNKLEGYNTVDFIGSYALPVGKLTFSIENLLNEDYVTIWGQRAPLLYSP
TYGSSSLYEYKGRGRTFGLNYALTF
SEQ ID NO: 12
MRINKILWSLTVLLVGLNSQVSVAKYSDDDNDETLVVEATAEQVLKQQPGVSVITSEDIKKTPP
VNDLSDIIRKMPGVNLTGNSASGTRGNNRQIDIRGMGPENTLILIDGVPVTSRNSVRYSWRGER
DTRGDTNWVPPEQVERIEVIRGPAAARYGSGAAGGVVNIITKRPTNDWHGSLSLYTNQPESSEE
GATRRANFSLSGPLAGDALTTRLYGNLNKTDADSWDINSPVGTKNAAGHEGVRNKDINGVVSWK
LNPQQILDFEVGYSRQGNIYAGDTQNSSSSAVTESLAKSGKETNRLYRQNYGITHNGIWDWGQS
RFGVYYEKTNNTRMNEGLSGGGEGRILAGEKFTTNRLSSWRTSGELNIPLNVMVDQTLTVGAEW
NRDKLDDPSSTSLTVNDRDISGISGSAADRSSKNHSQISALYIEDNIEPVPGTNIIPGLRFDYL
SDSGGNFSPSLNLSQELGDYFKVKAGVARTFKAPNLYQSSEGYLLYSKGNGCPKDITSGGCYLI
GNKDLDPEISVNKEIGLEFTWEDYHASVTYFRNDYQNKIVAGDNVIGQTASGAYILKWQNGGKA
LVDGIEASMSFPLVKERLNWNTNATWMITSEQKDTGNPLSVIPKYTINNSLNWTITQAFSASEN
WTLYGRQKPRTHAETRSEDTGGLSGKELGAYSLVGTNFNYDINKNLRLNVGVSNILNKQIFRSS
EGANTYNEPGRAYYAGVTASF
SEQ ID NO: 13
MRSPQYSVPTTLALVISSLLAPNAFAEESEQDTETMIVRSTAEEALKQQPGVSIITAEDIAKQS
PVNDLSDIIRKMPGVNLTGNSASGSRGNNRQIDIRGMGPENTLILIDGVPVTSRNAVRYSWRGE
RDTRGDSNWVPAEMVERIEVLRGPAAARYGSGAAGGVVNIITKRPTNTWHGSLSFFTNQPENNK
EGTTNRANFNLSGPLAGEALTMRLYGNINKTEPDAWDINHAQNGSYAAGREGVRNKDINTLLSW
KMTPQQILDFSYAYSRQGNIYAGDTQYSNGNLSPSGLVDSLYGHETNRLYRQSWGLTYNGLWDW
GQSKAGVYYEKTNNTRLQEGSTGRVEGMINSEDYATSRLESWRTTSELNVPFFWLADQTLTLGM
EWNHDQLDDPASMQATNSNGETIPGTSGDPTQRSTKNSATLTGIYLEDNIEAVPGTNLIPGIRF
DYHNQFGSNWSPSLNLSQELGDMFTLKAGIARVFKAPNLYQSSKGYLLSTRGNGCPNTIAEGSC
YLLGNPDLDPEISINKEIGIEFNLNGYAAGVTWFRNDYKNKIVSGTEVLGYTSSGNNILQWQNG
GKAVVEGLEGNLLIPVLRDVLSWRTNATWMLKSESKETGNPLSVIPKYTVNTMLDWQVNDALSA
NVNWTLYGRQKPRQYAEIRNETGTLATTEVGAYSIVGIGTQYQLNRDIRLNAGISNLFDKQLYR
ENAGASTYNEPGRAYYAGVTLSF
SEQ ID NO: 14
MSRPQFTSLRLSLLALAVSATLPTFAFATETMTVTATGNARSSFEAPMMVSVIDTSAPENQTAT
SATDLLRHVPGITLDGTGRINGQDVNMRGYDHRGVLVLVDGVRQGTDTGHLNGTFLDPALIKRV
EIVRGPSALLYGSGALGGVISYDTVDAKDLLQEGQSSGFRVFGTGGTGDHSLGLGASAFGRTEN
LDGIVAWSSRDRGDLRQSNGETAPNDESINNMLAKGTWQIDSAQSLSGLVRYYNNDAREPKNPQ
TVEASDSSNPMVDRSTIQRDAQLSYKLAPQGNDWLNADAKIYWSEVRINAQNTGSSGEYREQIT
KGARLENRSTLFADSFASHLLTYGGEYYRQEQHPGGATTGFPQAKIDESSGWLQDEITLRDLPI
TLLGGTRYDSYRGSSDGYKDVDADKWSSRAGMTINPTNWLMLEGSYAQAFRAPTMGEMYNDSKH
FSIGRFYTNYWVPNPNLRPETNETQEYGFGLRFDDLMLSNDALEFKASYFDTKAKDYISTTVDF
AAATTMSYNVPNAKIWGWDVMTKYTTDLFSLDVAYNRTRGKDTDTGEYISSINPDTVTSTLNIP
IAHSGFSVGWVGTFADRSTHISSSYSKQPGYGVNDFYVSYQGQQALKGMTTTLVLGNAFDKEYW
SPQGIPQDGRNGKIFVSYQW
SEQ ID NO: 15
MKMTRLYPLALGGLLLPAIANAQTSQQDESTLVVTASKQSSRSASANNVSSTVVSAPELSDAGV
TASDKLPRVLPGLNIENSGNMLFSTISLRGVSSAQDFYNPAVTLYVDGVPQLSTNTIQALTDVQ
SVELLRGPQGTLYGKSAQGGIINIVTQQPDSTPRGYIEGGVSSRDSYRSKENLSGPIQDGLLYG
SVTLLRQVDDGDMINPATGSDDLGGTRASIGNVKLRLAPDDQPWEMGFAASRECTRATQDAYVG
WNDIKGRKLSISDGSPDPYMRRCTDSQTLSGKYTTDDWVENLISAWQQQHYSRTFPSGSLIVNM
PQRWNQDVQELRAATLGDARTVDMVFGLYRQNTREKLNSAYDMPTMPYLSSTGYTTAETLAAYS
DLTWHLTDRFDIGGGVRFSHDKSSTQYHGSMLGNPFGDQGKSNDDQVLGQLSAGYMLTDDWRVY
TRVAQGYKPSGYNIVPTAGLDAKPFVAEKSINYELGTRYETADVTLQAATFYTHTKDMQLYSGP
VGMQTLSNAGKADATGVELEAKWRFAPGWSWDINGNVIRSEFTNDSELYHGNRVPFVPRYGAGS
SVNGVIDTRYGALMPRLAVNLVGPHYFDGDNQLRQGTYATLDSSLGWQATERMNISVYVDNLED
RRYRTYGYMNGSSAVAQVNMGRTVGINTRIDFF
SEQ ID NO: 16
NGTAIPIGGG
SEQ ID NO: 17
HNDYPETITD
SEQ ID NO: 18
SYPFPTTSETPRVVYNSRTDKP
SEQ ID NO: 19
SALALQADARNSDLTITQHGGGNGADVGQGSDDSSID
SEQ ID NO: 20
ATLDQWNGKDSTM
SEQ ID NO: 21
NGAAVDQTASNS
SEQ ID NO: 22
SQQENALWLATSQSRKLDKG
SEQ ID NO: 23
GEVKGRLVLDDRKRTEEVRPL
SEQ ID NO: 24
NKILSRK
SEQ ID NO: 25
VIDAKSYKVVKTED
SEQ ID NO: 26
KQKSTKQQEATQPD
SEQ ID NO: 27
WNQCNGPGFADGSWAY
SEQ ID NO: 28
KGSWSEENTGDN
SEQ ID NO: 29
AGNLCQKPGETTRLTEKF
SEQ ID NO: 30
DLPLGDYSVTI
SEQ ID NO: 31
GARFKIPYNVAKTLP
SEQ ID NO: 32
IVSVNGVDTGETTMRWYKAGTQN
SEQ ID NO: 33
IAYDNKGNASKRVQTEVVISG
SEQ ID NO: 34
PTLGEFTETEAGVYQSVETTGTQSGEA
SEQ ID NO: 35
WTAKDANDNPVTGLNPDAPSLSGAAAAGSTASGWTDNGDGTW
SEQ ID NO: 36
SWTEKGDGSYVATLTTGGKTGELRVMPLENGQP
SEQ ID NO: 37
AAQLTVIAGEMSSANSTLVADNKTPTVKTTTELT
SEQ ID NO: 38
SGAASTGSERPSAGNWTEKG
SEQ ID NO: 39
ESTGTPSNATYTYVIERWDPET
SEQ ID NO: 40
PIEGQTTHRGPSLN
SEQ ID NO: 41
PGSLCGIAPPPVGACKISEG
SEQ ID NO: 42
QTTWVIENAELEQQ
SEQ ID NO: 43
DPFNIDHIEVIS
SEQ ID NO: 44
GGLINIVTKKGQPE
SEQ ID NO: 45
TGTLNIDESRQL
SEQ ID NO: 46
DESLRFYPFPTVNAN
SEQ ID NO: 47
LNFSQGVELPDPG
SEQ ID NO: 48
IAAYYSISDKSVV
SEQ ID NO: 49
DETLVVEATAEQVLK
SEQ ID NO: 50
GPENTLILIDGVPVTSR
SEQ ID NO: 51
NDYQNKIVAGDN
SEQ ID NO: 52
VDGIEASMSFPLVK
SEQ ID NO: 53
INNSLNWTITQAFS
SEQ ID NO: 54
NLRLNVGVSNILNKQ
SEQ ID NO: 55
APMMVSVIDTSAPE
SEQ ID NO: 56
KGTWQIDSAQSL
SEQ ID NO: 57
DAKIYWSEVRINAQ
SEQ ID NO: 58
NPTNWLMLFGSYAQ
SEQ ID NO: 59
VPNAKIWGWDVMTKYT
SEQ ID NO: 60
ISSSYSKQPGYGVN
SEQ ID NO: 61
DFYVSYQGQQALK
SEQ ID NO: 62
GNMLFSTISLRGV
SEQ ID NO: 63
DGVPQLSTNTIQAL
SEQ ID NO: 64
DVQSVELLRGPQGTL
SEQ ID NO: 65
INIVTQQPDSTPR
SEQ ID NO: 66
GYTTAETLAAYSD
SEQ ID NO: 67
HTKDMQLYSGPVGMQT
SEQ ID NO: 68
GNVIRSEFTNDSE
SEQ ID NO: 69
NDSELYHGNRVPFVPR
SEQ ID NO: 70
QGTYATLDSSLGWQ
SEQ ID NO: 71
RVLPGLNIENSGNML
SEQ ID NO: 72
IENSGNMLESTISLR
SEQ ID NO: 73
LFSTISLRGVSSAQD
SEQ ID NO: 74
RGVSSAQDFYNPAVT
SEQ ID NO: 75
DFYNPAVTLYVDGVP
SEQ ID NO: 76
TLYVDGVPQLSINTI
SEQ ID NO: 77
PQLSTNTIQALTDVQ
SEQ ID NO: 78
IQALTDVQSVELLRG
SEQ ID NO: 79
QSVELLRGPQGTLYG
SEQ ID NO: 80
GPQGTLYGKSAQGGI
SEQ ID NO: 81
GKSAQGGIINIVTQQ
SEQ ID NO: 82
IINIVTQQPDSTPRG
SEQ ID NO: 83
SSRDSYRSKFNLSGP
SEQ ID NO: 84
SKFNLSGPIQDGLLY
SEQ ID NO: 85
PIQDGLLYGSVTLLR
SEQ ID NO: 86
YGSVTLLRQVDDGDM
SEQ ID NO: 87
YTTDDWVENLISAWQ
SEQ ID NO: 88
ENLISAWQQQHYSRT
SEQ ID NO: 89
QQQHYSRTFPSGSLI
SEQ ID NO: 90
TFPSGSLIVNMPQRW
SEQ ID NO: 91
MPYLSSTGYTTAETL
SEQ ID NO: 92
GYTTAETLAAYSDLT
SEQ ID NO: 93
LAAYSDLTWHLTDRF
SEQ ID NO: 94
RFSHDKSSTQYHGSM
SEQ ID NO: 95
DQVLGQLSAGYMLTD
SEQ ID NO: 96
SAGYMLTDDWRVYTR
SEQ ID NO: 97
RVAQGYKPSGYNIVP
SEQ ID NO: 98
PSGYNIVPTAGLDAK
SEQ ID NO: 99
PTAGLDAKPFVAEKS
SEQ ID NO: 100
KPFVAEKSINYELGT
SEQ ID NO: 101
SINYELGTRYETADV
SEQ ID NO: 102
TRYETADVTLQAATE
SEQ ID NO: 103
VTLQAATFYTHTKDM
SEQ ID NO: 104
FYTHTKDMQLYSGPV
SEQ ID NO: 105
MQLYSGPVGMQTLSN
SEQ ID NO: 106
VGMQTLSNAGKADAT
SEQ ID NO: 107
NAGKADATGVELEAK
SEQ ID NO: 108
TGVELEAKWRFAPGW
SEQ ID NO: 109
KWRFAPGWSWDINGN
SEQ ID NO: 110
WSWDINGNVIRSEFT
SEQ ID NO: 111
NVIRSEFTNDSELYH
SEQ ID NO: 112
TNDSELYHGNRVPFV
SEQ ID NO: 113
HGNRVPFVPRYGAGS
SEQ ID NO: 114
DTRYGALMPRLAVNL
SEQ ID NO: 115
MPRLAVNLVGPHYFD
SEQ ID NO: 116
LVGPHYFDGDNQLRQ
SEQ ID NO: 117
DGDNQLRQGTYATLD
SEQ ID NO: 118
QGTYATLDSSLGWQA
SEQ ID NO: 119
DSSLGWQATERMNIS
SEQ ID NO: 120
ATERMNISVYVDNLF
SEQ ID NO: 121
YGYMNGSSAVAQVNM
SEQ ID NO: 122
SAVAQVNMGRTVGIN
SEQ ID NO: 123
NMGRTVGINTRIDFF
SEQ ID NO: 124
GSGS
SEQ ID NO: 125
GPGP
SEQ ID NO: 126
LLSVGG
SEQ ID NO: 127
MWTAKDANDNPVTGLNPDAPSLSGAAAAGSTASGWTDNGDGTWGSGSKGTWQIDSAQSLGSGSA
AQLTVIAGEMSSANSTLVADNKTPTVKTTTELTGSGSDESLRFYPFPTVNANGSGSSALALQAD
ARNSDLTITQHGGGNGADVGQGSDDSSIDGSGSGNVIRSEFTNDSEGSGSHNDYPETITDGSGS
NLRLNVGVSNILNKQGSGSSYPFPTTSETPRVVYNSRTDKPGSGSDVQSVELLRGPQGTLGSGS
WNQCNGPGFADGSWAYGSGSGGLINIVTKKGQPEGSGSIVSVNGVDTGETTMRWYKAGTQNGSG
SAPMMVSVIDTSAPEGSGSGEVKGRLVLDDRKRTEEVRPLGSGSVDGIEASMSFPLVKGSGSVI
DAKSYKVVKTFDGSGSQGTYATLDSSLGWQGSGSATLDQWNGKDSTMGSGSQTTWVIENAELEQ
QGSGSESTGTPSNATYTYVIERWDPETGSGSDFYVSYQGQQALKGSGSPIEGQTTHRGPSLNGS
GSDETLVVEATAEQVLKHHHHHH
SEQ ID NO: 128
MKGTWQIDSAQSLGSGSDESLRFYPFPTVNANGSGSGNVIRSEFTNDSEGSGSNLRLNVGVSNI
LNKQGSGSDVQSVELLRGPQGTLGSGSGGLINIVTKKGQPEGSGSAPMMVSVIDTSAPEGSGSV
DGIEASMSFPLVKGSGSQGTYATLDSSLGWQGSGSQTTWVIENAELEQQGSGSDFYVSYQGQQA
LKGSGSDETLVVEATAEQVLKHHHHHH
SEQ ID NO: 129
MWTAKDANDNPVTGLNPDAPSLSGAAAAGSTASGWTDNGDGTWGSGSKGTWQIDSAQSLGSGSA
AQLTVIAGEMSSANSTLVADNKTPTVKTTTELTGSGSDESLRFYPFPTVNANGSGSSALALQAD
ARNSDLTITQHGGGNGADVGQGSDDSSIDGSGSGNVIRSEFTNDSEGSGSHNDYPETITDGSGS
NLRLNVGVSNILNKQGSGSSYPFPTTSETPRVVYNSRTDKPGSGSDVQSVELLRGPQGTLGSGS
WNQCNGPGFADGSWAYGSGSGGLINIVTKKGQPEGSGSIVSVNGVDTGETTMRWYKAGTQNGSG
SAPMMVSVIDTSAPEGSGSGEVKGRLVLDDRKRTEEVRPLGSGSVDGIEASMSFPLVKGSGSVI
DAKSYKVVKTFDGSGSQGTYATLDSSLGWQHHHHHH
SEQ ID NO: 130
MKGTWQIDSAQSLGSGSDESLRFYPFPTVNANGSGSGNVIRSEFTNDSEGSGSNLRLNVGVSNI
LNKQGSGSDVQSVELLRGPQGTLGSGSGGLINIVTKKGQPEGSGSAPMMVSVIDTSAPEGSGSV
DGIEASMSFPLVKGSGSQGTYATLDSSLGWQHHHHHH
SEQ ID NO: 131
MWTAKDANDNPVTGLNPDAPSLSGAAAAGSTASGWTDNGDGTWLLSVGGKGTWQIDSAQSLLLS
VGGAAQLTVIAGEMSSANSTLVADNKTPTVKTTTELTLLSVGGDESLRFYPFPTVNANLLSVGG
SALALQADARNSDLTITQHGGGNGADVGQGSDDSSIDLLSVGGGNVIRSEFTNDSELLSVGGHN
DYPETITDLLSVGGNLRLNVGVSNILNKQLLSVGGSYPFPTTSETPRVVYNSRTDKPLLSVGGD
VQSVELLRGPQGTLLLSVGGWNQCNGPGFADGSWAYLLSVGGGGLINIVTKKGQPELLSVGGIV
SVNGVDTGETTMRWYKAGTQNLLSVGGAPMMVSVIDTSAPELLSVGGGEVKGRLVLDDRKRTEE
VRPLLLSVGGVDGIEASMSFPLVKLLSVGGVIDAKSYKVVKTFDLLSVGGQGTYATLDSSLGWQ
LLSVGGATLDQWNGKDSTMLLSVGGQTTWVIENAELEQQLLSVGGESTGTPSNATYTYVIERWD
PETLLSVGGDFYVSYQGQQALKLLSVGGPIEGQTTHRGPSLNLLSVGGDETLVVEATAEQVLKH
HHHHH
SEQ ID NO: 132
MWTAKDANDNPVTGLNPDAPSLSGAAAAGSTASGWTDNGDGTWGSGSKGTWQIDSAQSLGSGSA
AQLTVIAGEMSSANSTLVADNKTPTVKTTTELTGSGSDESLRFYPFPTVNANGSGSSALALQAD
ARNSDLTITQHGGGNGADVGQGSDDSSIDGSGSHTKDMQLYSGPVGMQTGSGSHNDYPETITDG
SGSNLRLNVGVSNILNKQGSGSSYPFPTTSETPRVVYNSRTDKPGSGSENLISAWQQQHYSRTG
SGSWNQCNGPGFADGSWAYGSGSLNFSQGVELPDPGGSGSIVSVNGVDTGETTMRWYKAGTQNG
SGSAPMMVSVIDTSAPEGSGSGEVKGRLVLDDRKRTEEVRPLGSGSINNSLNWTITQAFSGSGS
VIDAKSYKVVKTFDGSGSGNMLFSTISLRGVGSGSATLDQWNGKDSTMGSGSQTTWVIENAELE
QQGSGSESTGTPSNATYTYVIERWDPETGSGSNPTNWLMLEGSYAQGSGSPIEGQTTHRGPSLN
GSGSNDYQNKIVAGDNHHHHHH
SEQ ID NO: 133
MKGTWQIDSAQSLGSGSDESLRFYPFPTVNANGSGSHTKDMQLYSGPVGMQTGSGSNLRLNVGV
SNILNKQGSGSFNLISAWQQQHYSRTGSGSLNFSQGVELPDPGGSGSAPMMVSVIDTSAPEGSG
SINNSLNWTITQAFSGSGSGNMLESTISLRGVGSGSQTTWVIENAELEQQGSGSNPTNWLMLFG
SYAQGSGSNDYQNKIVAGDNHHHHHH
SEQ ID NO: 134
MWTAKDANDNPVTGLNPDAPSLSGAAAAGSTASGWTDNGDGTWLLSVGGKGTWQIDSAQSLLLS
VGGAAQLTVIAGEMSSANSTLVADNKTPTVKTTTELTLLSVGGDESLRFYPFPTVNANLLSVGG
SALALQADARNSDLTITQHGGGNGADVGQGSDDSSIDLLSVGGHTKDMQLYSGPVGMQTLLSVG
GHNDYPETITDLLSVGGNLRLNVGVSNILNKQLLSVGGSYPFPTTSETPRVVYNSRTDKPLLSV
GGFNLISAWQQQHYSRTLLSVGGWNQCNGPGFADGSWAYLLSVGGLNFSQGVELPDPGLLSVGG
IVSVNGVDTGETTMRWYKAGTQNLLSVGGAPMMVSVIDTSAPELLSVGGGEVKGRLVLDDRKRT
EEVRPLLLSVGGINNSLNWTITQAFSLLSVGGVIDAKSYKVVKTFDLLSVGGGNMLESTISLRG
VLLSVGGATLDQWNGKDSTMLLSVGGQTTWVIENAELEQQLLSVGGESTGTPSNATYTYVIERW
DPETLLSVGGNPTNWLMLFGSYAQLLSVGGPIEGQTTHRGPSLNLLSVGGNDYQNKIVAGDNHH
HHHH
SEQ ID NO: 135
MWTAKDANDNPVTGLNPDAPSLSGAAAAGSTASGWTDNGDGTWGSGSINNSLNWTITQAFSGSG
SAAQLTVIAGEMSSANSTLVADNKTPTVKTTTELTGSGSNPTNWLMLFGSYAQGSGSSALALQA
DARNSDLTITQHGGGNGADVGQGSDDSSIDGSGSNDYQNKIVAGDNGSGSHNDYPETITDGSGS
DESLRFYPFPTVNANGSGSSYPFPTTSETPRVVYNSRTDKPGSGSINNSLNWTITQAFSGSGSW
NQCNGPGFADGSWAYGSGSNPTNWLMLFGSYAQGSGSIVSVNGVDTGETTMRWYKAGTQNGSGS
NDYQNKIVAGDNGSGSGEVKGRLVLDDRKRTEEVRPLGSGSDESLRFYPFPTVNANGSGSVIDA
KSYKVVKTFDGSGSINNSLNWTITQAFSGSGSATLDQWNGKDSTMGSGSNPTNWLMLFGSYAQG
SGSESTGTPSNATYTYVIERWDPETGSGSNDYQNKIVAGDNGSGSPIEGQTTHRGPSLNGSGSD
ESLRFYPFPTVNANHHHHHH
SEQ ID NO: 136
MWTAKDANDNPVTGLNPDAPSLSGAAAAGSTASGWTDNGDGTWGSGSKGTWQIDSAQSLGSGSA
AQLTVIAGEMSSANSTLVADNKTPTVKTTTELTGSGSDESLRFYPFPTVNANGSGSSALALQAD
ARNSDLTITQHGGGNGADVGQGSDDSSIDGSGSHTKDMQLYSGPVGMQTGSGSHNDYPETITDG
SGSNLRLNVGVSNILNKQGSGSSYPFPTTSETPRVVYNSRTDKPGSGSENLISAWQQQHYSRTG
SGSWNQCNGPGFADGSWAYGSGSLNFSQGVELPDPGGSGSIVSVNGVDTGETTMRWYKAGTQNG
SGSAPMMVSVIDTSAPEGSGSGEVKGRLVLDDRKRTEEVRPLGSGSINNSLNWTITQAFSGSGS
VIDAKSYKVVKTFDGSGSPIQDGLLYGSVTLLRGSGSATLDQWNGKDSTMGSGSQTTWVIENAE
LEQQGSGSESTGTPSNATYTYVIERWDPETGSGSNPTNWLMLEGSYAQGSGSPIEGQTTHRGPS
LNGSGSNDYQNKIVAGDNHHHHHH
SEQ ID NO: 137
MKGTWQIDSAQSLGSGSDESLRFYPFPTVNANGSGSHTKDMQLYSGPVGMQTGSGSNLRLNVGV
SNILNKQGSGSENLISAWQQQHYSRTGSGSLNFSQGVELPDPGGSGSAPMMVSVIDTSAPEGSG
SINNSLNWTITQAFSGSGSPIQDGLLYGSVTLLRGSGSQTTWVIENAELEQQGSGSNPTNWLML
FGSYAQGSGSNDYQNKIVAGDNHHHHHH
SEQ ID NO: 138
MWTAKDANDNPVTGLNPDAPSLSGAAAAGSTASGWTDNGDGTWGSGSKGTWQIDSAQSLGSGSA
AQLTVIAGEMSSANSTLVADNKTPTVKTTTELTGSGSDESLRFYPFPTVNANGSGSSALALQAD
ARNSDLTITQHGGGNGADVGQGSDDSSIDGSGSHTKDMQLYSGPVGMQTGSGSHNDYPETITDG
SGSNLRLNVGVSNILNKQGSGSSYPFPTTSETPRVVYNSRTDKPGSGSENLISAWQQQHYSRTG
SGSWNQCNGPGFADGSWAYGSGSLNFSQGVELPDPGGSGSIVSVNGVDTGETTMRWYKAGTQNG
SGSAPMMVSVIDTSAPEGSGSGEVKGRLVLDDRKRTEEVRPLGSGSINNSLNWTITQAFSGSGS
VIDAKSYKVVKTFDGSGSGNMLESTISLRGVGSGSATLDQWNGKDSTMGSGSQTTWVIENAELE
QQGSGSESTGTPSNATYTYVIERWDPETGSGSNPTNWLMLFGSYAQGSGSPIEGQTTHRGPSLN
GSGSNDYQNKIVAGDNGSGSTGATIPIGGGGSGSPIQDGLLYGSVTLLRHHHHHH
SEQ ID NO: 139
MKGTWQIDSAQSLGSGSDESLRFYPFPTVNANGSGSHTKDMQLYSGPVGMQTGSGSNLRLNVGV
SNILNKQGSGSENLISAWQQQHYSRTGSGSLNFSQGVELPDPGGSGSAPMMVSVIDTSAPEGSG
SINNSLNWTITQAFSGSGSGNMLESTISLRGVGSGSQTTWVIENAELEQQGSGSNPTNWLMLFG
SYAQGSGSNDYQNKIVAGDNGSGSPIQDGLLYGSVTLLRHHHHHH
SEQ ID NO: 140
MWTAKDANDNPVTGLNPDAPSLSGAAAAGSTASGWTDNGDGTWGSGSKGTWQIDSAQSLGSGSA
AQLTVIAGEMSSANSTLVADNKTPTVKTTTELTGSGSDESLRFYPFPTVNANGSGSSALALQAD
ARNSDLTITQHGGGNGADVGQGSDDSSIDGSGSHTKDMQLYSGPVGMQTGSGSHNDYPETITDG
SGSNLRLNVGVSNILNKQGSGSSYPFPTTSETPRVVYNSRTDKPGSGSENLISAWQQQHYSRTG
SGSWNQCNGPGFADGSWAYGSGSLNFSQGVELPDPGGSGSIVSVNGVDTGETTMRWYKAGTQNG
SGSAPMMVSVIDTSAPEGSGSGEVKGRLVLDDRKRTEEVRPLGSGSINNSLNWTITQAFSGSGS
VIDAKSYKVVKTFDGSGSPIQDGLLYGSVTLLRGSGSATLDQWNGKDSTMGSGSQTTWVIENAE
LEQQGSGSESTGTPSNATYTYVIERWDPETGSGSNPTNWLMLEGSYAQGSGSPIEGQTTHRGPS
LNGSGSNDYQNKIVAGDNGSGSTGATIPIGGGHHHHHH
SEQ ID NO: 141
MWTAKDANDNPVTGLNPDAPSLSGAAAAGSTASGWTDNGDGTWGSGSAAQLTVIAGEMSSANST
LVADNKTPTVKITTELTGSGSSALALQADARNSDLTITQHGGGNGADVGQGSDDSSIDGSGSHN
DYPETITDGSGSKGTWQIDSAQSLGSGSDESLRFYPFPTVNANGSGSHTKDMQLYSGPVGMQTG
SGSNLRLNVGVSNILNKQGSGSSYPFPTTSETPRVVYNSRTDKPGSGSWNQCNGPGFADGSWAY
GSGSIVSVNGVDTGETTMRWYKAGTQNGSGSGEVKGRLVLDDRKRTEEVRPLGSGSENLISAWQ
QQHYSRTGSGSLNFSQGVELPDPGGSGSAPMMVSVIDTSAPEGSGSINNSLNWTITQAFSGSGS
VIDAKSYKVVKTFDGSGSATLDQWNGKDSTMGSGSESTGTPSNATYTYVIERWDPETGSGSPIE
GQTTHRGPSLNGSGSPIQDGLLYGSVTLLRGSGSQTTWVIENAELEQQGSGSNPTNWLMLFGSY
AQGSGSNDYQNKIVAGDNHHHHHH
SEQ ID NO: 142
MKGTWQIDSAQSLGSGSDESLRFYPFPTVNANGSGSHTKDMQLYSGPVGMQTGSGSNLRLNVGV
SNILNKQGSGSWTAKDANDNPVTGLNPDAPSLSGAAAAGSTASGWTDNGDGTWGSGSAAQLTVI
AGEMSSANSTLVADNKTPTVKTTTELTGSGSSALALQADARNSDLTITQHGGGNGADVGQGSDD
SSIDGSGSHNDYPETITDGSGSFNLISAWQQQHYSRTGSGSLNFSQGVELPDPGGSGSAPMMVS
VIDTSAPEGSGSINNSLNWTITQAFSGSGSSYPFPTTSETPRVVYNSRTDKPGSGSWNQCNGPG
FADGSWAYGSGSIVSVNGVDTGETTMRWYKAGTQNGSGSGEVKGRLVLDDRKRTEEVRPLGSGS
PIQDGLLYGSVTLLRGSGSQTTWVIENAELEQQGSGSNPTNWLMLEGSYAQGSGSNDYQNKIVA
GDNGSGSVIDAKSYKVVKTFDGSGSATLDQWNGKDSTMGSGSESTGTPSNATYTYVIERWDPET
GSGSPIEGQTTHRGPSLNHHHHHH
SEQ ID NO: 143
MAAQLTVIAGEMSSANSTLVADNKTPTVKTTTELTGSGSKGTWQIDSAQSLGSGSWTAKDANDN
PVTGLNPDAPSLSGAAAAGSTASGWTDNGDGTWGSGSDESLRFYPFPTVNANGSGSSALALQAD
ARNSDLTITQHGGGNGADVGQGSDDSSIDGSGSHTKDMQLYSGPVGMQTGSGSHNDYPETITDG
SGSNLRLNVGVSNILNKQGSGSSYPFPTTSETPRVVYNSRTDKPGSGSENLISAWQQQHYSRTG
SGSWNQCNGPGFADGSWAYGSGSLNFSQGVELPDPGGSGSIVSVNGVDTGETTMRWYKAGTQNG
SGSAPMMVSVIDTSAPEGSGSGEVKGRLVLDDRKRTEEVRPLGSGSINNSLNWTITQAFSGSGS
VIDAKSYKVVKTFDGSGSPIQDGLLYGSVTLLRGSGSATLDQWNGKDSTMGSGSQTTWVIENAE
LEQQGSGSESTGTPSNATYTYVIERWDPETGSGSNPTNWLMLEGSYAQGSGSPIEGQTTHRGPS
LNGSGSNDYQNKIVAGDNHHHHHH
SEQ ID NO: 144
MWTAKDANDNPVTGLNPDAPSLSGAAAAGSTASGWTDNGDGTWGSGSDESLRFYPFPTVNANGS
GSAAQLTVIAGEMSSANSTLVADNKTPTVKTTTELTGSGSKGTWQIDSAQSLGSGSSALALQAD
ARNSDLTITQHGGGNGADVGQGSDDSSIDGSGSHTKDMQLYSGPVGMQTGSGSHNDYPETITDG
SGSNLRLNVGVSNILNKQGSGSSYPFPTTSETPRVVYNSRTDKPGSGSENLISAWQQQHYSRTG
SGSWNQCNGPGFADGSWAYGSGSLNFSQGVELPDPGGSGSIVSVNGVDTGETTMRWYKAGTQNG
SGSAPMMVSVIDTSAPEGSGSGEVKGRLVLDDRKRTEEVRPLGSGSINNSLNWTITQAFSGSGS
VIDAKSYKVVKTFDGSGSPIQDGLLYGSVTLLRGSGSATLDQWNGKDSTMGSGSQTTWVIENAE
LEQQGSGSESTGTPSNATYTYVIERWDPETGSGSNPTNWLMLEGSYAQGSGSPIEGQTTHRGPS
LNGSGSNDYQNKIVAGDNHHHHHH
SEQ ID NO: 145
MDESLRFYPFPTVNANGSGSKGTWQIDSAQSLGSGSHTKDMQLYSGPVGMQTGSGSNLRLNVGV
SNILNKQGSGSENLISAWQQQHYSRTGSGSLNFSQGVELPDPGGSGSAPMMVSVIDTSAPEGSG
SINNSLNWTITQAFSGSGSPIQDGLLYGSVTLLRGSGSQTTWVIENAELEQQGSGSNPTNWLML
FGSYAQGSGSNDYQNKIVAGDNHHHHHH
SEQ ID NO: 146
SGSG
SEQ ID NO: 147
SGSGSGSG
SEQ ID NO: 148
SGSGSGSGSGSG
SEQ ID NO: 149
SGSGSGSGSGSGSGSG
SEQ ID NO: 150
SGSGSGSGSGSGSGSGSGSG
SEQ ID NO: 151
SGSGSGSGSGSGSGSGSGSGSGSG
SEQ ID NO: 152
SGSGSGSGSGSGSGSGSGSGSGSGSGSG
SEQ ID NO: 153
SGSGSGSGSGSGSGSGSGSGSGSGSGSGSGSG
SEQ ID NO: 154
SGSGSGSGSGSGSGSGSGSGSGSGSGSGSGSGSGSG
SEQ ID NO: 155
SGSGSGSGSGSGSGSGSGSGSGSGSGSGSGSGSGSGSGSG
SEQ ID NO: 156
SSSS
SEQ ID NO: 157
GGGS
SEQ ID NO: 158
GGC
SEQ ID NO: 159
GGS
SEQ ID NO: 160
GGCGGCGGCGGCGGCGGCGGCGGC
SEQ ID NO: 161
GGGGSGGGGSGGGGS
SEQ ID NO: 162
GGAAY
SEQ ID NO: 163
SALALQADARNSDLT
SEQ ID NO: 164
QADARNSDLTITQHG
SEQ ID NO: 165
NSDLTITQHGGGNGA
SEQ ID NO: 166
ITQHGGGNGADVGQG
SEQ ID NO: 167
GGNGADVGQGSDDSS
SEQ ID NO: 168
NGADVGQGSDDSSID
SEQ ID NO: 169
ATLDQWNGKDSTM
SEQ ID NO: 170
NGAAVDQTASNS
SEQ ID NO: 171
IAYDNKGNASKRVQT
SEQ ID NO: 172
KGNASKRVQTEVVISG
SEQ ID NO: 173
PTLGEFTETEAGVYQ
SEQ ID NO: 174
AGVYQSVETTGTQSG
SEQ ID NO: 175
VYQSVFTTGTQSGEA
SEQ ID NO: 176
WTAKDANDNPVTGLN
SEQ ID NO: 177
ANDNPVTGLNPDAPS
SEQ ID NO: 178
VTGLNPDAPSLSGAA
SEQ ID NO: 179
PDAPSLSGAAAAGST
SEQ ID NO: 180
LSGAAAAGSTASGWT
SEQ ID NO: 181
AAGSTASGWTDNGDG
SEQ ID NO: 182
GSTASGWTDNGDGTW
SEQ ID NO: 183
SWTEKGDGSY
SEQ ID NO: 184
GDGSYVATLTTGGKT
SEQ ID NO: 185
VATLTTGGKTGELRV
SEQ ID NO: 186
TGGKTGELRVMPLEN
SEQ ID NO: 187
KTGELRVMPLENGQP
SEQ ID NO: 188
AAQLTVIAGEM
SEQ ID NO: 189
VIAGEMSSANSTLVA
SEQ ID NO: 190
MSSANSTLVADNKTP
SEQ ID NO: 191
STLVADNKTPTVKTT
SEQ ID NO: 192
ADNKTPTVKTTTELT
SEQ ID NO: 193
SGAASTGSERPSAGN
SEQ ID NO: 194
TGSERPSAGNWTEKG
SEQ ID NO: 195
TGATIPIGGG
SEQ ID NO: 196
HNDYPETITD
SEQ ID NO: 197
SYPFPTTSETPRVVY
SEQ ID NO: 198
TTSETPRVVYNSRTD
SEQ ID NO: 199
SETPRVVYNSRTDKP
SEQ ID NO: 200
ESTGTPSNATYTYVI
SEQ ID NO: 201
PSNATYTYVIERWDP
SEQ ID NO: 202
NATYTYVIERWDPET
SEQ ID NO: 203
PIEGQTTHRGPSLN
SEQ ID NO: 204
PGSLCGIAPPPVGAC
SEQ ID NO: 205
GIAPPPVGACKISEG
SEQ ID NO: 206
WNQCNGPGFADGSWAY
SEQ ID NO: 207
GPGFADGSWAY
SEQ ID NO: 208
KGSWSEENTGDN
SEQ ID NO: 209
AGNLCQKPGETTRLT
SEQ ID NO: 210
LCQKPGETTRLTEKF
SEQ ID NO: 211
DLPLGDYSVTI
SEQ ID NO: 212
GARFKIPYNVAKTLP
SEQ ID NO: 213
IVSVNGVDTGETTMR
SEQ ID NO: 214
GVDTGETTMRWYKAG
SEQ ID NO: 215
TGETTMRWYKAGTQN
SEQ ID NO: 216
SQQENALWLATSQSR
SEQ ID NO: 217
ALWLATSQSRKLDKG
SEQ ID NO: 218
GEVKGRLVLDDRKRT
SEQ ID NO: 219
RLVLDDRKRTEEVRPL
SEQ ID NO: 220
NKILSRK
SEQ ID NO: 221
VIDAKSYKVVKTFD
SEQ ID NO: 222
KQKSTKQQEATQPD
SEQ ID NO: 223
MWTAKDANDNPVTGLNPDAPSLSGAAAAGSTASGWTDNGDGTWGSGSKGTWQIDSAQSLGSGSA
AQLTVIAGEMSSANSTLVADNKTPTVKTTTELTGSGSDESLRFYPFPTVNANGSGSSALALQAD
ARNSDLTITQHGGGNGADVGQGSDDSSIDGSGSHTKDMQLYSGPVGMQTGSGSHNDYPETITDG
SGSNLRLNVGVSNILNKQGSGSSYPFPTTSETPRVVYNSRTDKPGSGSENLISAWQQQHYSRTG
SGSWNQCNGPGFADGSWAYGSGSLNFSQGVELPDPGGSGSIVSVNGVDTGETTMRWYKAGTQNG
SGSAPMMVSVIDTSAPEGSGSGEVKGRLVLDDRKRTEEVRPLGSGSINNSLNWTITQAFSGSGS
VIDAKSYKVVKTFDGSGSPIQDGLLYGSVTLLRGSGSATLDQWNGKDSTMGSGSQTTWVIENAE
LEQQGSGSESTGTPSNATYTYVIERWDPETGSGSNPTNWLMLEGSYAQGSGSPIEGQTTHRGPS
LNGSGSNDYQNKIVAGDN
SEQ ID NO: 224
MKGTWQIDSAQSLGSGSDESLRFYPFPTVNANGSGSHTKDMQLYSGPVGMQTGSGSNLRLNVGV
SNILNKQGSGSENLISAWQQQHYSRTGSGSLNFSQGVELPDPGGSGSAPMMVSVIDTSAPEGSG
SINNSLNWTITQAFSGSGSPIQDGLLYGSVTLLRGSGSQTTWVIENAELEQQGSGSNPTNWLML
FGSYAQGSGSNDYQNKIVAGDN
SEQ ID NO: 225
MWTAKDANDNPVTGLNPDAPSLSGAAAAGSTASGWTDNGDGTWGSGSKGTWQIDSAQSLGSGSA
AQLTVIAGEMSSANSTLVADNKTPTVKTTTELTGSGSDESLRFYPFPTVNANGSGSSALALQAD
ARNSDLTITQHGGGNGADVGQGSDDSSIDGSGSHTKDMQLYSGPVGMQTGSGSHNDYPETITDG
SGSNLRLNVGVSNILNKQGSGSSYPFPTTSETPRVVYNSRTDKPGSGSENLISAWQQQHYSRTG
SGSWNQCNGPGFADGSWAYGSGSLNFSQGVELPDPGGSGSIVSVNGVDTGETTMRWYKAGTQNG
SGSAPMMVSVIDTSAPEGSGSGEVKGRLVLDDRKRTEEVRPLGSGSINNSLNWTITQAFSGSGS
VIDAKSYKVVKTFDGSGSGNMLFSTISLRGVGSGSATLDQWNGKDSTMGSGSQTTWVIENAELE
QQGSGSESTGTPSNATYTYVIERWDPETGSGSNPTNWLMLEGSYAQGSGSPIEGQTTHRGPSLN
GSGSNDYQNKIVAGDNGSGSTGATIPIGGGGSGSPIQDGLLYGSVTLLR
SEQ ID NO: 226
MKGTWQIDSAQSLGSGSDESLRFYPFPTVNANGSGSHTKDMQLYSGPVGMQTGSGSNLRLNVGV
SNILNKQGSGSFNLISAWQQQHYSRTGSGSLNFSQGVELPDPGGSGSAPMMVSVIDTSAPEGSG
SINNSLNWTITQAFSGSGSGNMLESTISLRGVGSGSQTTWVIENAELEQQGSGSNPTNWLMLFG
SYAQGSGSNDYQNKIVAGDNGSGSPIQDGLLYGSVTLLR
SEQ ID NO: 227
MDESLRFYPFPTVNANGSGSKGTWQIDSAQSLGSGSHTKDMQLYSGPVGMQTGSGSNLRLNVGV
SNILNKQGSGSFNLISAWQQQHYSRTGSGSLNFSQGVELPDPGGSGSAPMMVSVIDTSAPEGSG
SINNSLNWTITQAFSGSGSPIQDGLLYGSVTLLRGSGSQTTWVIENAELEQQGSGSNPTNWLML
FGSYAQGSGSNDYQNKIVAGDN
SEQ ID NO: 228
MWTAKDANDNPVTGLNPDAPSLSGAAAAGSTASGWTDNGDGTWGSGSKGTWQIDSAQSLGSGSA
AQLTVIAGEMSSANSTLVADNKTPTVKTTTELTGSGSDESLRFYPFPTVNANGSGSSALALQAD
ARNSDLTITQHGGGNGADVGQGSDDSSIDGSGSGNVIRSEFTNDSEGSGSHNDYPETITDGSGS
NLRLNVGVSNILNKQGSGSSYPFPTTSETPRVVYNSRTDKPGSGSDVQSVELLRGPQGTLGSGS
WNQCNGPGFADGSWAYGSGSGGLINIVTKKGQPEGSGSIVSVNGVDTGETTMRWYKAGTQNGSG
SAPMMVSVIDTSAPEGSGSGEVKGRLVLDDRKRTEEVRPLGSGSVDGIEASMSFPLVKGSGSVI
DAKSYKVVKTFDGSGSQGTYATLDSSLGWQGSGSATLDQWNGKDSTMGSGSQTTWVIENAELEQ
QGSGSESTGTPSNATYTYVIERWDPETGSGSDFYVSYQGQQALKGSGSPIEGQTTHRGPSLNGS
GSDETLVVEATAEQVLK
SEQ ID NO: 229
MKGTWQIDSAQSLGSGSDESLRFYPFPTVNANGSGSGNVIRSEFTNDSEGSGSNLRLNVGVSNI
LNKQGSGSDVQSVELLRGPQGTLGSGSGGLINIVTKKGQPEGSGSAPMMVSVIDTSAPEGSGSV
DGIEASMSFPLVKGSGSQGTYATLDSSLGWQGSGSQTTWVIENAELEQQGSGSDFYVSYQGQQA
LKGSGSDETLVVEATAEQVLK
SEQ ID NO: 230
MWTAKDANDNPVTGLNPDAPSLSGAAAAGSTASGWTDNGDGTWGSGSKGTWQIDSAQSLGSGSA
AQLTVIAGEMSSANSTLVADNKTPTVKTTTELTGSGSDESLRFYPFPTVNANGSGSSALALQAD
ARNSDLTITQHGGGNGADVGQGSDDSSIDGSGSGNVIRSEFTNDSEGSGSHNDYPETITDGSGS
NLRLNVGVSNILNKQGSGSSYPFPTTSETPRVVYNSRTDKPGSGSDVQSVELLRGPQGTLGSGS
WNQCNGPGFADGSWAYGSGSGGLINIVTKKGQPEGSGSIVSVNGVDTGETTMRWYKAGTQNGSG
SAPMMVSVIDTSAPEGSGSGEVKGRLVLDDRKRTEEVRPLGSGSVDGIEASMSFPLVKGSGSVI
DAKSYKVVKTFDGSGSQGTYATLDSSLGWQ
SEQ ID NO: 231
MKGTWQIDSAQSLGSGSDESLRFYPFPTVNANGSGSGNVIRSEFTNDSEGSGSNLRLNVGVSNI
LNKQGSGSDVQSVELLRGPQGTLGSGSGGLINIVTKKGQPEGSGSAPMMVSVIDTSAPEGSGSV
DGIEASMSFPLVKGSGSQGTYATLDSSLGWQ
SEQ ID NO: 232
MWTAKDANDNPVTGLNPDAPSLSGAAAAGSTASGWTDNGDGTWLLSVGGKGTWQIDSAQSLLLS
VGGAAQLTVIAGEMSSANSTLVADNKTPTVKTTTELTLLSVGGDESLRFYPFPTVNANLLSVGG
SALALQADARNSDLTITQHGGGNGADVGQGSDDSSIDLLSVGGGNVIRSEFTNDSELLSVGGHN
DYPETITDLLSVGGNLRLNVGVSNILNKQLLSVGGSYPFPTTSETPRVVYNSRTDKPLLSVGGD
VQSVELLRGPQGTLLLSVGGWNQCNGPGFADGSWAYLLSVGGGGLINIVTKKGQPELLSVGGIV
SVNGVDTGETTMRWYKAGTQNLLSVGGAPMMVSVIDTSAPELLSVGGGEVKGRLVLDDRKRTEE
VRPLLLSVGGVDGIEASMSFPLVKLLSVGGVIDAKSYKVVKTFDLLSVGGQGTYATLDSSLGWQ
LLSVGGATLDQWNGKDSTMLLSVGGQTTWVIENAELEQQLLSVGGESTGTPSNATYTYVIERWD
PETLLSVGGDFYVSYQGQQALKLLSVGGPIEGQTTHRGPSLNLLSVGGDETLVVEATAEQVLK
SEQ ID NO: 233
MWTAKDANDNPVTGLNPDAPSLSGAAAAGSTASGWTDNGDGTWGSGSKGTWQIDSAQSLGSGSA
AQLTVIAGEMSSANSTLVADNKTPTVKTTTELTGSGSDESLRFYPFPTVNANGSGSSALALQAD
ARNSDLTITQHGGGNGADVGQGSDDSSIDGSGSHTKDMQLYSGPVGMQTGSGSHNDYPETITDG
SGSNLRLNVGVSNILNKQGSGSSYPFPTTSETPRVVYNSRTDKPGSGSENLISAWQQQHYSRTG
SGSWNQCNGPGFADGSWAYGSGSLNFSQGVELPDPGGSGSIVSVNGVDTGETTMRWYKAGTQNG
SGSAPMMVSVIDTSAPEGSGSGEVKGRLVLDDRKRTEEVRPLGSGSINNSLNWTITQAFSGSGS
VIDAKSYKVVKTFDGSGSGNMLFSTISLRGVGSGSATLDQWNGKDSTMGSGSQTTWVIENAELE
QQGSGSESTGTPSNATYTYVIERWDPETGSGSNPTNWLMLFGSYAQGSGSPIEGQTTHRGPSLN
GSGSNDYQNKIVAGDN
SEQ ID NO: 234
MKGTWQIDSAQSLGSGSDESLRFYPFPTVNANGSGSHTKDMQLYSGPVGMQTGSGSNLRLNVGV
SNILNKQGSGSENLISAWQQQHYSRTGSGSLNFSQGVELPDPGGSGSAPMMVSVIDTSAPEGSG
SINNSLNWTITQAFSGSGSGNMLESTISLRGVGSGSQTTWVIENAELEQQGSGSNPTNWLMLFG
SYAQGSGSNDYQNKIVAGDN
SEQ ID NO: 235
MWTAKDANDNPVTGLNPDAPSLSGAAAAGSTASGWTDNGDGTWLLSVGGKGTWQIDSAQSLLLS
VGGAAQLTVIAGEMSSANSTLVADNKTPTVKTTTELTLLSVGGDESLRFYPFPTVNANLLSVGG
SALALQADARNSDLTITQHGGGNGADVGQGSDDSSIDLLSVGGHTKDMQLYSGPVGMQTLLSVG
GHNDYPETITDLLSVGGNLRLNVGVSNILNKQLLSVGGSYPFPTTSETPRVVYNSRTDKPLLSV
GGFNLISAWQQQHYSRTLLSVGGWNQCNGPGFADGSWAYLLSVGGLNFSQGVELPDPGLLSVGG
IVSVNGVDTGETTMRWYKAGTQNLLSVGGAPMMVSVIDTSAPELLSVGGGEVKGRLVLDDRKRT
EEVRPLLLSVGGINNSLNWTITQAFSLLSVGGVIDAKSYKVVKTFDLLSVGGGNMLESTISLRG
VLLSVGGATLDQWNGKDSTMLLSVGGQTTWVIENAELEQQLLSVGGESTGTPSNATYTYVIERW
DPETLLSVGGNPTNWLMLEGSYAQLLSVGGPIEGQTTHRGPSLNLLSVGGNDYQNKIVAGDN
SEQ ID NO: 236
MWTAKDANDNPVTGLNPDAPSLSGAAAAGSTASGWTDNGDGTWGSGSINNSLNWTITQAFSGSG
SAAQLTVIAGEMSSANSTLVADNKTPTVKTTTELTGSGSNPTNWLMLFGSYAQGSGSSALALQA
DARNSDLTITQHGGGNGADVGQGSDDSSIDGSGSNDYQNKIVAGDNGSGSHNDYPETITDGSGS
DESLRFYPFPTVNANGSGSSYPFPTTSETPRVVYNSRTDKPGSGSINNSLNWTITQAFSGSGSW
NQCNGPGFADGSWAYGSGSNPTNWLMLFGSYAQGSGSIVSVNGVDTGETTMRWYKAGTQNGSGS
NDYQNKIVAGDNGSGSGEVKGRLVLDDRKRTEEVRPLGSGSDESLRFYPFPTVNANGSGSVIDA
KSYKVVKTFDGSGSINNSLNWTITQAFSGSGSATLDQWNGKDSTMGSGSNPTNWLMLEGSYAQG
SGSESTGTPSNATYTYVIERWDPETGSGSNDYQNKIVAGDNGSGSPIEGQTTHRGPSLNGSGSD
ESLRFYPFPTVNAN
SEQ ID NO: 237
MWTAKDANDNPVTGLNPDAPSLSGAAAAGSTASGWTDNGDGTWGSGSKGTWQIDSAQSLGSGSA
AQLTVIAGEMSSANSTLVADNKTPTVKTTTELTGSGSDESLRFYPFPTVNANGSGSSALALQAD
ARNSDLTITQHGGGNGADVGQGSDDSSIDGSGSHTKDMQLYSGPVGMQTGSGSHNDYPETITDG
SGSNLRLNVGVSNILNKQGSGSSYPFPTTSETPRVVYNSRTDKPGSGSENLISAWQQQHYSRTG
SGSWNQCNGPGFADGSWAYGSGSLNFSQGVELPDPGGSGSIVSVNGVDTGETTMRWYKAGTQNG
SGSAPMMVSVIDTSAPEGSGSGEVKGRLVLDDRKRTEEVRPLGSGSINNSLNWTITQAFSGSGS
VIDAKSYKVVKTFDGSGSPIQDGLLYGSVTLLRGSGSATLDQWNGKDSTMGSGSQTTWVIENAE
LEQQGSGSESTGTPSNATYTYVIERWDPETGSGSNPTNWLMLEGSYAQGSGSPIEGQTTHRGPS
LNGSGSNDYQNKIVAGDNGSGSTGATIPIGGG
SEQ ID NO: 238
MWTAKDANDNPVTGLNPDAPSLSGAAAAGSTASGWTDNGDGTWGSGSAAQLTVIAGEMSSANST
LVADNKTPTVKTTTELTGSGSSALALQADARNSDLTITQHGGGNGADVGQGSDDSSIDGSGSHN
DYPETITDGSGSKGTWQIDSAQSLGSGSDESLRFYPFPTVNANGSGSHTKDMQLYSGPVGMQTG
SGSNLRLNVGVSNILNKQGSGSSYPFPTTSETPRVVYNSRTDKPGSGSWNQCNGPGFADGSWAY
GSGSIVSVNGVDTGETTMRWYKAGTQNGSGSGEVKGRLVLDDRKRTEEVRPLGSGSENLISAWQ
QQHYSRTGSGSLNFSQGVELPDPGGSGSAPMMVSVIDTSAPEGSGSINNSLNWTITQAFSGSGS
VIDAKSYKVVKTFDGSGSATLDQWNGKDSTMGSGSESTGTPSNATYTYVIERWDPETGSGSPIE
GQTTHRGPSLNGSGSPIQDGLLYGSVTLLRGSGSQTTWVIENAELEQQGSGSNPTNWLMLEGSY
AQGSGSNDYQNKIVAGDN
SEQ ID NO: 239
MKGTWQIDSAQSLGSGSDESLRFYPFPTVNANGSGSHTKDMQLYSGPVGMQTGSGSNLRLNVGV
SNILNKQGSGSWTAKDANDNPVTGLNPDAPSLSGAAAAGSTASGWTDNGDGTWGSGSAAQLTVI
AGEMSSANSTLVADNKTPTVKTTTELTGSGSSALALQADARNSDLTITQHGGGNGADVGQGSDD
SSIDGSGSHNDYPETITDGSGSENLISAWQQQHYSRTGSGSLNFSQGVELPDPGGSGSAPMMVS
VIDTSAPEGSGSINNSLNWTITQAFSGSGSSYPFPTTSETPRVVYNSRTDKPGSGSWNQCNGPG
FADGSWAYGSGSIVSVNGVDTGETTMRWYKAGTQNGSGSGEVKGRLVLDDRKRTEEVRPLGSGS
PIQDGLLYGSVTLLRGSGSQTTWVIENAELEQQGSGSNPTNWLMLFGSYAQGSGSNDYQNKIVA
GDNGSGSVIDAKSYKVVKTFDGSGSATLDQWNGKDSTMGSGSESTGTPSNATYTYVIERWDPET
GSGSPIEGQTTHRGPSLN
SEQ ID NO: 240
MAAQLTVIAGEMSSANSTLVADNKTPTVKTTTELTGSGSKGTWQIDSAQSLGSGSWTAKDANDN
PVTGLNPDAPSLSGAAAAGSTASGWTDNGDGTWGSGSDESLRFYPFPTVNANGSGSSALALQAD
ARNSDLTITQHGGGNGADVGQGSDDSSIDGSGSHTKDMQLYSGPVGMQTGSGSHNDYPETITDG
SGSNLRLNVGVSNILNKQGSGSSYPFPTTSETPRVVYNSRTDKPGSGSENLISAWQQQHYSRTG
SGSWNQCNGPGFADGSWAYGSGSLNFSQGVELPDPGGSGSIVSVNGVDTGETTMRWYKAGTQNG
SGSAPMMVSVIDTSAPEGSGSGEVKGRLVLDDRKRTEEVRPLGSGSINNSLNWTITQAFSGSGS
VIDAKSYKVVKTFDGSGSPIQDGLLYGSVTLLRGSGSATLDQWNGKDSTMGSGSQTTWVIENAE
LEQQGSGSESTGTPSNATYTYVIERWDPETGSGSNPTNWLMLEGSYAQGSGSPIEGQTTHRGPS
LNGSGSNDYQNKIVAGDN
SEQ ID NO: 241
MWTAKDANDNPVTGLNPDAPSLSGAAAAGSTASGWTDNGDGTWGSGSDESLRFYPFPTVNANGS
GSAAQLTVIAGEMSSANSTLVADNKTPTVKTTTELTGSGSKGTWQIDSAQSLGSGSSALALQAD
ARNSDLTITQHGGGNGADVGQGSDDSSIDGSGSHTKDMQLYSGPVGMQTGSGSHNDYPETITDG
SGSNLRLNVGVSNILNKQGSGSSYPFPTTSETPRVVYNSRTDKPGSGSENLISAWQQQHYSRTG
SGSWNQCNGPGFADGSWAYGSGSLNFSQGVELPDPGGSGSIVSVNGVDTGETTMRWYKAGTQNG
SGSAPMMVSVIDTSAPEGSGSGEVKGRLVLDDRKRTEEVRPLGSGSINNSLNWTITQAFSGSGS
VIDAKSYKVVKIFDGSGSPIQDGLLYGSVTLLRGSGSATLDQWNGKDSTMGSGSQTTWVIENAE
LEQQGSGSESTGTPSNATYTYVIERWDPETGSGSNPTNWLMLEGSYAQGSGSPIEGQTTHRGPS
LNGSGSNDYQNKIVAGDN
SEQ ID NO: 242
WTAKDANDNPVTGLNPDAPSLSGAAAAGSTASGWTDNGDGTWGSGSKGTWQIDSAQSLGSGSAA
QLTVIAGEMSSANSTLVADNKTPTVKTTTELTGSGSDESLRFYPFPTVNANGSGSSALALQADA
RNSDLTITQHGGGNGADVGQGSDDSSIDGSGSHTKDMQLYSGPVGMQTGSGSHNDYPETITDGS
GSNLRLNVGVSNILNKQGSGSSYPFPTTSETPRVVYNSRTDKPGSGSENLISAWQQQHYSRTGS
GSWNQCNGPGFADGSWAYGSGSLNFSQGVELPDPGGSGSIVSVNGVDTGETTMRWYKAGTQNGS
GSAPMMVSVIDTSAPEGSGSGEVKGRLVLDDRKRTEEVRPLGSGSINNSLNWTITQAFSGSGSV
IDAKSYKVVKTFDGSGSPIQDGLLYGSVTLLRGSGSATLDQWNGKDSTMGSGSQTTWVIENAEL
EQQGSGSESTGTPSNATYTYVIERWDPETGSGSNPTNWLMLEGSYAQGSGSPIEGQTTHRGPSL
NGSGSNDYQNKIVAGDN
SEQ ID NO: 243
KGTWQIDSAQSLGSGSDESLRFYPFPTVNANGSGSHTKDMQLYSGPVGMQTGSGSNLRLNVGVS
NILNKQGSGSFNLISAWQQQHYSRTGSGSLNFSQGVELPDPGGSGSAPMMVSVIDTSAPEGSGS
INNSLNWTITQAFSGSGSPIQDGLLYGSVTLLRGSGSQTTWVIENAELEQQGSGSNPTNWLMLF
GSYAQGSGSNDYQNKIVAGDN
SEQ ID NO: 244
WTAKDANDNPVTGLNPDAPSLSGAAAAGSTASGWTDNGDGTWGSGSKGTWQIDSAQSLGSGSAA
QLTVIAGEMSSANSTLVADNKTPTVKTTTELTGSGSDESLRFYPFPTVNANGSGSSALALQADA
RNSDLTITQHGGGNGADVGQGSDDSSIDGSGSHTKDMQLYSGPVGMQTGSGSHNDYPETITDGS
GSNLRLNVGVSNILNKQGSGSSYPFPTTSETPRVVYNSRTDKPGSGSENLISAWQQQHYSRTGS
GSWNQCNGPGFADGSWAYGSGSLNFSQGVELPDPGGSGSIVSVNGVDTGETTMRWYKAGTQNGS
GSAPMMVSVIDTSAPEGSGSGEVKGRLVLDDRKRTEEVRPLGSGSINNSLNWTITQAFSGSGSV
IDAKSYKVVKTFDGSGSGNMLFSTISLRGVGSGSATLDQWNGKDSTMGSGSQTTWVIENAELEQ
QGSGSESTGTPSNATYTYVIERWDPETGSGSNPTNWLMLEGSYAQGSGSPIEGQTTHRGPSLNG
SGSNDYQNKIVAGDNGSGSTGATIPIGGGGSGSPIQDGLLYGSVTLLR
SEQ ID NO: 245
KGTWQIDSAQSLGSGSDESLRFYPFPTVNANGSGSHTKDMQLYSGPVGMQTGSGSNLRLNVGVS
NILNKQGSGSFNLISAWQQQHYSRTGSGSLNFSQGVELPDPGGSGSAPMMVSVIDTSAPEGSGS
INNSLNWTITQAFSGSGSGNMLESTISLRGVGSGSQTTWVIENAELEQQGSGSNPTNWLMLEGS
YAQGSGSNDYQNKIVAGDNGSGSPIQDGLLYGSVTLLR
SEQ ID NO: 246
DESLRFYPFPTVNANGSGSKGTWQIDSAQSLGSGSHTKDMQLYSGPVGMQTGSGSNLRLNVGVS
NILNKQGSGSFNLISAWQQQHYSRTGSGSLNFSQGVELPDPGGSGSAPMMVSVIDTSAPEGSGS
INNSLNWTITQAFSGSGSPIQDGLLYGSVTLLRGSGSQTTWVIENAELEQQGSGSNPTNWLMLF
GSYAQGSGSNDYQNKIVAGDN
SEQ ID NO: 247
WTAKDANDNPVTGLNPDAPSLSGAAAAGSTASGWTDNGDGTWGSGSKGTWQIDSAQSLGSGSAA
QLTVIAGEMSSANSTLVADNKTPTVKTTTELTGSGSDESLRFYPFPTVNANGSGSSALALQADA
RNSDLTITQHGGGNGADVGQGSDDSSIDGSGSGNVIRSEFTNDSEGSGSHNDYPETITDGSGSN
LRLNVGVSNILNKQGSGSSYPFPTTSETPRVVYNSRTDKPGSGSDVQSVELLRGPQGTLGSGSW
NQCNGPGFADGSWAYGSGSGGLINIVTKKGQPEGSGSIVSVNGVDTGETTMRWYKAGTQNGSGS
APMMVSVIDTSAPEGSGSGEVKGRLVLDDRKRTEEVRPLGSGSVDGIEASMSFPLVKGSGSVID
AKSYKVVKTFDGSGSQGTYATLDSSLGWQGSGSATLDQWNGKDSTMGSGSQTTWVIENAELEQQ
GSGSESTGTPSNATYTYVIERWDPETGSGSDFYVSYQGQQALKGSGSPIEGQTTHRGPSLNGSG
SDETLVVEATAEQVLK
SEQ ID NO: 248
KGTWQIDSAQSLGSGSDESLRFYPFPTVNANGSGSGNVIRSEFTNDSEGSGSNLRLNVGVSNIL
NKQGSGSDVQSVELLRGPQGTLGSGSGGLINIVTKKGQPEGSGSAPMMVSVIDTSAPEGSGSVD
GIEASMSFPLVKGSGSQGTYATLDSSLGWQGSGSQTTWVIENAELEQQGSGSDFYVSYQGQQAL
KGSGSDETLVVEATAEQVLK
SEQ ID NO: 249
WTAKDANDNPVTGLNPDAPSLSGAAAAGSTASGWTDNGDGTWGSGSKGTWQIDSAQSLGSGSAA
QLTVIAGEMSSANSTLVADNKTPTVKTTTELTGSGSDESLRFYPFPTVNANGSGSSALALQADA
RNSDLTITQHGGGNGADVGQGSDDSSIDGSGSGNVIRSEFTNDSEGSGSHNDYPETITDGSGSN
LRLNVGVSNILNKQGSGSSYPFPTTSETPRVVYNSRTDKPGSGSDVQSVELLRGPQGTLGSGSW
NQCNGPGFADGSWAYGSGSGGLINIVTKKGQPEGSGSIVSVNGVDTGETTMRWYKAGTQNGSGS
APMMVSVIDTSAPEGSGSGEVKGRLVLDDRKRTEEVRPLGSGSVDGIEASMSFPLVKGSGSVID
AKSYKVVKTFDGSGSQGTYATLDSSLGWQ
SEQ ID NO: 250
KGTWQIDSAQSLGSGSDESLRFYPFPTVNANGSGSGNVIRSEFTNDSEGSGSNLRLNVGVSNIL
NKQGSGSDVQSVELLRGPQGTLGSGSGGLINIVTKKGQPEGSGSAPMMVSVIDTSAPEGSGSVD
GIEASMSFPLVKGSGSQGTYATLDSSLGWQ
SEQ ID NO: 251
WTAKDANDNPVTGLNPDAPSLSGAAAAGSTASGWTDNGDGTWLLSVGGKGTWQIDSAQSLLLSV
GGAAQLTVIAGEMSSANSTLVADNKTPTVKTTTELTLLSVGGDESLRFYPFPTVNANLLSVGGS
ALALQADARNSDLTITQHGGGNGADVGQGSDDSSIDLLSVGGGNVIRSEFTNDSELLSVGGHND
YPETITDLLSVGGNLRLNVGVSNILNKQLLSVGGSYPFPTTSETPRVVYNSRTDKPLLSVGGDV
QSVELLRGPQGTLLLSVGGWNQCNGPGFADGSWAYLLSVGGGGLINIVTKKGQPELLSVGGIVS
VNGVDTGETTMRWYKAGTQNLLSVGGAPMMVSVIDTSAPELLSVGGGEVKGRLVLDDRKRTEEV
RPLLLSVGGVDGIEASMSFPLVKLLSVGGVIDAKSYKVVKTFDLLSVGGQGTYATLDSSLGWQL
LSVGGATLDQWNGKDSTMLLSVGGQTTWVIENAELEQQLLSVGGESTGTPSNATYTYVIERWDP
ETLLSVGGDFYVSYQGQQALKLLSVGGPIEGQTTHRGPSLNLLSVGGDETLVVEATAEQVLK
SEQ ID NO: 252
WTAKDANDNPVTGLNPDAPSLSGAAAAGSTASGWTDNGDGTWGSGSKGTWQIDSAQSLGSGSAA
QLTVIAGEMSSANSTLVADNKTPTVKTTTELTGSGSDESLRFYPFPTVNANGSGSSALALQADA
RNSDLTITQHGGGNGADVGQGSDDSSIDGSGSHTKDMQLYSGPVGMQTGSGSHNDYPETITDGS
GSNLRLNVGVSNILNKQGSGSSYPFPTTSETPRVVYNSRTDKPGSGSENLISAWQQQHYSRTGS
GSWNQCNGPGFADGSWAYGSGSLNFSQGVELPDPGGSGSIVSVNGVDTGETTMRWYKAGTQNGS
GSAPMMVSVIDTSAPEGSGSGEVKGRLVLDDRKRTEEVRPLGSGSINNSLNWTITQAFSGSGSV
IDAKSYKVVKTFDGSGSGNMLFSTISLRGVGSGSATLDQWNGKDSTMGSGSQTTWVIENAELEQ
QGSGSESTGTPSNATYTYVIERWDPETGSGSNPTNWLMLEGSYAQGSGSPIEGQTTHRGPSLNG
SGSNDYQNKIVAGDN
SEQ ID NO: 253
KGTWQIDSAQSLGSGSDESLRFYPFPTVNANGSGSHTKDMQLYSGPVGMQTGSGSNLRLNVGVS
NILNKQGSGSFNLISAWQQQHYSRTGSGSLNFSQGVELPDPGGSGSAPMMVSVIDTSAPEGSGS
INNSLNWTITQAFSGSGSGNMLFSTISLRGVGSGSQTTWVIENAELEQQGSGSNPTNWLMLEGS
YAQGSGSNDYQNKIVAGDN
SEQ ID NO: 254
WTAKDANDNPVTGLNPDAPSLSGAAAAGSTASGWTDNGDGTWLLSVGGKGTWQIDSAQSLLLSV
GGAAQLTVIAGEMSSANSTLVADNKTPTVKTTTELTLLSVGGDESLRFYPFPTVNANLLSVGGS
ALALQADARNSDLTITQHGGGNGADVGQGSDDSSIDLLSVGGHTKDMQLYSGPVGMQTLLSVGG
HNDYPETITDLLSVGGNLRLNVGVSNILNKQLLSVGGSYPFPTTSETPRVVYNSRTDKPLLSVG
GFNLISAWQQQHYSRTLLSVGGWNQCNGPGFADGSWAYLLSVGGLNFSQGVELPDPGLLSVGGI
VSVNGVDTGETTMRWYKAGTQNLLSVGGAPMMVSVIDTSAPELLSVGGGEVKGRLVLDDRKRTE
EVRPLLLSVGGINNSLNWTITQAFSLLSVGGVIDAKSYKVVKTFDLLSVGGGNMLFSTISLRGV
LLSVGGATLDQWNGKDSTMLLSVGGQTTWVIENAELEQQLLSVGGESTGTPSNATYTYVIERWD
PETLLSVGGNPTNWLMLFGSYAQLLSVGGPIEGQTTHRGPSLNLLSVGGNDYQNKIVAGDN
SEQ ID NO: 255
WTAKDANDNPVTGLNPDAPSLSGAAAAGSTASGWTDNGDGTWGSGSINNSLNWTITQAFSGSGS
AAQLTVIAGEMSSANSTLVADNKTPTVKTTTELTGSGSNPTNWLMLFGSYAQGSGSSALALQAD
ARNSDLTITQHGGGNGADVGQGSDDSSIDGSGSNDYQNKIVAGDNGSGSHNDYPETITDGSGSD
ESLRFYPFPTVNANGSGSSYPFPTTSETPRVVYNSRTDKPGSGSINNSLNWTITQAFSGSGSWN
QCNGPGFADGSWAYGSGSNPTNWLMLFGSYAQGSGSIVSVNGVDTGETTMRWYKAGTQNGSGSN
DYQNKIVAGDNGSGSGEVKGRLVLDDRKRTEEVRPLGSGSDESLRFYPFPTVNANGSGSVIDAK
SYKVVKTEDGSGSINNSLNWTITQAFSGSGSATLDQWNGKDSIMGSGSNPTNWLMLFGSYAQGS
GSESTGTPSNATYTYVIERWDPETGSGSNDYQNKIVAGDNGSGSPIEGQTTHRGPSLNGSGSDE
SLRFYPFPTVNAN
SEQ ID NO: 256
WTAKDANDNPVTGLNPDAPSLSGAAAAGSTASGWTDNGDGTWGSGSKGTWQIDSAQSLGSGSAA
QLTVIAGEMSSANSTLVADNKTPTVKTTTELTGSGSDESLRFYPFPTVNANGSGSSALALQADA
RNSDLTITQHGGGNGADVGQGSDDSSIDGSGSHTKDMQLYSGPVGMQTGSGSHNDYPETITDGS
GSNLRLNVGVSNILNKQGSGSSYPFPTTSETPRVVYNSRTDKPGSGSENLISAWQQQHYSRTGS
GSWNQCNGPGFADGSWAYGSGSLNFSQGVELPDPGGSGSIVSVNGVDTGETTMRWYKAGTQNGS
GSAPMMVSVIDTSAPEGSGSGEVKGRLVLDDRKRTEEVRPLGSGSINNSLNWTITQAFSGSGSV
IDAKSYKVVKTFDGSGSPIQDGLLYGSVTLLRGSGSATLDQWNGKDSTMGSGSQTTWVIENAEL
EQQGSGSESTGTPSNATYTYVIERWDPETGSGSNPTNWLMLEGSYAQGSGSPIEGQTTHRGPSL
NGSGSNDYQNKIVAGDNGSGSTGATIPIGGG
SEQ ID NO: 257
WTAKDANDNPVTGLNPDAPSLSGAAAAGSTASGWTDNGDGTWGSGSAAQLTVIAGEMSSANSTL
VADNKTPTVKTTTELTGSGSSALALQADARNSDLTITQHGGGNGADVGQGSDDSSIDGSGSHND
YPETITDGSGSKGTWQIDSAQSLGSGSDESLRFYPFPTVNANGSGSHTKDMQLYSGPVGMQTGS
GSNLRLNVGVSNILNKQGSGSSYPFPTTSETPRVVYNSRTDKPGSGSWNQCNGPGFADGSWAYG
SGSIVSVNGVDTGETTMRWYKAGTQNGSGSGEVKGRLVLDDRKRTEEVRPLGSGSFNLISAWQQ
QHYSRTGSGSLNFSQGVELPDPGGSGSAPMMVSVIDTSAPEGSGSINNSLNWTITQAFSGSGSV
IDAKSYKVVKTFDGSGSATLDQWNGKDSTMGSGSESTGTPSNATYTYVIERWDPETGSGSPIEG
QTTHRGPSLNGSGSPIQDGLLYGSVTLLRGSGSQTTWVIENAELEQQGSGSNPTNWLMLEGSYA
QGSGSNDYQNKIVAGDN
SEQ ID NO: 258
KGTWQIDSAQSLGSGSDESLRFYPFPTVNANGSGSHTKDMQLYSGPVGMQTGSGSNLRLNVGVS
NILNKQGSGSWTAKDANDNPVTGLNPDAPSLSGAAAAGSTASGWTDNGDGTWGSGSAAQLTVIA
GEMSSANSTLVADNKTPTVKTTTELTGSGSSALALQADARNSDLTITQHGGGNGADVGQGSDDS
SIDGSGSHNDYPETITDGSGSFNLISAWQQQHYSRTGSGSLNFSQGVELPDPGGSGSAPMMVSV
IDTSAPEGSGSINNSLNWTITQAFSGSGSSYPFPTTSETPRVVYNSRTDKPGSGSWNQCNGPGF
ADGSWAYGSGSIVSVNGVDTGETTMRWYKAGTQNGSGSGEVKGRLVLDDRKRTEEVRPLGSGSP
IQDGLLYGSVTLLRGSGSQTTWVIENAELEQQGSGSNPTNWLMLFGSYAQGSGSNDYQNKIVAG
DNGSGSVIDAKSYKVVKTFDGSGSATLDQWNGKDSTMGSGSESTGTPSNATYTYVIERWDPETG
SGSPIEGQTTHRGPSLN
SEQ ID NO: 259
AAQLTVIAGEMSSANSTLVADNKTPTVKTTTELTGSGSKGTWQIDSAQSLGSGSWTAKDANDNP
VTGLNPDAPSLSGAAAAGSTASGWTDNGDGTWGSGSDESLRFYPFPTVNANGSGSSALALQADA
RNSDLTITQHGGGNGADVGQGSDDSSIDGSGSHTKDMQLYSGPVGMQTGSGSHNDYPETITDGS
GSNLRLNVGVSNILNKQGSGSSYPFPTTSETPRVVYNSRTDKPGSGSENLISAWQQQHYSRTGS
GSWNQCNGPGFADGSWAYGSGSLNFSQGVELPDPGGSGSIVSVNGVDTGETTMRWYKAGTQNGS
GSAPMMVSVIDTSAPEGSGSGEVKGRLVLDDRKRTEEVRPLGSGSINNSLNWTITQAFSGSGSV
IDAKSYKVVKTFDGSGSPIQDGLLYGSVTLLRGSGSATLDQWNGKDSTMGSGSQTTWVIENAEL
EQQGSGSESTGTPSNATYTYVIERWDPETGSGSNPTNWLMLEGSYAQGSGSPIEGQTTHRGPSL
NGSGSNDYQNKIVAGDN
SEQ ID NO: 260
WTAKDANDNPVTGLNPDAPSLSGAAAAGSTASGWTDNGDGTWGSGSDESLRFYPFPTVNANGSG
SAAQLTVIAGEMSSANSTLVADNKTPTVKTTTELTGSGSKGTWQIDSAQSLGSGSSALALQADA
RNSDLTITQHGGGNGADVGQGSDDSSIDGSGSHTKDMQLYSGPVGMQTGSGSHNDYPETITDGS
GSNLRLNVGVSNILNKQGSGSSYPFPTTSETPRVVYNSRTDKPGSGSENLISAWQQQHYSRTGS
GSWNQCNGPGFADGSWAYGSGSLNFSQGVELPDPGGSGSIVSVNGVDTGETTMRWYKAGTQNGS
GSAPMMVSVIDTSAPEGSGSGEVKGRLVLDDRKRTEEVRPLGSGSINNSLNWTITQAFSGSGSV
IDAKSYKVVKTFDGSGSPIQDGLLYGSVTLLRGSGSATLDQWNGKDSTMGSGSQTTWVIENAEL
EQQGSGSESTGIPSNATYTYVIERWDPETGSGSNPTNWLMLEGSYAQGSGSPIEGQTTHRGPSL
NGSGSNDYQNKIVAGDN

### ENUMERATED EMBODIMENTS

The following exemplary embodiments are provided, the numbering of which is not to be construed as designating levels of importance:

Embodiment 1 provides a polypeptide comprising formula (I), or a salt or solvate thereof:

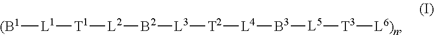

wherein:
each occurrence of B¹, B², B³, L¹, L², L³, L⁴, L⁵, L⁶, T¹, T², and T³ can be present or absent;
each occurrence of B¹, B², and B³, if present, independently comprise an immunogenic fragment of a bacterial surface protein;
each occurrence of T¹, T², and T³, if present, independently comprise an immunogenic fragment of an iron receptor protein,
   wherein at least two of T¹, T², and T³ are present or at least one of T¹, T² and T³ is present and n is at least 2;
each occurrence of L¹, L², L³, L⁴, L⁵, and L⁶, if present, independently comprise a polypeptide of 1-10 amino acids,
   wherein at least one of L¹, L², L³, L⁴, L⁵, and L⁶ is present, and
   wherein each occurrence of B¹, B², B³, T¹, T², and T³ are separated from one another by at least one of L¹, L², L³, L⁴, L⁵, and L⁶; and
n is an integer selected from the group consisting of 1, 2, 3, 4, and 5.

Embodiment 2 provides the polypeptide of Embodiment 1, wherein at least one of B¹, B², or B³ is present.

Embodiment 3 provides the polypeptide of Embodiment 1 or 2, wherein each bacterial surface protein has at least 85%, 90%, 95%, 96%, 97%, 98%, 99%, or 100% sequence homology to a polypeptide independently selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, and SEQ ID NO:9.

Embodiment 4 provides the polypeptide of Embodiment 3, wherein each immunogenic fragment of the bacterial surface protein shares at least 85%, 90%, 95%, 96%, 97%, 98%, 99%, or 100% sequence homology with a polypeptide independently selected from the group consisting of SEQ ID NO:16, SEQ ID NO:195, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, and SEQ ID NO:41.

Embodiment 5 provides the polypeptide of Embodiment 3 or 4, wherein each immunogenic fragment of the bacterial surface protein shares at least 85%, 90%, 95%, 96%, 97%, 98%, 99%, or 100% sequence homology with a polypeptide independently selected from the group consisting of SEQ ID NO:16, SEQ ID NO:195, SEQ ID NO:17, SEQ ID NO:18, SEQ ID NO:19, SEQ ID NO:20, SEQ ID NO:23, SEQ ID NO:25, SEQ ID NO:27, SEQ ID NO:32, SEQ ID NO:35, SEQ ID NO:37, SEQ ID NO:39, and SEQ ID NO:40.

Embodiment 6 provides the polypeptide of any one of Embodiments 1-5, wherein each iron receptor protein shares at least 85%, 90%, 95%, 96%, 97%, 98%, 99%, or 100% sequence homology with a polypeptide independently selected from the group consisting of SEQ ID NO:10, SEQ ID NO:11, SEQ ID NO:12, SEQ ID NO:13, SEQ ID NO:14, and SEQ ID NO:15.

Embodiment 7 provides the polypeptide of Embodiment 6, wherein each immunogenic fragment of the iron receptor protein shares at least 85%, 90%, 95%, 96%, 97%, 98%, 99%, or 100% sequence homology with a polypeptide independently selected from the group consisting of SEQ ID NO:42, SEQ ID NO:43, SEQ ID NO:4, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO: 112, SEQ ID NO: 113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, and SEQ ID NO:123.

Embodiment 8 provides the polypeptide of Embodiment 6 or 7, wherein each immunogenic fragment of the iron receptor protein shares at least 85%, 90%, 95%, 96%, 97%, 98%, 99%, or 100% sequence homology with a polypeptide independently selected from the group consisting of SEQ ID NO:42, SEQ ID NO:46, SEQ ID NO:47, SEQ ID NO:51, SEQ ID NO:53, SEQ ID NO:54, SEQ ID NO:55, SEQ ID NO:56, SEQ ID NO:58, SEQ ID NO:62, SEQ ID NO:67, SEQ ID NO:85, and SEQ ID NO:88.

Embodiment 9 provides the polypeptide of any one of Embodiments 1-8, wherein each of L¹, L², L³, L⁴, L⁵, and L⁶, if present, independently comprise a polypeptide wherein each amino acid residue side chain comprises a neutral (i.e., non-charged) substituent.

Embodiment 10 provides the polypeptide of any one of Embodiments 1-9, wherein each of L¹, L², L³, L⁴, L⁵, and L⁶, if present, independently comprise a polypeptide of 4 to 6 amino acids.

Embodiment 11 provides the polypeptide of any one of Embodiments 1-10, wherein each of L¹, L², L³, L⁴, L⁵, and L⁶, if present, is independently selected from the group consisting of GSGS (SEQ ID NO:124), GPGP (SEQ ID NO:125), LLSVGG (SEQ ID NO:126), (SGSG)₁₋₂ (SEQ ID NOs:146-147), SSSS (SEQ ID NO:156), GGGS (SEQ ID NO:157), GGC (SEQ ID NO:158), GGS (SEQ ID NO:159), (GGC)₈ (SEQ ID NO:160), (GGGGS)₃ (SEQ ID NO:161), and GGAAY (SEQ ID NO:162).

Embodiment 12 provides the polypeptide of any one of Embodiments 1-11, wherein n is 3.

Embodiment 13 provides the polypeptide of any one of Embodiments 1-12, wherein the polypeptide shares at least 85%, 90%, 95%, 96%, 97%, 98%, 99%, or 100% sequence homology with a polypeptide selected from the group consisting of SEQ ID NO:130 and SEQ ID NO:129.

Embodiment 14 provides the polypeptide of any one of Embodiments 1-11, wherein n is 4.

Embodiment 15 provides the polypeptide of any one of Embodiments 1-11 and 14, wherein the polypeptide shares at least 85%, 90%, 95%, 96%, 97%, 98%, 99%, or 100% sequence homology with a polypeptide selected from the group consisting of SEQ ID NO:127, SEQ ID NO:128, SEQ ID NO:131, SEQ ID NO:132, SEQ ID NO:133, SEQ ID NO:134, SEQ ID NO:135, SEQ ID NO:136, SEQ ID NO:137, SEQ ID NO:143, SEQ ID NO:144, SEQ ID NO:145, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, SEQ ID NO:242, SEQ ID NO:243, SEQ ID NO:244, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:253, SEQ ID NO:254, SEQ ID NO:255, SEQ ID NO:256, SEQ ID NO:257, SEQ ID NO:258, SEQ ID NO:259, and SEQ ID NO:260.

Embodiment 16 provides a polypeptide selected from the group consisting of:

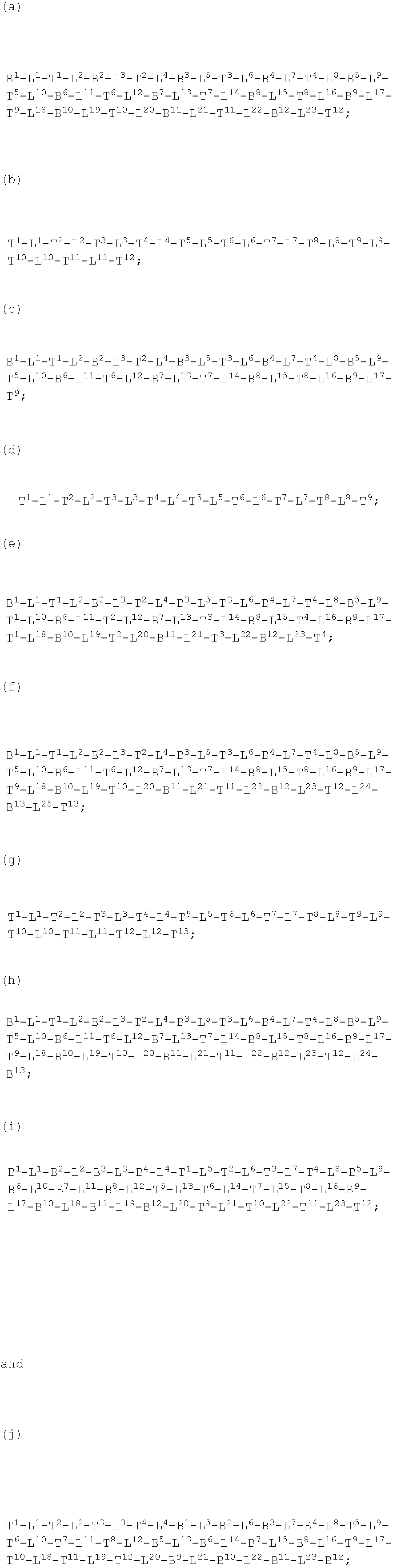

wherein:
each occurrence of B, if present, independently comprises an immunogenic fragment of a bacterial surface protein,
   wherein each instance of B has a C-terminus and a N-terminus;
each occurrence of T independently comprises an immunogenic fragment of an iron receptor protein,
   wherein each instance of T has a C-terminus and a N-terminus;
each occurrence of L independently comprises a polypeptide of 1-10 amino acids,
   wherein each instance of L has a C-terminus and a N-terminus;
each instance of B is covalently linked to one or two independent instances of L by a covalent peptide bond between the C-terminus of the B and the N-terminus of the L and/or the N-terminus of the B and the C-terminus of the L; and
each instance of T is covalently linked to one or two independent instances of L by a covalent peptide bond between the C-terminus of the T and the N-terminus of the L and/or the N-terminus of the T and the C-terminus of the L;
   or a salt or solvate thereof.

Embodiment 17 provides the polypeptide of Embodiment 16, wherein each bacterial surface protein has at least 85%, 90%, 95%, 96%, 97%, 98%, 99%, or 100% sequence homology to a polypeptide independently selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, and SEQ ID NO:9.

Embodiment 18 provides the polypeptide of Embodiment 17, wherein each immunogenic fragment of the bacterial surface protein shares at least 85%, 90%, 95%, 96%, 97%, 98%, 99%, or 100% sequence homology with a polypeptide independently selected from the group consisting of SEQ ID NO:16, SEQ ID NO:195, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, and SEQ ID NO:41.

Embodiment 19 provides the polypeptide of Embodiment 17 or 18, wherein each immunogenic fragment of the bacterial surface protein shares at least 85%, 90%, 95%, 96%, 97%, 98%, 99%, or 100% sequence homology with a polypeptide independently selected from the group consisting of SEQ ID NO:16, SEQ ID NO:195, SEQ ID NO:17, SEQ ID NO:18, SEQ ID NO:19, SEQ ID NO:20, SEQ ID NO:23, SEQ ID NO:25, SEQ ID NO:27, SEQ ID NO:32, SEQ ID NO:35, SEQ ID NO:37, SEQ ID NO:39, and SEQ ID NO:40.

Embodiment 20 provides the polypeptide of any one of Embodiments 16-19, wherein each iron receptor protein shares at least 85%, 90%, 95%, 96%, 97%, 98%, 99%, or 100% sequence homology with a polypeptide independently selected from the group consisting of SEQ ID NO:10, SEQ ID NO:11, SEQ ID NO:12, SEQ ID NO:13, SEQ ID NO:14, and SEQ ID NO:15.

Embodiment 21 provides the polypeptide of Embodiment 20, wherein each immunogenic fragment of the iron receptor protein shares at least 85%, 90%, 95%, 96%, 97%, 98%, 99%, or 100% sequence homology with a polypeptide independently selected from the group consisting of SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, and SEQ ID NO:123.

Embodiment 22 provides the polypeptide of Embodiment 20 or 21, wherein each immunogenic fragment of the iron receptor protein shares at least 85%, 90%, 95%, 96%, 97%, 98%, 99%, or 100% sequence homology with a polypeptide independently selected from the group consisting of SEQ ID NO:42, SEQ ID NO:46, SEQ ID NO:47, SEQ ID NO:51, SEQ ID NO:53, SEQ ID NO:54, SEQ ID NO:55, SEQ ID NO:56, SEQ ID NO:58, SEQ ID NO:62, SEQ ID NO:67, SEQ ID NO:85, and SEQ ID NO:88.

Embodiment 23 provides the polypeptide of any one of Embodiments 16-22, wherein each of L¹, L², L³, L⁴, L⁵, L⁶, L⁷, L⁸, L⁹, L¹⁰, L¹¹, L¹², L¹³, L¹⁴, L15, L¹⁶, L¹⁷, L¹⁸, L¹⁹, L²⁰, L²¹, L²², L²³, L²⁴, and L²⁵, if present, independently comprise a polypeptide wherein each amino acid residue side chain comprises a neutral (i.e., non-charged) substituent.

Embodiment 24 provides the polypeptide of any one of Embodiments 16-23, wherein each of L¹, L², L³, L⁴, L⁵, L⁶, L⁷, L⁸, L⁹, L¹⁰, L¹¹, L¹², L¹³, L¹⁴, L¹⁵, L¹⁶, L¹⁷, L¹⁸, L¹⁹, L²⁰, L²¹, L22, L²³, L²⁴, and L²³, if present, independently comprise a polypeptide of 4 to 6 amino acids.

Embodiment 25 provides the polypeptide of any one of Embodiments 16-24, wherein each of L¹, L², L³, L⁴, L⁵, L⁶, L⁷, L⁸, L⁹, L¹⁰, L¹¹, L¹², L¹³, L¹⁴, L¹⁵, L¹⁶, L¹⁷, L¹⁸, L¹⁹, L²⁰, L²¹, L²², L²³, L²⁴, and L²⁵, if present, is independently selected from the group consisting of GSGS (SEQ ID NO:124), GPGP (SEQ ID NO:125), LLSVGG (SEQ ID NO:126), (SGSG)₁₋₂ (SEQ ID NOs:146-147), SSSS (SEQ ID NO:156), GGGS (SEQ ID NO:157), GGC (SEQ ID NO:158), GGS (SEQ ID NO:159), (GGC)₈ (SEQ ID NO:160), (GGGGS)₃ (SEQ ID NO:161), and GGAAY (SEQ ID NO:162).

Embodiment 26 provides the polypeptide of any one of Embodiments 16-25, wherein the polypeptide shares at least 85%, 90%, 95%, 96%, 97%, 98%, 99%, or 100% sequence homology with a polypeptide selected from the group consisting of SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, SEQ ID NO:242, SEQ ID NO:243, SEQ ID NO:244, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:253, SEQ ID NO:254, SEQ ID NO:255, SEQ ID NO:256, SEQ ID NO:257, SEQ ID NO:258, SEQ ID NO:259, and SEQ ID NO:260.

Embodiment 27 provides the polypeptide of any one of Embodiments 1-26, wherein the polypeptide shares at least 85%, 90%, 95%, 96%, 97%, 98%, 99%, or 100% sequence homology with SEQ ID NO:225.

Embodiment 28 provides the polypeptide of any one of Embodiments 1-26, wherein the polypeptide shares at least 85%, 90%, 95%, 96%, 97%, 98%, 99%, or 100% sequence homology with SEQ ID NO:226.

Embodiment 29 provides an isolated messenger ribonucleic acid (mRNA) encoding the polypeptide of any one of Embodiments 1-28.

Embodiment 30 provides the isolated mRNA of Embodiment 29, wherein the mRNA is codon optimized for expression in:
(a) a prokaryote; or
(b) a mammal.

Embodiment 31 provides the isolated mRNA of Embodiment 30, wherein one of the following applies:
(a) the prokaryote is E. coli; or
(b) the mammal is a human.

Embodiment 32 provides the isolated deoxyribonucleic acid (DNA) encoding the polypeptide of any one of Embodiments 1-28.

Embodiment 33 provides the isolated DNA of Embodiment 32, wherein the DNA is codon optimized for expression in:
(a) a prokaryote; or
(b) a mammal.

Embodiment 34 provides the isolated DNA of Embodiment 33, wherein one of the following applies:
(a) the prokaryote is E. coli; or
(b) the mammal is a human.

Embodiment 35 provides an isolated polynucleotide encoding the mRNA of any one of Embodiments 29-31, wherein the polynucleotide comprises one or more promoters and/or a polyadenylation signal operably linked to a sequence encoding the mRNA.

Embodiment 36 provides a vector comprising the isolated mRNA of any one of Embodiments 29-31, the isolated DNA of any one of Embodiments 32-34, and/or the isolated polynucleotide of Embodiment 35.

Embodiment 37 provides the vector of Embodiment 36, wherein the vector is a viral vector.

Embodiment 38 provides the vector of Embodiment 37, wherein the viral vector is Adeno-Associated Virus (AAV), optionally wherein the AAV is AAV9.

Embodiment 39 provides the vector of Embodiment 36, wherein the vector is a bacterial expression vector.

Embodiment 40 provides the vector of Embodiment 39, wherein the bacterial expression vector is an E. coli vector.

Embodiment 41 provides a lipid nanoparticle (LNP) composition comprising the isolated mRNA of any one of Embodiments 29-31, the isolated DNA of any one of Embodiments 32-34, and/or the isolated polynucleotide of Embodiment 35.

Embodiment 42 provides the LNP of Embodiment 41, wherein at least one of the following applies:
(a) the LNP has a ratio of lipid to isolated mRNA or DNA ranging from about 5:1 to about 25:1; and
(b) the LNP has a ratio of lipid to isolated polynucleotide ranging from about 5:1 to about 25:1.

Embodiment 43 provides the LNP of Embodiment 41 or 42, wherein the LNP comprises:
(a) at least one ionizable lipid;
(b) at least one helper lipid;
(c) cholesterol, or a modified derivative thereof, and any combinations thereof, and
(d) at least one conjugated lipid.

Embodiment 44 provides the LNP of Embodiment 43, wherein the ionizable lipid is at least one selected from the group consisting of DLinDMA, DLenDMA, DLin-K-C2-DMA, D Lin-K-C3-D MA, DLin-K-C4-DMA, DLin-K6-DMA, DLin-K-MPZ, DLin-KDMA, D Lin-C-DAP, DLin-DAC, DLin-MA, DLinDAP, DLin-2-DMAP, DLin-TMA.Cl, DLin-TAP.Cl, D Lin-MPZ, D LinAP, DOAP, D Lin-EG-D MA, DODAC, DODMA, DSD MA, DOTMA, DDAB, DOTAP, DC-Chol, DMRIE, DOSPA, DOGS, CLinDMA, CpLinDMA, DMOBA, DOcarbDAP, and DLincarbDAP.

Embodiment 45 provides the LNP of Embodiment 43 or 44, wherein the at least one ionizable lipid comprises about 50 mol % to about 90 mol % of the LNP.

Embodiment 46 provides the LNP of any one of Embodiments 43-45, wherein the helper lipid is at least one selected from the group consisting of 1,2-distearoyl-sn-glycero-3-phosphocholine (DSPC), 1,2-di-(9Z-octadecenoyl)-sn-glycero-3-phosphoethanolamine (DOPE), 1-palmitoyl-2-oleoyl-sn-glycero-3-phosphocholin (POPC), and 1,2-dioleoyl-sn-glycero-3-phosphocholine (DOPC).

Embodiment 47 provides the LNP of any one of Embodiments 43-46, wherein the at least one helper lipid comprises about 1 to about 25 mol % of the LNP.

Embodiment 48 provides the LNP of any one of Embodiments 43-47, wherein the cholesterol comprises about 20 to about 60 mol % of the LNP.

Embodiment 49 provides the LNP of any one of Embodiments 43-48, wherein the conjugated lipid is at least one selected from the group consisting of 1,2-dimyristoyl-rac-glycero-3-methoxypolyethylene glycol-2000 (PEG-DMG), 1,2-Distearoyl-sn-glycerol, methoxypolyethylene glycol (PEG-DSG), 1,2-Dipalmitoyl-sn-glycerol, methoxypolyethylene glycol (PEG-DPG), mPEG-OH, mPEG-AA (mPEG-CM), mPEG-CH₂CH₂CH₂—NH₂, mPEG-DMG, mPEG-N,N-Ditetradecylacetamide (ALC-0159), mPEG-DSPE, and mPEG-DPPE.

Embodiment 50 provides the LNP of any one of Embodiments 43-49, wherein the at least one conjugated lipid comprises about 0.1 to about 5 mol % of the LNP.

Embodiment 51 provides the LNP of any one of Embodiments 41-50, wherein the isolated mRNA, isolated DNA, and/or isolated polynucleotide is at least partially encapsulated in the LNP.

Embodiment 52 provides a pharmaceutical composition comprising the LNP of any one of Embodiments 41-51 and a pharmaceutically acceptable carrier.

Embodiment 53 provides a vaccine composition comprising the LNP of any one of Embodiments 41-51 and/or the pharmaceutical composition of Embodiment 52.

Embodiment 54 provides a vaccine composition comprising the polypeptide of any one of Embodiments 1-28 and at least one pharmaceutically acceptable excipient.

Embodiment 55 provides the vaccine composition of Embodiment 54, wherein the polypeptide shares at least 85%, 90%, 95%, 96%, 97%, 98%, 99/a, or 100% sequence homology with SEQ ID NO:225.

Embodiment 56 provides the vaccine composition of Embodiment 54, wherein the polypeptide shares at least 85%, 90%, 95%, 96%, 97%, 98%, 99%, or 100% sequence homology with SEQ ID NO:226 Embodiment 57 provides the vaccine composition of any one of Embodiments 54-56, further comprising an adjuvant.

Embodiment 58 provides the vaccine composition of Embodiment 57, wherein the adjuvant comprises at least one selected from the group consisting of aluminum hydroxide (AlOH), double mutant heat-labile toxin (dmLT), CpG, and an oil-in-water emulsion adjuvant.

Embodiment 59 provides the vaccine composition of Embodiment 58, wherein at least one of the following applies:
(a) the vaccine comprises about 10 μg to about 100 μg of the polypeptide;
(b) the vaccine comprises about 100 μg to about 6000 μg of the CpG;
(c) the vaccine comprises about 250 μg to about 750 μg of the AlOH;
(d) the vaccine comprises about 1 μg to about 25 μg of the dmLT;
(e) the vaccine comprises about 25% (v/v) to about 75% (v/v) of the oil-in-water emulsion adjuvant.

Embodiment 60 provides a vaccine composition comprising:
(a) a polypeptide which shares at least 85%, 90%, 95%, 96%, 97%, 98%, 99%, or 100% sequence homology with SEQ ID NO:225 or SEQ ID NO:226, wherein the vaccine composition comprises about 10 μg to about 100 μg of the polypeptide;
(b) α-tocopherol, wherein the α-tocopherol comprises about 2.5% (v/v) of the vaccine composition;
(c) squalene, wherein the squalene comprises about 2.5% (v/v) of the vaccine composition; and
(d) polysorbate 80, wherein the polysorbate 80 comprises about 0.9% (v/v) of the vaccine composition;
wherein the polypeptide, α-tocopherol, squalene, and polysorbate are dissolved or suspended in a phosphate buffered saline (PBS) solution.

Embodiment 61 provides a vaccine composition comprising:
(a) a polypeptide which shares at least 85%, 90%, 95%, 96%, 97%, 98%, 99%, or 100% sequence homology with SEQ ID NO:225 or SEQ ID NO:226, wherein the vaccine composition comprises about 10 μg to about 100 μg of the polypeptide;
(b) α-tocopherol, wherein the α-tocopherol comprises about 2.5% (v/v) of the vaccine composition;
(c) squalene, wherein the squalene comprises about 2.5% (v/v) of the vaccine composition;
(d) polysorbate 80, wherein the polysorbate 80 comprises about 0.9% (v/v) of the vaccine composition; and
(e) CpG, wherein the vaccine comprises about 100 μg to about 6000 μg of the CpG; wherein the polypeptide, α-tocopherol, squalene, polysorbate, and CpG are dissolved or suspended in a phosphate buffered saline (PBS) solution.

Embodiment 62 provides a method of treating, preventing, and/or ameliorating a bacterial infection in a subject in need thereof, the method comprising administering to the subject at least one selected from the group consisting of:
(a) the polypeptide of any one of Embodiments 1-28;
(b) the isolated mRNA of any one of Embodiments 29-31;
(c) the isolated DNA of any one of Embodiments 32-34;
(d) the isolated polynucleotide of Embodiment 35;
(e) the vector of any one of Embodiments 36-40;
(f) the LNP of any one of Embodiments 41-51;
(g) the pharmaceutical composition of Embodiment 52; and
(h) the vaccine composition of any one of Embodiments 53-61.

Embodiment 63 provides the method of Embodiment 62, wherein the bacterial infection is a urinary tract infection (UTI).

Embodiment 64 provides the method of Embodiment 62, wherein the bacterial infection comprises a urinary tract infection (UTI) and sepsis.

Embodiment 65 provides the method of Embodiment 62, wherein the bacterial infection is sepsis.

Embodiment 66 provides the method of Embodiment 64 or 65, wherein the sepsis is neonatal sepsis.

Embodiment 67 provides the method of Embodiment 62, wherein the bacterial infection is pneumonia.

Embodiment 68 provides the method of any one of Embodiments 62-67, wherein the subject is a mammal.

Embodiment 69 provides the method of Embodiment 68, wherein the mammal is a human.

Embodiment 70 provides the method of any one of Embodiments 62-69, wherein the subject is pregnant with a fetus.

Embodiment 71 provides the method of Embodiment 70, wherein the bacterial infection is treated, prevented, and/or ameliorated in at least one of the subject and the fetus, or a neonate thereof.

Embodiment 72 provides the method of Embodiment 70, wherein the bacterial infection is treated, prevented, and/or ameliorated in both the subject and the fetus, or a neonate thereof.

Embodiment 73 provides a method of generating immunity to infection by one or more pathogenic bacteria in a subject, the method comprising administering to the subject at least one selected from the group consisting of:
(a) the polypeptide of any one of Embodiments 1-28;
(b) the isolated mRNA of any one of Embodiments 29-31;
(c) the isolated DNA of any one of Embodiments 32-34;
(d) the isolated polynucleotide of Embodiment 35;
(e) the vector of any one of Embodiments 36-40;
(f) the LNP of any one of Embodiments 41-51;
(g) the pharmaceutical composition of Embodiment 52; and
(h) the vaccine composition of any one of Embodiments 53-61.

Embodiment 74 provides the method of Embodiment 73, wherein the one or more pathogenic bacteria comprises at least one selected from the group consisting of Escherichia coli, Klebsiella pneumoniae, Proteus mirabilis, Shigella dysenteriae, Salmonella enterica, Streptococcus pneumoniae, Haemophilus influenzae, Chlamydophila pneumoniae, Mycoplasma pneumoniae, Staphylococcus aureus, Moraxella catarrhalis, and Legionella pneumophila, Streptococcus pyogenes, and Pseudomonas aerugmosa, and Salmonella bongori.

Embodiment 75 provides the method of Embodiment 73 or 74, wherein the immunity prevents a bacterial infection.

Embodiment 76 provides the method of Embodiment 75, wherein the bacterial infection is a urinary tract infection (UTI).

Embodiment 77 provides the method of Embodiment 75, wherein the bacterial infection comprises a urinary tract infection (UTI) and sepsis.

Embodiment 78 provides the method of Embodiment 75, wherein the bacterial infection is sepsis.

Embodiment 79 provides the method of Embodiment 77 or 78, wherein the sepsis is neonatal sepsis.

Embodiment 80 provides the method of Embodiment 75, wherein the bacterial infection is pneumonia

Embodiment 81 provides the method of any one of Embodiments 73-80, wherein the subject is a mammal.

Embodiment 82 provides the method of Embodiment 81, wherein the mammal is a human.

Embodiment 83 provides the method of any one of Embodiments 73-82, wherein the subject is pregnant with a fetus.

Embodiment 84 provides the method of Embodiment 83, wherein the immunity to infection by one or more pathogenic bacteria is generated in at least one of the subject and the fetus, or a neonate thereof.

Embodiment 85 provides the method of Embodiment 83 or 84, wherein the immunity to infection by one or more pathogenic bacteria is generated in both the subject and the fetus, or a neonate thereof.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

1. A 1 tide comprising formula (I), or a salt or solvate thereof: 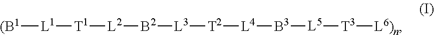
wherein:
each occurrence of B¹, B², B³, L¹, L², L³, L⁴, L⁵, L⁶, T¹, T², and T³ can be present or absent;
each occurrence of B¹, B², and B³, if present, independently comprise an immunogenic fragment of a bacterial surface protein;
each occurrence of T¹, T², and T³, if present, independently comprise an immunogenic fragment of an iron receptor protein,
wherein at least two of T¹, T², and T³ are present or at least one of T¹, T² and T³ is present and n is at least 2;
each occurrence of L¹, L², L³, L⁴, L⁵, and L⁶, if present, independently comprise a polypeptide of 1-10 amino acids,
wherein at least one of L¹, L², L³, L⁴, L⁵, and L⁶ is present, and
wherein each occurrence of B¹, B², B³, T¹, T², and T³ are separated from one another by at least one of L¹, L², L³, L⁴, L⁵, and L⁶; and
n is an integer selected from the group consisting of 1, 2, 3, 4, and 5.

2. The polypeptide of claim 1, wherein at least one of B¹, B², or B³ is present.

3. The polypeptide of claim 1, wherein each bacterial surface protein has at least 85%, 90%, 95%, 96%, 97%, 98%, 99%, or 100% sequence homology to a polypeptide independently selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, and SEQ ID NO:9.

4. The polypeptide of claim 3, wherein each immunogenic fragment of the bacterial surface protein shares at least 85%, 90%, 95%, 96%, 97%, 98%, 99%, or 100% sequence homology with a polypeptide independently selected from the group consisting of SEQ ID NO:16, SEQ ID NO:195, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, and SEQ ID NO:41.

5. (canceled)

6. The polypeptide of claim 1, wherein each iron receptor protein shares at least 85%, 90%, 95%, 96%, 97%, 98%, 99%, or 100% sequence homology with a polypeptide independently selected from the group consisting of SEQ ID NO:10, SEQ ID NO: 11, SEQ ID NO:12, SEQ ID NO:13, SEQ ID NO:14, and SEQ ID NO:15.

7. The polypeptide of claim 6, wherein each immunogenic fragment of the iron receptor protein shares at least 85%, 90%, 95%, 96%, 97%, 98%, 99%, or 100% sequence homology with a polypeptide independently selected from the group consisting of SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, and SEQ ID NO:123.

8. (canceled)

9. The polypeptide of claim 1, wherein at least one of the following applies:
(a) each of L¹, L², L³, L⁴, L⁵, and L⁶, if present, independently comprise a polypeptide wherein each amino acid residue side chain comprises a neutral (i.e., non-charged) substituents; and
(b) each of L¹, L², L³, L⁴, L⁵, and L⁶, if present, independently comprise a polypeptide of 4 to 6 amino acids.

10. (canceled)

11. The polypeptide of claim 1, wherein each of L¹, L², L³, L⁴, L⁵, and L⁶, if present, is independently selected from the group consisting of GSGS (SEQ ID NO:124), GPGP (SEQ ID NO:125), LLSVGG (SEQ ID NO:126), (SGSG)₁₋₂ (SEQ ID NOs:146-147), SSSS (SEQ ID NO:156), GGGS (SEQ ID NO:157), GGC (SEQ ID NO:158), GGS (SEQ ID NO:159), (GGC)₈ (SEQ ID NO:160), (GGGGS)₃ (SEQ ID NO:161), and GGAAY (SEQ ID NO:162).

12-14. (canceled)

15. The polypeptide of claim 1, wherein the polypeptide shares at least 85%, 90%, 95%, 96%, 97%, 98%, 99%, or 100% sequence homology with a polypeptide selected from the group consisting of SEQ ID NO:127, SEQ ID NO:128, SEO ID NO:129, SEO ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:143, SEQ ID NO:144, SEQ ID NO:145, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, SEQ ID NO:242, SEQ ID NO:243, SEQ ID NO:244, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:253, SEQ ID NO:254, SEQ ID NO:255, SEQ ID NO:256, SEQ ID NO:257, SEQ ID NO:258, SEQ ID NO:259, and SEQ ID NO:260.

16. A polypeptide selected from the group consisting of: 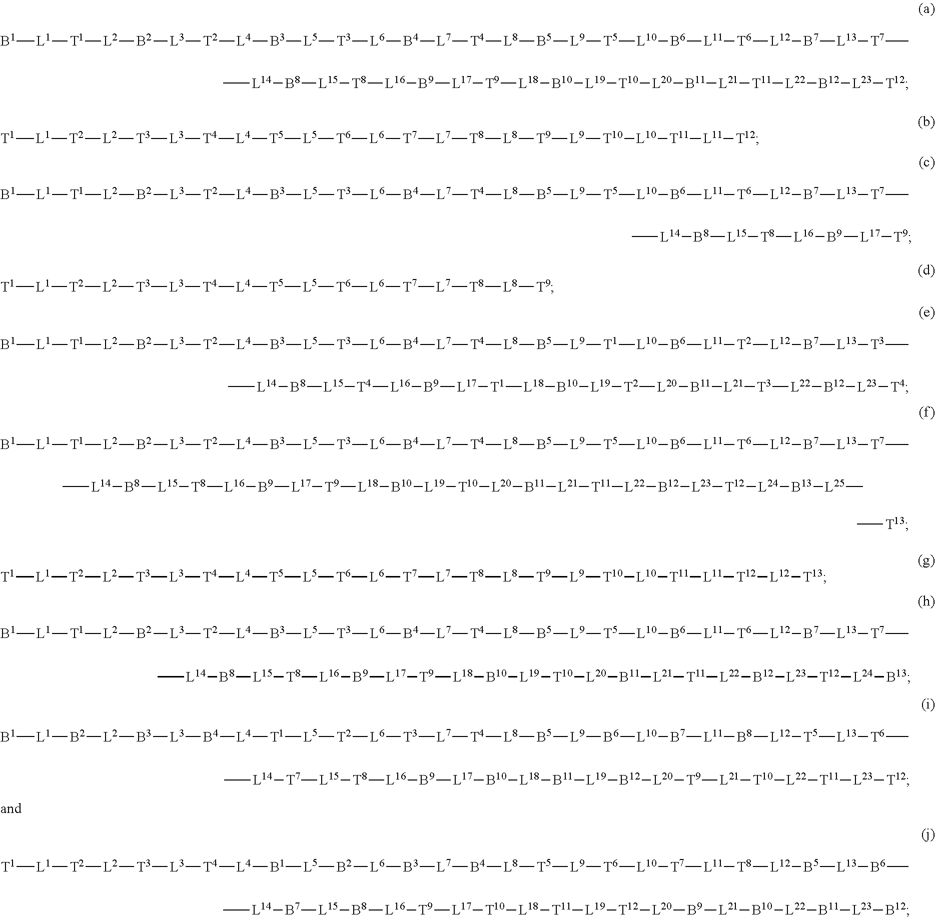
wherein:
each occurrence of B, if present, independently comprises an immunogenic fragment of a bacterial surface protein,
wherein each instance of B has a C-terminus and a N-terminus;
each occurrence of T independently comprises an immunogenic fragment of an iron receptor protein,
wherein each instance of T has a C-terminus and a N-terminus;
each occurrence of L independently comprises a polypeptide of 1-10 amino acids,
wherein each instance of L has a C-terminus and a N-terminus;
each instance of B is covalently linked to one or two independent instances of L by a covalent peptide bond between the C-terminus of the B and the N-terminus of the L and/or the N-terminus of the B and the C-terminus of the L; and
each instance of T is covalently linked to one or two independent instances of L by a covalent peptide bond between the C-terminus of the T and the N-terminus of the L and/or the N-terminus of the T and the C-terminus of the L;
or a salt or solvate thereof.

17-26. (canceled)

27. The polypeptide of claim 1, wherein the polypeptide shares at least 85%, 90%, 95%, 96%, 97%, 98%, 99%, or 100% sequence homology with SEQ ID NO:225.

28. The polypeptide of claim 1, wherein the polypeptide shares at least 85%, 90%, 95%, 96%, 97%, 98%, 99%, or 100% sequence homology with SEQ ID NO:226.

29-53. (canceled)

54. A vaccine composition comprising the polypeptide of claim 1 and at least one pharmaceutically acceptable excipient.

55-56. (canceled)

57. The vaccine composition of claim 54, further comprising an adjuvant.

58. The vaccine composition of claim 57, wherein the adjuvant comprises at least one selected from the group consisting of aluminum hydroxide (AlOH), double mutant heat-labile toxin (dmLT), CpG, and an oil-in-water emulsion adjuvant.

59. The vaccine composition of claim 58, wherein at least one of the following applies:
(a) the vaccine comprises about 10 μg to about 100 μg of the polypeptide;
(b) the vaccine comprises about 100 μg to about 6000 μg of the CpG;
(c) the vaccine comprises about 250 μg to about 750 μg of the AlOH;
(d) the vaccine comprises about 1 μg to about 25 μg of the dmLT; and
(e) the vaccine comprises about 25% (v/v) to about 75% (v/v) of the oil-in-water emulsion adjuvant.

60. A vaccine composition comprising:
(a) the polypeptide of claim 1, wherein the polypeptide shares at least 85%, 90%, 95%, 96%, 97%, 98%, 99%, or 100% sequence homology with SEQ ID NO:225 or SEQ ID NO:226, wherein the vaccine composition comprises about 10 μg to about 100 μg of the polypeptide;
(b) α-tocopherol, wherein the α-tocopherol comprises about 2.5% (v/v) of the vaccine composition;
(c) squalene, wherein the squalene comprises about 2.5% (v/v) of the vaccine composition; and
(d) polysorbate 80, wherein the polysorbate 80 comprises about 0.9% (v/v) of the vaccine composition;
wherein the polypeptide, α-tocopherol, squalene, and polysorbate are dissolved or suspended in a phosphate buffered saline (PBS) solution.

61. A vaccine composition comprising:
(a) the polypeptide of claim 1, wherein the polypeptide shares at least 85%, 90%, 95%, 96%, 97%, 98%, 99%, or 100% sequence homology with SEQ ID NO:225 or SEQ ID NO:226, wherein the vaccine composition comprises about 10 μg to about 100 μg of the polypeptide;
(b) α-tocopherol, wherein the α-tocopherol comprises about 2.5% (v/v) of the vaccine composition;
(c) squalene, wherein the squalene comprises about 2.5% (v/v) of the vaccine composition;
(d) polysorbate 80, wherein the polysorbate 80 comprises about 0.9% (v/v) of the vaccine composition; and
(e) CpG, wherein the vaccine comprises about 100 μg to about 6000 μg of the CpG;
wherein the polypeptide, α-tocopherol, squalene, polysorbate, and CpG are dissolved or suspended in a phosphate buffered saline (PBS) solution.

62. A method of treating, preventing, and/or ameliorating a bacterial infection in a subject in need thereof, the method comprising administering to the subject the polypeptide of claim 1 or a pharmaceutical composition or vaccine composition thereof.

63. The method of claim 62, wherein the bacterial infection is selected from the group consisting of a urinary tract infection (UTI), pneumonia, and sepsis, optionally wherein the sepsis is neonatal sepsis.

64-69. (canceled)

70. The method of claim 62, wherein the subject is pregnant with a fetus, optionally wherein the bacterial infection is treated, prevented, and/or ameliorated in at least one of the subject and the fetus, or a neonate thereof.

71-72. (canceled)

73. A method of generating immunity to infection by one or more pathogenic bacteria in a subject, the method comprising administering to the subject h polypeptide of claim 1 or a pharmaceutical composition or vaccine composition thereof.

74. The method of claim 73, wherein the one or more pathogenic bacteria comprises at least one selected from the group consisting of Escherichia coli, Klebsiella pneumoniae, Proteus mirabilis, Shigella dysenteriae, Salmonella enterica, Streptococcus pneumoniae, Haemophilus influenzae, Chlamydophila pneumoniae, Mycoplasma pneumoniae, Staphylococcus aureus, Moraxella catarrhalis, and Legionella pneumophila, Streptococcus pyogenes, and Pseudomonas aeruginosa, and Salmonella bongori.

75. The method of claim 73, wherein the immunity prevents a bacterial infection.

76. The method of claim 75, wherein the bacterial infection is selected from the group consisting of a urinary tract infection (UT), pneumonia, and sepsis, optionally wherein the sepsis is neonatal sepsis.

77-82. (canceled)

83. The method of claim 73, wherein the subject is pregnant with a fetus, optionally wherein the immunity to infection by one or more pathogenic bacteria is generated in at least one of the subject and the fetus, or a neonate thereof.

84-85. (canceled)
